SEQUENCE LISTING (216 sequences)

SEQ ID NO: 1 - protein, 8 aa, Artificial Sequence
  Synthetic ADAM28 cleavage site
KPAKFFRL

SEQ ID NO: 2 - protein, 8 aa, Artificial Sequence
  Synthetic ADAM28 cleavage site
DPAKFFRL

SEQ ID NO: 3 - protein, 8 aa, Artificial Sequence
  Synthetic ADAM28 cleavage site
KPMKFFRL

SEQ ID NO: 4 - protein, 8 aa, Artificial Sequence
  Synthetic ADAM28 cleavage site
LPAKFFRL

SEQ ID NO: 5 - protein, 8 aa, Artificial Sequence
  Synthetic ADAM28 cleavage site
LPMKFFRL

SEQ ID NO: 6 - protein, 8 aa, Artificial Sequence
  Synthetic ADAM28 cleavage site
KPAMFFRL

SEQ ID NO: 7 - protein, 8 aa, Artificial Sequence
  Synthetic ADAM28 cleavage site
YPAKFFRL

SEQ ID NO: 8 - protein, 8 aa, Artificial Sequence
  Synthetic ADAM28 cleavage site
KWAKFFRL

SEQ ID NO: 9 - protein, 8 aa, Artificial Sequence
  Synthetic ADAM28 cleavage site
DPMKFFRL

SEQ ID NO: 10 - protein, 8 aa, Artificial Sequence
  Synthetic ADAM28 cleavage site
DPAMFFRL

SEQ ID NO: 11 - protein, 8 aa, Artificial Sequence
  Synthetic ADAM28 cleavage site
DPMMFFRL

SEQ ID NO: 12 - protein, 8 aa, Artificial Sequence
  Synthetic ADAM28 cleavage site
KMAMFFRL

SEQ ID NO: 13 - protein, 8 aa, Artificial Sequence
  Synthetic ADAM28 cleavage site
KMAMFFIM

SEQ ID NO: 14 - protein, 8 aa, Artificial Sequence
  Synthetic ADAM28 cleavage site
KPAMFFIM

SEQ ID NO: 15 - protein, 8 aa, Artificial Sequence
  Synthetic ADAM28 cleavage site
LPAMFFRL

SEQ ID NO: 16 - protein, 8 aa, Artificial Sequence
  Synthetic ADAM28 cleavage site
LPMMFFRL

SEQ ID NO: 17 - protein, 8 aa, Artificial Sequence
  Synthetic ADAM28 cleavage site
LMAMFFRL

SEQ ID NO: 18 - protein, 8 aa, Artificial Sequence
  Synthetic ADAM28 cleavage site
LMAMFFIM

SEQ ID NO: 19 - protein, 8 aa, Artificial Sequence
  Synthetic ADAM28 cleavage site
LPAMFFIM

SEQ ID NO: 20 - protein, 8 aa, Artificial Sequence
  Synthetic ADAM28 cleavage site
LPAMFFYM

SEQ ID NO: 21 - protein, 8 aa, Artificial Sequence
  Synthetic ADAM28 cleavage site
KPMMFFRL

SEQ ID NO: 22 - protein, 8 aa, Artificial Sequence
  Synthetic ADAM28 cleavage site
KPAKFFYM

SEQ ID NO: 23 - protein, 8 aa, Artificial Sequence
  Synthetic ADAM28 cleavage site
KPAKFFIM

SEQ ID NO: 24 - protein, 8 aa, Artificial Sequence
  Synthetic ADAM28 cleavage site
IPMKFFRL

SEQ ID NO: 25 - protein, 8 aa, Artificial Sequence
  Synthetic ADAM28 cleavage site
IPAMFFRL

SEQ ID NO: 26 - protein, 8 aa, Artificial Sequence
  Synthetic ADAM28 cleavage site
IPMMFFRL

SEQ ID NO: 27 - protein, 8 aa, Artificial Sequence
  Synthetic ADAM28 cleavage site
IMAMFFRL

SEQ ID NO: 28 - protein, 8 aa, Artificial Sequence
  Synthetic ADAM28 cleavage site
IMAMFFIM

SEQ ID NO: 29 - protein, 8 aa, Artificial Sequence
  Synthetic ADAM28 cleavage site
IPAMFFIM

SEQ ID NO: 30 - protein, 8 aa, Artificial Sequence
  Synthetic ADAM28 cleavage site
IPAMFFYM

SEQ ID NO: 31 - protein, 2 aa, Artificial Sequence
  Synthetic cathepsin B cleavage site
FR

SEQ ID NO: 32 - protein, 2 aa, Artificial Sequence
  Synthetic cathepsin B cleavage site
FK

SEQ ID NO: 33 - protein, 2 aa, Artificial Sequence
  Synthetic cathepsin B cleavage site
VA

SEQ ID NO: 34 - protein, 2 aa, Artificial Sequence
  Synthetic cathepsin B cleavage site
VR

SEQ ID NO: 35 - protein, 1 aa, Artificial Sequence
  Synthetic cathepsin B cleavage site
  MOD_RES (1)..(1): citrulline
V

SEQ ID NO: 36 - protein, 8 aa, Artificial Sequence
  Synthetic cathepsin B cleavage site
HLVEALYL

SEQ ID NO: 37 - protein, 12 aa, Artificial Sequence
  Synthetic cathepsin B cleavage site
SLLKSRMVPNFN

SEQ ID NO: 38 - protein, 12 aa, Artificial Sequence
  Synthetic cathepsin B cleavage site
SLLIARRMPNFN

SEQ ID NO: 39 - protein, 4 aa, Artificial Sequence
  Synthetic cathepsin B cleavage site
KKFA

SEQ ID NO: 40 - protein, 4 aa, Artificial Sequence
  Synthetic cathepsin B cleavage site
AFKK

SEQ ID NO: 41 - protein, 3 aa, Artificial Sequence
  Synthetic cathepsin B cleavage site
QQQ

SEQ ID NO: 42 - protein, 9 aa, Artificial Sequence
  Synthetic cathepsin D cleavage site
PRSFFRLGK

SEQ ID NO: 43 - protein, 12 aa, Artificial Sequence
  Synthetic cathepsin D cleavage site
SGVVIATVIVIT

SEQ ID NO: 44 - protein, 3 aa, Artificial Sequence
  Synthetic cathepsin K cleavage site
GGP

SEQ ID NO: 45 - protein, 12 aa, Artificial Sequence
  Synthetic MMP1 cleavage site
SLGPQGIWGQFN

SEQ ID NO: 46 - protein, 7 aa, Artificial Sequence
  Synthetic MMP2 cleavage site
AIPVSLR

SEQ ID NO: 47 - protein, 12 aa, Artificial Sequence
  Synthetic MMP2 cleavage site
SLPLGLWAPNFN

SEQ ID NO: 48 - protein, 8 aa, Artificial Sequence
  Synthetic MMP2 cleavage site
HPVGLLAR

SEQ ID NO: 49 - protein, 8 aa, Artificial Sequence
  Synthetic MMP2/9 cleavage site
GPLGVRGK

SEQ ID NO: 50 - protein, 8 aa, Artificial Sequence
  Synthetic MMP2 cleavage site
GPLGLWAQ

SEQ ID NO: 51 - protein, 8 aa, Artificial Sequence
  Synthetic MMP3 cleavage site
STAVIVSA

SEQ ID NO: 52 - protein, 8 aa, Artificial Sequence
  Synthetic MMP7 cleavage site
GPLGLARK

SEQ ID NO: 53 - protein, 8 aa, Artificial Sequence
  Synthetic MMP7 cleavage site
RPLALWRS

SEQ ID NO: 54 - protein, 12 aa, Artificial Sequence
  Synthetic MMP7 cleavage site
SLRPLALWRSFN

SEQ ID NO: 55 - protein, 6 aa, Artificial Sequence
  Synthetic MMP2/9 cleavage site
GILGVP

SEQ ID NO: 56 - protein, 8 aa, Artificial Sequence
  Synthetic MMP2/9 cleavage site
GPLGIAGQ

SEQ ID NO: 57 - protein, 8 aa, Artificial Sequence
  Synthetic MMP9 cleavage site
AVRWLLTA

SEQ ID NO: 58 - protein, 7 aa, Artificial Sequence
  Synthetic MMP9 cleavage site
PLGLYAL

SEQ ID NO: 59 - protein, 9 aa, Artificial Sequence
  Synthetic MMP9 cleavage site
GPQGIAGQR

SEQ ID NO: 60 - protein, 8 aa, Artificial Sequence
  Synthetic MMP9 cleavage site
KPVSLSYR

SEQ ID NO: 61 - protein, 8 aa, Artificial Sequence
  Synthetic MMP11 cleavage site
AAATSIAM

SEQ ID NO: 62 - protein, 8 aa, Artificial Sequence
  Synthetic MMP11 cleavage site
AAGAMFLE

SEQ ID NO: 63 - protein, 12 aa, Artificial Sequence
  Synthetic MMP13 cleavage site
GPQGLAGQRGIV

SEQ ID NO: 64 - protein, 5 aa, Artificial Sequence
  Synthetic MMP14 cleavage site
PRHLR

SEQ ID NO: 65 - protein, 12 aa, Artificial Sequence
  Synthetic MMP14 cleavage site
PQGLLGAPGILG

SEQ ID NO: 66 - protein, 8 aa, Artificial Sequence
  Synthetic MMP14 cleavage site
PRSAKELR

SEQ ID NO: 67 - protein, 6 aa, Artificial Sequence
  Synthetic PSA / KLK3
HSSKLQ

SEQ ID NO: 68 - protein, 5 aa, Artificial Sequence
  Synthetic PSA / KLK3
SSKLQ

SEQ ID NO: 69 - protein, 4 aa, Artificial Sequence
  Synthetic KLK4
RQQR

SEQ ID NO: 70 - protein, 3 aa, Artificial Sequence
  Synthetic TMPRSS2
GGR

SEQ ID NO: 71 - protein, 3 aa, Artificial Sequence
  Synthetic Legumain
AAN

SEQ ID NO: 72 - protein, 3 aa, Artificial Sequence
  Synthetic ST14 (Matriptase)
QAR

SEQ ID NO: 73 - protein, 8 aa, Artificial Sequence
  Synthetic C1s cleavage site
YLGRSYKV

SEQ ID NO: 74 - protein, 6 aa, Artificial Sequence
  Synthetic C1s cleavage site
  misc_feature (6)..(6): Xaa can be any naturally occurring amino acid
MQLGRX

SEQ ID NO: 75 - protein, 8 aa, Artificial Sequence
  Synthetic MASP2 cleavage site
SLGRKIQI

SEQ ID NO: 76 - protein, 9 aa, Artificial Sequence
  Synthetic C2a and Bb cleavage site
GLARSNLDE

SEQ ID NO: 77 - protein, 8 aa, Artificial Sequence
  Synthetic uPa cleavage site
TYSRSRYL

SEQ ID NO: 78 - protein, 12 aa, Artificial Sequence
  Synthetic uPa cleavage site
KKSPGRVVGGSV

SEQ ID NO: 79 - protein, 8 aa, Artificial Sequence
  Synthetic uPa cleavage site
NSGRAVTY

SEQ ID NO: 80 - protein, 3 aa, Artificial Sequence
  Synthetic uPa cleavage site
AFK

SEQ ID NO: 81 - protein, 12 aa, Artificial Sequence
  Synthetic tissue-type plasminogen activator (tPA)
GGSGQRGRKALE

SEQ ID NO: 82 - protein, 10 aa, Artificial Sequence
  Synthetic ADAM10
PRYEAYKMGK

SEQ ID NO: 83 - protein, 5 aa, Artificial Sequence
  Synthetic ADAM12
LAQAF

SEQ ID NO: 84 - protein, 11 aa, Artificial Sequence
  Synthetic ADAM17
EHADLLAVVAK

SEQ ID NO: 85 - protein, 5 aa, Artificial Sequence
  Synthetic flexible amino acid linker (may be presented in repeating fashion)
GGGGS

SEQ ID NO: 86 - protein, 4 aa, Artificial Sequence
  Synthetic flexible amino acid linker (may be presented in repeating fashion)
GGGS

SEQ ID NO: 87 - protein, 2 aa, Artificial Sequence
  Synthetic flexible amino acid linker (may be presented in repeating fashion)
GS

SEQ ID NO: 88 - protein, 5 aa, Artificial Sequence
  Synthetic flexible amino acid linker (may be presented in repeating fashion)
GSGGS

SEQ ID NO: 89 - protein, 4 aa, Artificial Sequence
  Synthetic flexible amino acid linker (may be presented in repeating fashion)
GGSG

SEQ ID NO: 90 - protein, 5 aa, Artificial Sequence
  Synthetic flexible amino acid linker (may be presented in repeating fashion)
GGSGG

SEQ ID NO: 91 - protein, 5 aa, Artificial Sequence
  Synthetic flexible amino acid linker (may be presented in repeating fashion)
GSGSG

SEQ ID NO: 92 - protein, 5 aa, Artificial Sequence
  Synthetic flexible amino acid linker (may be presented in repeating fashion)
GSGGG

SEQ ID NO: 93 - protein, 5 aa, Artificial Sequence
  Synthetic flexible amino acid linker (may be presented in repeating fashion)
GGGSG

SEQ ID NO: 94 - protein, 5 aa, Artificial Sequence
  Synthetic flexible amino acid linker (may be presented in repeating fashion)
GSSSG

SEQ ID NO: 95 - RNA, 51 nt, Artificial Sequence
  Synthetic Anti-EGFR aptamer (tight binder with Kd=2.4 nM)
ugccgcuauaaugcacggauuuaaucgccguagaaaagcaugucaaagccg

SEQ ID NO: 96 - RNA, 51 nt, Artificial Sequence
  Synthetic Anti-EGFR aptamer
uggcgcuaaauagcacggaaauaaucgccguagaaaagcaugucaaagccg

SEQ ID NO: 97 - RNA, 51 nt, Artificial Sequence
  Synthetic Anti-EGFR aptamer
ugcuaguauaucgcacggauuuaaucgccguagaaaagcaugucaaagccg

SEQ ID NO: 98 - RNA, 51 nt, Artificial Sequence
  Synthetic Anti-EGFR aptamer
ugccgccauaucacacggauuuaaucgccguagaaaagcaugucaaagccg

SEQ ID NO: 99 - RNA, 51 nt, Artificial Sequence
  Synthetic Anti-EGFR aptamer
uuccgcuguauaacacggacuuaaucgccguaguaaagcaugucaaagccg

SEQ ID NO: 100 - RNA, 51 nt, Artificial Sequence
  Synthetic Anti-EGFR aptamer
ugucgcucuauugcacggauuuaaucgccguagaaaagcaugucaaagccg

SEQ ID NO: 101 - RNA, 51 nt, Artificial Sequence
  Synthetic Anti-EGFR aptamer
ugcugcuuuaucccacauauuuuuuccccucauaacaauauuucucccccc

SEQ ID NO: 102 - RNA, 51 nt, Artificial Sequence
  Synthetic Anti-EGFR aptamer
  misc_feature (4)..(4): n is a, c, g, or u
  misc_feature (15)..(15): n is a, c, g, or u
  misc_feature (45)..(45): n is a, c, g, or u
  misc_feature (47)..(47): n is a, c, g, or u
ugcngcuauaucgcncguauuuaaucgccguagaaaagcaugucnangccg

SEQ ID NO: 103 - RNA, 51 nt, Artificial Sequence
  Synthetic Anti-EGFR aptamer
ugcaaagaaaacgcacguauuuaaucgccguaguaaagcaugucaaagccg

SEQ ID NO: 104 - RNA, 53 nt, Artificial Sequence
  Synthetic Anti-EGFR aptamer
ugcaucacuaucgaaccuauuuaauccaccaaaauaauugcaaguccauacuc

SEQ ID NO: 105 - RNA, 51 nt, Artificial Sequence
  Synthetic Anti-EGFR aptamer
  misc_feature (5)..(6): n is a, c, g, or u
  misc_feature (17)..(17): n is a, c, g, or u
  misc_feature (48)..(48): n is a, c, g, or u
ugccnnaauaacacacnuauauaaucgccguacaaaaucaugucaaanccg

SEQ ID NO: 106 - RNA, 51 nt, Artificial Sequence
  Synthetic Anti-EGFR aptamer
ugcagcuguauugcacguauuuaaucgccguagaaaagcaugucaaagccg

SEQ ID NO: 107 - RNA, 50 nt, Artificial Sequence
  Synthetic Anti-EGFR aptamer
uuccgauaaucccgcguacuaaaucaccauagucaacaauuuccaaccuc

SEQ ID NO: 108 - RNA, 50 nt, Artificial Sequence
  Synthetic Anti-EGFR aptamer
uccacuauaucacacguauuuaaucgccguagaaaagcaugucaaagccg

SEQ ID NO: 109 - RNA, 51 nt, Artificial Sequence
  Synthetic Anti-EGFR aptamer
ucccucaaccucgcuacuauuuaaucgccguagaaaagcaugucaaagccu

SEQ ID NO: 110 - RNA, 51 nt, Artificial Sequence
  Synthetic Anti-EGFR aptamer
ugccgcuauaucacacgaauuuaaucgccguagaaaagcaugucaaagccg

SEQ ID NO: 111 - RNA, 51 nt, Artificial Sequence
  Synthetic Anti-EGFR aptamer
agccccuagaacacacggauuuaaucgccguagaaaagcaugucaaagccg

SEQ ID NO: 112 - RNA, 51 nt, Artificial Sequence
  Synthetic Anti-EGFR aptamer
ugccaauauauaacacggaauuaaucgccguagaaaagcaugucaaagccg

SEQ ID NO: 113 - RNA, 51 nt, Artificial Sequence
  Synthetic Anti-EGFR aptamer
ugccgcuauagcgcacggauuuaaucgccguagaaaagcaugucaaagccg

SEQ ID NO: 114 - RNA, 50 nt, Artificial Sequence
  Synthetic Anti-EGFR aptamer
ugcagauauaugucacucauuaauccccguauaaaaacauaacuaagcuc

SEQ ID NO: 115 - RNA, 51 nt, Artificial Sequence
  Synthetic Anti-EGFR aptamer
uguagcuguauugcacacauuaaaucgccguaguaaagcaugucaaagccg

SEQ ID NO: 116 - RNA, 50 nt, Artificial Sequence
  Synthetic Anti-EGFR aptamer
uaccaauauaucgccacacauaaucgccguagaaaagcaugucaaagccg

SEQ ID NO: 117 - RNA, 51 nt, Artificial Sequence
  Synthetic Anti-EGFR aptamer
ugccgcuaugcccacggaauuuaaucgccguagaaaaacaugucaaagucg

SEQ ID NO: 118 - RNA, 51 nt, Artificial Sequence
  Synthetic Anti-EGFR aptamer
ugccgcuauuuagcacggauuaaaucgccguagaaaagcaugucaaagccg

SEQ ID NO: 119 - RNA, 51 nt, Artificial Sequence
  Synthetic Anti-EGFR aptamer
  misc_feature (45)..(45): n is a, c, g, or u
ugccgcuauuuagcacggauuaaaucgccguagaaaagcaugucnaagccg

SEQ ID NO: 120 - RNA, 51 nt, Artificial Sequence
  Synthetic Anti-EGFR aptamer
  misc_feature (41)..(41): n is a, c, g, or u
uguaguaauaugacacggauuuaaucgccguagaaaagcangucaaagccu

SEQ ID NO: 121 - RNA, 51 nt, Artificial Sequence
  Synthetic Anti-EGFR aptamer
ugucgccauuacgcacggauuuaaucgccguagaaaagcaugucaaagccg

SEQ ID NO: 122 - RNA, 51 nt, Artificial Sequence
  Synthetic Anti-EGFR aptamer
ugcccccaaacuacacaaauuuaaucgccguauaaaagcaugucaaagccg

SEQ ID NO: 123 - RNA, 49 nt, Artificial Sequence
  Synthetic Anti-EGFR aptamer
ugcacuaucucacacguacuaaucgccguauaaaagcaugucaaagccg

SEQ ID NO: 124 - RNA, 51 nt, Artificial Sequence
  Synthetic Anti-EGFR aptamer
ugucgcaauaauacacuaauuuaaucgccguagaaaagcaugucaaagccg

SEQ ID NO: 125 - RNA, 49 nt, Artificial Sequence
  Synthetic Anti-EGFR aptamer
ugcaacaauauagcacguauuuaaucgccguaguaaagcaugucaaagg

SEQ ID NO: 126 - RNA, 51 nt, Artificial Sequence
  Synthetic Anti-EGFR aptamer
cuaccacaaaucccacauauuuaaucucccaaucaaaucuuguccauuccc

SEQ ID NO: 127 - RNA, 51 nt, Artificial Sequence
  Synthetic Anti-EGFR aptamer
ugcccuaaacucacacggauauaaucgccguagaaaagcaugucaaagccg

SEQ ID NO: 128 - RNA, 51 nt, Artificial Sequence
  Synthetic Anti-EGFR aptamer
uugucguaugucacacguauuaaaucgccguauaaaagcaugucaaagccg

SEQ ID NO: 129 - RNA, 51 nt, Artificial Sequence
  Synthetic Anti-EGFR aptamer
uuccgcuauaacacacggagaaaaucgccguaguaaagcaugucaaagccg

SEQ ID NO: 130 - RNA, 51 nt, Artificial Sequence
  Synthetic Anti-EGFR aptamer
ugccgauauaacgcacggauauaaucgccguagaaaagcaugucaaagccg

SEQ ID NO: 131 - RNA, 51 nt, Artificial Sequence
  Synthetic Anti-EGFR aptamer
ugccauuauacagcacggauuuaaucgccguagaaaagcaugucaaagccg

SEQ ID NO: 132 - RNA, 50 nt, Artificial Sequence
  Synthetic Anti-EGFR aptamer
uccagaaauaugcacacauuuaaucgccguagaaaagcaugucaaagccg

SEQ ID NO: 133 - RNA, 50 nt, Artificial Sequence
  Synthetic Anti-EGFR aptamer
uccgcuaaacaacacggauacaaucgccguagaaaagcauguccaagccg

SEQ ID NO: 134 - RNA, 49 nt, Artificial Sequence
  Synthetic Anti-EGFR aptamer
  misc_feature (46)..(48): n is a, c, g, or u
ugcacuaucucacacguacuaaucgccguauaaaagcaugucaaannng

SEQ ID NO: 135 - RNA, 50 nt, Artificial Sequence
  Synthetic Anti-EGFR aptamer
  misc_feature (3)..(3): n is a, c, g, or u
  misc_feature (6)..(6): n is a, c, g, or u
  misc_feature (8)..(10): n is a, c, g, or u
  misc_feature (21)..(21): n is a, c, g, or u
  misc_feature (45)..(45): n is a, c, g, or u
aungcnannnuacacguauunaaucgccguagaaaagcaugucanagccg

SEQ ID NO: 136 - RNA, 51 nt, Artificial Sequence
  Synthetic Anti-EGFR aptamer
ugcugcuauauugcaauuuuuuaaacuaaguagaaaaccauguacaagucg

SEQ ID NO: 137 - RNA, 51 nt, Artificial Sequence
  Synthetic Anti-EGFR aptamer
ugucgccauauugcacggauuuaaucgccguagaaaagcauguccaagccg

SEQ ID NO: 138 - RNA, 52 nt, Artificial Sequence
  Synthetic Anti-EGFR aptamer
ugccguuauaacccacggaauuuaaccuccguagaaaagcaugucaaagccg

SEQ ID NO: 139 - RNA, 52 nt, Artificial Sequence
  Synthetic Anti-EGFR aptamer
  misc_feature (40)..(40): n is a, c, g, or u
ugugaauauauaucacggauuuaaucgccguauaaaagcnaugucaaagccg

SEQ ID NO: 140 - RNA, 51 nt, Artificial Sequence
  Synthetic Anti-EGFR aptamer
  misc_feature (10)..(11): n is a, c, g, or u
  misc_feature (13)..(13): n is a, c, g, or u
ugccgauaunnancacggauuuaaucgccguagaaaagcauguccaagccg

SEQ ID NO: 141 - RNA, 50 nt, Artificial Sequence
  Synthetic Anti-EGFR aptamer
ugucacuaaauugcacguauauaaucgccguaguaagcaugucaaagccg

SEQ ID NO: 142 - RNA, 51 nt, Artificial Sequence
  Synthetic Anti-EGFR aptamer
ugcaaccauaaagcacguaauaaaucgccguauauaagcaugucaaagccg

SEQ ID NO: 143 - RNA, 50 nt, Artificial Sequence
  Synthetic Anti-EGFR aptamer
ugccgcuauauagcacguauuaaucgccguaguaaagcaugucaaagccg

SEQ ID NO: 144 - RNA, 52 nt, Artificial Sequence
  Synthetic Anti-EGFR aptamer
ugccgcuauagcacacggaauuuaaucgccguaguaaagcaugucaaagccg

SEQ ID NO: 145 - RNA, 51 nt, Artificial Sequence
  Synthetic Anti-EGFR aptamer
  misc_feature (15)..(15): n is a, c, g, or u
  misc_feature (45)..(45): n is a, c, g, or u
ugcagguauauaacncggauuuaaucgccguagaaaagcaugucnaagccg

SEQ ID NO: 146 - RNA, 51 nt, Artificial Sequence
  Synthetic Anti-EGFR aptamer
ugcuccuauaacacacggauuuaaucgccguagaaaagcauguccaagccg

SEQ ID NO: 147 - RNA, 51 nt, Artificial Sequence
  Synthetic Anti-EGFR aptamer
ugcccguaauugcacggauuuaaucgccguagaaaagcauguccaagccgg

SEQ ID NO: 148 - RNA, 51 nt, Artificial Sequence
  Synthetic Anti-EGFR aptamer
  misc_feature (12)..(12): n is a, c, g, or u
  misc_feature (44)..(44): n is a, c, g, or u
acucccuauaungcaacuacauaaucgccguaaauaagcauguncaagccg

SEQ ID NO: 149 - RNA, 54 nt, Artificial Sequence
  Synthetic Anti-EGFR aptamer
ugaagcuagaucacacuaaauuaaucgccguagaaaagcaugucaaaaaagccg

SEQ ID NO: 150 - RNA, 52 nt, Artificial Sequence
  Synthetic Anti-EGFR aptamer
ugacucuuuaucccccguacauuauucaccgaaccaaagcauuaccaucccc

SEQ ID NO: 151 - RNA, 51 nt, Artificial Sequence
  Synthetic Anti-EGFR aptamer
ugacgcccuaacacacguauauaaucgccguagaaaagcaugucaaagccg

SEQ ID NO: 152 - RNA, 51 nt, Artificial Sequence
  Synthetic Anti-EGFR aptamer
ugucgcaaaauagcacguauuuaaucgccguagaaaagcauguccaagccg

SEQ ID NO: 153 - RNA, 51 nt, Artificial Sequence
  Synthetic Anti-EGFR aptamer
ugaguguauaauucacguauuuaaucgccguagaaaagcaugucaaagccg

SEQ ID NO: 154 - RNA, 51 nt, Artificial Sequence
  Synthetic Anti-EGFR aptamer
ugcuacuauaucguagguaacuaaucgcccuacaaacucacucuaaaaccg

SEQ ID NO: 155 - RNA, 53 nt, Artificial Sequence
  Synthetic Anti-EGFR aptamer
uuacgcuauaucacacggaauuuuaaucgccguagaaaagcauguccaagccg

SEQ ID NO: 156 - RNA, 51 nt, Artificial Sequence
  Synthetic Anti-EGFR aptamer
cccaucuguacuacaggaauuuaaucgccguagaaaagcauguccaagccg

SEQ ID NO: 157 - RNA, 51 nt, Artificial Sequence
  Synthetic Anti-EGFR aptamer
ugcccauaaauagcacggauuuaaucgccguagaaaagcauguccaagccg

SEQ ID NO: 158 - RNA, 51 nt, Artificial Sequence
  Synthetic Anti-EGFR aptamer
ugccgcaauaacauacacauauaaucgccguagaaaagcaugucaaagccg

SEQ ID NO: 159 - RNA, 51 nt, Artificial Sequence
  Synthetic Anti-EGFR aptamer
ugcaacuauaucgcacguauguaaucgccguagaaaaagcaugucaaagcc

SEQ ID NO: 160 - RNA, 51 nt, Artificial Sequence
  Synthetic Anti-EGFR aptamer
uuccgcuauauagcacggaauuaaucgccguagaaaagcauguccaagccg

SEQ ID NO: 161 - RNA, 51 nt, Artificial Sequence
  Synthetic Anti-EGFR aptamer
uuccgcuaagucacacgaaauuaaucgccguagaaaagcauguccaagccg

SEQ ID NO: 162 - RNA, 51 nt, Artificial Sequence
  Synthetic Anti-EGFR aptamer
uguagcaauaucacacguaauuaaucgccguauauaagcaugucaaagccg

SEQ ID NO: 163 - RNA, 51 nt, Artificial Sequence
  Synthetic Anti-EGFR aptamer
ugccguuauauaucacggauuuaaucgccguagaaaagcauguccaagccg

SEQ ID NO: 164 - RNA, 49 nt, Artificial Sequence
  Synthetic Anti-EGFR aptamer
uaacacauauaucaaguaacuuaucuccuuaguaaccaucuccaagccg

SEQ ID NO: 165 - protein, 731 aa, Artificial Sequence
  Synthetic Construct 6248 single chain; scFv anti-EPCAM
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGAIPVSLRGSGGSGGADDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 728 aa, Artificial Sequence
  Synthetic Construct 6249 single chain; scFv anti-EPCAM
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGEGTSTGSGAIPVSLRGSGGSGGADDVQLVQSGAEVKKPGASVKVSCKASGYTFTGYYMHWVRQAPGQGLEWMGWINPNSGGTNYAQKFQGRVTITRDTSASTAYMELSSLRSEDTAVYYCARDFLSGYLDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 167 - protein, 600 aa, Artificial Sequence
  Synthetic Construct 9329 Glypican3 VHH-CD3E(VH-MMP2-VL)
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGAIPVSLRGSGGSGGADDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 728 aa, Artificial Sequence
  Synthetic Construct 9330
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKRGGGGSGGGGSGGGGSQVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGAIPVSLRGSGGSGGADDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 632 aa, Artificial Sequence
  Synthetic Construct 9334 EGFR VHH-CD3E(VH- MMP2-VL)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGAIPVSLRGSGGSGGADDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - protein, 632 aa, Artificial Sequence
  Synthetic Construct 9335 EGFR VHH-CD3E(VL- MMP2-VH)
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGGSGGGGSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGEGTSTGSGAIPVSLRGSGGSGGADDVQLVQSGAEVKKPGASVKVSCKASGYTFTGYYMHWVRQAPGQGLEWMGWINPNSGGTNYAQKFQGRVTITRDTSASTAYMELSSLRSEDTAVYYCARDFLSGYLDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 240 aa, Artificial Sequence
  Synthetic MP058, anti-Epcam kappa light chain
METDTLLLWVLLLWVPGSTGELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 512 aa, Artificial Sequence
  Synthetic MP111, anti-EGFR H-TEAC heavy chain
METDTLLLWVLLLWVPGSTGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGGGSGPLGVRGKAGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 173 - protein, 513 aa, Artificial Sequence
  Synthetic MP112, anti-EGFR L-TEAC heavy chain
METDTLLLWVLLLWVPGSTGEVQLVESGGGLVQPGGSLRLSCAASGFTFSSYAMNWVRQAPGKGLEWVARISGSGGSTYYADSVKDRFTISRDDSKNSLYLQMNSLKTEDTAVYYCVRGKGNTHKPYGYVRYFDVWGQGTLVTVSSGGGGSGPLGVRGKAGGGGSQAVVTQEPSLTVSPGGTVTLTCRSSTGAVTASNYANWVQQKPGQAPRGLIGGTNKRAPWTPARFSGSLLGGKAALTITGAQAEDEADYYCALWYSNLWVFGGGTKLTVLGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 174 - protein, 233 aa, Artificial Sequence
  Synthetic MP113, anti-EGFR kappa light chain
METDTLLLWVLLLWVPGSTGDIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 737 aa, Artificial Sequence
  Synthetic MP251, anti-Epcam H-TEAC SG3SG4S- linker Fc fusion heavy chain
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGGGSGPLGVRGKAGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 176 - protein, 742 aa, Artificial Sequence
  Synthetic MP252, anti-Epcam H-TEAC SG3SG4AG4S linker Fc fusion heavy chain
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGAGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGGGSGPLGVRGKAGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 177 - protein, 734 aa, Artificial Sequence
  Synthetic MP253, anti-Epcam H-TEAC SG3SG4AG4S linker Fc fusion heavy chain
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGAGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGPLGVRGKAGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 178 - protein, 732 aa, Artificial Sequence
  Synthetic MP254, anti-Epcam H-TEAC SG3SG4AG4S linker Fc fusion heavy chain
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGAGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGPLGVRGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 179 - protein, 748 aa, Artificial Sequence
  Synthetic MP268, anti-EGFR H-TEAC SG3SG4S- linker Fc fusion heavy chain
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGGGSGPLGVRGKAGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 180 - protein, 749 aa, Artificial Sequence
  Synthetic MP269, anti-EGFR L-TEAC SG3SG4S- linker Fc Fusion heavy chain
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGSEVQLVESGGGLVQPGGSLRLSCAASGFTFSSYAMNWVRQAPGKGLEWVARISGSGGSTYYADSVKDRFTISRDDSKNSLYLQMNSLKTEDTAVYYCVRGKGNTHKPYGYVRYFDVWGQGTLVTVSSGGGGSGPLGVRGKAGGGGSQAVVTQEPSLTVSPGGTVTLTCRSSTGAVTASNYANWVQQKPGQAPRGLIGGTNKRAPWTPARFSGSLLGGKAALTITGAQAEDEADYYCALWYSNLWVFGGGTKLTVLGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 181 - protein, 19 aa, Artificial Sequence
  Synthetic 19-mer protease linker
GGGGSGPLGVRGKAGGGGS

SEQ ID NO: 182 - protein, 11 aa, Artificial Sequence
  Synthetic 11-mer protease linker
GGPLGVRGKAG

SEQ ID NO: 183 - protein, 9 aa, Artificial Sequence
  Synthetic 9-mer protease linker
GGPLGVRGG

SEQ ID NO: 184 - protein, 15 aa, Artificial Sequence
  Synthetic SG3SG4AG4S linker
SGGGSGGGGAGGGGS

SEQ ID NO: 185 - protein, 10 aa, Artificial Sequence
  Synthetic SG3SG4S linker
SGGGSGGGGS

SEQ ID NO: 186 - protein, 737 aa, Artificial Sequence
  Synthetic Anti-EpCAM-CD3 VH ATTAC/TEAC component with half-life extension (Anti-EpCAM scFv with 3xG4S linker between VH-VL 1xG4S connector anti-CD3e VH (20G6) - MMP2 cleavage sequence Ig VL domain human IgG1 Fc)
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSGEGTSTGSGAIPVSLRGSGGSGGADDIVMTQTPLSLSVTPGQPASISCKSSQSIVHSSGNTYLSWYLQKPGQSPQLLIYKVSNRFSGVPDRFSGSGSGTDFTLKISRVEAEDVGVYYCGQGSHVGPTFGSGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 756 aa, Artificial Sequence
  Synthetic Anti-EpCAM-CD3 VH ATTAC/TEAC component with half-life extension moiety Anti-EpCAM scFv (with 3xG4S linker between VH-VL) 1xG4S connector 20G6 VH MMP2 cleavage tag inert binding partner (VL domain) 2xG4S linker IgG4 CH domains His
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSGEGTSTGSGAIPVSLRGSGGSGGADDIVMTQTPLSLSVTPGQPASISCKSSQSIVHSSGNTYLSWYLQKPGQSPQLLIYKVSNRFSGVPDRFSGSGSGTDFTLKISRVEAEDVGVYYCGQGSHVGPTFGSGTKVEIKGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSHHHHHH

SEQ ID NO: 188 - protein, 738 aa, Artificial Sequence
  Synthetic Anti-EpCAM-CD3 VL ATTAC/TEAC component with half-life extension moiety (Anti-EpCAM scFv with 3xG4S linker between VH-VL 1xG4S connector anti-CD3e VL (20G6) - MMP2 cleavage sequence Ig VH domain human IgG1 Fc)
EVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSGGGGSGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 754 aa, Artificial Sequence
  Synthetic Anti-EpCAM-CD3 VL ATTAC/TEAC component with half-life extension moiety Anti-EpCAM scFv (with 3xG4S linker between VL-VH) 1xG4S connector 20G6 VL MMP2 cleavage tag inert binding partner (VH domain) 2xG4S linker IgG4 CH domains EPEA
EVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSGGGGSGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGEPEA

SEQ ID NO: 190 - protein, 504 aa, Artificial Sequence
  Synthetic Anti-EpCAM-CD3 scFv (20G6) BiTE construct (anti-EpCAM scFv with 3xG4S linker between VH and VL 1xG4S connector anti-CD3 scFv with linker between VH and VL )
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGGSGGSGGADQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 191 - protein, 622 aa, Artificial Sequence
  Synthetic Anti-CD8-CD3 VL ATTAC component with half-life extension moiety (Anti-CD8 VHH 1xG4S connector anti-CD3e VL (20G6) - MMP2 cleavage sequence Ig VH domain human IgG1 Fc) (CD8 targeting VHH domain based upon WO_2017_134306 SEQ ID NO
QVQLQESGGGLVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLRTGQGTQVTVSSAAAYPYDVPDYGSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 640 aa, Artificial Sequence
  Synthetic Anti-CD8-CD3 VL ATTAC component with half-life extension moiety
QVQLQESGGGLVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLRTGQGTQVTVSSAAAYPYDVPDYGSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGHHHHHH

SEQ ID NO: 193 - protein, 730 aa, Artificial Sequence
  Synthetic Anti-CD8-CD3 VL ATTAC component with half-life extension moiety
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSSGGGGSGGGGSGGGGSDVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 194 - protein, 748 aa, Artificial Sequence
  Synthetic Anti-CD8-CD3 VL ATTAC component with half-life extension moiety
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSSGGGGSGGGGSGGGGSDVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGHHHHHH

SEQ ID NO: 195 - protein, 739 aa, Artificial Sequence
  Synthetic Anti-CD4-CD3 VL ATTAC component with half-life extension moiety
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSGGGGSGGGGSGGGGSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 196 - protein, 757 aa, Artificial Sequence
  Synthetic Anti-CD4-CD3 VL ATTAC component with half-life extension moiety
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSGGGGSGGGGSGGGGSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGHHHHHH

SEQ ID NO: 197 - protein, 677 aa, Artificial Sequence
  Synthetic Anti-CD8-CD3 VL ATTAC component (Anti-CD8 VHH 6xG4S connector anti-CD3e VL (20G6) - Enterokinase cleavage sequence Ig VH domain human IgG1 Fc) (CD8 targeting VHH domain based upon WO_2017_134306 SEQ ID NO 21)
QVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSAAAYPYDVPDYGSGGGGSGGGGSGGGGSGGGGSGGGGSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGGGGGSGGGGSDDDDKGGGGSGGGGSGSGGSGGADQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 198 - protein, 694 aa, Artificial Sequence
  Synthetic Anti-CD8-CD3 VL ATTAC component (Anti-CD8 VHH 6xG4S connector anti-CD3e VL (20G6) - Enterokinase cleavage sequence Ig VH domain 2xG4S connector IgG4 CH domains His tag) (CD8 targeting VHH domain based upon WO_2017_134306 SEQ ID NO 21)
QVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSAAAYPYDVPDYGSGGGGSGGGGSGGGGSGGGGSGGGGSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGGGGGSGGGGSDDDDKGGGGSGGGGSGSGGSGGADQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSHHHHHH

SEQ ID NO: 199 - protein, 745 aa, Artificial Sequence
  Synthetic Anti-CD33 TEAC component with half- life extension moiety CD33 20G6-VH Anti-CD33 scFv (with 3xG4S linker between VH-VL) 1xG4S connector 20G6 VH MMP2 cleavage tag inert binding partner (VL domain) 2xG4S linker IgG4 CH domains His
QIVLTQSPAIMSASPGEKVTITCSASSSISYMHWFQQKPGTSPKLWIYTTSNLASGVPARFSGSGSGTSYSLTISRMEAEDAATYYCHQRSTYPLTFGSGTKLELKGGGGSGGGGSSGGGSQVQLQQSGAELAKPGASVKMSCKASGYTFTSYRMHWVKQRPGQGLEWIGYINPSTGYTEYNQKFKDKATLTADKSSSTAYMQLSSLTFEDSAVYYCARGGGVFDYWGQGTTLTVSSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSGEGTSTGSGAIPVSLRGSGGSGGADDIVMTQTPLSLSVTPGQPASISCKSSQSIVHSSGNTYLSWYLQKPGQSPQLLIYKVSNRFSGVPDRFSGSGSGTDFTLKISRVEAEDVGVYYCGQGSHVGPTFGSGTKVEIKGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSHHHHHH

SEQ ID NO: 200 - protein, 621 aa, Artificial Sequence
  Synthetic Anti-CD123 TEAC component with half- life extension moiety CD123 20G6-VL  Anti-CD123 VHH domain 1xG4S connector 20G6 VL MMP2 cleavage tag inert binding partner (VH domain) 2xG4S linker IgG4 CH domains EPEA tag - Stop
EVQLVKSGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGEPEA

SEQ ID NO: 201 - protein, 754 aa, Artificial Sequence; identical to SEQ ID NO: 189
  Synthetic Anti-EpCAM VL TEAC component with half-life extension moiety

SEQ ID NO: 202 - protein, 756 aa, Artificial Sequence; identical to SEQ ID NO: 187
  Synthetic Anti-EpCAM VH TEAC component with half-life extension moiety

SEQ ID NO: 203 - protein, 717 aa, Artificial Sequence
  Synthetic RO258, anti-EpCAM H-TEAC SG3SG4S- linker Fc fusion
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGGGSGPLGVRGKAGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 204 - protein, 722 aa, Artificial Sequence
  Synthetic RO259, anti-EpCAM H-TEAC SG3SG4AG4S- linker Fc fusion
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGAGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGGGSGPLGVRGKAGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 205 - protein, 714 aa, Artificial Sequence
  Synthetic RO260, anti-EpCAM H-TEAC SG3SG4AG4S- linker Fc fusion with 11 residue protease linker
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGAGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGPLGVRGKAGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 206 - protein, 712 aa, Artificial Sequence
  Synthetic RO261, anti-EpCAM H-TEAC SG3SG4AG4S- linker Fc fusion with 9 residue protease linker
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGAGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGPLGVRGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 207 - protein, 728 aa, Artificial Sequence
  Synthetic RO268, anti-EGFR H-TEAC SG3SG4S- linker Fc fusion
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGGGSGPLGVRGKAGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 208 - protein, 729 aa, Artificial Sequence
  Synthetic RO269, anti-EGFR L-TEAC SG3SG4S- linker Fc Fusion
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGSGGGGSEVQLVESGGGLVQPGGSLRLSCAASGFTFSSYAMNWVRQAPGKGLEWVARISGSGGSTYYADSVKDRFTISRDDSKNSLYLQMNSLKTEDTAVYYCVRGKGNTHKPYGYVRYFDVWGQGTLVTVSSGGGGSGPLGVRGKAGGGGSQAVVTQEPSLTVSPGGTVTLTCRSSTGAVTASNYANWVQQKPGQAPRGLIGGTNKRAPWTPARFSGSLLGGKAALTITGAQAEDEADYYCALWYSNLWVFGGGTKLTVLGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 209 - protein, 488 aa, Artificial Sequence
  Synthetic RO132, anti-EGFR/anti-CD3 bispecific
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGSGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 210 - protein, 1102 aa, Artificial Sequence
  Synthetic Human serum albumin fusion of SP34 VH TEAC, MMP2 cleavage site, anti-EGFR Fab heavy chain
METDTLLLWVLLLWVPGSTGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLAVFGGGTKLTVLGGGGSGPLGVRGKAGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 211 - protein, 1103 aa, Artificial Sequence
  Synthetic Human serum albumin fusion of SP34 VL TEAC, MMP2 cleavage site, anti-EGFR Fab heavy chain
METDTLLLWVLLLWVPGSTGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSEVQLVESGGGLVQPGGSLRLSCAASGFTFSSYAMNWVRQAPGKGLEWVARISGSGGSTYYADSVKDRFTISRDDSKNSLYLQMNSLKTEDTAVYYCVRGKGNTHKPYGYVRYFDVWGQGTLVTVSSGGGGSGPLGVRGKAGGGGSQAVVTQEPSLTVSPGGTVTLTCRSSTGAVTASNYANWVQQKPGQAPRGLIGGTNKRAPWTPARFSGSLLGGKAALTITGAQAEDEADYYCALWYSNLWVFGGGTKLTVLGGGGSGGGGSQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 212 - protein, 692 aa, Artificial Sequence
  Synthetic Anti-CD8-CD3 VL ATTAC component (Anti-CD8 VHH 6xG4S connector anti-CD3e VL (20G6) - Enterokinase cleavage sequence Ig VH domain  2xG4S connector IgG4 CH domains EPEA tag) (CD8 targeting VHH domain based upon WO_2017_134306 SEQ ID NO 21)
QVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSAAAYPYDVPDYGSGGGGSGGGGSGGGGSGGGGSGGGGSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGGGGGSGGGGSDDDDKGGGGSGGGGSGSGGSGGADQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGSGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSEPEA

SEQ ID NO: 213 - protein, 8 aa, Artificial Sequence
  Synthetic peptide
YLGRSYKV

SEQ ID NO: 214 - protein, 6 aa, Artificial Sequence
  Synthetic peptide
  misc_feature (6)..(6): Xaa can be any naturally occurring amino acid
MQLGRX

SEQ ID NO: 215 - protein, 8 aa, Artificial Sequence
  Synthetic peptide
SLGRKIQI

SEQ ID NO: 216 - protein, 9 aa, Artificial Sequence
  Synthetic peptide
GLARSNLDE

(19) United States
(12) Patent Application Publication
(10) Pub. No.: US 2022/0323600 A1
(43) Pub. Date: Oct. 13, 2022

(54) TEAC AND ATTAC IMMUNOONCOLOGY COMPOSITIONS AND METHODS

(71) Applicants: The General Hospital Corporation, Boston, MA (US); Revitope Limited, Potters Bar Hertfordshire (GB)

(72) Inventors: Mark Cobbold, Winchester, MA (US); Martin Preyer, Somerville, MA (US); Allison Colthart, Cambridge, MA (US)

(21) Appl. No.: 17/608,101

(22) PCT Filed: Apr. 23, 2020
(86) PCT No.: PCT/US2020/029548

§ 371 Date: Nov. 1, 2021

Related U.S. Application Data
(60) Provisional application No. 62/841,960, filed on May 2, 2019.

(51) Int. Cl. A61K 47/68; A61K 47/64; A61K 47/60; A61P 35/00

(52) U.S. Cl. CPC A61K 47/6851; A61K 47/643; A61K 47/60; A61K 47/6849; A61K 47/6807; A61K 47/6813; A61K 47/644; A61K 47/6803; A61P 35/00

(57) ABSTRACT

The present disclosure provides targeted T-cell engaging agents (TEAC) and antibody tumor-targeting assembly complexes (ATTAC) for targeting to cancer. The TEAC or ATTAC described herein may have, for example, longer half-life or comprise multiple components in a single agent.

### CLAIM OF PRIORITY

This application claims the benefit of U.S. Provisional Application No. 62/841,960, filed May 2, 2019. The entire contents of the foregoing are hereby incorporated herein by reference.

### SEQUENCE LISTING

The present application is filed with a Sequence Listing in electronic format. The Sequence Listing is provided as a file entitled “2020-04-09_01131-0026-00PCT_ST25.txt” created on Apr. 9, 2020, which is 298,657 bytes in size. The information in the electronic format of the sequence listing is incorporated herein by reference in its entirety.

### DESCRIPTION

### Field

This application relates to targeted T-cell engaging agents (TEACs) and antibody tumor-targeting assembly complexes (ATTACs) for treating cancer.

The TEAC or ATTAC described herein may have, for example, longer half-life or comprise multiple components in a single agent.

### Background

Cancer creates significant loss of life, suffering, and economic impact. Immunotherapeutic strategies for targeting cancer have been an active area of translational clinical research.

A variety of other approaches have been explored for immunotherapy, but many of these prior approaches lack sufficient specificity to particular cancer cells. For example, demibodies have been designed each having an scFv portion binding to different antigens on a target cell, an Fc domain allowing pairing to a complementary demibody, and a binding partner capable of forming an association to another binding partner on a complementary demibody. WO 2007/062466. These demibodies, however, are not necessarily specific to cancer cells and could bind and have activity on other cells expressing the same antigens. See also WO 2013/104804, which provides a first polypeptide with a targeting moiety binding to a first antigen and a first fragment of a functional domain, along with a second polypeptide with a targeting moiety binding to a second antigen and a second fragment of a functional domain that is complementary to the first fragment of the functional domain. Likewise, this approach is not necessarily specific to cancer cells and could bind and have activity on other cells expressing the same antigens.

Bispecific T-cell Engaging Antibodies (BiTEs) have been proposed by others; however, these constructs are often not sufficiently specific to the tumor environment. Further, current bi-specific antibodies activate T cells via CD3. Although not widely discussed, these agents are incredibly potent and are given at extremely low doses compared with whole antibody therapies. This will be partly due to the fact that these reagents can theoretically activate every T cell by binding to CD3. When someone has a viral infection, around 1-10% of their T cells are activated and they feel lethargic and ill because of the immune response. When more T cells are activated, this can lead to larger problems including cytokine release syndrome (CRS) and death in rare cases. CRS can be triggered by release of cytokines from cells targeted by biologics, as well as by cytokine release from recruited immune effector cells. Therefore, there is a need to limit the total number of T cells that are activated using these systems.

This application describes a TEAC or ATTAC comprising a half-life extending moiety. Agents or components with a half-life extending moiety may simplify dosing regimens and allow lower doses of agents to be administered to achieve the same efficacy as agents without a half-life extending moiety, or may reduce dosing frequency required to achieve efficacy. Further, a single-agent TEAC or ATTAC may facilitate methods of making these agents or components and simplify administration to one active agent.

### SUMMARY

This disclosure describes TEACs and ATTACs that comprise half-life extension moieties. These TEACs and ATTACs may be two-component kits or compositions or a single component of a kit or composition.

This application also describes single-agent TEACs and ATTACs.

This disclosure describes an agent for treating cancer in a patient comprising a first component comprising a targeted T-cell engaging agent comprising: a first targeting moiety that binds a tumor antigen expressed by the cancer; a first T-cell engaging domain capable of T-cell engaging activity when binding a second T-cell engaging domain, wherein the second T-cell engaging domain is not part of the first component, and wherein the first T-cell engaging domain comprises either a VH domain or VL domain;

a first inert binding partner for the first T-cell engaging domain binding to the first T-cell engaging domain such that the first T-cell engaging domain does not bind to the second T-cell engaging domain unless the inert binding partner is removed, wherein if the first T-cell engaging domain comprises a VH domain, the inert binding partner comprises a VL domain and if the first T-cell engaging domain comprises a VL domain, the inert binding partner comprises a VH domain;

a first half-life extending moiety, wherein the first half-life extending moiety is attached (directly or indirectly) to the first inert binding partner; and a protease cleavage site separating the first T-cell engaging domain and the first inert binding partner, wherein the protease cleavage site is capable of releasing the inert binding partner and half-life extending moiety from the T-cell engaging domain in the presence of a protease (1) expressed by the cancer or in the cancer microenvironment or (2) colocalized to the cancer by a targeting moiety that binds a tumor antigen expressed by the cancer and that is the same or different from the targeting moiety in the agent, and

a second component comprising a targeted T-cell engaging agent comprising:

a second targeting moiety that binds a tumor antigen expressed by the cancer;

a second T-cell engaging domain capable of T-cell binding activity when binding a first T-cell engaging domain, wherein the first T-cell engaging domain is not part of the second component, and wherein the second T-cell engaging domain comprises either a VH domain or VL domain;

a second inert binding partner for the second T-cell engaging domain binding to the second T-cell engaging domain such that the second T-cell engaging domain does not bind to the first T-cell engaging domain unless the inert binding partner is removed, wherein if the second T-cell engaging domain comprises a VH domain, the inert binding partner comprises a VL domain and if the second T-cell engaging domain comprises a VL domain, the inert binding partner comprises a VH domain; and

a second half-life extending moiety, wherein the second half-life extending moiety is attached (directly or indirectly) to the second inert binding partner;

a protease cleavage site separating the second T-cell engaging domain and the second inert binding partner, wherein the protease cleavage site is capable of releasing the inert binding partner and half-life extending moiety from the T-cell engaging domain in the presence of a protease (1) expressed by the cancer or in the cancer microenvironment or (2) colocalized to the cancer by a targeting moiety that binds a tumor antigen expressed by the cancer and that is the same or different from the targeting moiety in the agent,
wherein the first and second T-cell engaging domains are capable of binding a T cell when neither is bound to an inert binding partner, and further wherein if the first T-cell engaging domain comprises a VH domain, the second T-cell engaging domain comprises a VL domain and if the first T-cell engaging domain comprises a VL domain, the second T-cell engaging domain comprises a VH domain.

This disclosure also describes an agent for treating cancer in a patient comprising:

a first component comprising a targeted immune cell engaging agent comprising:

a targeting moiety capable of targeting the cancer;

a first immune cell engaging domain capable of immune engaging activity when binding a second immune cell engaging domain, wherein the second immune cell engaging domain is not part of the first component, optionally wherein the first immune cell engaging domain is a T-cell engaging domain;

a first inert binding partner for the first immune cell engaging domain binding to the first immune cell engaging domain such that the first immune cell engaging domain does not bind to the second immune cell engaging domain unless the inert binding partner is removed, wherein if the first immune cell engaging domain comprises a VH domain, the inert binding partner comprises a VL domain and if the first immune cell engaging domain comprises a VL domain, the inert binding partner comprises a VH domain;

a first half-life extending moiety, wherein the first half-life extending moiety is attached (directly or indirectly) to the first inert binding partner; and

a protease cleavage site separating the first immune cell engaging domain and the first inert binding partner, wherein the protease cleavage site is capable of releasing the inert binding partner and half-life extending moiety from the immune cell engaging domain in the presence of a protease (1) expressed by the cancer or in the cancer microenvironment or (2) colocalized to the cancer by a targeting moiety that binds a tumor antigen expressed by the cancer and that is the same or different from the targeting moiety in the agent, and

a second component comprising a selective immune cell engaging agent comprising:

an immune cell selection moiety capable of selectively targeting an immune cell;

a second immune cell engaging domain capable of immune cell engaging activity when binding the first immune cell engaging domain, wherein the first and second immune cell engaging domains are capable of binding when neither is bound to an inert binding partner, optionally wherein the second immune cell engaging domain is a immune cell engaging domain;

a second inert binding partner for the second immune cell engaging domain binding to the second immune cell engaging domain such that the second immune cell engaging domain does not bind to the first immune cell engaging domain unless the inert binding partner is removed, wherein if the second immune cell engaging domain comprises a VH domain, the inert binding partner comprises a VL domain and if the second immune cell engaging domain comprises a VL domain, the inert binding partner comprises a VH domain; and

a second half-life extending moiety, wherein the second half-life extending moiety is attached (directly or indirectly) to the second inert binding partner;

a protease cleavage site separating the second immune cell engaging domain and the second inert binding partner, wherein the protease cleavage site is capable of releasing the inert binding partner and half-life extending moiety from the immune cell engaging domain in the presence of a protease (1) expressed by the cancer or in the cancer microenvironment or (2) colocalized to the cancer by a targeting moiety that binds a tumor antigen expressed by the cancer and that is the same or different from the targeting moiety in the agent,

wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

This disclosure also describes a component for use in a kit or composition for treating cancer in a patient comprising a first targeted immune cell engaging agent comprising:

a targeting moiety that binds a tumor antigen expressed by the cancer;

an immune cell engaging domain capable of immune cell binding activity when binding another immune cell engaging domain, wherein the other immune cell engaging domain is not part of the first component, and wherein the immune cell engaging domain comprises either a VH domain or VL domain, optionally wherein the immune cell engaging domain is a T-cell engaging domain;

an inert binding partner for the immune cell engaging domain binding to the immune cell engaging domain such that the immune cell engaging domain does not bind to the other immune cell engaging domain unless the inert binding partner is removed, wherein if the immune cell engaging domain comprises a VH domain, the inert binding partner comprises a VL domain, and if the immune cell engaging domain comprises a VL domain, the inert binding partner comprises a VH domain;

a half-life extending moiety, wherein the half-life extending moiety is attached (directly or indirectly) to the inert binding partner; and

a protease cleavage site separating the immune cell engaging domain and the inert binding partner, wherein the protease cleavage site is capable of releasing the inert binding partner and half-life extending moiety from the immune cell engaging domain in the presence of a protease (1) expressed by the cancer or (2) colocalized to the cancer by a targeting moiety that binds a tumor antigen expressed by the cancer and that is the same or different from the targeting moiety in the agent,

wherein cleavage of the protease cleavage site causes loss of the inert binding partner and allows for complementation with the other immune cell engaging domain that is not part of the agent, further wherein if the immune cell engaging domain comprises a VH domain, the other immune cell engaging domain comprises a VL domain and if the immune cell engaging domain comprises a VL domain, the other immune cell engaging domain comprises a VH domain.

This disclosure also describes a component for use in a kit or composition for treating cancer in a patient comprising a first targeted immune cell engaging agent comprising:

an immune cell selection moiety capable of selectively targeting an immune cell;

an immune cell engaging domain capable of immune cell binding activity when binding another immune cell engaging domain, wherein the other immune cell engaging domain is not part of the first component, and wherein the immune cell engaging domain comprises either a VH domain or VL domain, optionally wherein the immune cell engaging domain is a T-cell engaging domain;

an inert binding partner for the immune cell engaging domain binding to the immune cell engaging domain such that the immune cell engaging domain does not bind to the other immune cell engaging domain unless the inert binding partner is removed, wherein if the immune cell engaging domain comprises a VH domain, the inert binding partner comprises a VL domain and if the immune cell engaging domain comprises a VL domain, the inert binding partner comprises a VH domain;

a half-life extending moiety, wherein the half-life extending moiety is attached (directly or indirectly) to the inert binding partner; and

a protease cleavage site separating the immune cell engaging domain and the inert binding partner, wherein the protease cleavage site is capable of releasing the inert binding partner and half-life extending moiety from the immune cell engaging domain in the presence of a protease (1) expressed by the cancer or (2) colocalized to the cancer by a targeting moiety that binds a tumor antigen expressed by the cancer and that is the same or different from the targeting moiety in the agent,

wherein cleavage of the protease cleavage site causes loss of the inert binding partner and allows for complementation with the other immune cell engaging domain that is not part of the agent, further wherein if the immune cell engaging domain comprises a VH domain, the other immune cell engaging domain comprises a VL domain and if the immune cell engaging domain comprises a VL domain, the other immune cell engaging domain comprises a VH domain.

This application also describes an agent for treating cancer in a patient comprising:

a first targeting moiety that binds a tumor antigen expressed by the cancer;

a first T-cell engaging domain capable of T-cell binding activity when binding a second T-cell engaging domain, wherein the first T-cell engaging domain comprises either a VH domain or VL domain;

a second T-cell engaging domain capable of T-cell binding activity when binding a first T-cell engaging domain, wherein the second T-cell engaging domain comprises either a VH domain or VL domain;

a first inert binding partner for the first T-cell engaging domain binding to the first T-cell engaging domain such that the first T-cell engaging domain does not bind to the second T-cell engaging domain unless the inert binding partner is removed, wherein if the first T-cell engaging domain comprises a VH domain, the inert binding partner comprises a VL domain and if the first T-cell engaging domain comprises a VL domain, the inert binding partner comprises a VH domain; and

a protease cleavage site separating the first T-cell engaging domain and the first inert binding partner, wherein the protease cleavage site is capable of releasing the inert binding partner from the T-cell engaging domain in the presence of a protease (1) expressed by the cancer or in the cancer microenvironment; or (2) colocalized to the cancer by a targeting moiety that binds a tumor antigen expressed by the cancer and that is the same or different from the targeting moiety in the agent;

wherein the first and second T-cell engaging domains are capable of binding a T cell when neither is bound to an inert binding partner, and further wherein if the first T-cell engaging domain comprises a VH domain, the second T-cell engaging domain comprises a VL domain and if the first T-cell engaging domain comprises a VL domain, the second T-cell engaging domain comprises a VH domain.

This disclosure also describes an agent for treating cancer in a patient comprising:

an immune cell selection moiety capable of selectively targeting an immune cell;

a first immune cell engaging domain capable of immune cell binding activity when binding a second immune cell engaging domain, wherein the first immune cell engaging domain comprises either a VH domain or VL domain, optionally wherein the first immune cell engaging domain comprises a T-cell engaging domain;

a second immune cell engaging domain capable of immune cell binding activity when binding a first immune cell engaging domain, wherein the second immune cell engaging domain comprises either a VH domain or VL domain, optionally wherein the second immune cell engaging domain comprises a T-cell engaging domain;

a first inert binding partner for the first immune cell engaging domain binding to the first immune cell engaging domain such that the first immune cell engaging domain does not bind to the second immune cell engaging domain unless the inert binding partner is removed, wherein if the first immune cell engaging domain comprises a VH domain, the inert binding partner comprises a VL domain and if the first immune cell engaging domain comprises a VL domain, the inert binding partner comprises a VH domain; and

a protease cleavage site separating the first immune cell engaging domain and the first inert binding partner, wherein the protease cleavage site is capable of releasing the inert binding partner from the immune cell engaging domain in the presence of a protease (1) expressed by the cancer or in the cancer microenvironment; or (2) colocalized to the cancer by a targeting moiety that binds a tumor antigen expressed by the cancer and that is the same or different from the targeting moiety in the agent;

wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

In some embodiments, an agent further comprises a second targeting moiety that is capable of targeting the cancer.

In some embodiments, an agent further comprises a second inert binding partner for the second immune cell engaging domain binding to the second immune cell engaging domain such that the second immune cell engaging domain does not bind to the first immune cell engaging domain unless the inert binding partner is removed, wherein if the second immune cell or engaging domain comprises a VH domain, the inert binding partner comprises a VL domain and if the second immune cell engaging domain comprises a VL domain, the inert binding partner comprises a VH domain, optionally wherein the second immune cell engaging domain comprises a T-cell engaging domain; and a protease cleavage site separating the second immune cell engaging domain and the second inert binding partner, wherein the protease cleavage site is capable of releasing the inert binding partner from the immune cell engaging domain in the presence of a protease (1) expressed by the cancer or in the cancer microenvironment; or (2) colocalized to the cancer by a targeting moiety that binds a tumor antigen expressed by the cancer and that is the same or different from the targeting moiety in the agent, wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

In some embodiments, a linker attaches the first and second inert binding partners. In some embodiments, a linker comprises a half-life extending moiety. In some embodiments, a linker is capable of dissociation with the first and/or second inert binding partner upon cleavage of the protease cleavage sites.

In some embodiments, a second component further comprises a second half-life extending moiety, wherein the second half-life extending moiety is attached (directly or indirectly) to the second inert binding partner. In some embodiments, a first and/or second half-life extending moiety is directly attached to the first and/or second inert binding partner. In some embodiments, a first and/or second half-life extending moiety is indirectly attached to the first and/or second inert binding partner via a linker.

In some embodiments, a first component comprises two copies of a first targeting moiety; two copies of a first immune or T-cell engaging domain; and two copies of a first inert binding partner, wherein a protease cleavage site separates both inert binding partners from their respective immune or T-cell engaging domains.

In some embodiments, one end of the half-life extending moiety is attached (directly or indirectly) to one copy of the first inert binding partner and the other end of the half-life extending moiety is attached (directly or indirectly) to the other copy of the first inert binding partner.

In some embodiments, a second component comprises two copies of a second targeting moiety; two copies of a second immune or T-cell engaging domain; and two copies of a second inert binding partner, wherein a protease cleavage sites separates both inert binding partners from their respective immune or T-cell engaging domains.

In some embodiments, one end of the half-life extending moiety is attached (directly or indirectly) to one copy of the second inert binding partner and the other end of the half-life extending moiety is attached (directly or indirectly) to the other copy of the second inert binding partner.

In some embodiments, the two copies of the targeting moiety are the same. In some embodiments, the two copies of the immune or T-cell engaging domain are the same. In some embodiments, the two copies of the inert binding partner are the same. In some embodiments, the two copies of the protease cleavage site separating the inert binding partners from their respective immune or T-cell engaging domains are the same. In some embodiments, the two copies of a protease cleavage site separating the inert binding partners from their respective immune or T-cell engaging domains are different.

In some embodiments, the half-life is decreased after dissociation of one or more half-life extending moieties. In some embodiments, the half-life of the first and/or second component is longer than the half-life of a complex formed by the association of the first and second immune cell or T-cell engaging domains in the form capable of binding to an immune or T cell.

In some embodiments, the first component and/or second component has a half-life greater or equal to 2 days, 4 days, or 7 days. In some embodiments, the agent or component has a half-life greater or equal to 2 days, 4 days, or 7 days.

In some embodiments, the protease cleavage sites are different. In some embodiments, the protease cleavage sites are the same.

In some embodiments, one or more protease cleavage sites are cleaved by a protease expressed by the cancer. In some embodiments, one or more protease cleavage sites are cleaved by a protease that is colocalized to the cancer by a targeting moiety that binds a tumor antigen expressed by the cancer and that is the same or different from the targeting moiety in the agent.

In some embodiments, one or more first and second inert binding partners are capable of dissociation once at least one protease cleavage site for each inert binding partner has been cleaved and after dissociation the two immune cell or T-cell engaging domains that had been bound by the inert binding partners are capable of binding to each other and exhibiting immune cell or T-cell binding activity.

In some embodiments, one or more half-life extending moieties are capable of dissociation together with one or more inert binding partner to which it is attached.

In some embodiments, the one or more half-life extending moieties comprise all or part of an immunoglobulin constant (Fc) domain, serum albumin, serum albumin binding protein, an unstructured protein, and/or PEG. In some embodiments, the one or more half-life extending moieties comprise all or part of an immunoglobulin Fc domain. In some embodiments, the Fc domain comprises the sequence of a human immunoglobulin. In some embodiments, the immunoglobulin is IgG. In some embodiments, the IgG is IgG1, IgG2, or IgG4.

In some embodiments, the Fc domain comprises a naturally occurring sequence.

In some embodiments, the Fc domain comprises one or more mutations as compared to a naturally occurring sequence.

In some embodiments, the Fc domain is an Fc domain with a longer half-life compared to a naturally occurring sequence. In some embodiments, the Fc domain with a longer half-life has increased FcRn binding. In some embodiments, the increased FcRn binding is measured at pH 6.0. In some embodiments, the Fc domain with a longer half-life comprises M252Y/S254T/T256E substitutions. In some embodiments, the Fc domain with a longer half-life comprises M428L/N434S substitutions.

In some embodiments, one or more half-life extending moieties comprise all or part of serum albumin. In some embodiments, the serum albumin is human.

In some embodiments, one or more half-life extending moieties comprise all or part of a serum albumin binding protein. In some embodiments, the serum albumin binding protein is a DARPin, a nanobody, a single-chain variable fragment (scFv), or an antigen-binding fragment (Fab). In some embodiments, the serum albumin binding protein comprises all or part of an albumin binding domain.

In some embodiments, one or more half-life extending moieties comprise all or part of an unstructured protein. In some embodiments, the unstructured protein is an unstructured hydrophilic, biodegradable protein polymer. In some embodiments, the unstructured protein is XTEN.

In some embodiments, one or more half-life extending moieties comprise all or part of PEG.

In some embodiments, the first and second half-life extending moieties are different. In some embodiments, the first and second half-life extending moieties are the same. In some embodiments, the first component is not covalently bound to the second component. In some embodiments, the first component is covalently bound to the second component. In some embodiments, the first component is covalently bound to the second component by a linker comprising a a protease cleavage site.

In some embodiments, the immune cell selection moieties capable of selectively targeting an immune cell selectively targets a T cell, a macrophage, a natural killer cell, a neutrophil, an eosinophil, a basophil, a γδ T cell, a natural killer T cell (NKT cells), or an engineered immune cell.

In some embodiments, the immune cell selection moieties capable of selectively targeting an immune cell selectively targets a T cell, optionally where the T cell is a CD8+ or CD4+ T cell. In some embodiments, the immune cell selection moiety targets CD8, CD4, or CXCR3, or does not specifically bind regulatory T cells. In some embodiments, the immune cell selection moiety comprises an aptamer or an antibody or antigen-specific binding fragment thereof.

In some embodiments, the aptamer or antibody or antigen-specific binding fragment thereof specifically binds an antigen on a T cell. In some embodiments, the first and second T-cell or immune cell engaging domains are capable of binding CD3 or the T cell receptor (TCR) when neither is bound to an inert binding partner. In some embodiments, the first and second T-cell or immune cell engaging domains are capable of forming a Fv when not bound to an inert binding partner.

In some embodiments, the one or more targeting moieties are an antibody or antigen-binding fragment thereof. In some embodiments, the antibody or antigen-binding fragment thereof is (i) specific for any of 4-1BB, 5T4, ACVRL1, ALK1, AXL, B7-H3, BCMA, c-MET, CD133, C4.4a, CA6, CA9, Cadherin-6, CD123, CD133, CD138, CD19, CD20, CD22, CD25, CD27L, CD30, CD33, CD37, CD38, CD44v6, CD56, CD70, CD74(TROP2), CD79b, CEA, CEACAM5, cKit, CLL-1, Cripto, CS1, DLL3, EDNRB, EFNA4, EGFR, EGFRvIII, ENPP3, EpCAM, EPHA2, FGFR2, FGFR3, FLT3, FOLR, FOLR1, GD2, gpA33, GPC3, GPNMB, GUCY2C, HER2, HER3, HLAA2, IGF1-r, IL13RA2, Integrin alpha, LAMP-1, LewisY, LIV-1, LRRC15, MMP9, MSLN, MUC1, MUC16, NaPi2b, Nectin-4, NOTCH3, p-CAD, PD-L1, PSMA, PTK7, ROR1, SLC44A4, SLITRK6, SSTR2, STEAP1, TAG72, TF, TIM-1, or TROP-2, or (ii) an anti-epidermal growth factor receptor antibody; an anti-Her2 antibody; an anti-CD20 antibody; an anti-CD22 antibody; an anti-CD70 antibody; an anti-CD33 antibody; an anti-MUC1 antibody; an anti-CD40 antibody; an anti-CD74 antibody; an anti-P-cadherin antibody; an anti-EpCAM antibody; an anti-CD138 antibody; an anti-E-cadherin antibody; an anti-CEA antibody; an anti-FGFR3 antibody; an anti-mucin core protein antibody; an anti-transferrin antibody; an anti-gp95/97 antibody; an anti-p-glycoprotein antibody; an anti-TRAIL-R1 antibody; an anti-DR5 antibody; an anti-IL-4 antibody; an anti-IL-6 antibody; an anti-CD19 antibody; an anti-PSMA antibody; an anti-PSCA antibody; an anti-Cripto antibody; an anti-PD-L1 antibody; an anti-IGF-1R antibody; an anti-CD38 antibody; an anti-CD133 antibody; an anti-CD123 antibody; an anti-CDE49d antibody; an anti-glypican 3 antibody; an anti-cMET antibody; or an anti-IL-13R antibody.

In some embodiments, the antibody or antigen-binding fragment comprises all or part of the amino acid sequence of 1C1, (GS) 5745, ABBV-085, ABBV-399, ABBV-838, AbGn-107, ABT-414, ADCT-301, ADCT-402, AGS-16C3F, AGS62P1, AGS67E, AMG 172d, AMG 595d, Andecaliximab, Anetumab ravtansine, ARX788, ASG-15MEd, ASG-5MEk, Atezolizumab, AVE1642, AVE9633e, Avelumab, BAY1129980, BAY1187982e, BAY79-4620b, BIIB015d, Bivatuzumab mertansineb, BMS-986148, Brentuximab vedotin, Cantuzumab mertansine, CC49, CDX-014, Cirmtuzumab, Coltuximab ravtansine, DEDN6526Ae, Denintuzumab mafodotin, Depatuxizumab, DFRF4539Ad, DMOT4039Ae, DS-8201A, Durvalumab, Enfortumab vedotin, Farletuzumab, FLYSYN, Gatipotuzumab, Gemtuzumab ozogamicin, Glembatumumab vedotin, GSK2857916, HKT288, Hu3F8, HuMax-AXL-ADC, IDEC-159, IMGN289b, IMGN388a, IMGN529, Indatuximab ravtansine, Inotuzumab ozogamicin, Istiratumab, Labetuzumab govitecan, Lifastuzumab vedotin, LOP628h, Lorvotuzumab mertansine, LY3076226, MCLA-117 (CLEC-12AxCD3), MDX-1203d, MEDI-4276, MEDI-547b, Milatuzumab-doxorubicin, Mirvetuximab soravtansine, MLN0264, MLN2704e, MM-302i, Mosunetuzumab, MOv18 IgE, Ocrelizumab, Oportuzumab, Patritumab, PCA-062, PF-03446962, PF-06263507a, PF-06647020, PF-06647263, PF-06650808d, Pinatuzumab vedotin, Polatuzumab vedotin, PSMA ADC 301c, RC48-ADC, Rituximab, Rovalpituzumab tesirine, Sacituzumab, Sacituzumab govitecan, SAR408701, SAR428926, SAR566658, SC-002, SC-003, SGN-15a, SGN-CD123A, SGN-CD19B, SGN-CD70A, SGN-LIV1A, Sofituzumab vedotin, Solitomab, SSTR2xCD3 XmAb18087, STRO-002, SYD-985, Talacotuzumab, Tisotumab vedotin, Trastuzumab emtansine, U3-1402, Ublituximab, Vadastuximab talirine, Vandortuzumab vedotin, Vorsetuzumab mafodotin, XMT-1522, or Zenocutuzumab.

In some embodiments, one or more targeting moieties are an aptamer. In some embodiments, the aptamer comprises DNA. In some embodiments, the aptamer comprises RNA. In some embodiments, the aptamer is single-stranded. In some embodiments, the aptamer is a target cell-specific aptamer chosen from a random candidate library. In some embodiments, the aptamer is an anti-EGFR aptamer. In some embodiments, the aptamer binds to the antigen on the cancer cell with a Kd from 1 picomolar to 500 nanomolar. In some embodiments, the aptamer binds to the cancer with a Kd from 1 picomolar to 100 nanomolar.

In some embodiments, one or more targeting moieties comprise IL-2, IL-4, IL-6, α-MSH, transferrin, folic acid, EGF, TGF, PD1, IL-13, stem cell factor, insulin-like growth factor (IGF), or CD40. In some embodiments, one or more targeting moiety comprise a full-length sequence of IL-2, IL-4, IL-6, α-MSH, transferrin, folic acid, EGF, TGF, PD1, IL-13, stem cell factor, insulin-like growth factor (IGF), or CD40. In some embodiments, one or more targeting moiety comprise a truncated form, analog, variant, or derivative of IL-2, IL-4, IL-6, α-MSH, transferrin, folic acid, EGF, TGF, PD1, IL-13, stem cell factor, insulin-like growth factor (IGF), or CD40. In some embodiments, one or more targeting moiety bind a target on the cancer comprising IL-2 receptor, IL-4, IL-6, melanocyte stimulating hormone receptor (MSH receptor), transferrin receptor (TR), folate receptor 1 (FOLR), folate hydroxylase (FOLH1), EGF receptor, PD-L1, PD-L2, IL-13R, CXCR4, IGFR, or CD40L.

In some embodiments, the first and second targeting moieties bind the same antigen. In some embodiments, the first and second targeting moieties bind the same epitope.

In some embodiments, the first and second targeting moieties are the same. In some embodiments, the first and second targeting moieties are different.

In some embodiments, the first and second targeting moieties bind different antigens.

In some embodiments, the first and second targeting moieties bind different epitopes of the same antigen (i.e., protein).

This disclosure also describes a method of treating cancer expressing a tumor antigen that binds the first targeting moiety in a patient comprising administering an agent or component to the patient.

In some embodiments, the cancer expressing a tumor antigen that binds the first targeting moiety is any one of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, leukemia, myeloma, nonHodgkin lymphoma, Hodgkin lymphoma, acute myeloid leukemia, acute lymphoblastic leukemia, chronic lymphoblastic leukemia, lymphoproliferative disorder, myelodysplastic disorder, myeloproliferative disease or premalignant disease.

This disclosure also describes a method of targeting an immune response of a patient to cancer comprising administering the agent or component described herein to the patient.

In some embodiments, the T cells express CD3 or TCR and the T cell engaging domain binds CD3 or TCR.

In some embodiments, if the patient has regulatory T cells in the tumor, the selective immune cell engaging agent does not target markers present on regulatory immune cells (including, but not limited to CD4 and CD25).

This disclosure also describes a method of treating cancer expressing a tumor antigen in a patient comprising administering a composition comprising a component described in this disclosure, wherein the first targeting moiety binds the tumor antigen and a second component comprising a half-life extending moiety.

This disclosure also describes a method of treating cancer expressing a tumor antigen in a patient comprising administering a composition comprising a component described in this disclosure, wherein the first targeting moiety binds the tumor antigen and a second component not comprising a half-life extending moiety.

In some embodiments, one or more nucleic acid molecules encodes an agent or component.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1C show different embodiments of TEACs. FIG. 1A provides a two-component TEAC without a half-life extension moiety. One component comprises one T-cell engaging domain and the other component comprises a complementary T-cell engaging domain (shown in white versus diagonal striped domains). When the inert binding partners are cleaved, the T-cell engaging domains are available to bind to each other. The two hashed ovals represent that each TEAC component has a target moiety. Labeling in the other Figures is as described in FIG. 1A, unless noted otherwise.

FIG. 1B shows a two-component TEAC wherein each component comprises two copies of a targeting moiety, a T-cell engaging domain, and an inert binding partner. A linker comprising a half-life extension moiety (such as an Fc domain) links the two copies of the inert binding partner. After cleavage and release of the inert binding partners (together with the linker), a T-cell engaging domain of the first component can pair with a complementary T-cell engaging domain of the second component and bind and engage T cells.

FIG. 1C shows a single-agent TEAC comprising two different targeting moieties, a first T-cell engaging domain and a second T-cell engaging domain that are complementary, and a first and second inert binding partner that are attached by a linker comprising a half-life extension moiety. Following cleavage and release of the inert binding partners (together with the linker), the complementary first and second T-cell engaging domain of the agent can pair to bind and engage T cells.

FIGS. 2A-2E provide a comparison of two-component and single-agent TEACs targeted to CD33 and CD123. FIG. 2A shows results with the two-component (Dual IgG TEACs with structure shown in FIG. 2C). FIG. 2B shows results comparing the Dual IgG TEACs with the single-agent TEAC (DUO Ig-TEAC with structure shown in FIG. 2D) wherein the two targeting moieties are an anti-CD33 and an anti-CD123 antibody. In the two-component TEAC system, one component comprises two identical copies of an anti-C33 targeting moiety, and the other component comprises two identical copies of an anti-CD123 targeting moiety. FIG. 2E shows how the DUO and Dual TEACs allow specificity based on binding to two separate antigens (Antigen 1 and Antigen 2).

FIG. 3 provides results of a comparison of two-component and single-agent TEACs targeted to EpCAM. In the single-agent TEAC, the two targeting moieties are different anti-EpCAM antibodies. In the two-component TEAC system, each component comprises two identical copies of an anti-EpCAM targeting moiety, and these targeting moieties are different for the two components.

FIG. 4 shows pharmacology modeling (using quantitative systems pharmacology) of TEAC or ATTAC activation when the half-life extending moiety is fused to an inert binding partner and therefore removed during proteolytic activation following intermittent dosing of the TEAC or ATTAC. Tumor refers to activated TEAC or ATTAC levels in the tumor. “Toxicity” refers to levels of the activated TEAC or ATTAC outside the tumor, where they could lead to toxicity. The dashed line indicates the maximum number of CD3 molecules engaged in non-target tissue, wherein this engagement in non-target tissue can lead to toxicity. The difference between the maximum engagement in non-target tissue (i.e., toxicity) versus the efficacy in target tumor tissues can be used as a surrogate for the therapeutic index.

FIGS. 5A-5C show structure (FIG. 5A), expression data (FIG. 5B), and proteolytic cleavage data (FIG. 5C) of TEAC components comprising 2 copies each of a targeting moiety (a Fab in this representative TEAC), a T-cell engaging domain, and an inert binding partner, wherein the 2 copies of the inert binding partner are attached via a linker comprising an effectorless Fc (hu IgG1 N297Q). In FIG. 5A, the striped ovals represent the T-cell engaging domains. The gray ovals attached to the Fc domain represent the inert binding partners. Cleavage of the linkers between the T-cell engaging domains and the inert binding partners in the tumor microenvironment (TME) allows release of the Fc domain that is linked to the 2 copies of the inert binding partner. R0258-R0261 comprise SEQ ID NOs: 175-178, respectively.

FIG. 6 shows EGFR ELISA results for TEACs that comprise TEACs comprising Fc domains (RO268-RO269) in comparison to control constructs (RO130-RO132) that do not comprise half-life extension moieties.

FIGS. 7A-7B show T-cell activation (FIG. 7A, IFNgamma ELISA) and T-cell killing assay (FIG. 7B, LDH release) results for TEAC pairs that comprise Fc domains (RO268+RO269), control TEAC pairs that do not comprise half-life extension moities (RO130-RO131), or a BITE control (RO132).

FIGS. 8A-8B shows pharmacokinetic data for a TEAC comprising an Fc domain (RO269) versus a TEAC that does not comprise an FC domain (RO270) after intravenous (8A) versus intraperitoneal (8B) administration.

### DESCRIPTION OF THE SEQUENCES

Table 1A provides a listing of certain sequences referenced herein. Table 1B provides a listing of certain construct sequences used herein.

TABLE 1A
Description of the Sequences and SEQ ID NOs

| Description | Sequence | # |
|---|---|---|
| ADAM28 cleavage site | KPAKFFRL | 1 |
| ADAM28 cleavage site | DPAKFFRL | 2 |
| ADAM28 cleavage site | KPMKFFRL | 3 |
| ADAM28 cleavage site | LPAKFFRL | 4 |
| ADAM28 cleavage site | LPMKFFRL | 5 |
| ADAM28 cleavage site | KPAMFFRL | 6 |
| ADAM28 cleavage site | YPAKFFRL | 7 |
| ADAM28 cleavage site | KWAKFFRL | 8 |
| ADAM28 cleavage site | DPMKFFRL | 9 |
| ADAM28 cleavage site | DPAMFFRL | 10 |
| ADAM28 cleavage site | DPMMFFRL | 11 |
| ADAM28 cleavage site | KMAMFFRL | 12 |
| ADAM28 cleavage site | KMAMFFIM | 13 |
| ADAM28 cleavage site | KPAMFFIM | 14 |
| ADAM28 cleavage site | LPAMFFRL | 15 |
| ADAM28 cleavage site | LPMMFFRL | 16 |
| ADAM28 cleavage site | LMAMFFRL | 17 |
| ADAM28 cleavage site | LMAMFFIM | 18 |
| ADAM28 cleavage site | LPAMFFIM | 19 |
| ADAM28 cleavage site | LPAMFFYM | 20 |
| ADAM28 cleavage site | KPMMFFRL | 21 |
| ADAM28 cleavage site | KPAKFFYM | 22 |
| ADAM28 cleavage site | KPAKFFIM | 23 |
| ADAM28 cleavage site | IPMKFFRL | 24 |
| ADAM28 cleavage site | IPAMFFRL | 25 |
| ADAM28 cleavage site | IPMMFFRL | 26 |
| ADAM28 cleavage site | IMAMFFRL | 27 |
| ADAM28 cleavage site | IMAMFFIM | 28 |
| ADAM28 cleavage site | IPAMFFIM | 29 |
| ADAM28 cleavage site | IPAMFFYM | 30 |
| cathepsin B cleavage site | FR | 31 |
| cathepsin B cleavage site | FK | 32 |
| cathepsin B cleavage site | VA | 33 |
| cathepsin B cleavage site | VR | 34 |
| cathepsin B cleavage site | V{Cit} | 35 |
| {Cit} = citrulline | | |
| cathepsin B cleavage site | HLVEALYL | 36 |
| cathepsin B cleavage site | SLLKSRMVPNFN | 37 |
| cathepsin B cleavage site | SLLIARRMPNFN | 38 |
| cathepsin B cleavage site | KKFA | 39 |
| cathepsin B cleavage site | AFKK | 40 |
| cathepsin B cleavage site | QQQ | 41 |
| cathepsin D cleavage site | PRSFFRLGK | 42 |
| cathepsin D cleavage site | SGVVIATVIVIT | 43 |
| cathepsin K cleavage site | GGP | 44 |
| MMP1 cleavage site | SLGPQGIWGQFN | 45 |
| MMP2 cleavage site | AIPVSLR | 46 |
| MMP2 cleavage site | SLPLGLWAPNFN | 47 |
| MMP2 cleavage site | HPVGLLAR | 48 |
| MMP2/9 cleavage site | GPLGVRGK | 49 |
| MMP2 cleavage site | GPLGLWAQ | 50 |
| MMP3 cleavage site | STAVIVSA | 51 |
| MMP7 cleavage site | GPLGLARK | 52 |
| MMP7 cleavage site | RPLALWRS | 53 |
| MMP7 cleavage site | SLRPLALWRSFN | 54 |
| MMP2/9 cleavage site | GILGVP | 55 |
| MMP2/9 cleavage site | GPLGIAGQ | 56 |
| MMP9 cleavage site | AVRWLLTA | 57 |
| MMP9 cleavage site | PLGLYAL | 58 |
| MMP9 cleavage site | GPQGIAGQR | 59 |
| MMP9 cleavage site | KPVSLSYR | 60 |
| MMP11 cleavage site | AAATSIN | 61 |
| MMP11 cleavage site | AAGAMFLE | 62 |
| MMP13 cleavage site | GPQGLAGQRGIV | 63 |
| MMP14 cleavage site | PRHLR | 64 |
| MMP14 cleavage site | PQGLLGAPGILG | 65 |
| MMP14 cleavage site | PRSAKELR | 66 |
| PSA/KLK3 | HSSKLQ | 67 |
| PSA/KLK3 | SSKLQ | 68 |
| KLK4 | RQQR | 69 |
| TMPRSS2 | GGR | 70 |
| Legumain | AAN | 71 |
| ST14 (Matriptase) | QAR | 72 |
| Cls cleavage site | YLGRSYKV | 73 |
| Cls cleavage site | MQLGRX | 74 |
| MASP2 cleavage site | SLGRKIQI | 75 |
| C2a and Bb cleavage site | GLARSNLDE | 76 |
| uPa cleavage site | TYSRSRYL | 77 |
| uPa cleavage site | KKSPGRVVGGSV | 78 |
| uPa cleavage site | NSGRAVTY | 79 |
| uPa cleavage site | AFK | 80 |
| tissue-type plasminogen | GGSGQRGRKALE | 81 |
| activator (tPA) | | |
| ADAM10 | PRYEAYKMGK | 82 |
| ADAM12 | LAQAF | 83 |
| ADAM17 | EHADLLAVVAK | 84 |
| flexible amino acid linker (may | GGGGS | 85 |
| be presented in repeating | | |
| fashion) | | |
| flexible amino acid linker (may | GGGS | 86 |
| be presented in repeating | | |
| fashion) | | |
| flexible amino acid linker (may | GS | 87 |
| be presented in repeating | | |
| fashion) | | |
| flexible amino acid linker (may | GSGGS | 88 |
| be presented in repeating | | |
| fashion) | | |
| flexible amino acid linker (may | GGSG | 89 |
| be presented in repeating | | |
| fashion) | | |
| flexible amino acid linker (may | GGSGG | 90 |
| be presented in repeating | | |
| fashion) | | |
| flexible amino acid linker (may | GSGSG | 91 |
| be presented in repeating | | |
| fashion) | | |
| flexible amino acid linker (may | GSGGG | 92 |
| be presented in repeating | | |
| fashion) | | |
| flexible amino acid linker (may | GGGSG | 93 |
| be presented in repeating | | |
| fashion) | | |
| flexible amino acid linker (may | GSSSG | 94 |
| be presented in repeating | | |
| fashion) | | |
| Anti-EGFR aptamer (tight | UGCCGCUAUAAUGCACGGAUUUAAUCGCCGUA | 95 |
| binder with K_{d} = 2.4 nM) | GAAAAGCAUGUCAAAGCCG | |
| Anti-EGFR aptamer | UGGCGCUAAAUAGCACGGAAAUAAUCGCCGUA | 96 |
| | GAAAAGCAUGUCAAAGCCG | |
| Anti-EGFR aptamer | UGCUAGUAUAUCGCACGGAUUUAAUCGCCGUA | 97 |
| | GAAAAGCAUGUCAAAGCCG | |
| Anti-EGFR aptamer | UGCCGCCAUAUCACACGGAUUUAAUCGCCGUA | 98 |
| | GAAAAGCAUGUCAAAGCCG | |
| Anti-EGFR aptamer | UUCCGCUGUAUAACACGGACUUAAUCGCCGUA | 99 |
| | GUAAAGCAUGUCAAAGCCG | |
| Anti-EGFR aptamer | UGUCGCUCUAUUGCACGGAUUUAAUCGCCGUA | 100 |
| | GAAAAGCAUGUCAAAGCCG | |
| Anti-EGFR aptamer | UGCUGCUUUAUCCCACAUAUUUUUUCCCCUCA | 101 |
| | UAACAAUAUUUCUCCCCCC | |
| Anti-EGFR aptamer | UGCNGCUAUAUCGCNCGUAUUUAAUCGCCGUA | 102 |
| | GAAAAGCAUGUCNANGCCG | |
| Anti-EGFR aptamer | UGCAAAGAAAACGCACGUAUUUAAUCGCCGUA | 103 |
| | GUAAAGCAUGUCAAAGCCG | |
| Anti-EGFR aptamer | UGCAUCACUAUCGAACCUAUUUAAUCCACCAA | 104 |
| | AAUAAUUGCAAGUCCAUACUC | |
| Anti-EGFR aptamer | UGCCNNAAUAACACACNUAUAUAAUCGCCGUA | 105 |
| | CAAAAUCAUGUCAAANCCG | |
| Anti-EGFR aptamer | UGCAGCUGUAUUGCACGUAUUUAAUCGCCGUA | 106 |
| | GAAAAGCAUGUCAAAGCCG | |
| Anti-EGFR aptamer | UUCCGAUAAUCCCGCGUACUAAAUCACCAUAG | 107 |
| | UCAACAAUUUCCAACCUC | |
| Anti-EGFR aptamer | UCCACUAUAUCACACGUAUUUAAUCGCCGUAG | 108 |
| | AAAAGCAUGUCAAAGCCG | |
| Anti-EGFR aptamer | UCCCUCAACCUCGCUACUAUUUAAUCGCCGUA | 109 |
| | GAAAAGCAUGUCAAAGCCU | |
| Anti-EGFR aptamer | UGCCGCUAUAUCACACGAAUUUAAUCGCCGUA | 110 |
| | GAAAAGCAUGUCAAAGCCG | |
| Anti-EGFR aptamer | AGCCCCUAGAACACACGGAUUUAAUCGCCGUA | 111 |
| | GAAAAGCAUGUCAAAGCCG | |
| Anti-EGFR aptamer | UGCCAAUAUAUAACACGGAAUUAAUCGCCGUA | 112 |
| | GAAAAGCAUGUCAAAGCCG | |
| Anti-EGFR aptamer | UGCCGCUAUAGCGCACGGAUUUAAUCGCCGUA | 113 |
| | GAAAAGCAUGUCAAAGCCG | |
| Anti-EGFR aptamer | UGCAGAUAUAUGUCACUCAUUAAUCCCCGUAU | 114 |
| | AAAAACAUAACUAAGCUC | |
| Anti-EGFR aptamer | UGUAGCUGUAUUGCACACAUUAAAUCGCCGUA | 115 |
| | GUAAAGCAUGUCAAAGCCG | |
| Anti-EGFR aptamer | UACCAAUAUAUCGCCACACAUAAUCGCCGUAG | 116 |
| | AAAAGCAUGUCAAAGCCG | |
| Anti-EGFR aptamer | UGCCGCUAUGCCCACGGAAUUUAAUCGCCGUA | 117 |
| | GAAAAACAUGUCAAAGUCG | |
| Anti-EGFR aptamer | UGCCGCUAUUUAGCACGGAUUAAAUCGCCGUA | 118 |
| | GAAAAGCAUGUCAAAGCCG | |
| Anti-EGFR aptamer | UGCCGCUAUUUAGCACGGAUUAAAUCGCCGUA | 119 |
| | GAAAAGCAUGUCNAAGCCG | |
| Anti-EGFR aptamer | UGUAGUAAUAUGACACGGAUUUAAUCGCCGUA | 120 |
| | GAAAAGCANGUCAAAGCCU | |
| Anti-EGFR aptamer | UGUCGCCAUUACGCACGGAUUUAAUCGCCGUA | 121 |
| | GAAAAGCAUGUCAAAGCCG | |
| Anti-EGFR aptamer | UGCCCCCAAACUACACAAAUUUAAUCGCCGUA | 122 |
| | UAAAAGCAUGUCAAAGCCG | |
| Anti-EGFR aptamer | UGCACUAUCUCACACGUACUAAUCGCCGUAUA | 123 |
| | AAAGCAUGUCAAAGCCG | |
| Anti-EGFR aptamer | UGUCGCAAUAAUACACUAAUUUAAUCGCCGUA | 124 |
| | GAAAAGCAUGUCAAAGCCG | |
| Anti-EGFR aptamer | UGCAACAAUAUAGCACGUAUUUAAUCGCCGUA | 125 |
| | GUAAAGCAUGUCAAAGG | |
| Anti-EGFR aptamer | CUACCACAAAUCCCACAUAUUUAAUCUCCCAA | 126 |
| | UCAAAUCUUGUCCAUUCCC | |
| Anti-EGFR aptamer | UGCCCUAAACUCACACGGAUAUAAUCGCCGUA | 127 |
| | GAAAAGCAUGUCAAAGCCG | |
| Anti-EGFR aptamer | UUGUCGUAUGUCACACGUAUUAAAUCGCCGUA | 128 |
| | UAAAAGCAUGUCAAAGCCG | |
| Anti-EGFR aptamer | UUCCGCUAUAACACACGGAGAAAAUCGCCGUA | 129 |
| | GUAAAGCAUGUCAAAGCCG | |
| Anti-EGFR aptamer | UGCCGAUAUAACGCACGGAUAUAAUCGCCGUA | 130 |
| | GAAAAGCAUGUCAAAGCCG | |
| Anti-EGFR aptamer | UGCCAUUAUACAGCACGGAUUUAAUCGCCGUA | 131 |
| | GAAAAGCAUGUCAAAGCCG | |
| Anti-EGFR aptamer | UCCAGAAAUAUGCACACAUUUAAUCGCCGUAG | 132 |
| | AAAAGCAUGUCAAAGCCG | |
| Anti-EGFR aptamer | UCCGCUAAACAACACGGAUACAAUCGCCGUAG | 133 |
| | AAAAGCAUGUCCAAGCCG | |
| Anti-EGFR aptamer | UGCACUAUCUCACACGUACUAAUCGCCGUAUA | 134 |
| | AAAGCAUGUCAAANNNG | |
| Anti-EGFR aptamer | AUNGCNANNNUACACGUAUUNAAUCGCCGUAG | 135 |
| | AAAAGCAUGUCANAGCCG | |
| Anti-EGFR aptamer | UGCUGCUAUAUUGCAAUUUUUUAAACUAAGUA | 136 |
| | GAAAACCAUGUACAAGUCG | |
| Anti-EGFR aptamer | UGUCGCCAUAUUGCACGGAUUUAAUCGCCGUA | 137 |
| | GAAAAGCAUGUCCAAGCCG | |
| Anti-EGFR aptamer | UGCCGUUAUAACCCACGGAAUUUAACCUCCGU | 138 |
| | AGAAAAGCAUGUCAAAGCCG | |
| Anti-EGFR aptamer | UGUGAAUAUAUAUCACGGAUUUAAUCGCCGUA | 139 |
| | UAAAAGCNAUGUCAAAGCCG | |
| Anti-EGFR aptamer | UGCCGAUAUNNANCACGGAUUUAAUCGCCGUA | 140 |
| | GAAAAGCAUGUCCAAGCCG | |
| Anti-EGFR aptamer | UGUCACUAAAUUGCACGUAUAUAAUCGCCGUA | 141 |
| | GUAAGCAUGUCAAAGCCG | |
| Anti-EGFR aptamer | UGCAACCAUAAAGCACGUAAUAAAUCGCCGUA | 142 |
| | UAUAAGCAUGUCAAAGCCG | |
| Anti-EGFR aptamer | UGCCGCUAUAUAGCACGUAUUAAUCGCCGUAG | 143 |
| | UAAAGCAUGUCAAAGCCG | |
| Anti-EGFR aptamer | UGCCGCUAUAGCACACGGAAUUUAAUCGCCGU | 144 |
| | AGUAAAGCAUGUCAAAGCCG | |
| Anti-EGFR aptamer | UGCAGGUAUAUAACNCGGAUUUAAUCGCCGUA | 145 |
| | GAAAAGCAUGUCNAAGCCG | |
| Anti-EGFR aptamer | UGCUCCUAUAACACACGGAUUUAAUCGCCGUA | 146 |
| | GAAAAGCAUGUCCAAGCCG | |
| Anti-EGFR aptamer | UGCCCGUAAUUGCACGGAUUUAAUCGCCGUAG | 147 |
| | AAAAGCAUGUCCAAGCCGG | |
| Anti-EGFR aptamer | ACUCCCUAUAUNGCAACUACAUAAUCGCCGUA | 148 |
| | AAUAAGCAUGUNCAAGCCG | |
| Anti-EGFR aptamer | UGAAGCUAGAUCACACUAAAUUAAUCGCCGUA | 149 |
| | GAAAAGCAUGUCAAAAAAGCCG | |
| Anti-EGFR aptamer | UGACUCUUUAUCCCCCGUACAUUAUUCACCGA | 150 |
| | ACCAAAGCAUUACCAUCCCC | |
| Anti-EGFR aptamer | UGACGCCCUAACACACGUAUAUAAUCGCCGUA | 151 |
| | GAAAAGCAUGUCAAAGCCG | |
| Anti-EGFR aptamer | UGUCGCAAAAUAGCACGUAUUUAAUCGCCGUA | 152 |
| | GAAAAGCAUGUCCAAGCCG | |
| Anti-EGFR aptamer | UGAGUGUAUAAUUCACGUAUUUAAUCGCCGUA | 153 |
| | GAAAAGCAUGUCAAAGCCG | |
| Anti-EGFR aptamer | UGCUACUAUAUCGUAGGUAACUAAUCGCCCUA | 154 |
| | CAAACUCACUCUAAAACCG | |
| Anti-EGFR aptamer | UUACGCUAUAUCACACGGAAUUUUAAUCGCCG | 155 |
| | UAGAAAAGCAUGUCCAAGCCG | |
| Anti-EGFR aptamer | CCCAUCUGUACUACAGGAAUUUAAUCGCCGUA | 156 |
| | GAAAAGCAUGUCCAAGCCG | |
| Anti-EGFR aptamer | UGCCCAUAAAUAGCACGGAUUUAAUCGCCGUA | 157 |
| | GAAAAGCAUGUCCAAGCCG | |
| Anti-EGFR aptamer | UGCCGCAAUAACAUACACAUAUAAUCGCCGUA | 158 |
| | GAAAAGCAUGUCAAAGCCG | |
| Anti-EGFR aptamer | UGCAACUAUAUCGCACGUAUGUAAUCGCCGUA | 159 |
| | GAAAAAGCAUGUCAAAGCC | |
| Anti-EGFR aptamer | UUCCGCUAUAUAGCACGGAAUUAAUCGCCGUA | 160 |
| | GAAAAGCAUGUCCAAGCCG | |
| Anti-EGFR aptamer | UUCCGCUAAGUCACACGAAAUUAAUCGCCGUA | 161 |
| | GAAAAGCAUGUCCAAGCCG | |
| Anti-EGFR aptamer | UGUAGCAAUAUCACACGUAAUUAAUCGCCGUA | 162 |
| | UAUAAGCAUGUCAAAGCCG | |
| Anti-EGFR aptamer | UGCCGUUAUAUAUCACGGAUUUAAUCGCCGUA | 163 |
| | GAAAAGCAUGUCCAAGCCG | |
| Anti-EGFR aptamer | UAACACAUAUAUCAAGUAACUUAUCUCCUUAG | 164 |
| | UAACCAUCUCCAAGCCG | |

TABLE 1B
DESCRIPTION OF CONSTRUCT SEQUENCES AND SEQ ID NOS

| Description | Sequence | |
|---|---|---|
| Construct 6248 | ELVMTQSPSSLTVTAGEKVTMSCKSSQSL | 165 |
| single chain; scFv anti-EPCAM | LNSGNQKNYLTWYQQKPGQPPKLLIYW | |
| [Mus musculus V-KAPPA | ASTRESGVPDRFTGSGSGTDFTLTISSVQA | |
| (IGKV8-19*01 | EDLAVYYCQNDYSYPLTFGAGTKLEIKG | |
| (98.00%)-IGKJ5*01 L126+ > I | GGGSGGGGSGGGGSEVQLLEQSGAELVR | |
| (112))[12.3.9] (1-113)-15-mer | PGTSVKISCKASGYAFTNYWLGWVKQRP | |
| tris(tetraglycyl-seryl) linker (114- | GHGLEWIGDIFPGSGNIHYNEKFKGKATL | |
| 128)-Mus musculus VH | TADKSSSTAYMQLSSLTFEDSAVYFCARL | |
| (IGHV1-54*01 (85.90%)- | RNWDEPMDYWGQGTTVTVSSGGGGSD | |
| (IGHD)-IGHJ4*01, S 123 > T | VQLVQSGAEVKKPGASVKVSCKASGYTF | |
| (243)) [8.8.14] (129-248)]-5-mer | TRYTMHWVRQAPGQGLEWIGYINPSRG | |
| tetraglycyl-seryl linker (249-253)- | YTNYADSVKGRFTITTDKSTSTAYMELSS | |
| scFv anti-CD3E | LRSEDTATYYCARYYDDHYCLDWGQG | |
| [humanized VH (Homo sapiens | TTVTVSSGEGTSTGSGAIPVSLRGSGGSG | |
| IGHV1-46*01 (82.50%)-(IGHD)- | GADDIVLTQSPATLSLSPGERATLSCRAS | |
| IGHJ6*01) [8.8.12] (254-372)- | QSVSSSYLAWYQQKPGQAPRLLIYGASS | |
| 25-mer linker (373-397 | RATGVPARFSGSGSGTDFTLTISSLEPEDF | |
| containing MMP2 cleavage site | ATYYCLQIYNMPITFGQGTKVEIKDKTHT | |
| AIPVSLR (SEQ ID NO: 46))-V- | CPPCPAPELLGGPSVFLFPPKPKDTLMISR | |
| KAPPA | TPEVTCVVVDVSHEDPEVKFNWYVDGV | |
| (Homo sapiens V-KAPPA from | EVHNAKTKPREEQYNSTYRVVSVLTVLH | |
| gantenerumab, CAS: 1043556- | QDWLNGKEYKCKVSNKALPAPIEKTISK | |
| 46-2; 398-505); human IgG1 Fc | AKGQPREPQVYTLPPSRDELTKNQVSLTC | |
| (506-731) | LVKGFYPSDIAVEWESNGQPENNYKTTP | |
| | PVLDSDGSFFLYSKLTVDKSRWQQGNVF | |
| | SCSVMHEALHNHYTQKSLSLSPG | |
| Construct 6249 | ELVMTQSPSSLTVTAGEKVTMSCKSSQSL | 166 |
| single chain; scFv anti-EPCAM | LNSGNQKNYLTWYQQKPGQPPKLLIYW | |
| [Mus musculus V-KAPPA | ASTRESGVPDRFTGSGSGTDFTLTISSVQA | |
| (IGKV8-19*01 | EDLAVYYCQNDYSYPLTFGAGTKLEIKG | |
| (98.00%)-IGKJ5*01 L126 > I | GGGSGGGGSGGGGSEVQLLEQSGAELVR | |
| (112)) [12.3.9] (1-113)-15-mer | PGTSVKISCKASGYAFTNYWLGWVKQRP | |
| tris(tetraglycyl-seryl) linker (114- | GHGLEWIGDIFPGSGNIHYNEKFKGKATL | |
| 128)-Mus musculus VH | TADKSSSTAYMQLSSLTFEDSAVYFCARL | |
| (IGHV1-54*01 (85.90%)- | RNWDEPMDYWGQGTTVTVSSGGGGSDI | |
| (IGHD)-IGHJ4*01, S123 > T | VLTQSPATLSLSPGERATLSCRASQSVSY | |
| (243)) [8.8.14] (129-248)]-5-mer | MNWYQQKPGKAPKRWIYDTSKVASGVP | |
| tetraglycyl-seryl linker (249-253)- | ARFSGSGSGTDYSLTINSLEAEDAATYYC | |
| scFv anti-CD3E | QQWSSNPLTFGGGTKVEIKGEGTSTGSG | |
| V-KAPPA (Mus musculus | AIPVSLRGSGGSGGADDVQLVQSGAEVK | |
| IGKV4-59*01 (81.70%)- | KPGASVKVSCKASGYTFTGYYMHWVRQ | |
| IGKJ1*01 L124 > V (493)[5.3.9] | APGQGLEWMGWINPNSGGTNYAQKFQG | |
| (254-359)- | RVTITRDTSASTAYMELSSLRSEDTAVYY | |
| 25-mer linker (360-384 | CARDFLSGYLDYWGQGTLVTVSSDKTHT | |
| containing MMP2 cleavage site | CPPCPAPELLGGPSVFLFPPKPKDTLMISR | |
| AIPVSLR (SEQ ID NO: 46))-Ig | TPEVTCVVVDVSHEDPEVKFNWYVDGV | |
| heavy chain V region (clone | EVHNAKTKPREEQYNSTYRVVSVLTVLH | |
| alpha-MUC1-1, GenBank | QDWLNGKEYKCKVSNKALPAPIEKTISK | |
| Accession S36265; 385-502)- | AKGQPREPQVYTLPPSRDELTKNQVSLTC | |
| human IgG1 Fc (503-728) | LVKGFYPSDIAVEWESNGQPENNYKTTP | |
| | PVLDSDGSFFLYSKLTVDKSRWQQGNVF | |
| | SCSVMHEALHNHYTQKSLSLSPG | |
| Construct 9329 | QVQLVQSGGGLVQPGGSLRLSCAASYFD | 167 |
| Glypican3 V_{HH}-CD3ϵ(VH- | FDSYEMSWVRQAPGKGLEWIGSIYHSGS | |
| MMP2-V_{L}) | TYYNPSLKSRVTISRDNSKNTLYLQMNTL | |
| Anti-human Glypican-3 VHH | RAEDTATYYCARVNMDRFDYWGQGTL | |
| sequence from U.S. Patent | VTVSSSGGGGSDVQLVQSGAEVKKPGAS | |
| 2012145469; residues 1-116)-6- | VKVSCKASGYTFTRYTMHWVRQAPGQG | |
| mer (tetraglycyl-seryl) linker | LEWIGYINPSRGYTNYADSVKGRFTITTD | |
| (117-122)- | KSTSTAYMELSSLRSEDTATYYCARYYD | |
| scFv anti-CD3E | DHYCLDYWGQGTTVTVSSGEGTSTGSG | |
| [humanized VH (Homo sapiens | AIPVSLRGSGGSGGADDIVLTQSPATLSLS | |
| IGHV1-46*01 (82.50%)-(IGHD)- | PGERATLSCRASQSVSSSYLAWYQQKPG | |
| IGHJ6*01) [8.8.12] (123-241)- | QAPRLLIYGASSRATGVPARFSGSGSGTD | |
| 25-mer linker (242-266 | FTLTISSLEPEDFATYYCLQIYNMPITFGQ | |
| containing MMP2 cleavage site | GTKVEIKDKTHTCPPCPAPELLGGPSVFL | |
| AIPVSLR (SEQ ID NO: 46))-V- | FPPKPKDTLMISRTPEVTCVVVDVSHEDP | |
| KAPPA | EVKFNWYVDGVEVHNAKTKPREEQYNS | |
| (Homo sapiens V-KAPPA from | TYRVVSVLTVLHQDWLNGKEYKCKVSN | |
| gantenerumab, CAS: 1043556- | KALPAPIEKTISKAKGQPREPQVYTLPPSR | |
| 46-2; 267-374); human IgG1 Fc | DELTKNQVSLTCLVKGFYPSDIAVEWES | |
| (375-600) | NGQPENNYKTTPPVLDSDGSFFLYSKLTV | |
| | DKSRWQQGNVFSCSVMHEALHNHYTQK | |
| | SLSLSPG | |
| Construct 9330 | DIQMTQSTSSLSASLGDRVTISCSASQGIN | 168 |
| anti-[Homo sapiens SDC1 | NYLNWYQQKPDGTVELLIYYTSTLQSGV | |
| (syndecan-1, CD138), scFv, from | PSRFSGSGSGTDYSLTISNLEPEDIGTYYC | |
| indatuximab CAS: 1238517-16-2, | QQYSKLPRTFGGGTKLEIKRGGGGSGGG | |
| U.S. Patent US20140010828], | GSGGGGSQVQLQQSGSELMMPGASVKIS | |
| [Mus musculus V-KAPPA | CKATGYTFSNYWIEWVKQRPGHGLEWI | |
| (IGKV10-94*01-IGKJ1*01) | GEILPGTGRTIYNEKFKGKATFTADISSNT | |
| [6.3.9] (1-108)-15-mer | VQMQLSSLTSEDSAVYYCARRDYYGNF | |
| tris(tetraglycyl-seryl) linker (109- | YYAMDYWGQGTSVTVSSGGGGSDVQLV | |
| 123) [Mus musculus VH | QSGAEVKKPGASVKVSCKASGYTFTRYT | |
| (IGHV1-9*01-(IGHD)- | MHWVRQAPGQGLEWIGYINPSRGYTNY | |
| IGHJ4*01) [8.8.15] (124-245)-5- | ADSVKGRFTITTDKSTSTAYMELSSLRSE | |
| mer (tetraglycyl-seryl) linker | DTATYYCARYYDDHYCLDYWGQGTTVT | |
| (246-250)-scFv anti-CD3E | VSSGEGTSTGSGAIPVSLRGSGGSGGADD | |
| [humanized VH (Homo sapiens | IVLTQSPATLSLSPGERATLSCRASQSVSS | |
| IGHV1-46*01 (82.50%)-(IGHD)- | SYLAWYQQKPGQAPRLLIYGASSRATGV | |
| IGHJ6*01) [8.8.12] (251-369)- | PARFSGSGSGTDFTLTISSLEPEDFATYYC | |
| 25-mer linker (370-394 | LQIYNMPITFGQGTKVEIKDKTHTCPPCP | |
| containing MMP2 cleavage site | APELLGGPSVFLFPPKPKDTLMISRTPEVT | |
| AIPVSLR (SEQ ID NO: 46))-V- | CVVVDVSHEDPEVKFNWYVDGVEVHNA | |
| KAPPA | KTKPREEQYNSTYRVVSVLTVLHQDWL | |
| (Homo sapiens V-KAPPA from | NGKEYKCKVSNKALPAPIEKTISKAKGQP | |
| gantenerumab, CAS: 1043556- | REPQVYTLPPSRDELTKNQVSLTCLVKGF | |
| 46-2; 395-502); human IgG1 Fc | YPSDIAVEWESNGQPENNYKTTPPVLDS | |
| (503-728) | DGSFFLYSKLTVDKSRWQQGNVFSCSVM | |
| | HEALHNHYTQKSLSLSPG | |
| Construct 9334 | QVKLEESGGGSVQTGGSLRLTCAASGRT | 169 |
| EGFR V_{HH}-CD3ϵ(V_{H}-MMP2-V_{L}) | SRSYGMGWFRQAPGKEREFVSGISWRGD | |
| Anti-human EGFR V_{HH} sequence | STGYADSVKGRFTISRDNAKNTVDLQMN | |
| from 7D12 sequence from | SLKPEDTAIYYCAAAAGSAWYGTLYEYD | |
| Schmitz KR et al, Structure. 2013 | YWGQGTQVTVSSGGGGSGGGGSGGGGS | |
| Jul 2;21(7):1214-24; residues 1- | GGGGSGGGGSGGGGSDVQLVQSGAEVK | |
| 124) -30-mer hexa(tetraglycyl- | KPGASVKVSCKASGYTFTRYTMHWVRQ | |
| seryl) linker (125-154)- | APGQGLEWIGYINPSRGYTNYADSVKGR | |
| scFv anti-CD3E | FTITTDKSTSTAYMELSSLRSEDTATYYC | |
| [humanized VH (Homo sapiens | ARYYDDHYCLDYWGQGTTVTVSSGEGT | |
| IGHV1-46*01 (82.50%)-(IGHD)- | STGSGAIPVSLRGSGGSGGADDIVLTQSP | |
| IGHJ6*01) [8.8.12] (155-273)- | ATLSLSPGERATLSCRASQSVSSSYLAWY | |
| 25-mer linker (274-298 | QQKPGQAPRLLIYGASSRATGVPARFSGS | |
| containing MMP2 cleavage site | GSGTDFTLTISSLEPEDFATYYCLQIYNMP | |
| AIPVSLR (SEQ ID NO: 46))-V- | ITFGQGTKVEIKDKTHTCPPCPAPELLGGP | |
| KAPPA | SVFLFPPKPKDTLMISRTPEVTCVVVDVS | |
| (Homo sapiens V-KAPPA from | HEDPEVKFNWYVDGVEVHNAKTKPREE | |
| gantenerumab, CAS: 1043556- | QYNSTYRVVSVLTVLHQDWLNGKEYKC | |
| 46-2; 299-406); human IgG1 Fc | KVSNKALPAPIEKTISKAKGQPREPQVYT | |
| (407-632) | LPPSRDELTKNQVSLTCLVKGFYPSDIAV | |
| | EWESNGQPENNYKTTPPVLDSDGSFFLYS | |
| | KLTVDKSRWQQGNVFSCSVMHEALHNH | |
| | YTQKSLSLSPG | |
| Construct 9335 | EVQLVESGGGLVQAGGSLRLSCAASGRT | 170 |
| EGFR V_{HH}-CD3ϵ(V_{L}-MMP2-V_{H}) | FSSYAMGWFRQAPGKEREFVVAINWSSG | |
| Anti-human EGFR V_{HH} sequence | STYYADSVKGRFTISRDNAKNTMYLQM | |
| from 9G8 sequence from Schmitz | NSLKPEDTAVYYCAAGYQINSGNYNFKD | |
| KR et al, Structure. 2013 Jul | YEYDYWGQGTQVTVSSGGGGSGGGGSG | |
| 2;21(7):1214-24; residues 1-127)- | GGGSGGGGSGGGGSGGGGSDIVLTQSPA | |
| 30-mer hexa(tetraglycyl-seryl) | TLSLSPGERATLSCRASQSVSYMNWYQQ | |
| linker (128-157)- | KPGKAPKRWIYDTSKVASGVPARFSGSG | |
| scFv anti-CD3E | SGTDYSLTINSLEAEDAATYYCQQWSSN | |
| V-KAPPA (Mus musculus | PLTFGGGTKVEIKGEGTSTGSGAIPVSLR | |
| IGKV4-59*01 (81.70%)- | GSGGSGGADDVQLVQSGAEVKKPGASV | |
| IGKJ1*01 L124 > V (493) [5.3.9] | KVSCKASGYTFTGYYMHWVRQAPGQGL | |
| (158-263)]-25-mer linker (264- | EWMGWINPNSGGTNYAQKFQGRVTITR | |
| 288 containing MMP2 cleavage | DTSASTAYMELSSLRSEDTAVYYCARDF | |
| site AIPVSLR (SEQ ID NO: 46))- | LSGYLDYWGQGTLVTVSSDKTHTCPPCP | |
| Ig heavy chain V region (clone | APELLGGPSVFLFPPKPKDTLMISRTPEVT | |
| alpha-MUC1-1, GenBank | CVVVDVSHEDPEVKFNWYVDGVEVHNA | |
| Accession S36265; 289-406)- | KTKPREEQYNSTYRVVSVLTVLHQDWL | |
| human IgG1 Fc (407-632) | NGKEYKCKVSNKALPAPIEKTISKAKGQP | |
| | REPQVYTLPPSRDELTKNQVSLTCLVKGF | |
| | YPSDIAVEWESNGQPENNYKTTPPVLDS | |
| | DGSFFLYSKLTVDKSRWQQGNVFSCSVM | |
| | HEALHNHYTQKSLSLSPG | |
| MP058, anti-Epcam kappa light | METDTLLLWVLLLWVPGSTGELVMTQ | 171 |
| chain (signal sequence is in bold) | SPSSLTVTAGEKVTMSCKSSQSLLNSGNQ | |
| | KNYLTWYQQKPGQPPKLLIYWASTRESG | |
| | VPDRFTGSGSGTDFTLTISSVQAEDLAVY | |
| | YCQNDYSYPLTFGAGTKLEIKRTVAAPS | |
| | VFIFPPSDEQLKSGTASVVCLLNNFYPRE | |
| | AKVQWKVDNALQSGNSQESVTEQDSKD | |
| | STYSLSSTLTLSKADYEKHKVYACEVTH | |
| | QGLSSPVTKSFNRGEC | |
| MP111, anti-EGFR H-TEAC | METDTLLLWVLLLWVPGSTGQAVVTQ | 172 |
| heavy chain (signal sequence is in | EPSLTVSPGGTVTLTCRSSTGAVTTSNYA | |
| bold) | NWVQQKPGQAPRGLIGGTNKRAPWTPA | |
| | RFSGSLLGGKAALTITGAQAEDEADYYC | |
| | ALWYSNLAVFGGGTKLTVLGGGGSGPL | |
| | GVRGKAGGGGSEVQLVESGGGLVQPGG | |
| | SLRLSCAASGFTFNTYAMNWVRQAPGK | |
| | GLEWVARIRSKYNNYATYYADSVKDRF | |
| | TISRDDSKNSLYLQMNSLKTEDTAVYYC | |
| | VRHGNFGNSYVSWFAYWGQGTLVTVSS | |
| | GGGGSGGGGSQVQLVQSGAEVKKPGSS | |
| | VKVSCKASGFTFTDYKIHWVRQAPGQGL | |
| | EWMGYFNPNSGYSTYAQKFQGRVTITAD | |
| | KSTSTAYMELSSLRSEDTAVYYCARLSPG | |
| | GYYVMDAWGQGTTVTVSSASTKGPSVF | |
| | PLAPSSKSTSGGTAALGCLVKDYFPEPVT | |
| | VSWNSGALTSGVHTFPAVLQSSGLYSLSS | |
| | VVTVPSSSLGTQTYICNVNHKPSNTKVD | |
| | KKVEPKSCHHHHHH | |
| MP112, anti-EGFR L-TEAC | METDTLLLWVLLLWVPGSTGEVQLVE | 173 |
| heavy chain (signal sequence is in | SGGGLVQPGGSLRLSCAASGFTFSSYAM | |
| bold) | NWVRQAPGKGLEWVARISGSGGSTYYA | |
| | DSVKDRFTISRDDSKNSLYLQMNSLKTE | |
| | DTAVYYCVRGKGNTHKPYGYVRYFDV | |
| | WGQGTLVTVSSGGGGSGPLGVRGKAGG | |
| | GGSQAVVTQEPSLTVSPGGTVTLTCRSST | |
| | GAVTASNYANWVQQKPGQAPRGLIGGT | |
| | NKRAPWTPARFSGSLLGGKAALTITGAQ | |
| | AEDEADYYCALWYSNLWVFGGGTKLTV | |
| | LGGGGSGGGGSQVQLVQSGAEVKKPGSS | |
| | VKVSCKASGFTFTDYKIHWVRQAPGQGL | |
| | EWMGYFNPNSGYSTYAQKFQGRVTITAD | |
| | KSTSTAYMELSSLRSEDTAVYYCARLSPG | |
| | GYYVMDAWGQGTTVTVSSASTKGPSVF | |
| | PLAPSSKSTSGGTAALGCLVKDYFPEPVT | |
| | VSWNSGALTSGVHTFPAVLQSSGLYSLSS | |
| | VVTVPSSSLGTQTYICNVNHKPSNTKVD | |
| | KKVEPKSCHHHHHH | |
| MP113, anti-EGFR kappa light | METDTLLLWVLLLWVPGSTGDIQMTQ | 174 |
| chain (signal sequence is in bold) | SPSSLSASVGDRVTITCRASQGINNYLNW | |
| | YQQKPGKAPKRLIYNTNNLQTGVPSRFS | |
| | GSGSGTEFTLTISSLQPEDFATYYCLQHNS | |
| | FPTFGQGTKLEIKRTVAAPSVFIFPPSDEQ | |
| | LKSGTASVVCLLNNFYPREAKVQWKVD | |
| | NALQSGNSQESVTEQDSKDSTYSLSSTLT | |
| | LSKADYEKHKVYACEVTHQGLSSPVTKS | |
| | FNRGEC | |
| MP251, anti-Epcam H-TEAC | METDTLLLWVLLLWVPGSTGDKTHTC | 175 |
| SG₃SG₄S-linker Fc fusion heavy | PPCPAPELLGGPSVFLFPPKPKDTLMISRT | |
| chain (signal sequence is in bold) | PEVTCVVVDVSHEDPEVKFNWYVDGVE | |
| | VHNAKTKPREEQYQSTYRVVSVLTVLHQ | |
| | DWLNGKEYKCKVSNKALPAPIEKTISKA | |
| | KGQPREPQVYTLPPSRDELTKNQVSLTCL | |
| | VKGFYPSDIAVEWESNGQPENNYKTTPP | |
| | VLDSDGSFFLYSKLTVDKSRWQQGNVFS | |
| | CSVMHEALHNHYTQKSLSLSPGSGGGSG | |
| | GGGSQAVVTQEPSLTVSPGGTVTLTCRSS | |
| | TGAVTTSNYANWVQQKPGQAPRGLIGG | |
| | TNKRAPWTPARFSGSLLGGKAALTITGA | |
| | QAEDEADYYCALWYSNLAVFGGGTKLT | |
| | VLGGGGSGPLGVRGKAGGGGSEVQLVE | |
| | SGGGLVQPGGSLRLSCAASGFTFNTYAM | |
| | NWVRQAPGKGLEWVARIRSKYNNYATY | |
| | YADSVKDRFTISRDDSKNSLYLQMNSLK | |
| | TEDTAVYYCVRHGNFGNSYVSWFAYWG | |
| | QGTLVTVSSGGGGSEVQLLEQSGAELVR | |
| | PGTSVKISCKASGYAFTNYWLGWVKQRP | |
| | GHGLEWIGDIFPGSGNIHYNEKFKGKATL | |
| | TADKSSSTAYMQLSSLTFEDSAVYFCARL | |
| | RNWDEPMDWGQGTTVTVSSASTKGPS | |
| | VFPLAPSSKSTSGGTAALGCLVKDYFPEP | |
| | VTVSWNSGALTSGVHTFPAVLQSSGLYS | |
| | LSSVVTVPSSSLGTQTYICNVNHKPSNTK | |
| | VDKKVEPKSC | |
| MP252, anti-Epcam H-TEAC | METDTLLLWVLLLWVPGSTGDKTHTC | 176 |
| SG₃SG₄AG₄S linker FC fusion | PPCPAPELLGGPSVFLFPPKPKDTLMISRT | |
| heavy chain (signal sequence is in | PEVTCVVVDVSHEDPEVKFNWYVDGVE | |
| bold) | VHNAKTKPREEQYQSTYRVVSVLTVLHQ | |
| | DWLNGKEYKCKVSNKALPAPIEKTISKA | |
| | KGQPREPQVYTLPPSRDELTKNQVSLTCL | |
| | VKGFYPSDIAVEWESNGQPENNYKTTPP | |
| | VLDSDGSFFLYSKLTVDKSRWQQGNVFS | |
| | CSVMHEALHNHYTQKSLSLSPGSGGGSG | |
| | GGGAGGGGSQAVVTQEPSLTVSPGGTVT | |
| | LTCRSSTGAVTTSNYANWVQQKPGQAPR | |
| | GLIGGTNKRAPWTPARFSGSLLGGKAAL | |
| | TITGAQAEDEADYYCALWYSNLAVFGG | |
| | GTKLTVLGGGGSGPLGVRGKAGGGGSE | |
| | VQLVESGGGLVQPGGSLRLSCAASGFTF | |
| | NTYAMNWVRQAPGKGLEWVARIRSKYN | |
| | NYATYYADSVKDRFTISRDDSKNSLYLQ | |
| | MNSLKTEDTAVYYCVRHGNFGNSYVSW | |
| | FAYWGQGTLVTVSSGGGGSEVQLLEQSG | |
| | AELVRPGTSVKISCKASGYAFTNWLGW | |
| | VKQRPGHGLEWIGDIFPGSGNIHYNEKFK | |
| | GKATLTADKSSSTAYMQLSSLTFEDSAV | |
| | YFCARLRNWDEPMDWGQGTTVTVSSA | |
| | STKGPSVFPLAPSSKSTSGGTAALGCLVK | |
| | DYFPEPVTVSWNSGALTSGVHTFPAVLQ | |
| | SSGLYSLSSVVTVPSSSLGTQTYICNVNH | |
| | KPSNTKVDKKVEPKSC | |
| MP253, anti-Epcam H-TEAC | METDTLLLWVLLLWVPGSTGDKTHTC | 177 |
| SG₃SG₄AG₄S linker Fc fusion | PPCPAPELLGGPSVFLFPPKPKDTLMISRT | |
| heavy chain (signal sequence is in | PEVTCVVVDVSHEDPEVKFNWYVDGVE | |
| bold) | VHNAKTKPREEQYQSTYRVVSVLTVLHQ | |
| | DWLNGKEYKCKVSNKALPAPIEKTISKA | |
| | KGQPREPQVYTLPPSRDELTKNQVSLTCL | |
| | VKGFYPSDIAVEWESNGQPENNYKTTPP | |
| | VLDSDGSFFLYSKLTVDKSRWQQGNVFS | |
| | CSVMHEALHNHYTQKSLSLSPGSGGGSG | |
| | GGGAGGGGSQAVVTQEPSLTVSPGGTVT | |
| | LTCRSSTGAVTTSNYANWVQQKPGQAPR | |
| | GLIGGTNKRAPWTPARFSGSLLGGKAAL | |
| | TITGAQAEDEADYYCALWYSNLAVFGG | |
| | GTKLTVLGGPLGVRGKAGEVQLVESGG | |
| | GLVQPGGSLRLSCAASGFTFNTYAMNW | |
| | VRQAPGKGLEWVARIRSKYNNYATYYA | |
| | DSVKDRFTISRDDSKNSLYLQMNSLKTE | |
| | DTAVYYCVRHGNFGNSYVSWFAWGQ | |
| | GTLVTVSSGGGGSEVQLLEQSGAELVRP | |
| | GTSVKISCKASGYAFTNWLGWVKQRP | |
| | GHGLEWIGDIFPGSGNIHYNEKFKGKATL | |
| | TADKSSSTAYMQLSSLTFEDSAVYFCARL | |
| | RNWDEPMDWGQGTTVTVSSASTKGPS | |
| | VFPLAPSSKSTSGGTAALGCLVKDYFPEP | |
| | VTVSWNSGALTSGVHTFPAVLQSSGLYS | |
| | LSSVVTVPSSSLGTQTYICNVNHKPSNTK | |
| | VDKKVEPKSC | |
| MP254, anti-Epcam H-TEAC | METDTLLLWVLLLWVPGSTGDKTHTC | 178 |
| SG₃SG₄AG₄S linker Fc fusion | PPCPAPELLGGPSVFLFPPKPKDTLMISRT | |
| heavy chain (signal sequence is in | PEVTCVVVDVSHEDPEVKFNWYVDGVE | |
| bold) | VHNAKTKPREEQYQSTYRVVSVLTVLHQ | |
| | DWLNGKEYKCKVSNKALPAPIEKTISKA | |
| | KGQPREPQVYTLPPSRDELTKNQVSLTCL | |
| | VKGFYPSDIAVEWESNGQPENNYKTTPP | |
| | VLDSDGSFFLYSKLTVDKSRWQQGNVFS | |
| | CSVMHEALHNHYTQKSLSLSPGSGGGSG | |
| | GGGAGGGGSQAVVTQEPSLTVSPGGTVT | |
| | LTCRSSTGAVTTSNYANWVQQKPGQAPR | |
| | GLIGGTNKRAPWTPARFSGSLLGGKAAL | |
| | TITGAQAEDEADYYCALWYSNLAVFGG | |
| | GTKLTVLGGPLGVRGGEVQLVESGGGLV | |
| | QPGGSLRLSCAASGFTFNTYAMNWVRQ | |
| | APGKGLEWVARIRSKYNNYATYYADSV | |
| | KDRFTISRDDSKNSLYLQMNSLKTEDTA | |
| | VYYCVRHGNFGNSYVSWFAYWGQGTL | |
| | VTVSSGGGGSEVQLLEQSGAELVRPGTS | |
| | VKISCKASGYAFTNYWLGWVKQRPGHG | |
| | LEWIGDIFPGSGNIHYNEKFKGKATLTAD | |
| | KSSSTAYMQLSSLTFEDSAVYFCARLRN | |
| | WDEPMDWGQGTTVTVSSASTKGPSVF | |
| | PLAPSSKSTSGGTAALGCLVKDYFPEPVT | |
| | VSWNSGALTSGVHTFPAVLQSSGLYSLSS | |
| | VVTVPSSSLGTQTYICNVNHKPSNTKVD | |
| | KKVEPKSC | |
| MP268, anti-EGFR H-TEAC | METDTLLLWVLLLWVPGSTGDKTHTC | 179 |
| SG₃SG₄S-linker Fc fusion heavy | PPCPAPELLGGPSVFLFPPKPKDTLMISRT | |
| chain (signal sequence is in bold) | PEVTCVVVDVSHEDPEVKFNWYVDGVE | |
| | VHNAKTKPREEQYQSTYRVVSVLTVLHQ | |
| | DWLNGKEYKCKVSNKALPAPIEKTISKA | |
| | KGQPREPQVYTLPPSRDELTKNQVSLTCL | |
| | VKGFYPSDIAVEWESNGQPENNYKTTPP | |
| | VLDSDGSFFLYSKLTVDKSRWQQGNVFS | |
| | CSVMHEALHNHYTQKSLSLSPGSGGGSG | |
| | GGGSQAVVTQEPSLTVSPGGTVTLTCRSS | |
| | TGAVTTSNYANWVQQKPGQAPRGLIGG | |
| | TNKRAPWTPARFSGSLLGGKAALTITGA | |
| | QAEDEADYYCALWYSNLAVFGGGTKLT | |
| | VLGGGGSGPLGVRGKAGGGGSEVQLVE | |
| | SGGGLVQPGGSLRLSCAASGFTFNTYAM | |
| | NWVRQAPGKGLEWVARIRSKYNNYATY | |
| | YADSVKDRFTISRDDSKNSLYLQMNSLK | |
| | TEDTAVYYCVRHGNFGNSYVSWFAYWG | |
| | QGTLVTVSSGGGGSGGGGSQVQLVQSG | |
| | AEVKKPGSSVKVSCKASGFTFTDYKIHW | |
| | VRQAPGQGLEWMGYFNPNSGYSTYAQK | |
| | FQGRVTITADKSTSTAYMELSSLRSEDTA | |
| | VYYCARLSPGGYYVMDAWGQGTTVTVS | |
| | SASTKGPSVFPLAPSSKSTSGGTAALGCL | |
| | VKDYFPEPVTVSWNSGALTSGVHTFPAV | |
| | LQSSGLYSLSSVVTVPSSSLGTQTYICNV | |
| | NHKPSNTKVDKKVEPKSCHHHHHH | |
| MP269, anti-EGFR L-TEAC | METDTLLLWVLLLWVPGSTGDKTHTC | 180 |
| SG₃SG₄S-linker Fc Fusion heavy | PPCPAPELLGGPSVFLFPPKPKDTLMISRT | |
| chain (signal sequence is in bold) | PEVTCVVVDVSHEDPEVKFNWYVDGVE | |
| | VHNAKTKPREEQYQSTYRVVSVLTVLHQ | |
| | DWLNGKEYKCKVSNKALPAPIEKTISKA | |
| | KGQPREPQVYTLPPSRDELTKNQVSLTCL | |
| | VKGFYPSDIAVEWESNGQPENNYKTTPP | |
| | VLDSDGSFFLYSKLTVDKSRWQQGNVFS | |
| | CSVMHEALHNHYTQKSLSLSPGSGGGSG | |
| | GGGSEVQLVESGGGLVQPGGSLRLSCAA | |
| | SGFTFSSYAMNWVRQAPGKGLEWVARIS | |
| | GSGGSTYYADSVKDRFTISRDDSKNSLYL | |
| | QMNSLKTEDTAVYYCVRGKGNTHKPYG | |
| | YVRYFDVWGQGTLVTVSSGGGGSGPLG | |
| | VRGKAGGGGSQAVVTQEPSLTVSPGGTV | |
| | TLTCRSSTGAVTASNYANWVQQKPGQA | |
| | PRGLIGGTNKRAPWTPARFSGSLLGGKA | |
| | ALTITGAQAEDEADYYCALWYSNLWVF | |
| | GGGTKLTVLGGGGSGGGGSQVQLVQSG | |
| | AEVKKPGSSVKVSCKASGFTFTDYKIHW | |
| | VRQAPGQGLEWMGYFNPNSGYSTYAQK | |
| | FQGRVTITADKSTSTAYMELSSLRSEDTA | |
| | VYYCARLSPGGYYVMDAWGQGTTVTVS | |
| | SASTKGPSVFPLAPSSKSTSGGTAALGCL | |
| | VKDYFPEPVTVSWNSGALTSGVHTFPAV | |
| | LQSSGLYSLSSVVTVPSSSLGTQTYICNV | |
| | NHKPSNTKVDKKVEPKSCHHHHHH | |
| 19-mer protease linker | GGGGSGPLGVRGKAGGGGS | 181 |
| 11-mer protease linker | GGPLGVRGKAG | 182 |
| 9-mer protease linker | GGPLGVRGG | 183 |
| SG₃SG₄AG₄S linker | SGGGSGGGGAGGGGS | 184 |
| SG₃SG₄S linker | SGGGSGGGGS | 185 |
| Anti-EpCAM-CD3 VH | ELVMTQSPSSLTVTAGEKVTMSCKSSQSL | 186 |
| ATTAC/TEAC component with | LNSGNQKNYLTWYQQKPGQPPKLLIYW | |
| half-life extension | ASTRESGVPDRFTGSGSGTDFTLTISSVQA | |
| (Anti-EpCAM scFv with 3xG4S | EDLAVYYCQNDYSYPLTFGAGTKLEIKG | |
| linker between VH-VL-1xG4S | GGGSGGGGSGGGGSEVQLLEQSGAELV | |
| connector-anti-CD3e VH | RPGTSVKISCKASGYAFTNYWLGWVKQ | |
| (20G6)-MMP2 cleavage | RPGHGLEWIGDIFPGSGNIHYNEKFKGKA | |
| sequence-Ig VL domain- | TLTADKSSSTAYMQLSSLTFEDSAVYFCA | |
| human IgG1 Fc) | RLRNWDEPMDYWGQGTTVTVSSGGGG | |
| | SQVQLVESGGGVVQPGRSLRLSCAASGF | |
| | TFTKAWMHWVRQAPGKQLEWVAQIKD | |
| | KSNSYATYYADSVKGRFTISRDDSKNTL | |
| | YLQMNSLRAEDTAVYYCRGVYYALSPF | |
| | DYWGQGTLVTVSSGEGTSTGSGAIPVSL | |
| | RGSGGSGGADDIVMTQTPLSLSVTPGQP | |
| | ASISCKSSQSIVHSSGNTYLSWYLQKPGQ | |
| | SPQLLIYKVSNRFSGVPDRFSGSGSGTDFT | |
| | LKISRVEAEDVGVYYCGQGSHVGPTFGS | |
| | GTKVEIK [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] | |
| Anti-EpCAM-CD3 VH | ELVMTQSPSSLTVTAGEKVTMSCKSSQSL | 187 |
| ATTAC/TEAC component with | LNSGNQKNYLTWYQQKPGQPPKLLIYW | |
| half-life extension moiety | ASTRESGVPDRFTGSGSGTDFTLTISSVQA | |
| Anti-EpCAM scFv (with 3xG4S | EDLAVYYCQNDYSYPLTFGAGTKLEIKG | |
| linker between VH-VL)-1xG4S | GGGSGGGGSGGGGSEVQLLEQSGAELV | |
| connector-20G6 VH-MMP2 | RPGTSVKISCKASGYAFTNYWLGWVKQ | |
| cleavage tag-inert binding | RPGHGLEWIGDIFPGSGNIHYNEKFKGKA | |
| partner (VL domain)-2xG4S | TLTADKSSSTAYMQLSSLTFEDSAVYFCA | |
| linker-IgG4 CH domains-His | RLRNWDEPMDYWGQGTTVTVSSGGGG | |
| tag-Stop | SQVQLVESGGGVVQPGRSLRLSCAASGF | |
| | TFTKAWMHWVRQAPGKQLEWVAQIKD | |
| | KSNSYATYYADSVKGRFTISRDDSKNTL | |
| | YLQMNSLRAEDTAVYYCRGVYYALSPF | |
| | DYWGQGTLVTVSSGEGTSTGSGAIPVSL | |
| | RGSGGSGGADDIVMTQTPLSLSVTPGQP | |
| | ASISCKSSQSIVHSSGNTYLSWYLQKPGQ | |
| | SPQLLIYKVSNRFSGVPDRFSGSGSGTDFT | |
| | LKISRVEAEDVGVYYCGQGSHVGPTFGS | |
| | GTKVEIKGGGSGGGSESKYGPPCPPCPA | |
| | PEFLGGPSVFLEPPKPKDTLMISRTPEVTC | |
| | VVVDVSQEDPEVQFNWYVDGVEVHNAK | |
| | TKPREEQFNSTYRVVSVLTVLHQDWLNG | |
| | KEYKCKVSNKGLPSSIEKTISKAKGQPRE | |
| | PQVYTLPPSQEEMTKNQVSLTCLVKGFY | |
| | PSDIAVEWESNGQPENNYKTTPPVLDSD | |
| | GSFFLYSRLTVDKSRWQEGNVFSCSVMH | |
| | EALHNHYTQKSLSLSLGKGSHHHHHH | |
| Anti-EpCAM-CD3 VL | EVQLLEQSGAELVRPGTSVKISCKASGYA | 188 |
| ATTAC/TEAC component with | FTNYWLGWVKQRPGHGLEWIGDIFPGSG | |
| half-life extension moiety | NIHYNEKFKGKATLTADKSSSTAYMQLS | |
| (Anti-EpCAM scFv with 3xG4S | SLTFEDSAVYFCARLRNWDEPMDYWGQ | |
| linker between VH-VL-1xG4S | GTTVTVSSGGGGSGGGGSGGGGSELV | |
| connector-anti-CD3e VL | MTQSPSSLTVTAGEKVTMSCKSSQSLLNS | |
| (20G6)-MMP2 cleavage | GNQKNYLTWYQQKPGQPPKLLIYWAST | |
| sequence-Ig VH domain- | RESGVPDRFTGSGSGTDFTLTISSVQAED | |
| human IgG1 Fc) | LAVYYCQNDYSYPLTFGAGTKLEIKGGG | |
| | GSDIVMTQTPLSLSVTPGQPASISCKSSQS | |
| | LVHNNGNTYLSWYLQKPGQSPQSLIYKV | |
| | SNRFSGVPDRFSGSGSGTDFTLKISRVEAE | |
| | DVGVYYCGQGTQYPFTFGSGTKVEIKGE | |
| | GTSTGSGAIPVSLRGSGGSGGADQVQLV | |
| | ESGGGVVQPGRSLRLSCAASGFTFSSYG | |
| | IVIHWVRQAPGKQLEWVAQISFDGSNKYY | |
| | ADSVKGRFTISRDDSKNTLYLQMNSLRA | |
| | EDTAVYYCASERGHYYDSSAFDYWGQG | |
| | TLVTVSS [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] [symbol] [symbol] | |
| | [symbol] | |
| Anti-EpCAM-CD3 VL | EVQLLEQSGAELVRPGTSVKISCKASGYA | 189 |
| ATTAC/TEAC component with | FTNYWLGWVKQRPGHGLEWIGDIFPGSG | |
| half-life extension moiety | NIHYNEKFKGKATLTADKSSSTAYMQLS | |
| Anti-EpCAM scFv (with 3xG4S | SLTFEDSAVYFCARLRNWDEPMDYWGQ | |
| linker between VL-VH)-1xG4S | GTTVTVSSGGGGSGGGGSGGGGSELV | |
| connector-20G6 VL-MMP2 | MTQSPSSLTVTAGEKVTMSCKSSQSLLNS | |
| cleavage tag-inert binding | GNQKNYLTWYQQKPGQPPKLLIYWAST | |
| partner (VH domain)-2xG4S | RESGVPDRFTGSGSGTDFTLTISSVQAED | |
| linker-IgG4 CH domains- | LAVYYCQNDYSYPLTFGAGTKLEIKGGG | |
| EPEA tag-Stop | GSDIVMTQTPLSLSVTPGQPASISCKSSQS | |
| | LVHNNGNTYLSWYLQKPGQSPQSLIYKV | |
| | SNRFSGVPDRFSGSGSGTDFTLKISRVEAE | |
| | DVGVYYCGQGTQYPFTFGSGTKVEIKGE | |
| | GTSTGSGAIPVSLRGSGGSGGADQVQLV | |
| | ESGGGVVQPGRSLRLSCAASGFTFSSYG | |
| | IVIHWVRQAPGKQLEWVAQISFDGSNKYY | |
| | ADSVKGRFTISRDDSKNTLYLQMNSLRA | |
| | EDTAVYYCASERGHYYDSSAFDYWGQG | |
| | TLVTVSSGGGSGGGSESKYGPPCPPCPAP | |
| | EFLGGPSVFLFPPKPKDTLMISRTPEVTCV | |
| | VVDVSQEDPEVQFNWYVDGVEVHNAKT | |
| | KPREEQFNSTYRVVSVLTVLHQDWLNGK | |
| | EYKCKVSNKGLPSSIEKTISKAKGQPREP | |
| | QVYTLPPSQEEMTKNQVSLTCLVKGFYP | |
| | SDIAVEWESNGQPENNYKTTPPVLDSDG | |
| | SFELYSRLTVDKSRWQEGNVESCSVMHE | |
| | ALHNHYTQKSLSLSLGKGEPEA | |
| Anti-EpCAM-CD3 scFv (20G6) | ELVMTQSPSSLTVTAGEKVTMSCKSSQSL | 190 |
| BiTE construct (anti-EpCAM | LNSGNQKNYLTWYQQKPGQPPKLLIYW | |
| scFv with 3xG4S linker between | ASTRESGVPDRFTGSGSGTDFTLTISSVQA | |
| VH and VL-1xG4S connector- | EDLAVYYCQNDYSYPLTFGAGTKLEIKG | |
| anti-CD3 scFv with linker | GGGSGGGGSGGGGSEVQLLEQSGAELV | |
| between VH and VL-) | RPGTSVKISCKASGYAFTNYWLGWVKQ | |
| | RPGHGLEWIGDIFPGSGNIHYNEKFKGKA | |
| | TLTADKSSSTAYMQLSSLTFEDSAVYFCA | |
| | RLRNWDEPMDYWGQGTTVTVSSGGGG | |
| | SDIVMTQTPLSLSVTPGQPASISCKSSQSL | |
| | VHNNGNTYLSWYLQKPGQSPQSLIYKVS | |
| | NRFSGVPDRFSGSGSGTDFTLKISRVEAE | |
| | DVGVYYCGQGTQYPFTEGSGTKVEIKGE | |
| | GTSTGSGGSGGSGGADQVQLVESGGGV | |
| | VQPGRSLRLSCAASGFTFTKAWMHWVR | |
| | QAPGKQLEWVAQIKDKSNSYATYYADS | |
| | VKGRFTISRDDSKNTLYLQMNSLRAEDT | |
| | AVYYCRGVYYALSPFDYWGQGTLVTVS | |
| | S | |
| Anti-CD8-CD3 VL ATTAC | QVQLQESGGGLVQPGGSLRLSCAASGFT | 191 |
| component with half-life | FDDYAMSWVRQVPGKGLEWVSTINWNG | |
| extension moiety | GSAEYAEPVKGRFTISRDNAKNTVYLQM | |
| (Anti-CD8 VHH-1xG4S | NSLKLEDTAVYYCAKDADLVWYNLRTG | |
| connector-anti-CD3e VL | QGTQVTVSSAAAYPYDVPDYGSGGGGS | |
| (20G6)-MMP2 cleavage | DIVMTQTPLSLSVTPGQPASISCKSSQSLV | |
| sequence-Ig VH domain- | HNNGNTYLSWYLQKPGQSPQSLIYKVSN | |
| human IgG1 Fc) | RFSGVPDRFSGSGSGTDFTLKISRVEAED | |
| (CD8 targeting VHH domain | VGVYYCGQGTQYPFTEGSGTKVEIKGEG | |
| based upon WO_2017_134306 | TSTGSGAIPVSLRGSGGSGGADQVQLVE | |
| SEQ ID NO: 20) | SGGGVVQPGRSLRLSCAASGFTFSSYGM | |
| | HWVRQAPGKQLEWVAQISFDGSNKYYA | |
| | DSVKGRFTISRDDSKNTLYLQMNSLRAE | |
| | DTAVYYCASERGHYYDSSAFDYWGQGT | |
| | LVTVSS [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] | |
| Anti-CD8-CD3 VL ATTAC | QVQLQESGGGLVQPGGSLRLSCAASGFT | 192 |
| component with half-life | FDDYAMSWVRQVPGKGLEWVSTINWNG | |
| extension moiety | GSAEYAEPVKGRFTISRDNAKNTVYLQM | |
| Anti-CD8-CD3 VL ATTAC | NSLKLEDTAVYYCAKDADLVWYNLRTG | |
| component (Anti-CD8 VHH- | QGTQVTVSSAAAYPYDVPDYGSGGGGS | |
| 1xG4S connector-anti-CD3e | DIVMTQTPLSLSVTPGQPASISCKSSQSLV | |
| VL (20G6)-MMP2 cleavage | HNNGNTYLSWYLQKPGQSPQSLIYKVSN | |
| sequence-Ig VH domain- | RFSGVPDRFSGSGSGTDFTLKISRVEAED | |
| 2xG4S connector-IgG4 CH | VGVYYCGQGTQYPFTEGSGTKVEIKGEG | |
| domains-His tag) | TSTGSGAIPVSLRGSGGSGGADQVQLVE | |
| (CD8 targeting VHH domain | SGGGVVQPGRSLRLSCAASGFTFSSYGM | |
| based upon WO_2017_134306 | HWVRQAPGKQLEWVAQISFDGSNKYYA | |
| SEQ ID NO: 20) | DSVKGRFTISRDDSKNTLYLQMNSLRAE | |
| | DTAVYYCASERGHYYDSSAFDWGQGT | |
| | LVTVSSGGGSGGGSESKYGPPCPPCPAPE | |
| | FLGGPSVFLEPPKPKDTLMISRTPEVTCVV | |
| | VDVSQEDPEVQFNWYVDGVEVHNAKTK | |
| | PREEQFNSTYRVVSVLTVLHQDWLNGKE | |
| | YKCKVSNKGLPSSIEKTISKAKGQPREPQ | |
| | VYTLPPSQEEMTKNQVSLTCLVKGFYPS | |
| | DIAVEWESNGQPENNYKTTPPVLDSDGS | |
| | FFLYSRLTVDKSRWQEGNVFSCSVMHEA | |
| | LHNHYTQKSLSLSLGKGHHHHHH | |
| Anti-CD8-CD3 VL ATTAC | EVQLQQSGAELVKPGASVKLSCTASGFNI | 193 |
| component with half-life | KDTYIHFVRQRPEQGLEWIGRIDPANDNT | |
| extension moiety (Anti-CD8 scFv | LYASKFQGKATITADTSSNTAYMHLCSL | |
| with linker between VL-VH- | TSGDTAVYYCGRGYGYYVFDHWGQGTT | |
| 1xG4S connector-anti-CD3e | LTVSSGGGGSGGGGSGGGGSDVQINQS | |
| VL (20G6)-MMP2 cleavage | PSFLAASPGETITINCRTSRSISQYLAWYQ | |
| sequence-Ig VH domain- | EKPGKTNKLLIYSGSTLQSGIPSRFSGSGS | |
| human IgG1 Fc) | GTDFTLTISGLEPEDFAMYYCQQHNENPL | |
| (CD8 targeting scFv domain | TFGAGTKLELKGGGGSDIVMTQTPLSLS | |
| based upon OKT8 antibody) | VTPGQPASISCKSSQSLVHNNGNTYLSW | |
| | YLQKPGQSPQSLIYKVSNRFSGVPDRFSG | |
| | SGSGTDFTLKISRVEAEDVGVYYCGQGT | |
| | QYPFTEGSGTKVEIKGEGTSTGSGAIPVSL | |
| | RGSGGSGGADQVQLVESGGGVVQPGRS | |
| | LRLSCAASGFTFSSYGMHWVRQAPGKQL | |
| | EWVAQISEDGSNKYYADSVKGRETISRD | |
| | DSKNTLYLQMNSLRAEDTAVYYCASER | |
| | GHYYDSSAFDWGQGTLVTVSS [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| Anti-CD8-CD3 VL ATTAC | EVQLQQSGAELVKPGASVKLSCTASGFNI | 194 |
| component with half-life | KDTYIHFVRQRPEQGLEWIGRIDPANDNT | |
| extension moiety (Anti-CD8 | LYASKFQGKATITADTSSNTAYMHLCSL | |
| scFv with linker between VL-VH- | TSGDTAVYYCGRGYGYYVFDHWGQGTT | |
| 1xG4S connector-anti-CD3e | LTVSSGGGGSGGGGSGGGGSDVQINQS | |
| VL (20G6)-MMP2 cleavage | PSFLAASPGETITINCRTSRSISQYLAWYQ | |
| sequence-Ig VH domain- | EKPGKTNKLLIYSGSTLQSGIPSRFSGSGS | |
| 2xG4S connector-IgG4 CH | GTDFTLTISGLEPEDFAMYYCQQHNENPL | |
| domains-His tag) | TFGAGTKLELKGGGGSDIVMTQTPLSLS | |
| (CD8 targeting scFv domain | VTPGQPASISCKSSQSLVHNNGNTYLSW | |
| based upon OKT8 antibody) | YLQKPGQSPQSLIYKVSNRFSGVPDRFSG | |
| | SGSGTDFTLKISRVEAEDVGVYYCGQGT | |
| | QYPFTFGSGTKVEIKGEGTSTGSGAIPVSL | |
| | RGSGGSGGADQVQLVESGGGVVQPGRS | |
| | LRLSCAASGFTFSSYGMHWVRQAPGKQL | |
| | EWVAQISFDGSNKYYADSVKGRFTISRD | |
| | DSKNTLYLQMNSLRAEDTAVYYCASER | |
| | GHYYDSSAFDYWGQGTLVTVSSGGGSG | |
| | GGSESKYGPPCPPCPAPEFLGGPSVFLFPP | |
| | KPKDTLMISRTPEVTCVVVDVSQEDPEV | |
| | QFNWYVDGVEVHNAKTKPREEQFNSTY | |
| | RVVSVLTVLHQDWLNGKEYKCKVSNKG | |
| | LPSSIEKTISKAKGQPREPQVYTLPPSQEE | |
| | MTKNQVSLTCLVKGFYPSDIAVEWESNG | |
| | QPENNYKTTPPVLDSDGSFFLYSRLTVDK | |
| | SRWQEGNVFSCSVMHEALHNHYTQKSL | |
| | SLSLGKG [symbol] | |
| Anti-CD4-CD3 VL ATTAC | QVQLQQSGPEVVKPGASVKMSCKASGY | 195 |
| component with half-life | TFTSYVIEWVRQKPGQGLDWIGYINPYN | |
| extension moiety (Anti-CD4 scFv | DGTDYDEKFKGKATLTSDTSTSTAYMEL | |
| with linker between VL-VH- | SSLRSEDTAVYYCAREKDNYATGAWFA | |
| 1xG4 S connector-anti-CD3e | YWGQGTLVTVSSGGGGSGGGGSGGGG | |
| VL (20G6)-MMP2 cleavage | SDIVMTQSPDSLAVSLGERVTMNCKSSQS | |
| sequence-Ig VH domain- | LLYSTNQKNYLAWYQQKPGQSPKLLIY | |
| human IgG1 Fc) | WASTRESGVPDRFSGSGSGTDFTLTISSV | |
| (CD4 targeting scFv domain | QAEDVAVYYCQQYYSYRTFGGGTKLEIK | |
| based upon Ibalizumab antibody) | GGGGSDIVMTQTPLSLSVTPGQPASISCK | |
| | SSQSLVHNNGNTYLSWYLQKPGQSPQSLI | |
| | YKVSNRFSGVPDRFSGSGSGTDFTLKISR | |
| | VEAEDVGVYYCGQGTQYPFTFGSGTKVE | |
| | IKGEGTSTGSGAIPVSLRGSGGSGGADQ | |
| | VQLVESGGGVVQPGRSLRLSCAASGFTFS | |
| | SYGIVIEIWVRQAPGKQLEWVAQISFDGSN | |
| | KYYADSVKGRFTISRDDSKNTLYLQMNS | |
| | LRAEDTAVYYCASERGHYYDSSAFDYW | |
| | GQGTLVTVSS [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] | |
| Anti-CD4-CD3 VL ATTAC | QVQLQQSGPEVVKPGASVKMSCKASGY | 196 |
| component with half-life | TFTSYVIHWVRQKPGQGLDWIGYINPYN | |
| extension moiety | DGTDYDEKFKGKATLTSDTSTSTAYMEL | |
| Anti-CD4-CD3 VL ATTAC | SSLRSEDTAVYYCAREKDNYATGAWFA | |
| component (Anti-CD4 scFv with | YWGQGTLVTVSSGGGGSGGGGSGGGG | |
| linker between VL-VH-1xG4S | SDIVMTQSPDSLAVSLGERVTMNCKSSQS | |
| connector-anti-CD3e VL | LLYSTNQKNYLAWYQQKPGQSPKLLIY | |
| (20G6)-MMP2 cleavage | WASTRESGVPDRFSGSGSGTDFTLTISSV | |
| sequence-Ig VH domain- | QAEDVAVYYCQQYYSYRTFGGGTKLEIK | |
| 2xG4S connector-IgG4 CH | GGGGSDIVMTQTPLSLSVTPGQPASISCK | |
| domains-His tag) | SSQSLVHNNGNTYLSWYLQKPGQSPQSLI | |
| (CD4 targeting scFv domain | YKVSNRFSGVPDRFSGSGSGTDFTLKISR | |
| based upon Ibalizumab antibody) | VEAEDVGVYYCGQGTQYPFTFGSGTKVE | |
| | IKGEGTSTGSGAIPVSLRGSGGSGGADQ | |
| | VQLVESGGGVVQPGRSLRLSCAASGFTFS | |
| | SYGMHWVRQAPGKQLEWVAQISFDGSN | |
| | KYYADSVKGRFTISRDDSKNTLYLQMNS | |
| | LRAEDTAVYYCASERGHYYDSSAFDYW | |
| | GQGTLVTVSSGGGSGGGSESKYGPPCPP | |
| | CPAPEFLGGPSVFLFPPKPKDTLMISRTPE | |
| | VTCVVVDVSQEDPEVQFNWYVDGVEVH | |
| | NAKTKPREEQFNSTYRVVSVLTVLHQDW | |
| | LNGKEYKCKVSNKGLPSSIEKTISKAKGQ | |
| | PREPQVYTLPPSQEEMTKNQVSLTCLVK | |
| | GFYPSDIAVEWESNGQPENNYKTTPPVL | |
| | DSDGSFFLYSRLTVDKSRWQEGNVFSCS | |
| | VMHEALHNHYTQKSLSLSLGKG [symbol] | |
| | [symbol] | |
| Anti-CD8-CD3 VL ATTAC | QVQLQESGGGLVQAGGSLRLSCAASGFT | 197 |
| component | FDDYAIGWFRQAPGKEREGVSCIRVSDG | |
| (Anti-CD8 VHH-6xG4S | STYYADPVKGRFTISSDNAKNTVYLQMN | |
| connector-anti-CD3e VL | SLKPEDAAVYYCAAGSLYTCVQSIVWPA | |
| (20G6)-Enterokinase cleavage | RPYYDMDYWGKGTQVTVSSAAAYPYD | |
| sequence-Ig VH domain- | VPDYGSGGGGSGGGGSGGGGSGGGGS | |
| human IgG1 Fc) | GGGGSGGGGSDIVMTQTPLSLSVTPGQP | |
| (CD8 targeting VHH domain | ASISCKSSQSLVHNNGNTYLSWYLQKPG | |
| based upon WO_2017_134306 | QSPQSLIYKVSNRFSGVPDRFSGSGSGTD | |
| SEQ ID NO: 21) | FTLKISRVEAEDVGVYYCGQGTQYPFTF | |
| | GSGTKVEIKGEGTSTGSGGGGGSGGGGS | |
| | DDDDKGGGGSGGGGSGSGGSGGADQVQ | |
| | LVQSGAEVKKPGASVKVSCKASGYTFTS | |
| | YYIHWVRQAPGQGLEWIGCIYPGNVNTN | |
| | YNEKFKDRATLTVDTSISTAYMELSRLRS | |
| | DDTAVYFCTRSHYGLDWNFDVWGQGTT | |
| | VTVSSGS [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] [symbol] | |
| | [symbol] [symbol] [symbol] | |
| | [symbol] | |
| Anti-CD8-CD3 VL ATTAC | QVQLQESGGGLVQAGGSLRLSCAASGFT | 198 |
| component | FDDYAIGWFRQAPGKEREGVSCIRVSDG | |
| (Anti-CD8 VHH-6xG4S | STYYADPVKGRFTISSDNAKNTVYLQMN | |
| connector-anti-CD3e VL | SLKPEDAAVYYCAAGSLYTCVQSIVWPA | |
| (20G6)-Enterokinase cleavage | RPYYDMDYWGKGTQVTVSSAAAYPYD | |
| sequence-Ig VH domain- | VPDYGSGGGGSGGGGSGGGGSGGGGS | |
| 2xG4S connector-IgG4 CH | GGGGSGGGGSDIVMTQTPLSLSVTPGQP | |
| domains-His tag) | ASISCKSSQSLVHNNGNTYLSWYLQKPG | |
| (CD8 targeting VHH domain | QSPQSLIYKVSNRFSGVPDRFSGSGSGTD | |
| based upon WO_2017_134306 | FTLKISRVEAEDVGVYYCGQGTQYPFTF | |
| SEQ ID NO: 21) | GSGTKVEIKGEGTSTGSGGGGGSGGGGS | |
| | DDDDKGGGGSGGGGSGSGGSGGADQVQ | |
| | LVQSGAEVKKPGASVKVSCKASGYTFTS | |
| | YYIHWVRQAPGQGLEWIGCIYPGNVNTN | |
| | YNEKFKDRATLTVDTSISTAYMELSRLRS | |
| | DDTAVYFCTRSHYGLDWNFDVWGQGTT | |
| | VTVSSGGGSGGGSESKYGPPCPPCPAPEF | |
| | LGGPSVFLFPPKPKDTLMISRTPEVTCVV | |
| | VDVSQEDPEVQFNWYVDGVEVHNAKTK | |
| | PREEQFNSTYRVVSVLTVLHQDWLNGKE | |
| | YKCKVSNKGLPSSIEKTISKAKGQPREPQ | |
| | VYTLPPSQEEMTKNQVSLTCLVKGFYPS | |
| | DIAVEWESNGQPENNYKTTPPVLDSDGS | |
| | FFLYSRLTVDKSRWQEGNVFSCSVMHEA | |
| | LHNHYTQKSLSLSLGKGSHHHHHH | |
| Anti-CD33 TEAC component | QIVLTQSPAIIVISASPGEKVTITCSASSSISY | 199 |
| with half-life extension moiety | MHWFQQKPGTSPKLWIYTTSNLASGVPA | |
| CD33 20G6-VH | RFSGSGSGTSYSLTISRMEAEDAATYYCH | |
| Anti-CD33 scFv (with 3xG4S | QRSTYPLTFGSGTKLELKGGGGSGGGGS | |
| linker between VH-VL)-1xG4S | SGGGSQVQLQQSGAELAKPGASVKMSC | |
| connector120G6 VH1MMP2 | KASGYTFTSYRMHWVKQRPGQGLEWIG | |
| cleavage tag1inert binding | YINPSTGYTEYNQKFKDKATLTADKSSST | |
| partner (VL domain)12xG4S | AYMQLSSLTFEDSAVYYCARGGGVFDY | |
| linker1IgG4 CH domains1His | WGQGTTLTVSSGGGGSQVQLVESGGGV | |
| tag1Stop | VQPGRSLRLSCAASGFTFTKAWMHWVR | |
| | QAPGKQLEWVAQIKDKSNSYATYYADS | |
| | VKGRFTISRDDSKNTLYLQMNSLRAEDT | |
| | AVYYCRGVYYALSPFDYWGQGTLVTVS | |
| | SGEGTSTGSGAIPVSLRGSGGSGGADDIV | |
| | MTQTPLSLSVTPGQPASISCKSSQSIVHSS | |
| | GNTYLSWYLQKPGQSPQLLIYKVSNRFS | |
| | GVPDRFSGSGSGTDFTLKISRVEAEDVGV | |
| | YYCGQGSHVGPTFGSGTKVEIKGGGSGG | |
| | GSESKYGPPCPPCPAPEFLGGPSVFLFPPK | |
| | PKDTLMISRTPEVTCVVVDVSQEDPEVQF | |
| | NWYVDGVEVHNAKTKPREEQFNSTYRV | |
| | VSVLTVLHQDWLNGKEYKCKVSNKGLP | |
| | SSIEKTISKAKGQPREPQVYTLPPSQEEMT | |
| | KNQVSLTCLVKGFYPSDIAVEWESNGQP | |
| | ENNYKTTPPVLDSDGSFFLYSRLTVDKSR | |
| | WQEGNVFSCSVMHEALHNHYTQKSLSLS | |
| | LGKGSHHHHHH | |
| Anti-CD123 TEAC component | EVQLVKSGGGLVQAGGSLRLSCAASGIT | 200 |
| with half-life extension moiety | SKINDMGWYRQTPGNYREWVASITATGT | |
| CD123 20G6-VL | TNYRDSVKGRFTISRDNAKSTVYLQMNS | |
| Anti-CD123 VHH domain- | LKPEDTTVYYCNTFPPISNFWGQGTLVTV | |
| 1xG4S connector-20G6 VL- | SSGGGGSDIVMTQTPLSLSVTPGQPASIS | |
| MMP2 cleavage tag-inert | CKSSQSLVHNNGNTYLSWYLQKPGQSPQ | |
| binding partner (VH domain)- | SLIYKVSNRFSGVPDRFSGSGSGTDFTLKI | |
| 2xG4S linker-IgG4 CH domains- | SRVEAEDVGVYYCGQGTQYPFTFGSGTK | |
| EPEA tag-Stop | VEIKGEGTSTGSGAIPVSLRGSGGSGGAD | |
| | QVQLVESGGGVVQPGRSLRLSCAASGFT | |
| | FSSYGMHWVRQAPGKQLEWVAQISFDG | |
| | SNKYYADSVKGRFTISRDDSKNTLYLQM | |
| | NSLRAEDTAVYYCASERGHYYDSSAFDY | |
| | WGQGTLVTVSSGGGSGGGSESKYGPPC | |
| | PPCPAPEFLGGPSVFLFPPKPKDTLMISRT | |
| | PEVTCVVVDVSQEDPEVQFNWYVDGVE | |
| | VHNAKTKPREEQFNSTYRVVSVLTVLHQ | |
| | DWLNGKEYKCKVSNKGLPSSIEKTISKA | |
| | KGQPREPQVYTLPPSQEEMTKNQVSLTC | |
| | LVKGFYPSDIAVEWESNGQPENNYKTTP | |
| | PVLDSDGSFFLYSRLTVDKSRWQEGNVF | |
| | SCSVMHEALHNHYTQKSLSLSLGKGEPE | |
| | A | |
| Anti-EpCAM VL TEAC | EVQLLEQSGAELVRPGTSVKISCKASGYA | 201 |
| component with half-life | FTNYWLGWVKQRPGHGLEWIGDIFPGSG | |
| extension moiety | NIHYNEKFKGKATLTADKSSSTAYMQLS | |
| EpCAM 20G6-VL | SLTFEDSAVYFCARLRNWDEPMDYWGQ | |
| Anti-EpCAM scFv (with 3xG4S | GTTVTVSSGGGGSGGGGSGGGGSELV | |
| linker between VL-VH)-1xG4S | MTQSPSSLTVTAGEKVTMSCKSSQSLLNS | |
| connector-20G6 VL-MMP2 | GNQKNYLTWYQQKPGQPPKLLIYWAST | |
| cleavage tag-inert binding | RESGVPDRFTGSGSGTDFTLTISSVQAED | |
| partner (VH domain)-2xG4S | LAVYYCQNDYSYPLTFGAGTKLEIKGGG | |
| linker-IgG4 CH domains- | GSDIVMTQTPLSLSVTPGQPASISCKSSQS | |
| EPEA tag-Stop | LVHNNGNTYLSWYLQKPGQSPQSLIYKV | |
| | SNRFSGVPDRFSGSGSGTDFTLKISRVEAE | |
| | DVGVYYCGQGTQYPFTFGSGTKVEIKGE | |
| | GTSTGSGAIPVSLRGSGGSGGADQVQLV | |
| | ESGGGVVQPGRSLRLSCAASGFTFSSYG | |
| | MHWVRQAPGKQLEWVAQISFDGSNKYY | |
| | ADSVKGRFTISRDDSKNTLYLQMNSLRA | |
| | EDTAVYYCASERGHYYDSSAFDYWGQG | |
| | TLVTVSSGGGSGGGSESKYGPPCPPCPAP | |
| | EFLGGPSVFLFPPKPKDTLMISRTPEVTCV | |
| | VVDVSQEDPEVQFNWYVDGVEVHNAKT | |
| | KPREEQFNSTYRVVSVLTVLHQDWLNGK | |
| | EYKCKVSNKGLPSSIEKTISKAKGQPREP | |
| | QVYTLPPSQEEMTKNQVSLTCLVKGFYP | |
| | SDIAVEWESNGQPENNYKTTPPVLDSDG | |
| | SFFLYSRLTVDKSRWQEGNVFSCSVMHE | |
| | ALHNHYTQKSLSLSLGKGEPEA | |
| Anti-EpCAM VH TEAC | ELVMTQSPSSLTVTAGEKVTMSCKSSQSL | 202 |
| component with half-life | LNSGNQKNYLTWYQQKPGQPPKLLIYW | |
| extension moiety | ASTRESGVPDRFTGSGSGTDFTLTISSVQA | |
| EpCAM 20G6-VH | EDLAVYYCQNDYSYPLTFGAGTKLEIKG | |
| Anti-EpCAM scFv (with 3xG4S | GGGSGGGGSGGGGSEVQLLEQSGAELV | |
| linker between VH-VL)-1xG4S | RPGTSVKISCKASGYAFTNYWLGWVKQ | |
| connector-20G6 VH-MMP2 | RPGHGLEWIGDIFPGSGNIHYNEKFKGKA | |
| cleavage tag-inert binding | TLTADKSSSTAYMQLSSLTFEDSAVYFCA | |
| partner (VL domain)-2xG4S | RLRNWDEPMDYWGQGTTVTVSSGGGG | |
| linker-IgG4 CH domains-His | SQVQLVESGGGVVQPGRSLRLSCAASGF | |
| tag-Stop | TFTKAWMHWVRQAPGKQLEWVAQIKD | |
| | KSNSYATYYADSVKGRFTISRDDSKNTL | |
| | YLQMNSLRAEDTAVYYCRGVYYALSPF | |
| | DYWGQGTLVTVSSGEGTSTGSGAIPVSL | |
| | RGSGGSGGADDIVMTQTPLSLSVTPGQP | |
| | ASISCKSSQSIVHSSGNTYLSWYLQKPGQ | |
| | SPQLLIYKVSNRFSGVPDRFSGSGSGTDFT | |
| | LKISRVEAEDVGVYYCGQGSHVGPTFGS | |
| | GTKVEIKGGGSGGGSESKYGPPCPPCPA | |
| | PEFLGGPSVFLFPPKPKDTLMISRTPEVTC | |
| | VVVDVSQEDPEVQFNWYVDGVEVHNAK | |
| | TKPREEQFNSTYRVVSVLTVLHQDWLNG | |
| | KEYKCKVSNKGLPSSIEKTISKAKGQPRE | |
| | PQVYTLPPSQEEMTKNQVSLTCLVKGFY | |
| | PSDIAVEWESNGQPENNYKTTPPVLDSD | |
| | GSFFLYSRLTVDKSRWQEGNVFSCSVMH | |
| | EALHNHYTQKSLSLSLGKGSHHHHHH | |
| RO258, anti Ep C AM H-TEAC | DKTHTCPPCPAPELLGGPSVFLFPPKPKD | 203 |
| SG₃SG₄S-linker Fc fusion | TLMISRTPEVTCVVVDVSHEDPEVKFNW | |
| | YVDGVEVHNAKTKPREEQYQSTYRVVS | |
| | VLTVLHQDWLNGKEYKCKVSNKALPAPI | |
| | EKTISKAKGQPREPQVYTLPPSRDELTKN | |
| | QVSLTCLVKGFYPSDIAVEWESNGQPEN | |
| | NYKTTPPVLDSDGSFFLYSKLTVDKSRW | |
| | QQGNVFSCSVMHEALHNHYTQKSLSLSP | |
| | GSGGGSGGGGSQAVVTQEPSLTVSPGGT | |
| | VTLTCRSSTGAVTTSNYANWVQQKPGQ | |
| | APRGLIGGTNKRAPWTPARFSGSLLGGK | |
| | AALTITGAQAEDEADYYCALWYSNLAVF | |
| | GGGTKLTVLGGGGSGPLGVRGKAGGGG | |
| | SEVQLVESGGGLVQPGGSLRLSCAASGFT | |
| | FNTYAMNWVRQAPGKGLEWVARIRSKY | |
| | NNYATYYADSVKDRFTISRDDSKNSLYL | |
| | QMNSLKTEDTAVYYCVRHGNFGNSYVS | |
| | WFAYWGQGTLVTVSSGGGGSEVQLLEQ | |
| | SGAELVRPGTSVKISCKASGYAFTNWL | |
| | GWVKQRPGHGLEWIGDIFPGSGNIHYNE | |
| | KFKGKATLTADKSSSTAYMQLSSLTFEDS | |
| | AVYFCARLRNWDEPMDWGQGTTVTVS | |
| | SASTKGPSVFPLAPSSKSTSGGTAALGCL | |
| | VKDYFPEPVTVSWNSGALTSGVHTFPAV | |
| | LQSSGLYSLSSVVTVPSSSLGTQTYICNV | |
| | NHKPSNTKVDKKVEPKSC | |
| RO259, anti-EpCAM H-TEAC | DKTHTCPPCPAPELLGGPSVFLFPPKPKD | 204 |
| SG₃SG₄AG₄S-linker Fc fusion | TLMISRTPEVTCVVVDVSHEDPEVKFNW | |
| | YVDGVEVHNAKTKPREEQYQSTYRVVS | |
| | VLTVLHQDWLNGKEYKCKVSNKALPAPI | |
| | EKTISKAKGQPREPQVYTLPPSRDELTKN | |
| | QVSLTCLVKGFYPSDIAVEWESNGQPEN | |
| | NYKTTPPVLDSDGSFFLYSKLTVDKSRW | |
| | QQGNVFSCSVMHEALHNHYTQKSLSLSP | |
| | GSGGGSGGGGAGGGGSQAVVTQEPSLT | |
| | VSPGGTVTLTCRSSTGAVTTSNYANWVQ | |
| | QKPGQAPRGLIGGTNKRAPWTPARFSGS | |
| | LLGGKAALTITGAQAEDEADYYCALWYS | |
| | NLAVFGGGTKLTVLGGGGSGPLGVRGK | |
| | AGGGGSEVQLVESGGGLVQPGGSLRLSC | |
| | AASGFTFNTYAMNWVRQAPGKGLEWV | |
| | ARIRSKYNNYATYYADSVKDRFTISRDDS | |
| | KNSLYLQMNSLKTEDTAVYYCVRHGNF | |
| | GNSYVSWFAYWGQGTLVTVSSGGGGSE | |
| | VQLLEQSGAELVRPGTSVKISCKASGYAF | |
| | TNWLGWVKQRPGHGLEWIGDIFPGSG | |
| | NIHYNEKFKGKATLTADKSSSTAYMQLS | |
| | SLTFEDSAVYFCARLRNWDEPMDWGQ | |
| | GTTVTVSSASTKGPSVFPLAPSSKSTSGGT | |
| | AALGCLVKDYFPEPVTVSWNSGALTSGV | |
| | HTFPAVLQSSGLYSLSSVVTVPSSSLGTQ | |
| | TYICNVNHKPSNTKVDKKVEPKSC | |
| RO260, anti-EpCAM H-TEAC | DKTHTCPPCPAPELLGGPSVFLFPPKPKD | 205 |
| SG₃SG₄AG₄S-linker Fc fusion | TLMISRTPEVTCVVVDVSHEDPEVKFNW | |
| with 11 residue protease linker | YVDGVEVHNAKTKPREEQYQSTYRVVS | |
| | VLTVLHQDWLNGKEYKCKVSNKALPAPI | |
| | EKTISKAKGQPREPQVYTLPPSRDELTKN | |
| | QVSLTCLVKGFYPSDIAVEWESNGQPEN | |
| | NYKTTPPVLDSDGSFFLYSKLTVDKSRW | |
| | QQGNVFSCSVMHEALHNHYTQKSLSLSP | |
| | GSGGGSGGGGAGGGGSQAVVTQEPSLT | |
| | VSPGGTVTLTCRSSTGAVTTSNYANWVQ | |
| | QKPGQAPRGLIGGTNKRAPWTPARFSGS | |
| | LLGGKAALTITGAQAEDEADYYCALWYS | |
| | NLAVFGGGTKLTVLGGPLGVRGKAGEV | |
| | QLVESGGGLVQPGGSLRLSCAASGFTFNT | |
| | YAMNWVRQAPGKGLEWVARIRSKYNN | |
| | YATYYADSVKDRFTISRDDSKNSLYLQM | |
| | NSLKTEDTAVYYCVRHGNFGNSYVSWF | |
| | AYWGQGTLVTVSSGGGGSEVQLLEQSG | |
| | AELVRPGTSVKISCKASGYAFTNYWLGW | |
| | VKQRPGHGLEWIGDIFPGSGNIHYNEKFK | |
| | GKATLTADKSSSTAYMQLSSLTFEDSAV | |
| | YFCARLRNWDEPMDYWGQGTTVTVSSA | |
| | STKGPSVFPLAPSSKSTSGGTAALGCLVK | |
| | DYFPEPVTVSWNSGALTSGVHTFPAVLQ | |
| | SSGLYSLSSVVTVPSSSLGTQTYICNVNH | |
| | KPSNTKVDKKVEPKSC | |
| RO261, anti-EpCAM H-TEAC | DKTHTCPPCPAPELLGGPSVFLFPPKPKD | 206 |
| SG₃SG₄AG₄S-linker Fc fusion | TLMISRTPEVTCVVVDVSHEDPEVKFNW | |
| with 9 residue protease linker | YVDGVEVHNAKTKPREEQYQSTYRVVS | |
| | VLTVLHQDWLNGKEYKCKVSNKALPAPI | |
| | EKTISKAKGQPREPQVYTLPPSRDELTKN | |
| | QVSLTCLVKGFYPSDIAVEWESNGQPEN | |
| | NYKTTPPVLDSDGSFFLYSKLTVDKSRW | |
| | QQGNVFSCSVMHEALHNHYTQKSLSLSP | |
| | GSGGGSGGGGAGGGGSQAVVTQEPSLT | |
| | VSPGGTVTLTCRSSTGAVTTSNYANWVQ | |
| | QKPGQAPRGLIGGTNKRAPWTPARFSGS | |
| | LLGGKAALTITGAQAEDEADYYCALWYS | |
| | NLAVFGGGTKLTVLGGPLGVRGGEVQL | |
| | VESGGGLVQPGGSLRLSCAASGFTFNTY | |
| | AMNWVRQAPGKGLEWVARIRSKYNNY | |
| | ATYYADSVKDRFTISRDDSKNSLYLQMN | |
| | SLKTEDTAVYYCVRHGNFGNSYVSWFA | |
| | YWGQGTLVTVSSGGGGSEVQLLEQSGAE | |
| | LVRPGTSVKISCKASGYAFTNYWLGWVK | |
| | QRPGHGLEWIGDIFPGSGNIHYNEKFKGK | |
| | ATLTADKSSSTAYMQLSSLTFEDSAVYFC | |
| | ARLRNWDEPMDYWGQGTTVTVSSASTK | |
| | GPSVFPLAPSSKSTSGGTAALGCLVKDYF | |
| | PEPVTVSWNSGALTSGVHTFPAVLQSSGL | |
| | YSLSSVVTVPSSSLGTQTYICNVNHKPSN | |
| | TKVDKKVEPKSC | |
| RO268, anti-EGFR H-TEAC | DKTHTCPPCPAPELLGGPSVFLFPPKPKD | 207 |
| SG₃SG₄S-linker Fc fusion | TLMISRTPEVTCVVVDVSHEDPEVKFNW | |
| | YVDGVEVHNAKTKPREEQYQSTYRVVS | |
| | VLTVLHQDWLNGKEYKCKVSNKALPAPI | |
| | EKTISKAKGQPREPQVYTLPPSRDELTKN | |
| | QVSLTCLVKGFYPSDIAVEWESNGQPEN | |
| | NYKTTPPVLDSDGSFFLYSKLTVDKSRW | |
| | QQGNVFSCSVMHEALHNHYTQKSLSLSP | |
| | GSGGGSGGGGSQAVVTQEPSLTVSPGGT | |
| | VTLTCRSSTGAVTTSNYANWVQQKPGQ | |
| | APRGLIGGTNKRAPWTPARFSGSLLGGK | |
| | AALTITGAQAEDEADYYCALWYSNLAVF | |
| | GGGTKLTVLGGGGSGPLGVRGKAGGGG | |
| | SEVQLVESGGGLVQPGGSLRLSCAASGFT | |
| | FNTYAMNWVRQAPGKGLEWVARIRSKY | |
| | NNYATYYADSVKDRFTISRDDSKNSLYL | |
| | QMNSLKTEDTAVYYCVRHGNFGNSYVS | |
| | WFAYWGQGTLVTVSSGGGGSGGGGSQV | |
| | QLVQSGAEVKKPGSSVKVSCKASGFTFT | |
| | DYKIHWVRQAPGQGLEWMGYFNPNSGY | |
| | STYAQKFQGRVTITADKSTSTAYMELSSL | |
| | RSEDTAVYYCARLSPGGYYVMDAWGQG | |
| | TTVTVSSASTKGPSVFPLAPSSKSTSGGTA | |
| | ALGCLVKDYFPEPVTVSWNSGALTSGVH | |
| | TFPAVLQSSGLYSLSSVVTVPSSSLGTQT | |
| | YICNVNHKPSNTKVDKKVEPKSCHHHHH | |
| | H | |
| RO269, anti-EGFR L-TEAC | DKTHTCPPCPAPELLGGPSVFLFPPKPKD | 208 |
| SG₃SG₄S-linker Fc Fusion | TLMISRTPEVTCVVVDVSHEDPEVKFNW | |
| | YVDGVEVHNAKTKPREEQYQSTYRVVS | |
| | VLTVLHQDWLNGKEYKCKVSNKALPAPI | |
| | EKTISKAKGQPREPQVYTLPPSRDELTKN | |
| | QVSLTCLVKGFYPSDIAVEWESNGQPEN | |
| | NYKTTPPVLDSDGSFFLYSKLTVDKSRW | |
| | QQGNVFSCSVMHEALHNHYTQKSLSLSP | |
| | GSGGGSGGGGSEVQLVESGGGLVQPGGS | |
| | LRLSCAASGFTFSSYAMNWVRQAPGKGL | |
| | EWVARISGSGGSTYYADSVKDRFTISRDD | |
| | SKNSLYLQMNSLKTEDTAVYYCVRGKG | |
| | NTHKPYGYVRYFDVWGQGTLVTVSSGG | |
| | GGSGPLGVRGKAGGGGSQAVVTQEPSLT | |
| | VSPGGTVTLTCRSSTGAVTASNYANWVQ | |
| | QKPGQAPRGLIGGTNKRAPWTPARFSGS | |
| | LLGGKAALTITGAQAEDEADYYCALWYS | |
| | NLWVFGGGTKLTVLGGGGSGGGGSQVQ | |
| | LVQSGAEVKKPGSSVKVSCKASGFTFTD | |
| | YKIHWVRQAPGQGLEWMGYFNPNSGYS | |
| | TYAQKFQGRVTITADKSTSTAYMELSSLR | |
| | SEDTAVYYCARLSPGGYYVMDAWGQGT | |
| | TVTVSSASTKGPSVFPLAPSSKSTSGGTA | |
| | ALGCLVKDYFPEPVTVSWNSGALTSGVH | |
| | TFPAVLQSSGLYSLSSVVTVPSSSLGTQT | |
| | YICNVNHKPSNTKVDKKVEPKSCHHHHH | |
| | H | |
| RO132, anti -EGFR/anti-CD3 | QAVVTQEPSLTVSPGGTVTLTCRSSTGAV | 209 |
| bispecific | TTSNYANWVQQKPGQAPRGLIGGTNKR | |
| | APWTPARFSGSLLGGKAALTITGAQAED | |
| | EADYYCALWYSNLWVFGGGTKLTVLGG | |
| | GGSGGGGSGGGGSEVQLVESGGGLVQP | |
| | GGSLRLSCAASGFTFNTYAMNWVRQAP | |
| | GKGLEWVARIRSKYNNYATYYADSVKD | |
| | RFTISRDDSKNSLYLQMNSLKTEDTAVY | |
| | YCVRHGNFGNSYVSWFAYWGQGTLVTV | |
| | SSGGGGSGGGGSQVQLVQSGAEVKKPGS | |
| | SVKVSCKASGFTFTDYKIHWVRQAPGQG | |
| | LEWMGYFNPNSGYSTYAQKFQGRVTITA | |
| | DKSTSTAYMELSSLRSEDTAVYYCARLSP | |
| | GGYYVMDAWGQGTTVTVSSASTKGPSV | |
| | FPLAPSSKSTSGGTAALGCLVKDYFPEPV | |
| | TVSWNSGALTSGVHTFPAVLQSSGLYSLS | |
| | SVVTVPSSSLGTQTYICNVNHKPSNTKVD | |
| | KKVEPKSCHHHHHH | |
| Human serum albumin fusion of | METDTLLLWVLLLWVPGSTGDAHKSE | 210 |
| SP34 VH TEAC, MMP2 | VAHRFKDLGEENFKALVLIAFAQYLQQC | |
| cleavage site, anti-EGFR Fab | PFEDHVKLVNEVTEFAKTCVADESAENC | |
| heavy chain (signal sequence is in | DKSLHTLFGDKLCTVATLRETYGEMADC | |
| bold) | CAKQEPERNECFLQHKDDNPNLPRLVRP | |
| | EVDVMCTAFHDNEETFLKKYLYEIARRH | |
| | PYFYAPELLFFAKRYKAAFTECCQAADK | |
| | AACLLPKLDELRDEGKASSAKQRLKCAS | |
| | LQKFGERAFKAWAVARLSQRFPKAEFAE | |
| | VSKLVTDLTKVHTECCHGDLLECADDRA | |
| | DLAKYICENQDSISSKLKECCEKPLLEKS | |
| | HCIAEVENDEMPADLPSLAADFVESKDV | |
| | CKNYAEAKDVFLGMFLYEYARRHPDYS | |
| | VVLLLRLAKTYETTLEKCCAAADPHECY | |
| | AKVFDEFKPLVEEPQNLIKQNCELFEQLG | |
| | EYKFQNALLVRYTKKVPQVSTPTLVEVS | |
| | RNLGKVGSKCCKHPEAKRMPCAEDYLS | |
| | VVLNQLCVLHEKTPVSDRVTKCCTESLV | |
| | NRRPCFSALEVDETYVPKEFNAETFTFHA | |
| | DICTLSEKERQIKKQTALVELVKHKPKAT | |
| | KEQLKAVMDDFAAFVEKCCKADDKETC | |
| | FAEEGKKLVAASQAALGLGGGGSQAVV | |
| | TQEPSLTVSPGGTVTLTCRSSTGAVTTSN | |
| | YANWVQQKPGQAPRGLIGGTNKRAPWT | |
| | PARFSGSLLGGKAALTITGAQAEDEADY | |
| | YCALWYSNLAVFGGGTKLTVLGGGGSG | |
| | PLGVRGKAGGGGSEVQLVESGGGLVQP | |
| | GGSLRLSCAASGFTFNTYAMNWVRQAP | |
| | GKGLEWVARIRSKYNNYATYYADSVKD | |
| | RFTISRDDSKNSLYLQMNSLKTEDTAVY | |
| | YCVRHGNFGNSYVSWFAYWGQGTLVTV | |
| | SSGGGGSGGGGSQVQLVQSGAEVKKPGS | |
| | SVKVSCKASGFTFTDYKIHWVRQAPGQG | |
| | LEWMGYFNPNSGYSTYAQKFQGRVTITA | |
| | DKSTSTAYMELSSLRSEDTAVYYCARLSP | |
| | GGYYVMDAWGQGTTVTVSSASTKGPSV | |
| | FPLAPSSKSTSGGTAALGCLVKDYFPEPV | |
| | TVSWNSGALTSGVHTFPAVLQSSGLYSLS | |
| | SVVTVPSSSLGTQTYICNVNHKPSNTKVD | |
| | KKVEPKSCHHHHHH | |
| Human serum albumin fusion of | METDTLLLWVLLLWVPGSTGDAHKSE | 211 |
| SP34 VL TEAC, MMP2 cleavage | VAHRFKDLGEENFKALVLIAFAQYLQQC | |
| site, anti-EGFR Fab heavy chain | PFEDHVKLVNEVTEFAKTCVADESAENC | |
| (signal sequence is in bold) | DKSLHTLFGDKLCTVATLRETYGEMADC | |
| | CAKQEPERNECFLQHKDDNPNLPRLVRP | |
| | EVDVMCTAFHDNEETFLKKYLYEIARRH | |
| | PYFYAPELLFFAKRYKAAFTECCQAADK | |
| | AACLLPKLDELRDEGKASSAKQRLKCAS | |
| | LQKFGERAFKAWAVARLSQRFPKAEFAE | |
| | VSKLVTDLTKVHTECCHGDLLECADDRA | |
| | DLAKYICENQDSISSKLKECCEKPLLEKS | |
| | HCIAEVENDEMPADLPSLAADFVESKDV | |
| | CKNYAEAKDVFLGMFLYEYARRHPDYS | |
| | VVLLLRLAKTYETTLEKCCAAADPHECY | |
| | AKVFDEFKPLVEEPQNLIKQNCELFEQLG | |
| | EYKFQNALLVRYTKKVPQVSTPTLVEVS | |
| | RNLGKVGSKCCKHPEAKRMPCAEDYLS | |
| | VVLNQLCVLHEKTPVSDRVTKCCTESLV | |
| | NRRPCFSALEVDETYVPKEFNAETFTFHA | |
| | DICTLSEKERQIKKQTALVELVKHKPKAT | |
| | KEQLKAVMDDFAAFVEKCCKADDKETC | |
| | FAEEGKKLVAASQAALGLGGGGSEVQL | |
| | VESGGGLVQPGGSLRLSCAASGFTFSSYA | |
| | MNWVRQAPGKGLEWVARISGSGGSTYY | |
| | ADSVKDRFTISRDDSKNSLYLQMNSLKT | |
| | EDTAVYYCVRGKGNTHKPYGYVRYFDV | |
| | WGQGTLVTVSSGGGGSGPLGVRGKAGG | |
| | GGSQAVVTQEPSLTVSPGGTVTLTCRSST | |
| | GAVTASNYANWVQQKPGQAPRGLIGGT | |
| | NKRAPWTPARFSGSLLGGKAALTITGAQ | |
| | AEDEADYYCALWYSNLWVFGGGTKLTV | |
| | LGGGGSGGGGSQVQLVQSGAEVKKPGSS | |
| | VKVSCKASGFTFTDYKIHWVRQAPGQGL | |
| | EWMGYFNPNSGYSTYAQKFQGRVTITAD | |
| | KSTSTAYMELSSLRSEDTAVYYCARLSPG | |
| | GYYVMDAWGQGTTVTVSSASTKGPSVF | |
| | PLAPSSKSTSGGTAALGCLVKDYFPEPVT | |
| | VSWNSGALTSGVHTFPAVLQSSGLYSLSS | |
| | VVTVPSSSLGTQTYICNVNHKPSNTKVD | |
| | KKVEPKSCHHHHHH | |
| Anti-CD8-CD3 VL ATTAC | QVQLQESGGGLVQAGGSLRLSCAASGFT | 212 |
| component | FDDYAIGWFRQAPGKEREGVSCIRVSDG | |
| (Anti-CD8 VHH-6xG4S | STYYADPVKGRFTISSDNAKNTVYLQMN | |
| connector-anti-CD3e VL | SLKPEDAAVYYCAAGSLYTCVQSIVWPA | |
| (20G6)-Enterokinase cleavage | RPYYDMDYWGKGTQVTVSSAAAYPYD | |
| sequence-Ig VH domain- | VPDYGSGGGGSGGGGSGGGGSGGGGS | |
| 2xG4S connector-IgG4 CH | GGGGSGGGGSDIVMTQTPLSLSVTPGQP | |
| domains-EPEA tag) | ASISCKSSQSLVHNNGNTYLSWYLQKPG | |
| (CD8 targeting VHH domain | QSPQSLIYKVSNRFSGVPDRFSGSGSGTD | |
| based upon WO_2017_134306 | FTLKISRVEAEDVGVYYCGQGTQYPFTF | |
| SEQ ID NO: 21) | GSGTKVEIKGEGTSTGSGGGGGSGGGGS | |
| | DDDDKGGGGSGGGGSGSGGSGGADQVQ | |
| | LVQSGAEVKKPGASVKVSCKASGYTFTS | |
| | YYIHWVRQAPGQGLEWIGCIYPGNVNTN | |
| | YNEKFKDRATLTVDTSISTAYMELSRLRS | |
| | DDTAVYFCTRSHYGLDWNFDVWGQGTT | |
| | VTVSSGGGSGGGSESKYGPPCPPCPAPEF | |
| | LGGPSVFLFPPKPKDTLMISRTPEVTCVV | |
| | VDVSQEDPEVQFNWYVDGVEVHNAKTK | |
| | PREEQFNSTYRVVSVLTVLHQDWLNGKE | |
| | YKCKVSNKGLPSSIEKTISKAKGQPREPQ | |
| | VYTLPPSQEEMTKNQVSLTCLVKGFYPS | |
| | DIAVEWESNGQPENNYKTTPPVLDSDGS | |
| | FFLYSRLTVDKSRWQEGNVFSCSVMHEA | |
| | LHNHYTQKSLSLSLGKGSEPEA | |

### DESCRIPTION OF THE EMBODIMENTS

### I. Two-Component Kits or Compositions of TEACs and ATTACs Comprising Half-Life Extending Moieties

Two-component TEACs and ATTACs described in this invention may comprise half-life extending moieties.

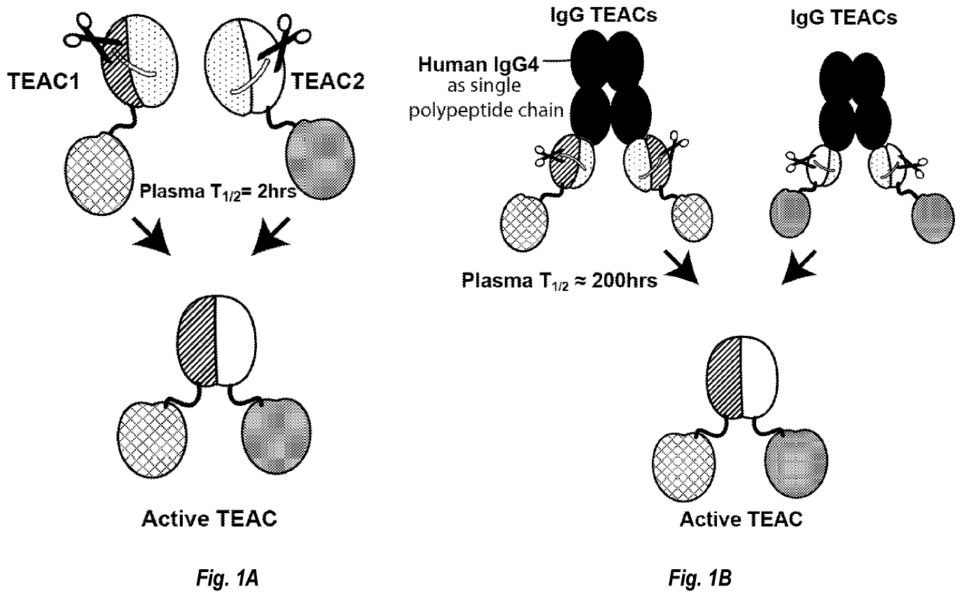

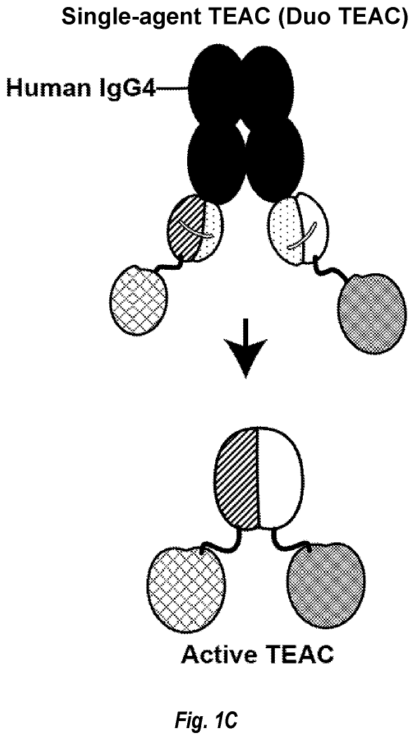

Two-component TEACs are described in U.S. Pat. No. 10,035,856, the contents of which are incorporated herein by reference in their entirety. A two-component TEAC comprises two components, wherein at least one component targets a tumor antigen. Exemplary TEACs in U.S. Pat. No. 10,035,086 include SEQ ID NOs: 165-177. FIGS. 1-4C of U.S. Pat. No. 10,035,856 demonstrate how TEACs mediate T-cell activation.

In contrast, two-component ATTACs comprise at least one targeting moiety that binds a tumor antigen and one immune selection moiety capable of selectively targeting an immune cell. The term ATTAC refers to an antibody tumor-targeting assembly complex.

Two-component ATTACs refer to using one ATTAC component that binds to a cancer antigen and one ATTAC component that does not bind to a cancer antigen, but instead selectively targets an immune cell. Thus, the ATTAC components do not have a “parallel” configuration (as in TEACs), but instead have a “trans” configuration.

In two-component TEACs or ATTACs at least one of the components will be bound to an inert binding partner that may be removed by a cleavage at a cleavage site, wherein the cleavage site is:

a. cleaved by an enzyme expressed by the cancer cells;
b. cleaved through a pH-sensitive cleavage reaction inside the cancer cell;
c. cleaved by a complement-dependent cleavage reaction; or
d. cleaved by a protease that is colocalized to the cancer cell by a targeting moiety that is the same or different from the targeting moiety in the agent.

In an ATTAC or TEAC component or pair, a first component, namely a targeted immune cell binding agent, comprises:

i. a targeting moiety capable of targeting the cancer;
ii. a first immune cell engaging domain capable of immune cell engaging activity when binding a second immune cell engaging domain, wherein the second immune cell engaging domain is not part of the first component. If an inert binding partner is on the targeted immune cell binding agent of the first component, it prevents the first immune cell engaging domain from binding to the second immune cell engaging domain unless the inert binding partner is removed.

In a two-component TEAC described herein, a second component comprises a second targeting moiety and a second immune cell engaging domain capable of immune cell engaging activity when binding the first immune cell engaging domain, wherein the first and second immune cell engaging domains are capable of binding when neither is bound to an inert binding partner. If an inert binding partner is on the targeted immune cell binding agent of the second component, it prevents the first immune cell engaging domain from binding to the second immune cell engaging domain unless the inert binding partner is removed.

In a two-component ATTAC described herein, a second component comprises an immune cell selection moiety capable of selectively targeting an immune cell and a second immune cell engaging domain capable of immune cell engaging activity when binding the first immune cell engaging domain, wherein the first and second immune cell engaging domains are capable of binding when neither is bound to an inert binding partner. If an inert binding partner is on second component in an ATTAC, it prevents the first immune cell engaging domain from binding to the second immune cell engaging domain unless the inert binding partner is removed.

Thus, both components of a two-component TEAC described herein may bind a tumor antigen expressed by the cancer. In contrast, one component of a two-component ATTAC described herein may bind a tumor antigen expressed by the cancer, while the other component selectively targets an immune cell. In this way, a component that binds a tumor antigen expressed by the cancer may be one component of a two-component TEAC or ATTAC, and the agent is either a TEAC or ATTAC based on the nature of the second component.

The moieties described below may be comprised in the ATTACs and TEACs described herein.

A. Half-Life Extending Moieties

A “half-life extending moiety,” as used herein, refers to a moiety that increases the in vivo half-life of an agent or component. A number of different methodologies have been described for increasing the in vivo half-life of biologic therapies (see Wang et al., Protein Cell 9(1):63-73 (2018) and Strohl BioDrugs 29:215-239 (2015)); however, the location of a half-life extending moiety in this context can impact its function and relationship to the composition and therapeutic strategy for treating a patient.

In some embodiments, a first component of a two-component TEAC or ATTAC comprises a half-life extending moiety.

In some embodiments, a half-life extending moiety is attached (directly or indirectly) to an inert binding partner. By “attached directly,” it is meant that there are no amino acids between a half-life extending moiety and the inert binding partner. By “attached indirectly,” it is meant that there are additional amino acids between a half-life extending moiety and the inert binding partner. In some embodiments, a half-life extending moiety is indirectly attached to the first and/or second inert binding partner via a linker.

In some embodiments, the second component of a two-component TEAC or ATTAC further comprises a second half-life extending moiety, wherein the second half-life extending moiety is attached (directly or indirectly) to the second inert binding partner. In some embodiments, the first and second half-life extending moieties are the same. In some embodiments, the first and second half-life extending moieties are different.

In some embodiments, one end of the half-life extending moiety is attached (directly or indirectly) to one copy of the first inert binding partner and the other end of the half-life extending moiety is attached (directly or indirectly) to the other copy of the first inert binding partner.

In some embodiments, one or more half-life extending moieties are capable of dissociation together with one or more inert binding partner to which it is attached.

In some embodiments, the half-life of the agent is decreased after dissociation of one or more half-life extending moieties. In some embodiments, the half-life of the first and/or second component is longer than the half-life of a complex formed by the association of the first and second immune cell or T-cell engaging domains in the form capable of binding to an immune or T cell.

In some embodiments, the first component and/or second component has a half-life greater or equal to 2 days, 4 days, or 7 days. In some embodiments, the agent or component has a half-life greater or equal to 2 days, 4 days, or 7 days.

In some embodiments, the half-life extending moiety is attached to one or more inert binding partners. In some embodiments, when a cleavage site is cleaved, the inert binding partner that is released from the agent/component dissociates together with the half-life extending moiety.

In some embodiments, dissociation of the half-life extending moiety together with the inert binding partner reduces the half-life of the agent or component. In this way, the half-life of an “activated” agent or component (i.e., an agent or component following cleavage to release the inert binding partner and half-life extending moiety) are relatively short, while the half-life of the agent or component prior to cleavage is longer based on the presence of the half-life extending moiety.

In this way, the relatively short half-life of an agent or component in the post-cleavage phase is maintained. This reduces the risk of over-activation of the immune system by active post-cleaved agents or components with long half-lives.

In contrast, the half-life of an agent or component before cleavage is longer based on the presence of a half-life extending moiety. In some embodiments, a longer half-life allows longer time periods between administration of doses of the agent or component. In some embodiments, a longer half-life allows lower doses of an agent or component to be administered.

In a two-component TEAC or ATTAC, both components may comprise a half-life extending moiety. In this way, both components of a two-component TEAC or ATTAC have a longer half-life based on the presence of a half-life extending moiety in each component. In some embodiments, both half-life extending moieties are the same. In some embodiments, the half-life extension moieties are different.

In some embodiments, one or more half-life extending moieties comprise all or part of an immunoglobulin constant (Fc) domain, serum albumin, serum albumin binding protein, an unstructured protein, and/or PEG.

1. Fc Domains

In some embodiments, the one or more half-life extending moieties comprises all or part of an Fc domain. In some embodiments, the Fc domain comprises the sequence of a human immunoglobulin. In some embodiments, the immunoglobulin is IgG. In some embodiments, the IgG is IgG1, IgG2, or IgG4.

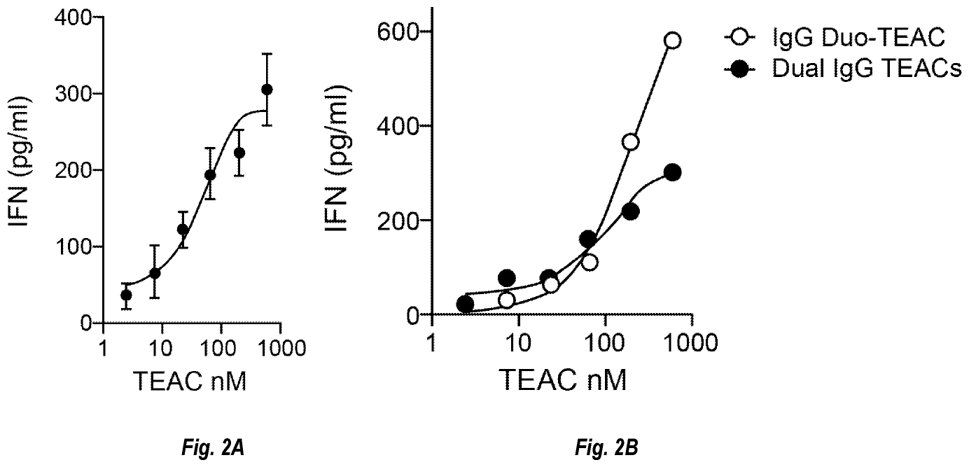

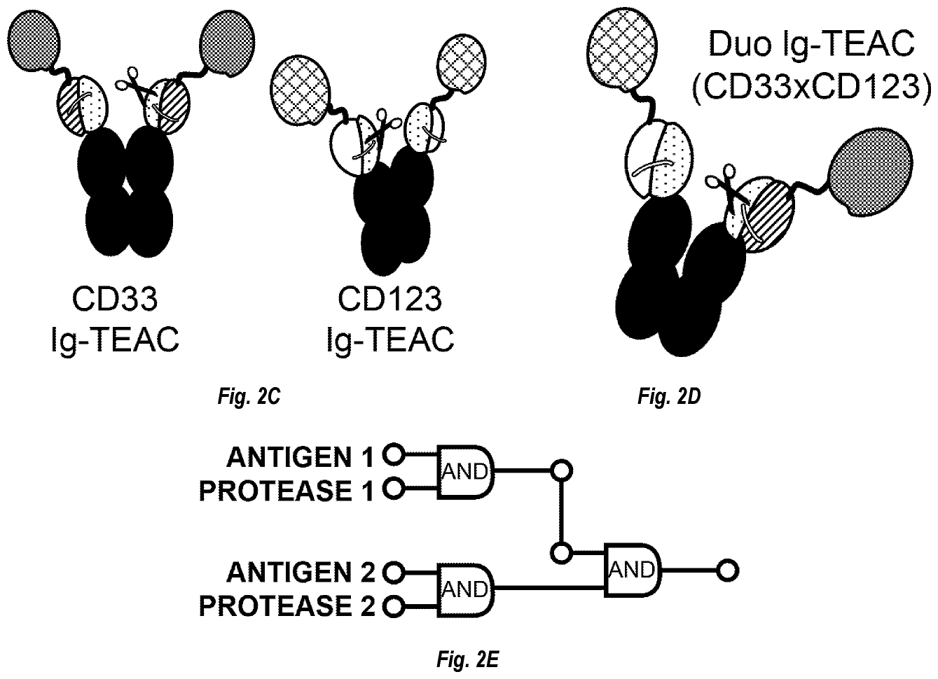

In some embodiments, one or both component of a two-component TEAC or ATTAC may comprise an Fc domain as a half-life extending moiety. When a single component of a two-component TEAC or ATTAC comprises an Fc domain, it may be referred to as an “IgG TEAC” or “IgG ATTAC.” When both components of a two-component TEAC or ATTAC comprise an Fc domain, the two components may be referred to as “Dual IgG TEACs,” or “Dual IgG ATTACs.” Using the term Dual refers to having two separate components, each utilizing an IgG such as the two constructs in FIG. 2C.

In some embodiments, a TEAC component comprising a CH domain can pair with an identical TEAC component in a cell based on pairing of the CH domains.

In some embodiments, the Fc domain comprises a naturally occurring sequence.

In some embodiments, the Fc domain comprises one or more mutations as compared to a naturally occurring sequence. An Fc domain comprising one or more mutation in an Fc domain may be referred to as an engineered Fc domain. A variety of engineered Fc domains have been described that may comprised in a TEAC or ATTAC (see Wang et al., Protein Cell 9(1):63-73 (2018)).

In some embodiments, the Fc domain is an IgG4/IgG1 hybrid or an IgG2 with IgG1 hinge.

In some embodiments, the Fc domain is an Fc domain with a longer half-life compared to a naturally occurring sequence (See Robbie et al., Antimicrob Agents Chemother 57(12):6147-6153 (2013) and Zalevsky et al., Nat Biotechnol 28(2):157-159 (2010)). In some embodiments, the Fc domain with a longer half-life has increased FcRn binding. In some embodiments, the increased FcRn binding is measured at pH 6.0. In some embodiments, the Fc domain with a longer half-life comprises M252Y/S254T/T256E substitutions. In some embodiments, the Fc domain with a longer half-life comprises M428L/N434S substitutions.

2. Serum Albumin or Albumin-Binding Protein

In some embodiments, one or more half-life extending moiety comprises all or part of serum albumin. In some embodiments, the serum albumin is human. SEQ ID NOs: 210-211 represent exemplary TEACs comprising human serum albumin.

In some embodiments, one or more half-life extending moiety comprises all or part of a serum albumin binding protein. In some embodiments, the serum albumin binding protein is a DARPin, a nanobody, a single-chain variable fragment (scFv), or an antigen-binding fragment (Fab). In some embodiments, the serum albumin binding protein comprises all or part of an albumin binding domain.

For example, half-life extension using serum albumin-binding DARPin domains has been described in Steiner et al., Protein Engineering, Design & Selection 30(9):583-591 (2017). Similarly, nanobodies that bind serum albumin have been shown to increase the half-life of linked nanobodies (see Hoefman et al., Antibodies 4:141-156 (2015)).

3. Unstructured Proteins or PEG

In some embodiments, one or more half-life extending moiety comprises all or part of an unstructured protein. In some embodiments, the unstructured protein is an unstructured hydrophilic, biodegradable protein polymer. In some embodiments, the unstructured protein is XTEN.

In some embodiments, one or more half-life extending moiety comprises all or part of polyethylene glycol (PEG).

B. Targeting Moiety Capable of Targeting the Cancer

Targeting moieties for TEACs have been described in U.S. Pat. No. 10,035,856, the contents of which are incorporated herein by reference in their entirety, including non-antibody binding partners and aptamers that may be comprised as targeting moieties capable of targeting cancer (such as SEQ ID NOs: 95-164).

The first component as a two-component ATTAC can comprise the same targeting moieties that can be comprised in a TEAC.

The targeting moiety functions in the ATTAC or TEAC to deliver the agent to the local environment of unwanted cells, enabling a localized treatment strategy. In some embodiments, the unwanted cells are cancer cells. In certain embodiments, the targeting moiety targets the cancer cells by specifically binding to the cancer cells.

In some embodiments comprising two targeting moieties, the first and second targeting moieties bind the same antigen. In some embodiments comprising two targeting moieties, the first and second targeting moieties bind the same epitope. In some embodiments comprising two targeting moieties, the first and second targeting moieties are the same. In some embodiments comprising two targeting moieties, the first and second targeting moieties are different. In some embodiments comprising two targeting moieties, the first and second targeting moieties bind different antigens. In some embodiments comprising two targeting moieties, the first and second targeting moieties bind different epitopes of the same antigen (i.e., protein).

In some embodiments, the targeting moiety is an antibody or antigen-binding fragment thereof. By antigen-binding fragment, we mean any antibody fragment that retains its binding activity to the target on the cancer cell, such as an scFv or other functional fragment including an immunoglobulin devoid of light chains, VHH, VNAR, Fab, Fab′, F(ab′)₂, Fv, antibody fragment, diabody, scAB, single-domain heavy chain antibody, single-domain light chain antibody, Fd, CDR regions, or any portion or peptide sequence of the antibody that is capable of binding antigen or epitope. VHH and VNAR are alternatives to classical antibodies and even though they are produced in different species (camelids and sharks, respectively), we will also include them in antigen-binding fragments of antibodies. Unless specifically noted as “full length antibody,” when the application refers to antibody it inherently includes a reference to an antigen-binding fragment thereof.

Table 2A provide additional information about cancers that may be targeting with different targeting moieties, including the fact that some targeting moieties may be able to target a number of different types of cancer.

TABLE 2A
Potential Targeting Moieties

| Targeting Moiety for | |
|---|---|
| First Component | Cancer Type |
| Antibody against CD20 | Lymphoma |
| (such as Rituximab) | |
| Antibody against CD80 | Lymphoma |
| Antibody against CD22 | Lymphoma |
| (such as Inotuzumab) | |
| Antibody against CD70 | Lymphoma |
| Antibody against CD30 | Lymphoma (Hodgkin, T-cell, and B-cell) |
| Antibody against CD19 | Lymphoma |
| Antibody against CD74 | Lymphoma |
| Antibody against CD40 | Lymphoma |
| Antibody against HER2 | Epithelial malignancies, breast cancer, sarcoma |
| Antibody against EpCAM | Epithelial malignancies, hepatocellular carcinoma, |
| | lung cancer, pancreatic cancer, colorectal carcinoma |
| Antibody against EGFR | Breast cancer, epithelial malignancies, gliomas, lung |
| (such as Cetuximab) | cancer, colorectal carcinoma, ovarian carcinoma, brain |
| | cancer |
| Antibody against mucin | Breast cancer |
| protein core | |
| Antibody against | Gliomas |
| transferrin receptor | |
| Antibody against | Drug-resistant melanomas |
| gp95/gp97 | |
| Antibody against p- | Drug-resistant melanomas |
| glycoprotein | |
| Antibody against TRAIL- | Multiple malignancies, including ovarian |
| R1 | and colorectal carcinoma |
| Antibody against DR5 | Multiple malignancies, including ovarian |
| | and colorectal carcinoma |
| Antibody against IL-4 | Lymphomas and leukemias |
| Antibody against IL-6 | Lymphomas and leukemias |
| Antibody against PSMA | Prostate carcinoma |
| Antibody against PSCA | Prostate carcinoma |
| Antibody against P- | Epithelial malignancies |
| cadherin (CDH3) | |
| Antibody against LI- | Gastrointestinal malignancies |
| cadherin (CDH17) | |
| Antibody against | Epithelial malignancies |
| CEACAM5 | |
| Antibody against | Epithelial malignancies |
| CEACAM6 | |
| Antibody against | Epithelial malignancies |
| CEACAM7 | |
| Antibody against | Epithelial malignancies |
| TMPRSS4 | |
| Antibody against CA9 | Epithelial malignancies |
| Antibody against GPA33 | Epithelial malignancies |
| Antibody against STEAP1 | Epithelial malignancies, particularly prostate |
| Antibody against CLDN6 | Epithelial malignancies, particularly ovarian |
| Antibody against CLDN16 | Epithelial malignancies, particularly ovarian |
| Antibody against LRRC15 | Epithelial malignancies |
| Antibody against TREM2 | Epithelial malignancies |
| Antibody against CLDN18 | Epithelial malignancies, particularly pancreatic |
| Antibody against Cripto | Epithelial malignancies |
| (TDGF1) | |
| Antibody against PD1L | Epithelial adenocarcinoma |
| Antibody against IGF-1R | Epithelial adenocarcinoma |
| Antibody against CD38 | Myeloma |
| Antibody against BCMA | Myeloma |
| Antibody against CD138 | Myeloma |
| Antibody against CD33 | Myeloid malignancies, such as AML |
| Antibody against CD37 | B-cell malignancies |
| Antibody against CD123 | Myeloid malignancies such as AML |
| Antibody against CD133 | Myeloid malignancies such as AML |
| Antibody against CD49d | Myeloid malignancies such as AML |
| Antibody against Glypican 3 | Hepatocellular carcinoma |
| Antibody against TM4SF5 | Hepatocellular carcinoma, pancreatic cancer |
| Antibody against cMet | Hepatocellular carcinoma |
| Antibody against MUC1 | Pancreatic cancer, ovarian carcinoma |
| Antibodies against | Pancreatic, ovarian and epithelial cancers |
| mesothelin (MSLN) | and mesothelioma |
| Antibody against GD2 | Sarcoma, brain cancers |
| Antibody against HER3 | Breast cancer |
| Antibody against IL-13R | Brain cancer |
| Antibody against DLL3 | Small-cell carcinoma, brain cancer |
| Antibody against MUC16 | Ovarian cancer |
| Antibodies against TFR2 | Liver cancer |
| Antibodies against TCR | T-cell malignancies |
| B1 or TCRB2 constant | |
| region | |
| Antibodies against TSHR | Thyroid malignancies |

Antibodies that have bind tumor antigens and that have specificity for tumor cells are well-known in the art. Table 2B summarizes selected publications on exemplary antibodies that bind tumor antigens and that could be used as targeting moieties in the invention.

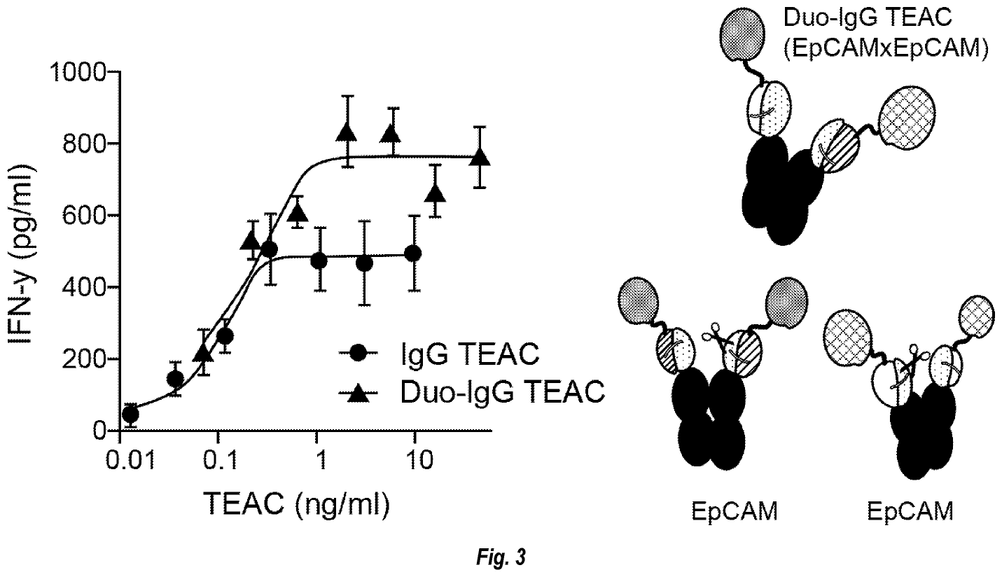

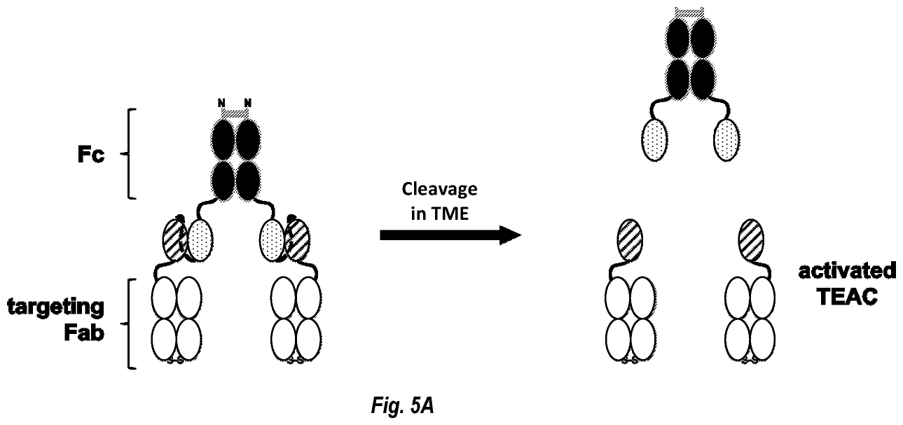

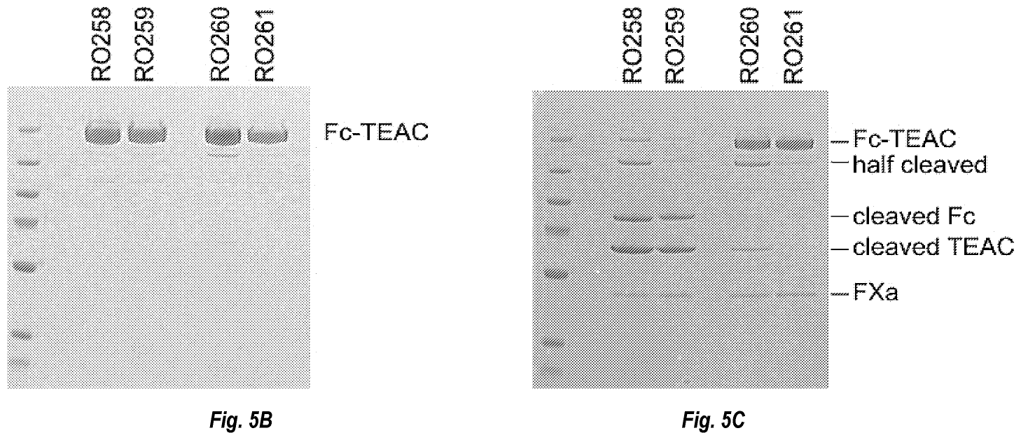

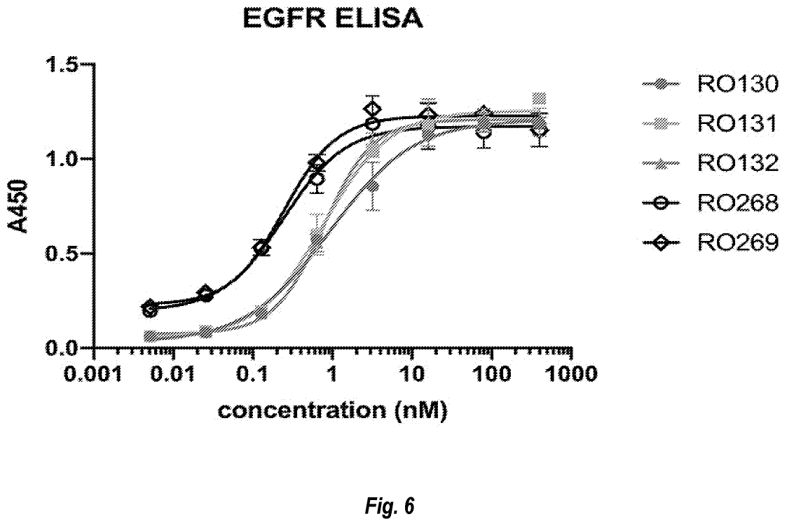

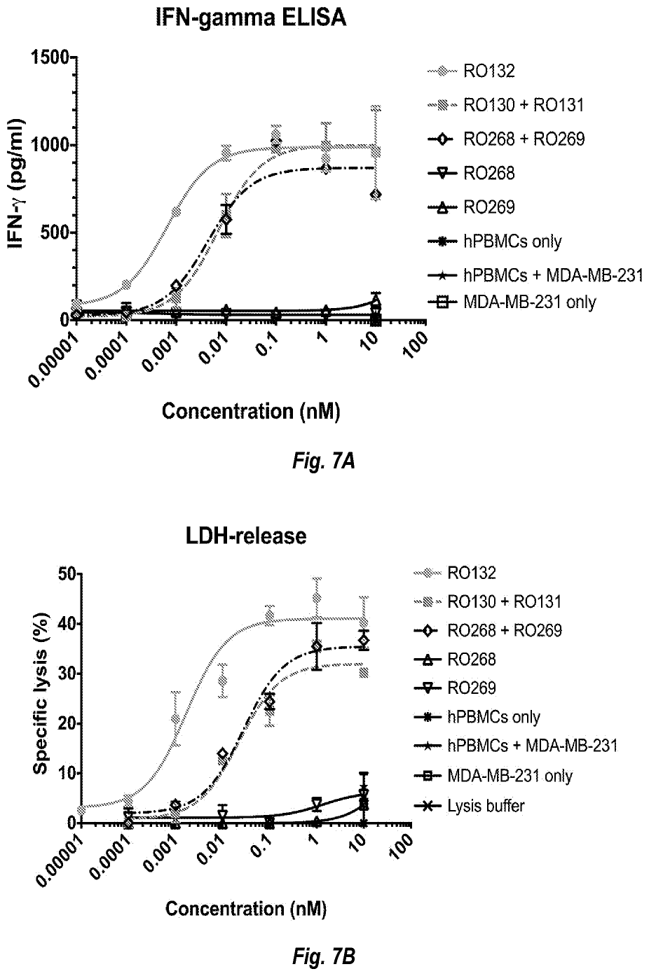

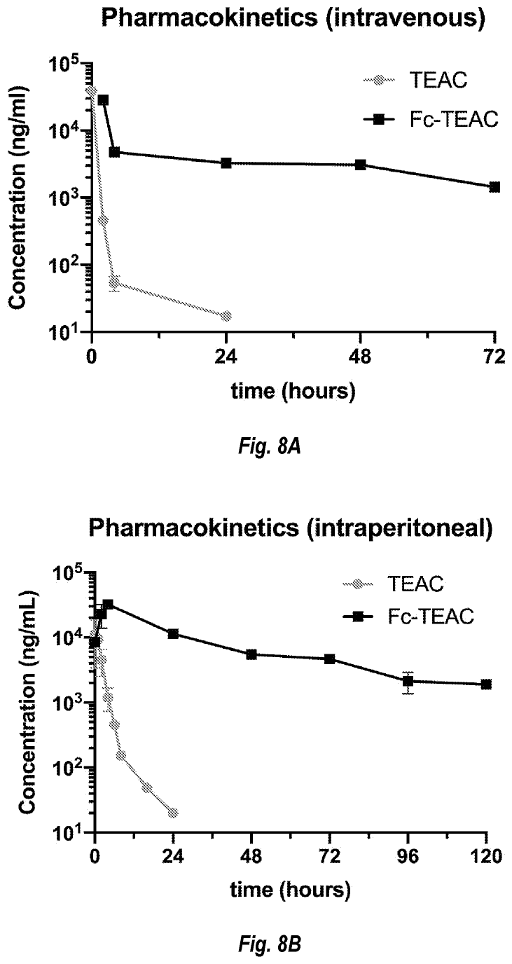

TABLE 2B
Selected publications on antibodies that bind tumor antigens

| Antigen | Publications |
|---|---|
| Her2/Neu | Carter P et al., Humanization of an anti-p185HER2 antibody for human |
| | cancer therapy, Proc Natl Acad Sci USA 89(10): 4285-9 (1992). This paper |
| | discloses the heavy and light chain sequences in its FIG. 1B. |
| | US20090202546 (Composition comprising antibody that binds to domain |
| | II of her2 and acidic variants thereof). This application discloses the variable |
| | light and variable heavy chain sequences in its claim 8. |
| | Olafsen T et al., Characterization of engineered anti-p185HER-2 (scFv- |
| | CH3)2 antibody fragments (minibodies) for tumor targeting, Protein Eng |
| | Des Sel (4): 315-23 (2004). This paper discloses light and heavy chain |
| | variable region sequences in its FIG. 1. |
| EpCAM/ | WO2008122551 (Anti-epcam antibody and uses thereof). This |
| CD326 | application discloses CDR sequences in claims 1-7. |
| | WO2010142990 A1 (Anti-EpCAM Antibodies). This application |
| | discloses CDR sequences in its claims 1-5 and 7. |
| | U.S. Pat. No. 6,969,517 (Recombinant tumor specific antibody and use thereof). This |
| | application discloses light and heavy chain sequences in its claims 1-4. |
| EGFR | Garrett J et al., Antibodies specifically targeting a locally misfolded |
| | region of tumor associated EGFR, Proc Natl Acad Sci USA 106(13): 5082- |
| | 5087 and pages 1-7 of Supporting Information including FIGS. S1-S5 |
| | (2009). This paper discloses CDR sequences in its Supplemental Information |
| | FIG. S1. (A). |
| | U.S. Pat. No. 5,844,093 Anti-EGFR single-chain fvs and anti EGFR |
| | antibodies). This patent discloses CDR sequences in its FIG. 1. |
| PSMA | US20110028696 A1 (Monoclonal antibodies against prostate specific |
| | membrane antigen (PSMA) lacking in fucosyl residues). This application |
| | discloses CDR sequences in claims 3-4. |
| | WO2003064606 (Human monoclonal antibodies to prostate specific |
| | membrane antigen (PSMA)). This application discloses CDR sequences in its |
| | claim 1. |
| CA125 | WO2011119979 A2 (Antibodies to muc16 and methods of use thereof). |
| | This application discloses VH and VL sequences in its claim 6. |
| | US20080311134 A1 (Cysteine engineered anti-muc16 antibodies and |
| | antibody drug conjugates). FIGS. 1-4 of this application show heavy and |
| | light chain sequences. |
| Carbonic | WO2007065027 A2 (Carbonic anhydrase ix (g250) antibodies and |
| Anhydrase | methods of use thereof). This application discloses CDR sequences in its |
| IX | claims 4-10. |
| | U.S. Pat. No. 7,378,091B2 (Antibodies against carbonic anhydrase IX (CA IX) tumor |
| | antigen). This application discloses CDR sequences in its FIGS. 26-29. |
| c-met/ | US20050054019 A1 (Antibodies to c-MET). This application discloses |
| HGFR | heavy and light chain sequences in its claim 6 and CDR sequences in its |
| | claim 7. |
| | US 20090175860 A1 (Compositions and methods of use for antibodies of |
| | c-Met). This application discloses CDRs in its FIGS. 1-3 and heavy and |
| | light chain sequences in its claims 12-13. |
| TRAIL- | US20040214235 A1 (Anti-TRAIL-R antibodies). This application |
| R1/DR4 | discloses heavy and light chain sequences in its claims 54-55. |
| | US20060062786 A1 (Antibodies that immunospecifically bind to TRAIL |
| | receptors). This application discloses VH and VL sequences in its claims 1-2. |
| TRAIL- | US20070031414A1 (DR5 antibodies and uses thereof). This application |
| R2/DR5 | discloses heavy and light chain sequences in its claim 1. |
| | U.S. Pat. No. 7,790,165B2 (Antibody selective for a tumor necrosis factor-related |
| | apoptosis-inducing ligand receptor and uses thereof). This application |
| | discloses heavy and light chains sequences in its claims 1-5. |
| IGF-1R | US 20040086503 A1 (Antibodies to insulin-like growth factor receptor). |
| | This application discloses light and heavy chain variable region sequences |
| | and CDR sequences in its claims 11-14. |
| | US 20070196376 A1 (Binding proteins specific for insulin-like growth factors |
| | and uses thereof). This application discloses CDR sequence data in |
| | its claims 46-47. |
| | WHO Drug Information Vol. 24, No. 2, 2010 INN PL103. This |
| | document discloses the sequence of ganitumab on pages 144-145. |
| VEGF-R2 | Rinderknecht M et al., Phage-Derived Fully Human Monoclonal |
| | Antibody Fragments to Human Vascular Endothelial Growth Factor-C Block |
| | Its Interaction with VEGF Receptor-2 and 3, PLoS One 5(8): e11941 (2010). |
| | This paper discloses CDR sequences in its Table 2. |
| | WO1998045331 A2 (Anti-VEGF antibodies). This application discloses |
| | CDR sequences in its claims 6, 8, and 9. |
| Prostate | US20090181034 A1 (Antibodies and related molecules that bind to psca |
| stem cell | proteins). This application discloses VH and VL sequences in its claim 17. |
| antigen | U.S. Pat. No. 6,790,939 B2 (Anti-PSCA antibodies). This application discloses |
| (PSCA) | CDR sequences in its FIG. 61. |
| | WO2009032949 A2 (High affinity anti-prostate stem cell antigen (psca) |
| | antibodies for cancer targeting and detection). This application discloses |
| | CDR sequences in its FIG. 2. |
| MUC1 | Thie H et al., Rise and Fall of an Anti-MUC1 Specific Antibody, PLoS |
| | One Jan 14; 6(1): e15921 (2011). This paper discloses CDR sequences in its |
| | FIG. 1. |
| | Henderikx H et al., Human Single-Chain Fv Antibodies to MUC1 Core |
| | Peptide Selected from Phage Display Libraries Recognize Unique Epitopes |
| | and Predominantly Bind Adenocarcinoma, Cancer Res. 58(19): 4324-32 |
| | (1998). This paper discloses CDR sequences in its Table 2. |
| CanAg | US20080138898 A1 (Methods for improving antibody production). This |
| | application discloses CDR sequences in its FIG. 5. |
| Mesothelin | WO2009068204 A1 (Anti-mesothelin antibodies and uses therefor). This |
| | application discloses CDR sequences in its Table 7. |
| P-cadherin | WO2010001585 A1 (Anti-CDH3 antibodies labeled with radioisotope |
| | label and uses thereof). This application discloses VH and VL variable |
| | region sequences disclosed in its paragraph [0033] and CDR sequences in |
| | claim 2-7. |
| Myostatin/ | U.S. Pat. No. 7,632,499 B2 (Anti-myostatin antibodies). This application discloses |
| GDF8 | CDR sequences in its claim 1. |
| | US 20090148436 A1 (Antibody to GDF8 and uses thereof). This |
| | application discloses CDR, VH, and VL sequences in its claims 2-8. |
| Cripto/ | US20100008906 A1 (Cripto binding molecules). This application |
| TDGF1 | discloses light and heavy chain sequences in its paragraph [0491] and CDR |
| | sequences in its paragraph [0492]. |
| | U.S. Pat. No. 7,531,174 B2 (Cripto blocking antibodies and uses thereof). This |
| | application discloses a list of hybridomas that secrete anti-Cripto antibodies |
| | in its Tables 1 and 2. These hybridomas were available for purchase from the ATCC. |
| MUC5AC | Chung W C et al., CREB mediates prostaglandin F2alpha-induced |
| | MUC5AC overexpression, J Immunol 182(4): 2349-56 (2009) at page 3, |
| | second paragraph discloses that clone 45M1 was an anti-MUC5AC antibody |
| | available for purchase. |
| CEACAM | Pavoni E. et al., Selection, affinity maturation, and characterization of a |
| | human scFv antibody against CEA protein, BMC Cancer 6: 41 (2006). This |
| | paper discloses CDR sequences of clone E8 in its FIG. 3. Reactivity of E8 |
| | with CEACAM is shown in its FIG. 6. |
| SLC44A4 | US20090175796 A1 (Antibodies and related molecules that bind to |
| (formerly | 24p4c12 Proteins). This application discloses light and heavy chain variable |
| known as | domain sequences in its FIGS. 2 and 3. |
| protein | U.S. Pat. No. 8,039,597 B2 (Antibodies and related molecules that bind to 24p4c12 |
| 24P4C12 | Proteins). This application discloses light and heavy chain variable domain |
| which was | sequences in its claim 1 and in its FIGS. 2 and 3. |
| renamed | U.S. Pat. No. 8,309,093 B2 (Antibody drug conjugates (ADC) that bind to 24P4C12 |
| SLC44A4 by | proteins). This application iscloses light and was heavy chain variable domain |
| the Hugo | d sequences in its claim 1 and in its FIGS. 2 and 3. |
| Convention | US20100330107 A1 (Antibody drug conjugates (ADC) that bind to |
| (see | 24P4C12 proteins). This application discloses light and heavy chain variable |
| U.S. Pat. No. | domain sequences in its claims 1 and 2, and in its FIGS. 2 and 3. |
| 8,039,497 at | WO2010111018 A1 (Antibody drug conjugates (ADC) that bind to |
| 114: 56-62)) | 24P4C12 proteins). This application discloses light and heavy chain variable |
| | domain sequences in its claims 1 and 2, and in its FIGS. 2 and 3. |
| Neuropilin 1 | U.S. Pat. No. 8,318,163 B2 (Anti-pan neuropilin antibody and binding fragments |
| | thereof). This application discloses light and heavy chain variable domain |
| | sequences in its claim 1 and in its FIGS. 7 and 8. |
| | WO 2008/143666 (Crystal structures of neuropilin fragments and |
| | neuropilin-antibody complexes). This application discloses light and heavy |
| | chain variable domain sequences in its claim 8 and in its FIGS. 7 and 8. |
| Glypican | U.S. Pat. No. 7,867,734 B2 (Anti-glypican 3 antibody having modified sugar |
| | chain). This application discloses the heavy chain variable region in its claim |
| | 1. CDR sequences are disclosed in Table 1 of this application. |
| | U.S. Pat. No. 7,871,613 B2 (Adjuvant therapy with the use of anti-glypican 3 |
| | antibody). This application discloses the heavy chain sequence in its claim 6 |
| | and the light chain sequence in its claim 7. |
| EphA2 | US20100298545 A1. (Epha2 agonistic monoclonal antibodies and methods of |
| | use thereof). This application discloses CDR sequences in its |
| | claim 50. |
| | US20100278838 A1. (Epha2 monoclonal antibodies and methods of use thereof). This application |
| | discloses VH/VL and CDR sequences in its claim 101. |
| | US20100183618 A1 (Anti-epha2 antibody). This application discloses CDR sequences in its claim 11. |
| E-cadherin | U.S. Pat. No. 5,610,281 (Antibodies for modulating heterotypic E-cadherin |
| | interactions with human T lymphocytes). This application discloses that anti- |
| | E-cadherin clone E4.6 is available for the ATCC (HB 11996) in its claim 4. |
| CEA | WO2004032962 A1 (Combination therapy with class iii anti-cea |
| | monoclonal antibodies and therapeutic agents). This application discloses |
| | CDR sequences in its claim 6 and its claim 14. |
| | U.S. Pat. No. 5,877,293 (CDR grafted anti-CEA antibodies and their production). |
| | This application discloses antibody sequences in its claims 1-5. |
| | US20080069816 A1 (Humanized anti-cea t84.66 antibody and uses |
| | thereof). This application discloses antibody sequence in its claims 22-23. |
| FGFR3 | US20080044419 A1 (Treatment of T Cell Mediated Diseases by |
| | Inhibition of Fgfr3). This application discloses scFv sequences in claim 6 |
| | and VH/VL sequences in its claims 7-10. |
| | US20090175866 A1 (Treatment of B-cell malignancies). This |
| | application discloses Vh, Vl and CDR sequences in its claims 11-12. |
| | Martinez-Torrecuadrada J et al. Targeting the extracellular domain of |
| | fibroblast growth factor receptor 3 with human single-chain Fv |
| | antibodies inhibits bladder carcinoma cell line proliferation. Clin Cancer Res |
| | 11(17): 6280-90 (2005). This publication shows VH and VL sequences of a |
| | scFv in its FIG. 2. |
| HER3 | Lee-Hoeflich S T et al. A Central Role for HER3 in HER2-Amplified |
| | Breast Cancer: Implications for Targeted Therapy. Cancer Res. 68(14): 5878- |
| | 5887 (2008). |
| | Scartozzi M et al. The role of HER-3 expression in the prediction of |
| | clinical outcome for advanced colorectal cancer patients receiving irinotecan |
| | and cetuximab.Oncologist. 16(1): 53-60 (Epub Jan. 6, 2011). |
| | Sheng Q et al. An activated ErbB3/NRG1 autocrine loop supports in vivo |
| | proliferation in ovarian cancer cells. CancerCell. 17(3): 298-310 (2010). |
| | Schoeberl B et al. An ErbB3 antibody, MM-121, is active in cancers with |
| | ligand dependent activation. Cancer Res. 70(6): 2485-2494 (2010). |
| | Khan I H et al. Microbead arrays for the analysis of ErbB receptor |
| | tyrosine kinase activation and dimerization in breast cancer cells. Assay |
| | Drug Dev Technol. 8(1): 27-36. (2010). |
| | Robinson M K et al. Targeting ErbB2 and ErbB3 with a bispecific single- |
| | chain Fv enhances targeting selectivity and induces a therapeutic effect in |
| | vitro. British Journal of Cancer 99: 1415-1425 (2008). |
| | Reschke Metal. HER3 is a determinant for poor prognosis in |
| | melanoma.Clin Cancer Res. 14(16): 5188-97 (2008). |
| PDGFRa | Martinhoe, O. et al. Expression, mutation and copy number analysis of |
| | platelet-derived growth factor receptor A (PDGFRA) and its ligand PDGFA |
| | in gliomas. Br J Cancer 101: 973-982 (2009). |
| | Loizos N et al. Targeting the platelet-derived growth factor receptor |
| | alpha with a neutralizing human monoclonal antibody inhibits the growth of |
| | tumor xenografts: implications as a potential therapeutic target. Mol Cancer |
| | Ther. 4(3): 369-79 (2005). |
| | Russell M R et al. Targeting the {alpha} receptor for platelet-derived |
| | growth factor as a primary or combination therapy in a preclinical model of |
| | prostate cancer skeletal metastasis. Clin Cancer Res. 16(20): 5002-10 (2010). |
| | Shah G D et al. Rationale for the development of IMC-3G3, a fully |
| | human immunoglobulin G subclass 1 monoclonal antibody targeting the |
| | platelet-derived growth factor receptor alpha.Cancer. 116(4 Suppl): 1018-26 |
| | (2010). |
| | Dolloff N G et al. Human bone marrow activates the Akt pathway in |
| | metastatic prostate cells through transactivation of the alpha-platelet-derived |
| | growth factor receptor.Cancer Res. 67(2): 555-62 (2007). |
| CS1 | Tai-Y T et al. Anti-CS1 humanized monoclonal antibody HuLuc63 |
| | inhibits myeloma cell adhesion and induces antibody-dependent cellular |
| | cytotoxicity in the bone marrow milieu. Blood 112(4): 1329-1337 (2008). |
| | Van Rhee F et al. Combinatorial efficacy of anti-CS1 monoclonal |
| | antibody elotuzumab (HuLuc63) and bortezomib against multiple myeloma. |
| | Mol Cancer Ther. 8(9): 2616-2624 (2009). |
| | Hsi E D et al. CS1, a potential new therapeutic antibody target for the |
| | treatment of multiple myeloma. Clin Cancer Res. 14(9): 2775-2784 (2008). |
| | Lee J K et al. CS1 (CRACC, CD319) induces proliferation and autocrine cytokine |
| | expression on human B lymphocytes. J Immunol 179: 4672-4678 |
| | (2007). |
| CD137 | Broll K et al. CD137 Expression in Tumor Vessel Walls: High |
| (4-1BB) | Correlation with Malignant Tumors. Am J Clin Pathol115(4)543-549 |
| | (2001). |
| | Melero I et al. Monoclonal antibodies against the 4-1BB T-cell activation |
| | molecule eradicate established tumors. Nat Med3: 682-5 (1997) (abstract). |
| | Niu L et al. Cytokine-mediated disruption of lymphocyte trafficking, |
| | hemopoiesis, and induction of lymphopenia, anemia, and thrombocytopenia |
| | in anti-CD137-treated mice. J Immunol. 178(7): 4194-4213 (2007). |
| | Palazon A et al. Agonist anti-CD137 mAb act on tumor endothelial cells |
| | to enhance recruitment of activated T lymphocytes. Cancer Res. 71(3): 801- |
| | 11 (February 2011). |
| CXCR4 | Akashi-T et al. Chemokine receptor CXCR4 expression and prognosis in |
| | patients with metastatic prostate cancer.Cancer Sci 99(3): 539-542 (2008). |
| | Mirisola-V. et al. CXCL12/SDF1 expression by breast cancers is an |
| | independent prognostic marker of disease-free and overall surviva.,Eur J |
| | Cancer 45(14): 2579-87 (2009) (abstract). |
| | Gassmann P et al. CXCR4 regulates the early extravasation of metastatic |
| | tumor cells in vivo. Neoplasia. 11(7): 651-61. (2009). |
| | Roland Jet al. Role of the intracellular domains of CXCR4 in SDF-1- |
| | mediated signaling.Blood. 101: 399-406 (2003). |
| | Fischer T et al. Reassessment of CXCR4 chemokine receptor expression |
| | in human normal and neoplastic tissues using the novel rabbit monoclonal |
| | antibody UMB-2. PLoS One. 3(12): e4069 (2008). |
| | Otsuka S and Bebb G. The CXCR4/SDF-1 Chemokine Receptor Axis. |
| | J Thorac Oncol. 3: 1379-1383 (2008). |
| | Xu C et al. Human anti-CXCR4 antibodies undergo VH replacement, |
| | exhibit functional V-region sulfation, and define CXCR4 antigenic |
| | heterogeneity. JImmunol 179(4): 2408-2418 (2007). |
| ACVRL1/ | Goff L et al. Phase I study of pf-03446962, a fully human mab against |
| ALK1 | alk 1, a TGFbeta receptor involved in tumor angiogenesis J Clin Oncol |
| | 28(15 suppl): 3034 (2010) (abstract). |
| | Hu-Lowe D D et al. Targeting activin receptor-like kinase 1 inhibits |
| | angiogenesis and tumorigenesis through a mechanism of action |
| | complementary to anti-VEGF therapies.Cancer Res; 71: 1362-73 (2011). |
| | Mancuso P, et al. Validation of a standardized method for enumerating |
| | circulating endothelial cells and progenitors: flow cytometry and molecular |
| | and ultrastructural analysesClin Cancer Res 15: 267-73 (2009). |
| | Naeem S et al. Bone marrow involvement in systemic ALK+ anaplastic |
| | large cell lymphoma: morphological resemblance with Hodgkin's lymphoma. |
| | J Coll Physicians Surg Pak 16(2): 148-9 (2006) (abstract). |
| PD-1 | Iwai Y et al. Involvement of PD-L1 on tumor cells in the escape from |
| | host immune system and tumor immunotherapy by PD-L1 blockade.Proc |
| | Natl Acad Sci 19(19): 12293-12297 (2002). |
| | Toshiro I et al. Analysis of the Role of Negative T Cell Costimulatory |
| | Pathways in CD4 and CD8 T Cell-Mediated Alloimmune Responses In |
| | Vivo. JImmunol, 174: 6648-6656 (2005). |
| | Brahmer JR et al. Phase I study of single-agent anti-programmed death-1 |
| | (MDX-1106) in refractory solid tumors: safety, clinical activity, |
| | pharmacodynamics, and immunologic correlates.J Clin Oncol 28: 3167-3175 |
| | (2010). |
| | Tsushima F et al. Interaction between B7-H1 and PD-1 Determines |
| | Initiation and Reversal of T-Cell Anergy. Blood 110(10): 180-185 (2007). |
| PD-L1 | Blank C et al. Blockade of PD-L1 (B7-H1) augments human tumor- |
| | specific T cell responses in vitro. Int J Cancer 119: 317-327 (2006) |
| | (abstract). |
| | Ishida M et al. Differential expression of PD-L1 and PD-L2, ligands for |
| | an inhibitory receptor PD-1, in the cells of lymphohematopoietic tissues. |
| | Immunol Lett 84(1): 57-62 (2002) (abstract). |
| | Thompson Hit et al. Tumor B7-H1 Is Associated with Poor Prognosis in |
| | Renal Cell Carcinoma Patients with Long-term Follow-up.Cancer Res |
| | 66(7): 3381-3385 (2006). |
| | Latchman YE et al. PD-L1-deficient mice show that PD-L1 on T cells, |
| | antigen-presenting cells, and host tissues negative lyregulates T cells. Proc |
| | Natl Acad Sci 101(29): 10691-10696 (2004). |
| | Dong H et al. Costimulating aberrant T cell responses by B7-H1 |
| | autoantibodies in rheumatoid arthritis. J Clin Invest 111: 363-370 (2003), |
| | Brahmer J R et al. Phase I study of single-agent anti-programmed death-1 |
| | (MDX-1106) in refractory solid tumors: safety, clinical activity, |
| | pharmacodynamics, and immunologic correlates.J Clin Oncol 28: 3167-3175 |
| | (2010). |
| | Hamanishi J et al. Programmed cell death 1 ligand 1 and tumor |
| | infiltrating CD8 T lymphocytes are prognostic factors of human ovarian |
| | cancer. Proc Natl Acad Sci 105(9): 3360-65 (2007). |
| CD70 | Israel B F et al. Anti-CD70 antibodies: a potential treatment for EBV+ |
| | CD70-expressing lymphoma., Mol Cancer Ther 4(12): 2037-2044 (2005). |
| | Lens S M et al. Aberrant expression and reverse signalling of CD70 on |
| | malignant B cells. Br J Haematol 106: 491-503 (1999). |
| | Ranheim E A et al, Expression of CD27 and its ligand, CD70, on chronic |
| | lymphocytic leukemia B cells.Blood 85: 3556-65 (1995). |
| | Zambello R et al. Analysis of TNF-receptor and ligand superfamily |
| | molecules in patients with lymphoproliferative disease of granular |
| | lymphocytes. Blood 96: 647-54 (2000). |
| | Bullock T N et al. Induction of CD70 on dendritic cells through CD40 or |
| | TLR stimulation contributes to the development of CD8+ T cell responses in |
| | the absence of CD4+ T cells.J Immunol 174: 710-7 (2005). |
| CD74 | Stein Ret al. CD74: A New Candidate Target for the Immunotherapy of |
| | B-Cell Neoplasms Clin Cancer Res 13(18): 5556s-5563s (2007). |
| | Starlets D et al. Cell surface CD74 initiates a signaling cascade leading to |
| | cell proliferation and survival. Blood 107: 4807-16 (2006). |
| | Stein R et al. Anti-proliferative activity of a humanized anti-CD74 |
| | monoclonal antibody, hLL1, on B-cell malignancies. Blood 104: 3705-11 |
| | (2004). |
| | Chang C H et al. Effective therapy of human lymphoma xenografts with a |
| | novel recombinant ribonuclease/anti-CD74 humanized IgG4 antibody |
| | immunotoxin.Blood 106: 4308-14 (2005). |
| | Burton J D et al. CD74 Is Expressed by Multiple Myeloma and Is a |
| | Promising Target for Therapy. Clin Cancer Res 10(19): 6606-6611 (2004). |
| CD56 | Fossella V et al. Phase II trial of BB-10901 (huN901-DM1) given weekly |
| | for four consecutive weeks every 6 weeks in patients with relapsed SCLC |
| | and CD56-positive small cell carcinoma. J Clin Oncol 23(16_suppl): 7159- |
| | 7159 (2005) (abstract). |
| | Roguska M A et al. Humanization of murine monoclonal antibodies |
| | through variable domain resurfacing. Proc Natl Acad Sci 91(3): 969-73 |
| | (1994). |
| | Cooper MA et al. Human natural killer cells: a unique innate |
| | immunoregulatory role for the CD56(bright) subset. Blood 97(10): 3146-51 |
| | (2001). |
| | Campbell J J et al. Unique subpopulations of CD56+ NK and NK-T |
| | peripheral blood lymphocytes identified by chemokiner eceptor expression |
| | repertoire. J Immunol 166(11): 6477-82 (2001). |
| | De Maria A et al. Revisiting human natural killer cell subset function |
| | revealed cytolytic CD56(dim)CD16+ rNK cells as apid producers of |
| | abundant IFN-gamma on activation. Proc Natl Acad Sci 108: 728-32 (2011). |
| | Cho E Y et al. Immunohistochemical study of the expression of adhesion |
| | molecules in ovarian serous neoplasms. Pathol Int 56(2): 62-70 (2006) |
| | (abstract). |
| CD40 | Luqman M et al. The antileukemia activity of a human anti-CD40 |
| | antagonist antibody, HCD122, on human chronic lymphocytic leukemia cells |
| | Blood 112(3): 711-720 (2008). |
| | Uckum F M et al. Temporal association of CD40 antigen expression with |
| | discrete stages of human B-cell ontogeny and the efficacy of anti-CD40 |
| | immunotoxins against clonogenic B-lineage acute lymphoblastic leukemia as |
| | well as B- lineage non-Hodgkin's lymphoma cells Blood 76 (12) 2449-2456 |
| | (1990). |
| | Vyth-Dreese FA et al. Localization in situ of costimulatory molecules |
| | and cytokines in B-cell non-Hodgkin's lymphoma. Immunology 94: 580-586 |
| | (1998). |
| | Hulkkonen J et al. Surface antigen expression in chronic lymphocytic |
| | leukemia: clustering analysis, interrelationships and effects of chromosomal |
| | abnormalities. Leukemia 16: 178-185 (2002). |
| | Kater A P et al. CD40 stimulation of B-cell chronic lymphocytic |
| | leukaemia cells enhances the anti-apoptotic profile, but also Bid expression |
| | and cells remain susceptible to autologous cytotoxic T-lymphocyte attack. Br |
| | J Haematol 127: 404-415 (2004) (abstract). |
| | Melter M et al. Ligation of CD40 induces the expression of vascular |
| | endothelial growth factor by endothelial cells and monocytes and promotes |
| | angiogenesis in vivo. Blood 96: 3801-3808 (2000). |
| CD19 | Blanc V et al. 5AR3419: An Anti-CD19-Maytansinoid |
| | Immunoconjugate for the Treatment of B-Cell Malignancies. Clin Cancer |
| | Res 17(20): 6448-6458 (2011). |
| | Herbst R et al. B-cell depletion in vitro and in vivo with an afucosylated |
| | anti-CD19 antibody. J Pharmacol Exp Ther 335: 213-22 (2010). |
| | D'Arena G et al. Quantitative flow cytometry for the differential |
| | diagnosis of leukemic B-cell chronic lymphoproliferative disorders. Am J |
| | Hemat 64: 275-281 (2000) (abstract). |
| | Johnson N A et al. Diffuse large B-cell lymphoma: reduced CD20 |
| | expression is associated with an inferior survival. Blood 113: 3773-3780 |
| | (2009). |
| | Sato S et al. Altered blood B lymphocyte homeostasis in systemic |
| | sclerosis: expanded naive B cells and diminished but activated memory B |
| | cell.Arthritis Rheum 50: 1918-1927 (2004) (abstract). |
| | Kansas G S et al. Transmembrane signals generated through MHC class |
| | II, CD19, CD20, CD39, and CD40 antigens induce LFA-1-dependent and |
| | independent adhesion in human B cells through a tyrosine kinase-dependent |
| | pathway. J Immunol 147: 4094-4102 (1991) (abstract). |
| CD80 | Leonard J W et al. A phase I/II study of galiximab (an anti-CD80 |
| | monoclonal antibody) in combination with rituximab for relapsed or |
| | refractory, follicular lymphoma. Ann Oncol 18(7): 1216-1223 (2007). |
| | Vyth-Dreese F A et al. Localization in situ of costimulatory molecules |
| | and cytokines in B-cell non-Hodgkin's lymphoma.Immunology 94: 580-586 |
| | (1998). |
| | Dorfman D M et al. In vivo expression of B7-1 and B7-2 by follicular |
| | lymphoma cells can prevent induction of T-cell anergy but is insufficient to |
| | induce significant T-cell proliferation. Blood 90: 4297-4306 (1997). |
| | Dogan A et al. Follicular lymphomas contain a clonally linked but |
| | phenotypically distinct neoplastic B-cell population in the interfollicular |
| | zoneBlood 91: 4708-4714 (1998). |
| | Suvas S et al. Distinct role of CD80 and CD86 in the regulation of the |
| | activation of B cell and B cell lymphoma. J Biol Chem 277: 7766-7775 |
| | (2002). |
| CD86 | Vincenti, F. What's in the pipeline? New immunosuppressive drugs in |
| | transplantation. Am J Transplant 2: 898-903 (2002) (abstract). |
| | Vyth-Dreese FA et al. Localization in situ of costimulatory molecules |
| | and cytokines in B-cell non-Hodgkin's lymphoma. Immunology 94: 580-586 |
| | (1998). |
| | Dorfman D M et al. In vivo expression of B7-1 and B7-2 by follicular |
| | lymphoma cells can prevent induction of T-cell anergy but is insufficient to |
| | induce significant T-cell proliferation.Blood 90: 4297-4306 (1997). |
| | Dogan A et al. Follicular lymphomas contain a clonally linked but |
| | phenotypically distinct neoplastic B-cell population in the interfollicular |
| | zone. Blood 91: 4708-4714 (1998). |
| | Suvas S et al. Distinct role of CD80 and CD86 in the regulation of the |
| | activation of B cell and B cell lymphoma. J Biol Chem 277: 7766-7775 |
| | (2002). |
| CD2 | Matthews J B et al. Clinical Trials of Transplant Tolerance: Slow But |
| | Steady Progress. Am J Transplant 3: 794-803 (2003). |
| | Przepiorka D et al. A phase II study of BTI-322, a monoclonal anti-CD2 |
| | antibody, for treatment of steroid-resistant acute graft-versus-host disease. |
| | Blood 92: 4066-4071 (1998). |
| | Latinne D et al. An anti-CD2 mAb induces immunosuppression and |
| | hyporesponsiveness of CD2+ human T cells in vitro. Int Immunol 8: 1113 |
| | (1996) (abstract). |
| | Guckel B et. Anti-CD2 antibodies induce T cell unresponsiveness in |
| | vivo. J Exp Med 174: 957, (1991). |
| | Bromberg J S et al. Anti-CD2 monoclonal antibodies alter cell-mediated |
| | immunity in vivo. Transplantation 51: 219 (1991) (abstract). |
| CD30 | Maeda-N. Susceptibility of human T-cell leukemia virus type I-infected |
| | cells to humanized anti-CD30 monoclonal antibodies in vitro and in vivo. |
| | Cancer Sci 101(1): 224-30 (2010) (epub 2009 Sep. 8) (abstract). |
| | Schlapschy M et al. Functional humanization of an anti-CD30 Fab |
| | fragment for the immunotherapy of Hodgkin's lymphoma using an in vitro |
| | evolution approach. Protein Eng Des Sel 17(12): 847-860 (2004). |
| | da Costa L et al. Immunoscintigraphy in Hodgkin's disease and |
| | anaplastic large cell lymphomas: results in 18 patients using the iodine |
| | radiolabeled monoclonal antibody FIRS-3. Ann Oncol. Sep; 3 Suppl 4: 53-7 |
| | (1992) (abstract). |
| | Su C C et al. CD30 Is Involved in Inhibition of T-Cell Proliferation by |
| | Hodgkin's Reed-Sternberg Cells, Cancer Res 64(6): 2148-2152 (2004). |
| | Pinto A et al. Human eosinophils express functional CD30 ligand and |
| | stimulate proliferation of a Hodgkin's disease cell line. Blood 88 (9) 3299- |
| | 3305 (1996). |
| | Barth-S et al. Ki-4(scFv)-ETA', a new recombinant anti-CD30 |
| | immunotoxin with highly specific cytotoxic activity against disseminated |
| | Hodgkin tumors in SCID mice. Blood 95 (12): 3909-3914 (2000). |
| CD20 | McLaughlin P et al. Rituximab chimeric anti-CD20 monoclonal antibody |
| | therapy for relapsed indolent lymphoma: half of patients respond to a four- |
| | dose treatment program. J Clin Oncol 16: 2825-33 (1998) (abstract). |
| | Kaminski M S et al. Radioimmunotherapy with iodine (131)I |
| | tositumomab for relapsed or refractory B-cell non-Hodgkin lymphoma: |
| | updated results and long-term follow-up of the University of Michigan |
| | experience. Blood 96: 1259-66 (2000). |
| | Coiffier B et al. Rituximab in combination with CHOP improves survival |
| | in elderly patients with aggressive non-Hodgkin's lymphoma. Semin Oncol |
| | 29(2 Suppl 6): 18-22 (2002) (abstract). |
| | Witzig T E et al. Randomized controlled trial of yttrium-90-labeled |
| | ibritumomab tiuxetan radioimmunotherapy versus rituximab immunotherapy |
| | for patients with relapsed or refractory low-grade, follicular, or transformed |
| | B-cell non-Hodgkin's lymphoma.J Clin Oncol 20: 2453-6 (2003) (abstract). |
| | Maddipatla-S et al. Augmented Antitumor Activity against B-Cell |
| | Lymphoma by a Combination of Monoclonal Antibodies Targeting TRAIL- |
| | R1 and CD20. Clin Cancer Res 13(15): 4556-4564 (2007). |
| CD33 | Sievers E L et al. Selective ablation of acute myeloid leukemia using |
| | antibody-targeted chemotherapy: a phase I study of an anti-CD33 |
| | calicheamicin immunoconjugate.Blood 93: 3678-84 (1999). |
| | Hauswirth A W et al. The Target Receptor Siglec-3 (CD33) Is Expressed |
| | on AML Stem Cells in a Majority of All Patients with AML Blood 106 |
| | (11): 4324 (2005) (abstract). |
| | Caron PC et al. Biological and Immunological Features of Humanized |
| | M195 (Anti-CD33) Monoclonal Antibodies. Cancer Res 52(24): 6761-6767 |
| | (1992). |
| | Stiff P J et al. Anti-CD33 monoclonal antibody and etoposide/cytosine |
| | arabinoside combinations for the ex vivo purification of bone marrow in |
| | acute nonlymphocytic leukemia. Blood 77 (2): 355-362 (1991). |
| | Roy D C et al. Anti-MY9-blocked-ricin: an immunotoxin for selective |
| | targeting of acute myeloid leukemia cells. Blood 77 (11): 2404-2412 (1991). |
| CD22 | Carnahan J et al. Epratuzumab, a humanized monoclonal antibody |
| | targeting CD22: characterization of in vitro properties. Clin Cancer Res 9(10 |
| | Pt 2): 39825-905 (2003) (abstract). |
| | Kreitman R J et al. Efficacy of the anti-CD22 recombinant immunotoxin |
| | BL22 in chemotherapy-resistant hairy-cell leukemia. N Engl J Med 345: 241- |
| | 47 (2001). |
| | Robbins B A et al. Diagnostic application of two-color flow cytometry in |
| | 161 cases of hairy cell leukemia. Blood 82: 1277-87 (1993). |
| | Cordone I et al. Diagnostic relevance of peripheral blood |
| | Immunocytochemistry in hairy cell leukemia. J Clin Pathol 48: 955-960 (1995). |
| | Amlot P L et al. A phase I study of an anti-CD22-deglycosylated ricin A |
| | chain immunotoxin in the treatment of B-cell lymphomas resistant to |
| | conventional therapy. Blood 82: 2624-2633 (1993). |

The FDA maintains listings of approved antibody drugs for treating cancer, many of which bind to cancer antigens and can be employed in this context. See The Orange Book Online or Drugs@FDA on the FDA website. The FDA also maintains listings of clinical trials in progress in the clinicaltrials.gov database, which may be searched by disease names. Table 2C provides a representative list of approved antibodies with specificity for tumor cells. Table 2D provides a representative list of antibodies in development with specificity for tumor cells.

TABLE 2C
Representative antibodies approved for cancer indications

| International | | 1st indication |
|---|---|---|
| Nonproprietary Name | Target; Format | approved/reviewed |
| Ado- | HER2; Humanized IgG1, | Breast cancer |
| trastuzumab | ADC | |
| emtansine | | |
| Alemtuzumab | CD52; Humanized IgG1 | Chronic myeloid |
| | | leukemia; |
| | | multiple sclerosis |
| Atezolizumab | PD-L1 Humanized IgG1 | Bladder cancer |
| Avelumab | PD-L1; Human IgG1 | Merkel cell carcinoma |
| Bevacizumab | VEGF; Humanized IgG1 | Colorectal cancer |
| Blinatumomab | CD19, CD3; Murine | Acute lymphoblastic |
| | bispecific tandem scFv | leukemia |
| Brentuximab | CD30; Chimeric IgG1, | Hodgkin lymphoma, |
| vedotin | ADC | systemic anaplastic |
| | | large cell lymphoma |
| Catumaxomab | EPCAM/CD3; Rat/mouse | Malignant ascites |
| | bispecific mAb | |
| Cemiplimab | PD-1; Human mAb | Cutaneous squamous |
| | | cell carcinoma |
| Cetuximab | EGFR; Chimeric IgG1 | Colorectal cancer |
| Daratumumab | CD38; Human IgG1 | Multiple myeloma |
| Dinutuximab | GD2; Chimeric IgG1 | Neuroblastoma |
| Durvalumab | PD-L1; Human IgG1 | Bladder cancer |
| Edrecolomab | EpCAM; Murine IgG2a | Colorectal cancer |
| Elotuzumab | SLAMF7; Humanized IgG1 | Multiple myeloma |
| Gemtuzumab | CD33; Humanized IgG4, | Acute myeloid |
| | ADC | leukemia |
| Ibritumomab | CD20; Murine IgG1 | Non-Hodgkin |
| tiuxetan | | lymphoma |
| Inotuzumab | CD22; Humanized IgG4, | Hematological |
| | ADC | malignancy |
| Ipilimumab | CTLA-4; Human IgG1 | Metastatic melanoma |
| Mogamuizumab | CCR4; Humanized IgG1 | Cutaneous T-cell |
| | | lymphoma |
| Moxetumomab | CD22; Murine IgG1 dsFy | Hairy cell leukemia |
| pasudotox | immunotoxin | |
| Necitumumab | EGFR; Human IgG1 | Non-small cell lung |
| | | cancer |
| Nivolumab | PD-1; Human IgG4 | Melanoma, non-small |
| | | cell lung cancer |
| Obinutuzumab | CD20; Humanized IgGl; | Chronic lymphocytic |
| | Glycoengineered | leukemia |
| Ofatumumab | CD20; Human IgG1 | Chronic lymphocytic |
| | | leukemia |
| Olaratumab | PDGRFα; Human IgG1 | Soft tissue sarcoma |
| Panitumumab | EGFR; Human IgG2 | Colorectal cancer |
| Pembrolizumab | PD-1; Humanized IgG4 | Melanoma |
| Pertuzumab | HER2; Humanized IgG1 | Breast Cancer |
| Ramucirumab | VEGFR2; Human IgG1 | Gastric cancer |
| Rituximab | CD20; Chimeric IgG1 | Non-Hodgkin lymphoma |
| Sacituzumab | TROP-2; Humanized IgG1 | Triple-negative |
| govitecan | ADC | breast cancer |
| Tositumomab- | CD20; Murine IgG2a | Non-Hodgkin lymphoma |
| I131 | | |
| Trastuzumab | HER2; Humanized IgG1 | Breast cancer |

TABLE 2D
Antibodies in development for cancer indications

| INN or code | Molecular | | Late-stage |
|---|---|---|---|
| name | format | Target | study indication(s) |
| Utomilumab | Human IgG2 | CD137 | Diffuse large B-cell |
| | | (4-1BB) | lymphoma |
| XMAB-5574, | Humanized | CD19 | Diffuse large B-cell |
| MOR208 | IgG1 | | lymphoma |
| Ublituximab | Chimeric IgG1 | CD20 | Chronic |
| | | | lymphocytic |
| | | | Leukemia, non- |
| | | | Hodgkin |
| | | | lymphoma, multiple |
| | | | sclerosis |
| Moxetumomab | Murine IgG1 | CD22 | Hairy cell leukemia |
| pasudotox | dsFy | | |
| | immunotoxin | | |
| Isatuximab | Humanized | CD38 | Multiple myeloma |
| | IgG1 | | |
| Polatuzumab | Humanized | CD79b | Diffuse large B-cell |
| vedotin | IgG1 ADC | | lymphoma |
| Tremelimumab | Human IgG2 | CTLA-4 | Non-small cell lung, |
| | | | head & neck, |
| | | | urothelial cancer, |
| | | | hepatocellular |
| | | | carcinoma |
| Rovalpituzumab | Humanized | DLL3 | Small cell lung |
| tesirine | IgG1 ADC | | cancer |
| Depatuxizumab | IgG1 ADC | EGFR | Glioblastoma |
| mafodotin | | | |
| Carotuximab | Chimeric IgG1 | Endoglin | Soft tissue sarcoma, |
| | | | angiosarcoma, |
| | | | renal cell carcinoma, |
| | | | wet age- |
| | | | related macular |
| | | | degeneration |
| Oportuzumab | Humanized | EpCAM | Bladder cancer |
| monatox | scEv | | |
| | immunotoxin | | |
| L19IL2/ | scEv immuno- | Fibronectin | Melanoma |
| L19TNF | conjugates | extra- | |
| | | domain B | |
| Mirvetuximab | IgG1 ADC | Folate | Epithelial ovarian |
| soravtansine | | receptor 1 | cancer, peritoneal |
| | | | carcinoma, |
| | | | fallopian tube cancer |
| Glembatumumab | Human IgG2 | gpNMB | gpNMB+ breast |
| vedotin | ADC | | cancer, melanoma |
| Margetuximab | Chimeric IgG1 | HER2 | Breast cancer |
| (vic-) | Humanized | HER2 | Breast cancer |
| trastuzumab | IgG1 ADC | | |
| duocarmazine | | | |
| DS-8201 | Humanized | HER2 | HER2+ gastric or |
| | ADC | | gastroesophageal |
| | | | junction |
| | | | adenocarcinoma |
| Andecaliximab | Humanized | MMP-9 | Gastric cancer or |
| | IgG4 | | gastroesophageal |
| | | | junction |
| | | | adenocarcinoma |
| Racotumomab | Murine IgG1 | NGcGM3 | Non-small cell |
| | | | lung cancer |
| Camrelizumab | Humanized | PD-1 | Hepatocellular |
| | IgG4 | | carcinoma, |
| | | | esophageal |
| | | | carcinoma |
| Cemiplimab | Human mAb | PD-1 | Cutaneous |
| | | | squamous cell |
| | | | carcinoma; non- |
| | | | small cell lung |
| | | | cancer, |
| | | | cervical cancer |
| IBI308 | Human mAb | PD-1 | Squamous cell |
| | | | non-small cell |
| | | | lung cancer |
| BGB-A317 | Humanized | PD-1 | Non-small cell lung |
| | mAb | | cancer |
| BCD-100 | Human mAb | PD-1 | Melanoma |
| PDR001 | Humanized | PD-1 | Melanoma |
| | IgG4 | | |
| Sacituzumab | IgG1 ADC | TROP-2 | Triple-neg. breast |
| govitecan | | (epithelial | cancer |
| | | glyco- | |
| | | protein-1) | |

Other antibodies well-known in the art may be used as targeting moieties to target to a given cancer. The antibodies and their respective antigens include nivolumab (anti-PD-1 Ab), TA99 (anti-gp75), 3F8 (anti-GD2), 8H9 (anti-B7-H3), abagovomab (anti-CA-125 (imitation)), adecatumumab (anti-EpCAM), afutuzumab (anti-CD20), alacizumab pegol (anti-VEGFR2), altumomab pentetate (anti-CEA), amatuximab (anti-mesothelin), AME-133 (anti-CD20), anatumomab mafenatox (anti-TAG-72), apolizumab (anti-HLA-DR), arcitumomab (anti-CEA), bavituximab (anti-phosphatidylserine), bectumomab (anti-CD22), belimumab (anti-BAFF), besilesomab (anti-CEA-related antigen), bevacizumab (anti-VEGF-A), bivatuzumab mertansine (anti-CD44 v6), blinatumomab (anti-CD19), BMS-663513 (anti-CD137), brentuximab vedotin (anti-CD30 (TNFRSF8)), cantuzumab mertansine (anti-mucin CanAg), cantuzumab ravtansine (anti-MUC1), capromab pendetide (anti-prostatic carcinoma cells), carlumab (anti-MCP-1), catumaxomab (anti-EpCAM, CD3), cBR96-doxorubicin immunoconjugate (anti-Lewis-Y antigen), CC49 (anti-TAG-72), cedelizumab (anti-CD4), Ch. 14.18 (anti-GD2), ch-TNT (anti-DNA associated antigens), citatuzumab bogatox (anti-EpCAM), cixutumumab (anti-IGF-1 receptor), clivatuzumab tetraxetan (anti-MUC1), conatumumab (anti-TRAIL-R2), CP-870893 (anti-CD40), dacetuzumab (anti-CD40), daclizumab (anti-CD25), dalotuzumab (anti-insulin-like growth factor I receptor), daratumumab (anti-CD38 (cyclic ADP ribose hydrolase)), demcizumab (anti-DLL4), detumomab (anti-B-lymphoma cell), drozitumab (anti-DR5), duligotumab (anti-HER3), dusigitumab (anti-ILGF2), ecromeximab (anti-GD3 ganglioside), edrecolomab (anti-EpCAM), elotuzumab (anti-SLAMF7), elsilimomab (anti-IL-6), enavatuzumab (anti-TWEAK receptor), enoticumab (anti-DLL4), ensituximab (anti-SAC), epitumomab cituxetan (anti-episialin), epratuzumab (anti-CD22), ertumaxomab (anti-HER2/neu, CD3), etaracizumab (anti-integrin αvβ3), faralimomab (anti-Interferon receptor), farletuzumab (anti-folate receptor 1), FBTA05 (anti-CD20), ficlatuzumab (anti-HGF), figitumumab (anti-IGF-1 receptor), flanvotumab (anti-TYRP1 (glycoprotein 75)), fresolimumab (anti-TGF (3), futuximab (anti-EGFR), galiximab (anti-CD80), ganitumab (anti-IGF-I), gemtuzumab ozogamicin (anti-CD33), girentuximab (anti-carbonic anhydrase 9 (CAIX)), glembatumumab vedotin (anti-GPNMB), guselkumab (anti-IL13), ibalizumab (anti-CD4), ibritumomab tiuxetan (anti-CD20), icrucumab (anti-VEGFR-1), igovomab (anti-CA-125), IMAB362 (anti-CLDN18.2), IMC-CS4 (anti-CSF1R), IMC-TR1 (TGFβRII), imgatuzumab (anti-EGFR), inclacumab (anti-selectin P), indatuximab ravtansine (anti-SDC1), inotuzumab ozogamicin (anti-CD22), intetumumab (anti-CD51), ipilimumab (anti-CD152), iratumumab (anti-CD30 (TNFRSF8)), KM3065 (anti-CD20), KW-0761 (anti-CD194), LY2875358 (anti-MET) labetuzumab (anti-CEA), lambrolizumab (anti-PDCD1), lexatumumab (anti-TRAIL-R2), lintuzumab (anti-CD33), lirilumab (anti-KIR2D), lorvotuzumab mertansine (anti-CD56), lucatumumab (anti-CD40), lumiliximab (anti-CD23 (IgE receptor)), mapatumumab (anti-TRAIL-R1), margetuximab (anti-ch4D5), matuzumab (anti-EGFR), mavrilimumab (anti-GMCSF receptor a-chain), milatuzumab (anti-CD74), minretumomab (anti-TAG-72), mitumomab (anti-GD3 ganglioside), mogamulizumab (anti-CCR4), moxetumomab pasudotox (anti-CD22), nacolomab tafenatox (anti-C242 antigen), naptumomab estafenatox (anti-5T4), narnatumab (anti-RON), necitumumab (anti-EGFR), nesvacumab (anti-angiopoietin 2), nimotuzumab (anti-EGFR), nivolumab (anti-IgG4), nofetumomab merpentan, ocrelizumab (anti-CD20), ocaratuzumab (anti-CD20), olaratumab (anti-PDGF-R a), onartuzumab (anti-c-MET), ontuxizumab (anti-TEM1), oportuzumab monatox (anti-EpCAM), oregovomab (anti-CA-125), otlertuzumab (anti-CD37), pankomab (anti-tumor specific glycosylation of MUC1), parsatuzumab (anti-EGFL7), pascolizumab (anti-IL-4), patritumab (anti-HER3), pemtumomab (anti-MUC1), pertuzumab (anti-HER2/neu), pidilizumab (anti-PD-1), pinatuzumab vedotin (anti-CD22), pintumomab (anti-adenocarcinoma antigen), polatuzumab vedotin (anti-CD79B), pritumumab (anti-vimentin), PRO131921 (anti-CD20), quilizumab (anti-IGHE), racotumomab (anti-N-glycolylneuraminic acid), radretumab (anti-fibronectin extra domain-B), ramucirumab (anti-VEGFR2), rilotumumab (anti-HGF), robatumumab (anti-IGF-1 receptor), roledumab (anti-RHD), rovelizumab (anti-CD11 & CD18), samalizumab (anti-CD200), satumomab pendetide (anti-TAG-72), seribantumab (anti-ERBB3), SGN-CD19A (anti-CD19), SGN-CD33A (anti-CD33), sibrotuzumab (anti-FAP), siltuximab (anti-IL-6), solitomab (anti-EpCAM), sontuzumab (anti-episialin), tabalumab (anti-BAFF), tacatuzumab tetraxetan (anti-alpha-fetoprotein), taplitumomab paptox (anti-CD19), telimomab aritox, tenatumomab (anti-tenascin C), teneliximab (anti-CD40), teprotumumab (anti-CD221), TGN1412 (anti-CD28), ticilimumab (anti-CTLA-4), tigatuzumab (anti-TRAIL-R2), TNX-650 (anti-IL-13), tositumomab (anti-CS20), tovetumab (anti-CD140a), TRBS07 (anti-GD2), tregalizumab (anti-CD4), tremelimumab (anti-CTLA-4), TRU-016 (anti-CD37), tucotuzumab celmoleukin (anti-EpCAM), ublituximab (anti-CD20), urelumab (anti-4-1BB), vantictumab (anti-Frizzled receptor), vapaliximab (anti-AOC3 (VAP-1)), vatelizumab (anti-ITGA2), veltuzumab (anti-CD20), vesencumab (anti-NRP1), visilizumab (anti-CD3), volociximab (anti-integrin α5β1), vorsetuzumab mafodotin (anti-CD70), votumumab (anti-tumor antigen CTAA16.88), zalutumumab (anti-EGFR), zanolimumab (anti-CD4), zatuximab (anti-HER1), ziralimumab (anti-CD147 (basigin)), RG7636 (anti-ETBR), RG7458 (anti-MUC16), RG7599 (anti-NaPi2b), MPDL3280A (anti-PD-L1), RG7450 (anti-STEAP1), and GDC-0199 (anti-Bcl-2).

Antibodies that bind these antigens may also be used as targeting moieties, especially for the types of cancers noted: aminopeptidase N (CD13), annexin A1, B7-H3 (CD276, various cancers), CA125 (ovarian cancers), CA15-3 (carcinomas), CA19-9 (carcinomas), L6 (carcinomas), Lewis Y (carcinomas), Lewis X (carcinomas), alpha fetoprotein (carcinomas), CA242 (colorectal cancers), placental alkaline phosphatase (carcinomas), prostate s7pecific antigen (prostate), prostatic acid phosphatase (prostate), epidermal growth factor (carcinomas), CD2 (Hodgkin's disease, NHL lymphoma, multiple myeloma), CD3 epsilon (T-cell lymphoma, lung, breast, gastric, ovarian cancers, autoimmune diseases, malignant ascites), CD19 (B cell malignancies), CD20 (non-Hodgkin's lymphoma, B-cell neoplasmas, autoimmune diseases), CD21 (B-cell lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (metastatic melanoma, sarcoma), CD52 (leukemia), CD56 (small cell lung cancers, ovarian cancer, Merkel cell carcinoma, and the liquid tumor, multiple myeloma), CD66e (carcinomas), CD70 (metastatic renal cell carcinoma and non-Hodgkin lymphoma), CD74 (multiple myeloma), CD80 (lymphoma), CD98 (carcinomas), CD123 (leukemia), mucin (carcinomas), CD221 (solid tumors), CD22 (breast, ovarian cancers), CD262 (NSCLC and other cancers), CD309 (ovarian cancers), CD326 (solid tumors), CEACAM3 (colorectal, gastric cancers), CEACAM5 (CEA, CD66e) (breast, colorectal and lung cancers), DLL4 (A-like-4), EGFR (various cancers), CTLA4 (melanoma), CXCR4 (CD 184, heme-oncology, solid tumors), Endoglin (CD 105, solid tumors), EPCAM (epithelial cell adhesion molecule, bladder, head, neck, colon, NHL prostate, and ovarian cancers), ERBB2 (lung, breast, prostate cancers), FCGR1 (autoimmune diseases), FOLR (folate receptor, ovarian cancers), FGFR (carcinomas), GD2 ganglioside (carcinomas), G-28 (a cell surface antigen glycolipid, melanoma), GD3 idiotype (carcinomas), heat shock proteins (carcinomas), HER1 (lung, stomach cancers), HER2 (breast, lung and ovarian cancers), HLA-DR10 (NHL), HLA-DRB (NHL, B cell leukemia), human chorionic gonadotropin (carcinomas), IGF1R (solid tumors, blood cancers), IL-2 receptor (T-cell leukemia and lymphomas), IL-6R (multiple myeloma, RA, Castleman's disease, IL6 dependent tumors), integrins (αvβ3, α5β1, α6β4, α11β3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 (breast, ovarian, cervix, bronchus and gastrointestinal cancer), MUC16 (CA125) (ovarian cancers), CEA (colorectal cancer), gp100 (melanoma), MARTI (melanoma), MPG (melanoma), MS4A1 (membrane-spanning 4-domains subfamily A, small cell lung cancers, NHL), nucleolin, Neu oncogene product (carcinomas), P21 (carcinomas), nectin-4 (carcinomas), paratope of anti-(N-glycolylneuraminic acid, breast, melanoma cancers), PLAP-like testicular alkaline phosphatase (ovarian, testicular cancers), PSMA (prostate tumors), PSA (prostate), ROB04, TAG 72 (tumour associated glycoprotein 72, AML, gastric, colorectal, ovarian cancers), T-cell transmembrane protein (cancers), Tie (CD202b), tissue factor, TNFRSF10B (tumor necrosis factor receptor superfamily member 10B, carcinomas), TNFRSF13B (tumor necrosis factor receptor superfamily member 13B, multiple myeloma, NHL, other cancers, RA and SLE), TPBG (trophoblast glycoprotein, renal cell carcinoma), TRAIL-R1 (tumor necrosis apoptosis inducing ligand receptor 1, lymphoma, NHL, colorectal, lung cancers), VCAM-1 (CD106, Melanoma), VEGF, VEGF-A, VEGF-2 (CD309) (various cancers). Some other tumor associated antigen targets have been reviewed (Gerber, et al, mAbs 2009 1:247-253; Novellino et al, Cancer Immunol Immunother. 2005 54:187-207, Franke, et al, Cancer Biother Radiopharm. 2000, 15:459-76, Guo, et al., Adv Cancer Res. 2013; 119: 421-475, Parmiani et al. J Immunol. 2007 178:1975-9). Examples of these antigens include Cluster of Differentiations (CD4, CDS5, CD6, CD7, CD8, CD9, CD10, CD11a, CD11b, CD11c, CD12w, CD14, CD15, CD16, CDw17, CD18, CD21, CD23, CD24, CD25, CD26, CD27, CD28, CD29, CD31, CD32, CD34, CD35, CD36, CD37, CD41, CD42, CD43, CD44, CD45, CD46, CD47, CD48, CD49b, CD49c, CD53, CD54, CD55, CD58, CD59, CD61, CD62E, CD62L, CD62P, CD63, CD68, CD69, CD71, CD72, CD79, CD81, CD82, CD83, CD86, CD87, CD88, CD89, CD90, CD91, CD95, CD96, CD100, CD103, CD105, CD106, CD109, CD117, CD120, CD127, CD133, CD134, CD135, CD138, CD141, CD142, CD143, CD144, CD147, CD151, CD152, CD154, CD156, CD158, CD163, CD166, CD168, CD184, CDw186, CD195, CD202 (a, b), CD209, CD235a, CD271, CD303, CD304), annexin A1, nucleolin, endoglin (CD105), ROB04, amino-peptidase N, -like-4 (DLL4), VEGFR-2 (CD309), CXCR4 (CD184), Tie2, B7-H3, WT1, MUC1, LMP2, HPV E6 E7, EGFRvIII, HER-2/neu, idiotype, MAGE A3, p53 nonmutant, NY-ESO-1, GD2, CEA, MelanA/MART1, Ras mutant, gp100, p53 mutant, proteinase3 (PR1), bcr-abl, tyrosinase, survivin, hTERT, sarcoma translocation breakpoints, EphA2, PAP, ML-IAP, AFP, EpCAM, ERG (TMPRSS2 ETS fusion gene), NA17, PAX3, ALK, androgen receptor, cyclin B 1, polysialic acid, MYCN, RhoC, TRP-2, GD3, fucosyl GM1, mesothelin, PSCA, MAGE A1, sLe(a), CYPIB I, PLAC1, GM3, BORIS, Tn, GloboH, ETV6-AML, NY-BR-1, RGS5, SART3, STn, carbonic anhydrase IX, PAXS, OY-TES1, sperm protein 17, LCK, HMWMAA, AKAP-4, SSX2, XAGE 1, B7H3, legumain, Tie 2, Page4, VEGFR2, MAD-CT-1, FAP, PDGFR-β, MAD-CT-2, and Fos-related antigen 1.

In some embodiments, the antibody or antigen-binding fragment thereof is specific for 4-1BB, 5T4, ACVRL1, ALK1, AXL, B7-H3, BCMA, c-MET, CD133, C4.4a, CA6, CA9, Cadherin-6, CD123, CD133, CD138, CD19, CD20, CD22, CD25, CD27L, CD30, CD33, CD37, CD38, CD44v6, CD56, CD70, CD74(TROP2), CD79b, CEA, CEACAM5, cKit, CLL-1, Cripto, CS1, DLL3, EDNRB, EFNA4, EGFR, EGFRvIII, ENPP3, EpCAM, EPHA2, FGFR2, FGFR3, FLT3, FOLR, FOLR1, GD2, gpA33, GPC3, GPNMB, GUCY2C, HER2, HER3, HLAA2, IGF1-r, IL13RA2, Integrin alpha, LAMP-1, LewisY, LIV-1, LRRC15, MMP9, MSLN, MUC1, MUC16, NaPi2b, Nectin-4, NOTCH3, p-CAD, PD-L1, PSMA, PTK7, ROR1, SLC44A4, SLITRK6, SSTR2, STEAP1, TAG72, TF, TIM-1, or TROP-2.

In some embodiments, the antibody or antigen-binding fragment thereof is specific for Her2/Neu, CD22, PSMA, CD30, CD2, CD33, CD8, CD86, CD, CA125, Carbonic Anhydrase IX, CD70, CD74, CD56, CD40, CD19, e-met/HGFR, TRAIL-R1, DRS, PD-1, IGF-1R, VEGF-R2, PSCA, MUC1, CanAg, Mesothelin, P-cadherin, Myostatin, Cripto, ACVRL 1/ALK1, MUCSAC, CEACAM, CD137, CXCR4, Neuropilin 1, Glypicans, HERS/EGFR, PDGFRa, EphA2, CD38, CD138/Syndecanl, A4-integrin, EpCAM, ADAM17, CD59, Integrin aVf33, MCP-1, PCLA, RANKL, RG1, SLC44A4, STEAP-1, VEGF-C, CCN1, CD44, CD98, c-RET, DLL4, Episialin, GPNMB, Integrin α6β4, LFL2, LIV-1, Ly6E, MUC18, NRP1, Phosphatidylserine, PRLR, TACSTD-2, Tenascin C, TWEAKR, VANGL2, PD-L1, PD-L2, BCMA, DKK-1, ICAM-q, GRP78, FGFR3, SLAMF6, CD48, CD71, APRIL, DR5, CD37, HLA-DR, CD70b, CAIX, TPBG, ENPP3, FGFR1, VEGFR-2, CLDN18, GCC, C242, FGFR2, GPR49, IGFR, ALK, GC2, EGFRvIII, CD4, CD5, IL-3Ra, Integrin α5β1, Lewis y/b antigen, EGFL7, NaPi2b, flt4, CD133, CD123, CD45, c-Kit, Lewis Y, Siglec-15, FLT-3, CEACAM1, Cadherin-19, GM3, TYRP1, GD3, MUC5A, CLDN6, Glypican-3, FGFR4, PIVKA-II, PLVAP, Progastrin, CEA, CLDN1, A33, CK8, or FAP.

In some embodiments, the antibody or antigen-binding fragment thereof is an anti-epidermal growth factor receptor antibody; an anti-Her2 antibody; an anti-CD20 antibody; an anti-CD22 antibody; an anti-CD70 antibody; an anti-CD33 antibody; an anti-MUC1 antibody; an anti-CD40 antibody; an anti-CD74 antibody; an anti-P-cadherin antibody; an anti-EpCAM antibody; an anti-CD138 antibody; an anti-E-cadherin antibody; an anti-CEA antibody; an anti-FGFR3 antibody; an anti-mucin core protein antibody; an anti-transferrin antibody; an anti-gp95/97 antibody; an anti-p-glycoprotein antibody; an anti-TRAIL-R1 antibody; an anti-DR5 antibody; an anti-IL-4 antibody; an anti-IL-6 antibody; an anti-CD19 antibody; an anti-PSMA antibody; an anti-PSCA antibody; an anti-Cripto antibody; an anti-PD-L1 antibody; an anti-IGF-1R antibody; an anti-CD38 antibody; an anti-CD133 antibody; an anti-CD123 antibody; an anti-CDE49d antibody; an anti-glypican 3 antibody; an anti-cMET antibody; or an anti-IL-13R antibody.

In some embodiments, the antibody or antigen-binding fragments comprises all or part of the amino acid sequence of 1C1, (GS) 5745, ABBV-085, ABBV-399, ABBV-838, AbGn-107, ABT-414, ADCT-301, ADCT-402, AGS-16C3F, AGS62P1, AGS67E, AMG 172d, AMG 595d, Andecaliximab, Anetumab ravtansine, ARX788, ASG-15MEd, ASG-5MEk, Atezolizumab, AVE1642, AVE9633e, Avelumab, BAY1129980, BAY1187982e, BAY79-4620b, BIIB015d, Bivatuzumab mertansineb, BMS-986148, Brentuximab vedotin, Cantuzumab mertansine, CC49, CDX-014, Cirmtuzumab, Coltuximab ravtansine, DEDN6526Ae, Denintuzumab mafodotin, Depatuxizumab, DFRF4539Ad, DMOT4039Ae, DS-8201A, Durvalumab, Enfortumab vedotin, Farletuzumab, FLYSYN, Gatipotuzumab, Gemtuzumab ozogamicin, Glembatumumab vedotin, GSK2857916, HKT288, Hu3F8, HuMax-AXL-ADC, IDEC-159, IMGN289b, IMGN388a, IMGN529, Indatuximab ravtansine, Inotuzumab ozogamicin, Istiratumab, Labetuzumab govitecan, Lifastuzumab vedotin, LOP628h, Lorvotuzumab mertansine, LY3076226, MCLA-117 (CLEC-12AxCD3), MDX-1203d, MEDI-4276, MEDI-547b, Milatuzumab-doxorubicin, Mirvetuximab soravtansine, MLN0264, MLN2704e, MM-302i, Mosunetuzumab, MOv18 IgE, Ocrelizumab, Oportuzumab, Patritumab, PCA-062, PF-03446962, PF-06263507a, PF-06647020, PF-06647263, PF-06650808d, Pinatuzumab vedotin, Polatuzumab vedotin, PSMA ADC 301c, RC48-ADC, Rituximab, Rovalpituzumab tesirine, Sacituzumab, Sacituzumab govitecan, SAR408701, SAR428926, SAR566658, SC-002, SC-003, SGN-15a, SGN-CD123A, SGN-CD19B, SGN-CD70A, SGN-LIV1A, Sofituzumab vedotin, Solitomab, SSTR2xCD3 XmAb18087, STRO-002, SYD-985, Talacotuzumab, Tisotumab vedotin, Trastuzumab emtansine, U3-1402, Ublituximab, Vadastuximab talirine, Vandortuzumab vedotin, Vorsetuzumab mafodotin, XMT-1522, or Zenocutuzumab.

In some embodiments, the targeting moiety capable of targeting a cancer is not an antibody, but is another type of targeting moiety. A wide range of targeting moieties capable of targeting cancer are known, including DNA aptamers, RNA aptamers, albumins, lipocalins, fibronectins, ankyrins, CH1/2/3 scaffolds (including abdurins (IgG CH2 scaffolds)), fynomers, Obodies, DARPins, knotins, avimers, atrimers, anticallins, affilins, affibodies, bicyclic peptides, cys-knots, FN3 (adnectins, centryrins, pronectins, TN3), and Kunitz domains. These and other non-antibody scaffold structures may be used for targeting to a cancer cell. Smaller non-antibody scaffolds are rapidly removed from the bloodstream and have a shorter half-life than monocolonal antibodies. They also show faster tissue penetration owing to fast extravasation from the capillary lumen through the vascular endothelium and basement membrane. See Vazquez-Lombardi et al., Drug Discovery Today 20(1):1271-1283 (2015). A number of non-antibody scaffolds targeting cancer are already under clinical development, with other candidates in the preclinical stage, as shown in Table 2E. See Vazquez-Lombardi, Table 1.

TABLE 2E
Non-Antibody Scaffolds and Corresponding Targets

| Scaffold | Demonstrated Targets |
|---|---|
| Adnectin | EGFR, IGF-1R |
| Affibodies | HER2, EGFR, IGF-1R, HER3 |
| Affinlins | CTLA-4 |
| Anticalins | CD137/HER2 (a bispecific) |
| Atrimers | DR4 |
| Avimers | IL6 (could be used in oncology to block growth) |
| Bicyclic peptides | HER2 |
| Cys-knots | NaV1.7 (proof of concept) |
| DARPins | VEGF-a, HER2, VEGF/HGF (bispecific) |
| Fynomers | HER2 |
| Pronectins | VEGFR2 |
| TN3 | TRAILR2 |

In some embodiments, one or more targeting moiety comprises IL-2, IL-4, IL-6, α-MSH, transferrin, folic acid, EGF, TGF, PD1, IL-13, stem cell factor, insulin-like growth factor (IGF), or CD40. In some embodiments, one or more targeting moiety comprises a full-length sequence of IL-2, IL-4, IL-6, α-MSH, transferrin, folic acid, EGF, TGF, PD1, IL-13, stem cell factor, insulin-like growth factor (IGF), or CD40. In some embodiments, one or more targeting moiety comprises a truncated form, analog, variant, or derivative of IL-2, IL-4, IL-6, α-MSH, transferrin, folic acid, EGF, TGF, PD1, IL-13, stem cell factor, insulin-like growth factor (IGF), or CD40. In some embodiments, one or more targeting moiety binds a target on the cancer comprising IL-2 receptor, IL-4, IL-6, melanocyte stimulating hormone receptor (MSH receptor), transferrin receptor (TR), folate receptor 1 (FOLR), folate hydroxylase (FOLH1), EGF receptor, PD-L1, PD-L2, IL-13R, CXCR4, IGFR, or CD40L.

In some embodiments, one or more targeting moiety is an aptamer. In some embodiments, the aptamer comprises DNA. In some embodiments, the aptamer comprises RNA. In some embodiments, the aptamer is single-stranded.

In some embodiments, the aptamer is a target cell-specific aptamer chosen from a random candidate library. In some embodiments, the aptamer is an anti-EGFR aptamer. In some embodiments, the aptamer binds to the antigen on the cancer cell with a Kd from 1 picomolar to 500 nanomolar. In some embodiments, the aptamer binds to the cancer with a Kd from 1 picomolar to 100 nanomolar.

Additional specific targeting moieties include those provided in Table 3.

TABLE 3
Selected examples of non-immunoglobulin and antigen-binding
fragments of antibodies that can serve as targeting molecules

| Target Antigen | Format Scaffold | Reference |
|---|---|---|
| DKK1 | VHH | WO2010/130832 |
| c-Met | VHH | US2012/0244164 |
| TfR (CD71) | VNAR | US2017/0348416 |
| CD33 | Fynomer | WO2014/170063 |
| HLA-A*02:01 | TCR | IMCgp100 |
| gp100 | | |
| HLA-A*02:01NY- | TCR | US2018/0072788 |
| ESO | | |
| HER3 | Affibody | WO2014/053586A1 |
| HER2 | Affibody | US2010/0254899A1 |
| VEGF, HGF | DARPin | MP0250 |
| EGFR/HER2 | DARPin | U.S. Pat. No.9,499,622B2 |
| EphA2 | Abdurin (CH2) | US2015/0353943 |

C. Immune Cell Selection Moiety

In some embodiments, an ATTAC comprises a targeting moiety and an immune cell selection moiety. In other words, an ATTAC may comprise one component that binds to an antigen on a cancer cell and another component that binds to an immune cell.

In some embodiments, an ATTAC comprises an immune cell selection moiety specific for a particular immune cell. In some embodiments, the immune cell selection moiety is specific for CD8+ T cells, CD4+ T cells, natural killer (NK) cells, macrophages, neutrophils, eosinophils, basophils, γδ T cells, natural killer T cells (NKT cells), or engineered immune cells. Engineered immune cells refers to immune cells with engineered receptors with new specificity. Examples of engineered immune cells include chimeric antigen receptor (CAR) T cells, NK, NKT, or γδ T cells.

In some embodiments, the immune cell selection moiety targets an immune cell marker that is not a tumor antigen. In some embodiments, the immune cell selection moiety allows targeting of an ATTAC to an immune cell, wherein the immune cell is not a cancer cell. In some embodiments, the immune cell selection moiety does not target the ATTAC to a lymphoma, myeloma, or leukemia. In some embodiments, the ATTAC targets a solid tumor (in other words any tumor not of an immune cell).

In some embodiments, the immune cell selection moiety does not specifically bind regulatory T cells. In some embodiments, the immune cell selection moiety does not specifically bind TH17 cells. In some embodiments, the selective immune cell binding agent does not target markers present on regulatory immune cells (including, but not limited to CD4 and CD25).

Table 4 lists some representative immune cell selection moieties for different desired immune cells.

TABLE 4
Immune Cell Selection Moiety

| Desired | Immune Cell | Immune Cell | |
|---|---|---|---|
| Immune Cell | Marker | Selection Moiety | Citations for Representative Species |
| CD8+T | CD8 | Antibodies | El Menshawy N et al. CD58; Leucocyte Function |
| Cells | | or antigen | Adhesion-3 (LFA-3) Could Be Used as a |
| | | binding | Differentiating Marker between Immune and Non- |
| | | fragments | Immune Thyroid Disorders. Comparative Clinical |
| | | thereof to | Pathology 27.3: 721-727 (2018). |
| | | CD8 | Guo Y et al. Immune checkpoint inhibitor PD-1 |
| | | | pathway is down-regulated in synovium at various |
| | | | stages of rheumatoid arthritis disease progression. |
| | | | Heymann D, ed. PLoS ONE. 2018; 13(2): e0192704. |
| | | | Tavaré R, Escuin-Ordinas H, Mok S, et al. An |
| | | | effective immuno-PET imaging method to monitor |
| | | | CD8-dependent responses to immunotherapy. Cancer |
| | | | Research. 76(1): 73-82 (2016). |
| | | | Darmochwal-Kolarz D et al. |
| | | | CD3+CD8+ Lymphocytes Are More Susceptible for |
| | | | Apoptosis in the First Trimester of Normal Human |
| | | | Pregnancy. Journal of Immunology Research. |
| | | | 2014: 670524 (2014). |
| | | | Chen G et al. Cigarette Smoke Disturbs the |
| | | | Survival of CD8+ Tc/Tregs Partially through |
| | | | Muscarinic Receptors-Dependent Mechanisms in |
| | | | Chronic Obstructive Pulmonary Disease. Su Y, ed. |
| | | | PLoS ONE. 11(1): e0147232 (2016). |
| | | | Brazowski E et al. FOXP3 expression in duodenal |
| | | | mucosa in pediatric patients with celiac disease. |
| | | | Pathobiology. 77(6): 328-34 (2010). |
| | | | Clement M et al. Anti-CD8 antibodies can trigger |
| | | | CD8+ T cell effector function in the absence of TCR |
| | | | engagement and improve peptide-MHCI tetramer |
| | | | staining. J Immunol. 187(2): 654-63 (2011). |
| | | Aptamers | Wang C W et al. A new nucleic acid-based agent |
| | | to CD8 | inhibits cytotoxic T lymphocyte-mediated immune |
| | | | disorders. J Allergy Clin Immunol. 132(3): 713-722 |
| | | | (2013). |
| | CXCR3 | Antibodies | Robert R et al. A fully humanized IgG-like |
| | | or antigen | bispecific antibody for effective dual targeting of |
| | | binding | CXCR3 and CCR6. PLoS One. 12(9): e0184278 |
| | | fragments | (2017). |
| | | thereof to | Lintermans L L, Rutgers A, Stegeman C A, |
| | | CXCR3 | Heeringa P, Abdulahad W H. Chemokine receptor co- |
| | | | expression reveals aberrantly distributed TH effector |
| | | | memory cells in GPA patients. Arthritis Research & |
| | | | Therapy. 19: 136(2017). |
| | | | Rojas-Dotor S et al. Expression of resistin, |
| | | | CXCR3, IP-10, CCR5 and MIP-1α in obese patients |
| | | | with different severity of asthma. |
| | | | Biol Res. 46(1): 13-20 (2013). |
| | | | Agostini C et al. Involvement of the IP-10 |
| | | | Chemokine in Sarcoid Granulomatous Reactions. J |
| | | | Immunol. 161 (11) 6413-6420 (1998). |
| | | | Jiskra J et al. CXCR3, CCR5, and CRTH2 |
| | | | Chemokine Receptor Expression in Lymphocytes |
| | | | Infiltrating Thyroid Nodules with Coincident |
| | | | Hashimoto's Thyroiditis Obtained by Fine Needle |
| | | | Aspiration Biopsy. J Immunol Res. 2016: 2743614 |
| | | | (2016). |
| | | | Lübbers J et al. Changes in peripheral blood |
| | | | lymphocyte subsets during arthritis development in |
| | | | arthralgia patients. Arthritis Research & Therapy. |
| | | | 18:205 (2016). |
| CD4+T | CD4 | Antibodies | De Graav G N et al. Follicular T helper cells and |
| Cells | | or antigen | humoral reactivity in kidney transplant patients. Clin |
| | | binding | Exp Immunol. 180(2): 329-340 (2015). |
| | | fragments | Duluc D et al. Induction and activation of human |
| | | thereof to | Th17 by targeting antigens to dendritic cells via |
| | | CD4 | Dectin-1. J Immunol 192(12):5776-5788 (2014). |
| | | | Flamar, Anne-Laure et al. “Targeting |
| | | | Concatenated HIV Antigens to Human CD40 |
| | | | Expands a Broad Repertoire of Multifunctional CD4+ |
| | | | and CD8+ T Cells.” AIDS. 27:13 (2013). |
| | | | Almanzar G et al. Autoreactive HSP60 epitope- |
| | | | specific T-cells in early human atherosclerotic |
| | | | lesions. J Autoimmun. 39(4):441-50 (2012). |
| | | | Babaei A et al. Production of a recombinant anti- |
| | | | human CD4 single-chain variable-fragment antibody |
| | | | using phage display technology and its expression in |
| | | | Escherichia coli. J Microbiol Biotechnol. 21(5): 529- |
| | | | 35 (2011). |
| | | Aptamers | Davis K A et al. Staining of cell surface human |
| | | to CD4 | CD4 with 2′-F-pyrimidine-containing RNA aptamers |
| | | | for flow cytometry. Nucleic Acids Research. |
| | | | 26(17): 3915-3924 (1998). |
| | | | Zhou Q et al. Aptamer-containing surfaces for |
| | | | selective capture of CD4 expressing cells. Langmuir. |
| | | | 28(34): 12544-9 (2012). |
| | | | Zhao N et al. Blocking interaction of viral gp120 |
| | | | and CD4-expressing T cells by single-stranded DNA |
| | | | aptamers. Int J Biochem Cell Biol. 51: 10-8 (2014). |
| | | | Peng Z et al. Combination of an Aptamer Probe to |
| | | | CD4 and Antibodies for Multicolored Cell |
| | | | Phenotyping. American Journal of Clinical |
| | | | Pathology, 134(4): 586-593 (2010). |
| | | | Cong-Qiu C et al. CD4 Aptamer-RORγt shRNA |
| | | | Chimera Inhibits IL-17 Synthesis By Human CD4+ T |
| | | | cells. American College of Rheumatology 2014 |
| | | | Annual Meeting Abstract Number 1751 (2014). |
| | CXCR3 | see above | see above |
| Natural | CD56 | Antibody | Whiteman et al. Lorvotuzumab mertansine, a |
| Killer Cells | | to CD56 | CD56-targeting antibody-drug conjugate with potent |
| | | | antitumor activity against small cell lung cancer in |
| | | | human xenograft models. MAbs. 6(2): 556-66 (2014). |
| | | | Shah et al. Phase I study of IMGN901, a CD56- |
| | | | targeting antibody-drug conjugate, in patients with |
| | | | CD56-positive solid tumors. Invest New Drugs. |
| | | | 34:290-299 (2016). |
| | | | Feng et al. Differential killing of CD56- |
| | | | expressing cells by drug-conjugated human |
| | | | antibodies targeting membrane-distal and membrane- |
| | | | proximal non-overlapping epitopes. MAbs. 8(4): 799- |
| | | | 810(2016). |
| | | | Galli et al. In Vivo Imaging of Natural Killer Cell |
| | | | Trafficking in Tumors. J Nucl Med. 56(10): 1575-80 |
| | | | (2015). |
| | | | Merkt et al. Peripheral blood natural killer cell |
| | | | percentages in granulomatosis with polyangiitis |
| | | | correlate with disease inactivity and stage. Arthritis |
| | | | Res Ther. 17: 337 (2015). |
| | | | Park et al. Gene expression analysis of ex vivo |
| | | | expanded and freshly isolated NK cells from cancer |
| | | | patients. J Immunother. 33(9): 945-55 (2010). |
| | | | Kimura et al. Tumor-draining lymph nodes of |
| | | | primary lung cancer patients: a potent source of |
| | | | tumor-specific killer cells and dendritic cells. |
| | | | Anticancer Res. 25(1A): 85-94 (2005). |
| | | | Mavoungou et al. Natural killer (NK) cell- |
| | | | mediated cytolysis of Plasmodium falciparum- |
| | | | infected human red blood cells in vitro. Eur Cytokine |
| | | | Netw. 14(3): 134-42 (2003). |
| | | | Yanagihara et al. Natural killer (NK) T cells are |
| | | | significantly decreased in the peripheral blood of |
| | | | patients with rheumatoid arthritis (RA). Clin Exp |
| | | | Immunol. 118(l):131-6 (1999). |
| | | | Roguska et al. Humanization of murine |
| | | | monoclonal antibodies through variable domain |
| | | | resurfacing. Proc Natl Acad Sci USA. 91(3): 969-73 |
| | | | (1994). |
| | | | Nitta et al. Involvement of CD56 (NKH-1/Leu-19 |
| | | | antigen) as an adhesion molecule in natural killer- |
| | | | target cell interaction. J Exp Med. 170(5): 1757-61 |
| | | | (1989). |
| | CD2 | Antibody | Listed in Table 2B |
| | | to CD2 | |
| Macrophages | CD14 | Antibody | Spek et al. Treatment with an anti-CD14 |
| | | to CD14 | monoclonal antibody delays and inhibits |
| | | | lipopolysaccharide-induced gene expression in |
| | | | humans in vivo. J Clin Immunol. 23(2): 132-40 |
| | | | (2003). |
| | | | Nakamura et al. Anti-human CD14 monoclonal |
| | | | antibody improves survival following sepsis induced |
| | | | by endotoxin, but not following polymicrobial |
| | | | infection. Eur J Pharmacol. 806: 18-24 (2017). |
| | | | Egge et al. The anti-inflammatory effect of |
| | | | combined complement and CD14 inhibition is |
| | | | preserved during escalating bacterial load. Clin Exp |
| | | | Immunol. 181(3): 457-67 (2015). |
| | | | Yidrim et al. Galectin-2 induces a |
| | | | proinflammatory, anti-arteriogenic phenotype in |
| | | | monocytes and macrophages. PLoS One. |
| | | | 10(4): e0124347 (2015). |
| | | | Hermansson et al. Macrophage CD14 expression |
| | | | in human carotid plaques is associated with |
| | | | complicated lesions, correlates with thrombosis, and |
| | | | is reduced by angiotensin receptor blocker treatment. |
| | | | Int Immunopharmacol. 22(2): 318-23 (2014). |
| | | | Genth-Zotz et al. The anti-CD14 antibody IC14 |
| | | | suppresses ex vivo endotoxin stimulated tumor |
| | | | necrosis factor-alpha in patients with chronic heart |
| | | | failure. Eur J Heart Fail. 8(4): 366-72 (2006). |
| | | | Olszyna et al. Effect of IC14, an anti-CD14 |
| | | | antibody, on plasma and cell-associated chemokines |
| | | | during human endotoxemia. Eur Cytokine Netw. |
| | | | 14(3): 158-62 (2003). |
| | | | Bondeson et al. The role of synovial macrophages |
| | | | and macrophage-produced cytokines in driving |
| | | | aggrecanases, matrix metalloproteinases, and other |
| | | | destructive and inflammatory responses in |
| | | | osteoarthritis. Arthritis Res Ther. 8(6): R187 (2006). |
| | | | Streit et al. 3D parallel coordinate systems--a new |
| | | | data visualization method in the context of |
| | | | microscopy-based multicolor tissue cytometry. |
| | | | Cytometry A. 69(7): 601-11 (2006). |
| | | | Ueki et al. Self-heat shock protein 60 induces |
| | | | tumour necrosis factor-alpha in monocyte-derived |
| | | | macrophage: possible role in chronic inflammatory |
| | | | periodontal disease. Clin Exp Immunol. 127(1): 72-7 |
| | | | (2002). |
| | CD11b | Antibodies | Gordon et al. Both anti-CD11a(LFA-1) and anti- |
| | | to CD11b | CD11b (MAC-1) therapy delay the onset and |
| | | | diminish the severity of experimental autoimmune |
| | | | encephalomyelitis. J Neroimmunol. 62(2): 153-160 |
| | | | (1995). |
| | | | Nakagawa et al. Optimum immunohistochemical |
| | | | procedures for analysis of macrophages in human and |
| | | | mouse formalin fixed paraffin-embedded tissue |
| | | | samples. J Clin Exp Hematop. 57(1): 31-36 (2017). |
| | | | Duarte et al. Generation of Immunity against |
| | | | Pathogens via Single-Domain Antibody-Antigen |
| | | | Constructs. J Immunol. 197(12): 4838-4847 (2016). |
| | | | Lau et al. Myeloperoxidase mediates neutrophil |
| | | | activation by association with CD11b/CD18 |
| | | | integrins. Proc Natl Acad Sci USA. 102(2): 431-6 |
| | | | (2005). |
| | | | May et al. Urokinase receptor surface expression |
| | | | regulates monocyte adhesion in acute myocardial |
| | | | infarction. Blood. 100(10): 3611-7 (2002). |
| | | | Ribbens et al. CD40-CD40 ligand (CD154) |
| | | | engagement is required but may not be sufficient for |
| | | | human T helper 1 cell induction of interleukin-2- or |
| | | | interleukin-15-driven, contact-dependent, interleukin- |
| | | | 1beta production by monocytes. Immunology. |
| | | | 99(2): 279-86 (2000). |
| | | | Olivieri et al. Increased neutrophil adhesive |
| | | | capability in Cohen syndrome, an autosomal |
| | | | recessive disorder associated with granulocytopenia. |
| | | | Haematologica. 83(9): 778-82 (1998). |
| | | | Rambaldi et al. Innovative two-step negative |
| | | | selection of granulocyte colony-stimulating factor- |
| | | | mobilized circulating progenitor cells: adequacy for |
| | | | autologous and allogeneic transplantation. Blood. |
| | | | 91(6): 2189-96 (1998). |
| | | | Lechner et al. Peripheral blood mononuclear cells |
| | | | from neovascular age-related macular degeneration |
| | | | patients produce higher levels of chemokines CCL2 |
| | | | (MCP-1) and CXCL8 (IL-8). J Neuroinflammation. |
| | | | 14(1): 42 (2017). |
| | | | Mizee et al. Isolation of primary microglia from |
| | | | the human post-mortem brain: effects of ante- and |
| | | | post-mortem variables. Acta Neuropathol Commun. |
| | | | 17; 5(1): 16 (2007). |
| | CD40 | Antibodies | French et al. CD40 antibody evokes a cytotoxic |
| | | to CD40 | T-cell response that eradicates lymphoma and |
| | | | bypasses T-cell help. Nature Medicine. 5: 548-553 |
| | | | (1999). |
| | | | Beatty et al. CD40 Agonists Alter Tumor Stroma |
| | | | and Show Efficacy Against Pancreatic Carcinoma in |
| | | | Mice and Humans. Science. 331(6024): 1612-1616 |
| | | | (2011). |
| | | | Velasquez et al. Targeting Mycobacterium |
| | | | tuberculosis Antigens to Dendritic Cells via the DC- |
| | | | Specific-ICAM3-Grabbing-Nonintegrin Receptor |
| | | | Induces Strong T-Helper 1 Immune Responses. Front |
| | | | Immunol. 9: 471 (2018). |
| | | | McDonnell et al. Serial immunomonitoring of |
| | | | cancer patients receiving combined antagonistic anti- |
| | | | CD40 and chemotherapy reveals consistent and |
| | | | cyclical modulation of T cell and dendritic cell |
| | | | parameters. BMC Cancer. 17(1): 417 (2017). |
| | | | Dahan et al. Therapeutic Activity of Agonistic, |
| | | | Human Anti-CD40 Monoclonal Antibodies Requires |
| | | | Selective FcγR Engagement. Cancer Cell. 29(6): 820- |
| | | | 831 (2016). |
| | | | Bankert et al. Induction of an altered CD40 |
| | | | signaling complex by an antagonistic human |
| | | | monoclonal antibody to CD40. J Immunol. |
| | | | 194(9): 4319-27 (2015). |
| | | | Pinelli et al. Novel insights into anti- |
| | | | CD40/CD154 immunotherapy in transplant tolerance. |
| | | | Immunotherapy. 7(4):399-410 (2015). |
| | | | Bajor et al. Immune activation and a 9-year |
| | | | ongoing complete remission following CD40 |
| | | | antibody therapy and metastasectomy in a patient |
| | | | with metastatic melanoma. Cancer Immunol Res. |
| | | | 2(11): 1051-8 (2014). |
| | | | Beatty et al. A phase I study of an agonist CD40 |
| | | | monoclonal antibody (CP-870,893) in combination |
| | | | with gemcitabine in patients with advanced |
| | | | pancreatic ductal adenocarcinoma. Clin Cancer Res. |
| | | | 19(22): 6286-95 (2013). |
| | | | Ruter et al. Immune modulation with weekly |
| | | | dosing of an agonist CD40 antibody in a phase I |
| | | | study of patients with advanced solid tumors. Cancer |
| | | | Biol Ther. 10(10): 983-93 (2010). |
| NKT-cells | T cell | Antibody | Tachibana et al. Increased IntratumorVA24- |
| | receptor | to T cell | Positive Natural Killer T Cells: A Prognostic Factor |
| | Vα24 | receptor | for Primary Colorectal Carcinomas. Clin Can Res. |
| | | Vα24 | 11(20), 7322-27 (2005). |
| | | | Nair et al. Type II NKT-TFH cells against |
| | | | Gaucher lipids regulate B-cell immunity and |
| | | | inflammation. Blood. 125(8): 1256-1271 (2015). |
| | | | Nieda et al. Therapeutic activation of V24V11 |
| | | | NKT cells in human subjects results in highly |
| | | | coordinated secondary activation of acquired and |
| | | | innate immunity. Blood. 103: 383-389 (2004). |
| | CD56 | Antibody | Listed in 2B |
| | | to CD56 | |
| Neutrophil | CD15 | Antibody | Ball et al. Initial trial of bispecific antibody- |
| | | to CD15 | mediated immunotherapy of CD15-bearing tumors: |
| | | | cytotoxicity of human tumor cells using a bispecific |
| | | | antibody comprised of anti-CD15 (MoAb PM81) and |
| | | | anti-CD64/Fc gamma RI (MoAb 32). J Haematother. |
| | | | 1(1); 85-94(1992). |
| | | | Rubin et al. A combination of anti-CD15 |
| | | | monoclonal antibody PM-81 and 4- |
| | | | hydroperoxycyclophosphamide augments tumor |
| | | | cytotoxicity while sparing normal progenitor cells. J |
| | | | Haematother. 3(2), 121-27 (1994). |
| Basophils | 2D7 | Antibody | Siracusa et al. Basophils and allergic |
| | | to 2D7 | inflammation. J Allergy Clin Immunol. 132(4); 789- |
| | | | 98 (2013). |
| | | | Agis et al. Enumeration and |
| | | | immunohistochemical characterisation of bone |
| | | | marrow basophils in myeloproliferative disorders |
| | | | using the basophil specific monoclonal antibody 2D7. |
| | | | J Clin Pathol 59: 396-402 (2006). |
| | | | Raap et al. Human basophils are a source of and |
| | | | are differentially activated by IL-31. Clin Exp |
| | | | Allergy. Vol 47(4): 499-508 (2017). |
| | CD203c | Antibody | MacGlashan Jr. Expression of CD203c and CD63 |
| | | to CD203c | in Human Basophils: Relationship to Differential |
| | | | Regulation of Piecemeal and Anaphylactic |
| | | | Degranulation Processes. Clin Exp Allergy. 40(9): |
| | | | 1365-1377 (2010). |
| | | | Gernez et al. Basophil CD203c Levels Are |
| | | | Increased at Baseline and Can Be Used to Monitor |
| | | | Omalizumab Treatment in Subjects with Nut Allergy. |
| | | | Int Arch Allergy Immunol 154: 318-327 (2011). |
| | | | Khanolkar et al. Evaluation of CCR3 as a |
| | | | Basophil Activation Marker. Am J Clin Pathol |
| | | | 140: 293-300 (2013). |
| | FcεRIα | Antibody | Listed in Table 10 |
| | | to FcεRIα | |
| Eosinophils | CD193 | Antibody | Takeda Y et al. Augmentation of the expression |
| | | to CD 193 | of the eotaxin receptor on duodenal neutrophils by |
| | | | IL-21. Cytokine 110:194-203 (2018). |
| | Siglec-8 | Antibody | Yu H et al. Siglec-8 and Siglec-9 binding |
| | | to Siglec-8 | specificities and endogenous airway ligand |
| | | | distributions and properties. Glycobiology. |
| | | | 27(7): 657-668 (2017). |
| | EMR1 | Antibody | Legrand F et al. The eosinophil surface receptor |
| | | to EMR1 | epidermal growth factor-like module containing |
| | | | mucin-like hormone receptor 1 (EMR1): a novel |
| | | | therapeutic target for eosinophilic disorders. J Allergy |
| | | | Clin Immunol. 133(5): 1439-47 (2014). |
| γδ T-cells | γδ TCR | Antibodies | Vantourout P and Hayday A. Six-of-the-best: |
| | | to γδ TCR | unique contributions of γδ T cells to immunology. |
| | | | Nat Rev Immunol. 13(2): 88-100 (2013). |
| | | | Hayday A and Tigelaar R. Immunoregulation in |
| | | | the tissues by gammadelta T cells. Nat Rev Immunol. |
| | | | 3(3): 233-42 (2003). |
| | | | Hayday AC. γδ cells: a right time and a right |
| | | | place for a conserved third way of protection. Annu |
| | | | Rev Immunol. 18: 975-1026(2000). |
| Engineered | Marker | Antibody | Examples of marker antigens, including LNGFR |
| immune cells | antigen, eg. | to marker | or CD20. |
| (e.g., CAR-T | CD20, LNGFR, | antigen | The marker antigen may also be an antigen |
| cells) | or scFv | | expressed by the engineered immune cell (for |
| | fragment | | example a T cell antigen, if a CAR T-cell is used). |

D. Immune Cell Engaging Domain

The immune cell engaging domain functions are capable of immune cell engaging activity when a first immune cell engaging domain binds to a second immune cell engaging domain. When the first and second immune cell engaging domains are paired together, when the inert binding partner is removed, they can bind to an immune cell. This binding can lead to activation of the immune cell.

In the absence of pairing of the first and second immune cell engaging domain, neither the first nor the second immune cell engaging domain alone can bind to an immune cell.

In some embodiments, the first and second immune cell engaging domains are capable of forming an Fv when not bound to an inert binding partner.

In some embodiments, the immune cell is a T cell, natural killer cell, macrophage, neutrophil, eosinophil, basophil, γδ T cell, NKT cell, or engineered immune cell. In some embodiments, the first and second immune cell engaging domains when paired together can activate an immune cell.

An ATTAC can engage a range of immune cells. In some embodiments, a TEAC or ATTAC engages a T cell.

1. T-Cell Engaging Domains

In some embodiments, the immune cell engaging domain is a T-cell engaging domain. The targeted T-cell engaging agent comprises a first T-cell engaging domain that is unable of engaging a T-cell alone. Instead, the first T-cell engaging domain is capable of activity when binding a second T-cell engaging domain, which is not part of the targeted T-cell engaging agent. Thus, the first and second T-cell engaging domains may be any two moieties that do not possess T-cell engaging activity alone, but do possess it when paired with each other. In other words, the first and second T-cell engaging domains are complementary halves of a functional active protein.

In some embodiments, the first and second T-cell or immune cell engaging domains are capable of forming a Fv when not bound to an inert binding partner

In some embodiments, the first and second T-cell or immune cell engaging domains are capable of binding CD3 or the T cell receptor (TCR) when neither is bound to an inert binding partner. When the two T-cell engaging domains are associated together in the two-component system, they may bind to the CD3 antigen and/or T-cell receptor on the surface of the T-cell as these activate T cells. CD3 is present on all T cells and consists of subunits designated γ, δ, ε, ζ, and η. The cytoplasmic tail of CD3 is sufficient to transduce the signals necessary for T cell activation in the absence of the other components of the TCR receptor complex. Normally, activation of T cell cytotoxicity depends first on binding of the TCR with a major histocompatibility complex (MHC) protein, itself bound to a foreign antigen, located on a separate cell. In a normal situation, only when this initial TCR-MHC binding has taken place can the CD3 dependent signally cascade responsible for T cell clonal expansion and, ultimately, T cell cytotoxicity ensue. In some of the present embodiments, however, when the two-component system binds to CD3 and/or the TCR, activation of cytotoxic T cells in the absence of independent TCR-MHC can take place by virtue of the crosslinking of the CD3 and/or TCR molecules mimicking an immune synapse formation. This means that T cells may be cytotoxically activated in a clonally independent fashion, i.e. in a manner that is independent of the specific TCR clone carried by the T cell. This allows for activation of the entire T cell compartment rather than only specific T cells of a certain clonal identity.

In some embodiments, the first T-cell engaging domain comprises a VH domain and the second T-cell engaging domain comprises a VL domain. In other embodiments, the first T-cell engaging domain comprises a VL domain and the second T-cell engaging domain comprises a VH domain. In such embodiments, when paired together the first and second T-cell engaging domains may comprise an scFv (by this we mean equivalent to an scFv but for the fact that the VH and VL are not in a single-chain configuration).

If the first and second T-cell engaging domains are a pair of VH and VL domains, the VH and VL domains may be specific for an antigen expressed on the surface of a T cell, such as CD3 or TCR. If the antigen is CD3, one potential T-cell engaging domain may be derived from muromonab (muromonab-CD3 or OKT3), otelixizumab, teplizumab, visilizumab, foralumab, or SP34. One skilled in the art would be aware of a wide range of anti-CD3 antibodies, some of which are approved therapies or have been clinically tested in human patients (see Kuhn and Weiner Immunotherapy 8(8):889-906 (2016)). Table 5 presents selected publications on exemplary anti-CD3 antibodies.

TABLE 5
Selected References Showing Specificity of Exemplary Anti-CD3 Antibodies

| | |
|---|---|
| Muromonab/ | Herold K C et al. A single course of anti-CD3 monoclonal antibody |
| OKT3 | hOKT3gamma1(Ala-Ala) results in improvement in C-peptide responses |
| | and clinical parameters for at least 2 years after onset of type 1 diabetes. |
| | Diabetes. 54(6): 1763-9 (2005). |
| | Richards J et al. Phase I evaluation of humanized OKT3: toxicity and |
| | immunomodulatory effects of hOKT3gamma4. Cancer Res. 59(9): 2096-10 |
| | (1999). |
| | Kuhn C and Weiner H L. Therapeutic anti-CD3 monoclonal antibodies: |
| | from bench to bedside. Immunotherapy 8(8): 889-906 (2016). |
| Otelixizumab | Kuhn C et al. Human CD3 transgenic mice: preclinical testing of |
| | antibodies promoting immune tolerance. Sci Transl Med. 3(68): 68ra10 |
| | (2011). |
| | Kuhn C and Weiner H L. Therapeutic anti-CD3 monoclonal antibodies: |
| | from bench to bedside. Immunotherapy 8(8): 889-906 (2016). |
| | Dean Y et al. Combination therapies in the context of anti-CD3 |
| | antibodies for the treatment of autoimmune diseases. Swiss Med Wkly. |
| | 142: w13711 (2012). |
| | Daifotis A G et al. Anti-CD3 clinical trials in type 1 diabetes mellitus. |
| | Clin Immunol. 149(3): 268-78 (2013) (abstract). |
| | Chatenoud L and Waldmann H. CD3 monoclonal antibodies: a first step |
| | towards operational immune tolerance in the clinic. Rev Diabet Stud. |
| | 9(4): 372-81. (2012). |
| Teplizumab | Masharani U B and Becker J. Teplizumab therapy for type 1 diabetes. |
| | Expert Opin Biol Ther. 10(3): 459-65 (2010). |
| | Herold K C et al. Treatment of patients with new onset Type 1 diabetes |
| | with a single course of anti-CD3 mAb Teplizumab preserves insulin |
| | production for up to 5 years. Clin Immunol. 132(2): 166-73 (2009). |
| | Kuhn C and Weiner H L. Therapeutic anti-CD3 monoclonal antibodies: |
| | from bench to bedside. Immunotherapy 8(8): 889-906 (2016). |
| | Dean Y et al. Combination therapies in the context of anti-CD3 |
| | antibodies for the treatment of autoimmune diseases. Swiss Med Wkly. |
| | 142: w13711 (2012). |
| | Daifotis A G et al. Anti-CD3 clinical trials in type 1 diabetes mellitus. |
| | Clin Immunol. 149(3): 268-78 (2013) (abstract). |
| | Chatenoud L and Waldmann H. CD3 monoclonal antibodies: a first step |
| | towards operational immune tolerance in the clinic. Rev Diabet Stud. |
| | 9(4): 372-81 (2012). |
| Visilizumab | Kuhn C and Weiner H L. Therapeutic anti-CD3 monoclonal antibodies: |
| | from bench to bedside. Immunotherapy 8(8): 889-906 (2016). |
| | Shan L. ^{99m}Tc-Labeled succinimidyl-6-hydrazinonicotinate |
| | hydrochloride (SHNH)-conjugated visilizumab. Molecular Imaging and |
| | Contrast Agent Database (MICAD) [Internet]. Created: Dec. 7, 2009; |
| | Last Update: Jan. 12, 2010; Downloaded May 3, 2018. |
| | Dean Y et al. Combination therapies in the context of anti-CD3 |
| | antibodies for the treatment of autoimmune diseases. Swiss Med Wkly. |
| | 142: w13711 (2012). |
| Foralumab | Kuhn C and Weiner H L. Therapeutic anti-CD3 monoclonal antibodies: |
| | from bench to bedside. Immunotherapy 8(8): 889-906 (2016). |
| | Dean Y et al. Combination therapies in the context of anti-CD3 |
| | antibodies for the treatment of autoimmune diseases. Swiss Med Wkly. |
| | 142: w13711 (2012). |
| SP34 | Pessano S et al. The T3/T cell receptor complex: antigenic distinction |
| | between the two 20-kd T3 (T3-6 and T3-E) subunits. EMBO Journal |
| | 4(2): 337-344 (1985). |
| 20G6 | WO2016/116626 |

Antibodies with specificity to the TCR, including the αβ and γδ TCRs, are also well-known. Table 6 presents selected publications on exemplary anti-TCR antibodies.

TABLE 6
Selected References Showing Specificity of Exemplary Anti-TCR Antibodies

Verma-B. et al. TCR Mimic Monoclonal Antibody Targets a Specific Peptide/HLA Class I
Complex and Significantly Impedes Tumor Growth In Vivo Using Breast Cancer Models
J Immunol. 184: 2156-2165 (2010).
Conrad M L et al. TCR and CD3 antibody cross-reactivity in 44 species. Cytometry A.
71(11): 925-33 (2007).
Koenecke C et al. In vivo application of mAb directed against the gammadelta TCR does
not deplete but generates “invisible” gammadelta T cells. Eur J Immunol. 39(2): 372-9 (2009).
Exley M A et al. Selective activation, expansion, and monitoring of human iNKT cells with a
monoclonal antibody specific for the TCR alpha-chain CDR3 loop. Eur J Immunol. 38(6): 1756-
66 (2008).
Deetz C O et al. Gamma interferon secretion by human Vgamma2Vdelta2 T cells after
stimulation with antibody against the T-cell receptor plus the Toll-Like receptor 2 agonist
Pam3Cys. Infection and Immunity. 74(8): 4505-4511 (2006).
Tang X et al. Anti-TCR antibody treatment activates a novel population of nonintestinal
CD8 alpha alpha+ TCR alpha beta+ regulatory T cells and prevents experimental autoimmune
encephalomyelitis. J Immunol. 178(10): 6043-50 (2007).
Lavasani S et al. Monoclonal antibody against T-cell receptor alphabeta induces self-
tolerance in chronic experimental autoimmune encephalomyelitis. Scand J Immunol. 65(1): 39-
47 (2007).
Nasreen M et al. In vivo treatment of class II MHC-deficient mice with anti-TCR antibody
restores the generation of circulating CD4 T cells and optimal architecture of thymic medulla.
J Immunol. 171(7): 3394-400 (2003).

2. Natural Killer Cell Engaging Domains

In some embodiments, the immune cell engaging domain is a natural killer cell engaging domain. When the two natural killer cell engaging domains are associated together in the two-component system, they may bind to an antigen on the surface of the NK cell to engage these cells. In some embodiments, the antigen on the surface of the NK cell may be NKG2D, CD16, NKp30, NKp44, NKp46 or DNAM.

In some embodiments, having one half of the two-component system bind to a surface protein on the natural killer cell and having the other half of the system bind to cancer cells allows specific engagement of natural killer cells. Engagement of natural killer cells can lead to their activation and induce natural killer cell-mediated cytotoxicity and cytokine release.

When the two natural killer cell engaging domains are associated together in the ATTAC, the natural killer cell may specifically lyse the cancer cells bound by the cancer-specific ATTAC component. Killing of a cancer cell may be mediated by either the perforin/granzyme system or by FasL-Fas engagement. As well as this potential cytotoxic function, natural killer cells are also able to secrete proinflammatory cytokines including interferon gamma and tumor necrosis factor alpha which can activate macrophages and dendritic cells in the immediate vicinity to enhance the anti-cancer immune response.

In some embodiments, the first natural killer cell engaging domain comprises a VH domain and the second natural killer cell engaging domain comprises a VL domain. In other embodiments, the first natural killer cell engaging domain comprises a VL domain and the second natural killer cell engaging domain comprises a VH domain. In such embodiments, when paired together the first and second natural killer cell engaging domains may comprise an scFv (by this we mean equivalent to an scFv but for the fact that the VH and VL are not in a single-chain configuration).

If the first and second natural killer cell engaging domains are a pair of VH and VL domains, the VH and VL domains may be specific for an antigen expressed on the surface of a natural killer cell, such as NKG2D, CD16, NKp30, NKp44, NKp46 and DNAM.

Table 7 presents selected publications on some exemplary antibodies specific for an antigen expressed on the surface of a natural killer cell.

TABLE 7
Selected References Showing Specificity of Exemplary Antibodies
for Surface Antigens on Natural Killer Cells

| | |
|---|---|
| NKG2D | Vadstrup et al. Anti-NKG2D mAb: A New Treatment for Crohn's Disease? |
| | Int J Mol Sci. 18(9) (2017). |
| | Rong et al. Recognition and killing of cancer stem-like cell population in |
| | hepatocellular carcinoma cells by cytokine-induced killer cells via NKG2d- |
| | ligands recognition. Oncoimmunology. 5(3): e1086060 (2015). |
| | Shen et al. Possible association of decreased NKG2D expression levels and |
| | suppression of the activity of natural killer cells in patients with colorectal cancer. |
| | Int J Oncol. 40(4): 1285-90 (2012). |
| | Kim et al. Suppression of human anti-porcine natural killer cell xenogeneic |
| | responses by combinations of monoclonal antibodies specific to CD2 and |
| | NKG2D and extracellular signal-regulated kinase kinase inhibitor. |
| | Immunology. 130(4): 545-55 (2010). |
| | Steigerwald et al. Human IgG1 antibodies antagonizing activating receptor |
| | NKG2D on natural killer cells. MAbs. 1(2): 115-27 (2009). |
| | Paidipally et al. NKG2D-dependent IL-17 production by human T cells in |
| | response to an intracellular pathogen. J Immunol. 183(3): 1940-5 (2009). |
| | Kwong et al. Generation, affinity maturation, and characterization of a human |
| | anti-human NKG2D monoclonal antibody with dual antagonistic and agonistic |
| | activity. J Mol Biol. 384(5): 1143-56 (2008). |
| | Wrobel et al. Lysis of a broad range of epithelial tumour cells by human |
| | gamma delta T cells: involvement of NKG2D ligands and T-cell receptor- versus |
| | NKG2D-dependent recognition. Scand J Immunol. 66(2-3): 320-8 (2007). |
| | Andre et al. Comparative analysis of human NK cell activation induced by |
| | NKG2D and natural cytotoxicity receptors. Eur J Immunol. 34(4): 961-71 (2004). |
| | Regunathan et al. NKG2D receptor-mediated NK cell function is regulated by |
| | inhibitory Ly49 receptors. Blood. 105(1): 233-40 (2005). |
| CD16 | Lee et al. Expansion of cytotoxic natural killer cells using irradiated autologous |
| | peripheral blood mononuclear cells and anti-CD16 antibody. Sci Rep. 7(1): 11075 |
| | (2017). |
| | Parsons et al. Anti-HIV antibody-dependent activation of NK cells impairs |
| | NKp46 expression. J Immunol. 192(1): 308-15 (2014). |
| | Vallera et al. Heterodimeric bispecific single-chain variable-fragment |
| | antibodies against EpCAM and CD16 induce effective antibody-dependent |
| | cellular cytotoxicity against human carcinoma cells. Cancer Biother Radiopharm. |
| | 28(4): 274-82 (2013). |
| | Asano et al. Construction and humanization of a functional bispecific EGFR × |
| | CD16 diabody using a refolding system. FEBS J. 279(2): 223-33 (2012). |
| | Jewett et al. Strategies to rescue mesenchymal stem cells (MSCs) and dental |
| | pulp stem cells (DPSCs) from NK cell mediated cytotoxicity. PLoS One. |
| | 5(3): e9874 (2010). |
| | Congy-Jolivet et al. Fc gamma RIIIa expression is not increased on natural |
| | killer cells expressing the Fc gamma RIIIa-158V allotype. Cancer Res. |
| | 68(4): 976-80 (2008). |
| | Jewett et al. Coengagement of CD16 and CD94 receptors mediates secretion of |
| | chemokines and induces apoptotic death of naive natural killer cells. |
| | Clin Cancer Res. 12(7 Pt 1): 1994-2003 (2006). |
| | Yamaguchi et al. HER2-specific cytotoxic activity of lymphokine-activated |
| | killer cells in the presence of trastuzumab. Anticancer Res. 25(2A): 827-32 |
| | (2005). |
| | Shahied et al. Bispecific minibodies targeting HER2/neu and CD16 exhibit |
| | improved tumor lysis when placed in a divalent tumor antigen binding format. |
| | J Biol Chem. 279(52): 53907-14 (2004). |
| | Dall'Ozzo et al. Rituximab-dependent cytotoxicity by natural killer cells: |
| | influence of FCGR3A polymorphism on the concentration-effect relationship. |
| | Cancer Res. 64(13): 4664-9 (2004). |
| NKp30 | Hervier et al. Involvement of NK Cells and NKp30 Pathway in Antisynthetase |
| | Syndrome. J Immunol. 197(5): 1621-30 (2016). |
| | Zou et al. NKP30-B7-H6 Interaction Aggravates Hepatocyte Damage through |
| | Up-Regulation of Interleukin-32 Expression in Hepatitis B Virus-Related Acute- |
| | On-Chronic Liver Failure. PLoS One. 10(8): e0134568 (2015). |
| | Ferrari de Andrade et al. Natural killer cells are essential for the ability of |
| | BRAF inhibitors to control BRAFV600E-mutant metastatic melanoma. |
| | Cancer Res. 74(24): 7298-308 (2014). |
| | Warren et al. Evidence that the cellular ligand for the human NK cell activation |
| | receptor NKp30 is not a heparan sulfate glycosaminoglycan. J Immunol. |
| | 175(1): 207-12 (2005). |
| | Holder et al. Hepatitis C virus-infected cells downregulate NKp30 and inhibit |
| | ex vivo NK cell functions. J Immunol. 191(6): 3308-18 (2013). |
| | Laufer et al. CD4+ T cells and natural killer cells: Biomarkers for hepatic |
| | fibrosis in human immunodeficiency virus/hepatitis C virus-coinfected patients. |
| | World J Hepatol. 9(25): 1073-1080 (2017). |
| | Chretien et al. NKp30 expression is a prognostic immune biomarker for |
| | stratification of patients with intermediate-risk acute myeloid leukemia. |
| | Oncotarget. 8(30): 49548-49563 (2017). |
| | Spaggiari et al. Mesenchymal stem cells inhibit natural killer-cell proliferation, |
| | cytotoxicity, and cytokine production: role of indoleamine 2,3-dioxygenase and |
| | prostaglandin E2. Blood. 111: 1327-1333 (2008). |
| | Fiegler et al. Downregulation of the activating NKp30 ligand B7-H6 by HDAC |
| | inhibitors impairs tumor cell recognition by NK cells. Blood. 122(5): 684-693 |
| | (2013). |
| | Salimi et al. Group 2 Innate Lymphoid Cells Express Functional NKp30 |
| | Receptor Inducing Type 2 Cytokine Production. J Immunol. 196: 45-54 (2016). |
| NKp44 | Esin et al. Interaction of Mycobacterium tuberculosis cell wall components |
| | with the human natural killer cell receptors NKp44 and Toll-like receptor 2. |
| | Scand J Immunol. 77(6): 460-9 (2013). |
| | Hershkovitz et al. NKp44 receptor mediates interaction of the envelope |
| | glycoproteins from the West Nile and dengue viruses with NK cells. J Immunol. |
| | 2009 Aug. 15; 183(4): 2610-21. |
| | Sivori et al. 2B4 functions as a co-receptor in human NK cell activation. |
| | Eur J Immunol. 30(3): 787-93 (2000). |
| | Vitale et al. NKp44, a Novel Triggering Surface Molecule Specifically |
| | Expressed by Activated Natural Killer Cells, Is Involved in Non-Major |
| | Histocompatibility Complex-restricted Tumor Cell Lysis. J Exp. Med. |
| | 187(12): 2065-2072 (1998). |
| | Campbell et al. NKp44 Triggers NK Cell Activation through DAP12 |
| | Association That Is Not Influenced by a Putative Cytoplasmic Inhibitory |
| | Sequence. J Immunol. 172: 899-906 (2004). |
| | Fuchs et al. Paradoxic inhibition of human natural interferon-producing cells |
| | by the activating receptor NKp44. Blood. 106: 2076-2082 (2005). |
| | Vacca et al. Regulatory role of NKp44, NKp46, DNAM-1 and NKG2D |
| | receptors in the interaction between NK cells and trophoblast cells. Evidence for |
| | divergent functional profiles of decidual versus peripheral NK cells. |
| | International Immunology 20(11): 1395-1405 (2008). |
| | Cantoni et al. NKp44, A Triggering Receptor Involved in Tumor Cell Lysis by |
| | Activated Human Natural Killer Cells, Is a Novel Member of the |
| | Immunoglobulin Superfamily. J Exp Med. 189(5): 787-795 (1999). |
| | Vieillard et al. NK cytotoxicity against CD4+ T cells during HIV-1 infection: |
| | A gp41 peptide induces the expression of an NKp44 ligand. Proc Natl Acad Sci USA. |
| | 102(31): 10981-10986. |
| | Glatzer et al. RORγt + Innate Lymphoid Cells Acquire a Proinflammatory |
| | Program upon Engagement of the Activating Receptor NKp44. Immunity. |
| | 38: 1223-1235 (2013). |
| NKp46 | Shemer-Avni et al. Expression of NKp46 Splice Variants in Nasal Lavage |
| | Following Respiratory Viral Infection: Domain 1-Negative Isoforms Predominate |
| | and Manifest Higher Activity. Front Immunol. 8: 161 (2017). |
| | Crome et al. A distinct innate lymphoid cell population regulates tumor- |
| | associated T cell Nat Med. 23(3): 368-375 (2017). |
| | Li et al. Natural Killer p46 Controls Hepatitis B Virus Replication and |
| | Modulates Liver Inflammation. PLoS One. 10(8): e0135874 (2015). |
| | Dou et el. Influenza vaccine induces intracellular immune memory of human |
| | NK cells. PLoS One. 10(3): e0121258 (2015). |
| | Vego et al. Monomethyl fumarate augments NK cell lysis of tumor cells |
| | through degranulation and the upregulation of NKp46 and CD107a. |
| | Cell Mol Immunol. 13(1): 57-64 (2016). |
| | Vankayalapati et al. Role of NK cell-activating receptors and their ligands in |
| | the lysis of mononuclear phagocytes infected with an intracellular bacterium. |
| | J Immunol. 175(7): 4611-7 (2005). |
| | Laufer et al. CD4+ T cells and natural killer cells: Biomarkers for hepatic |
| | fibrosis in human immunodeficiency virus/hepatitis C virus-coinfected patients. |
| | World J Hepatol. 9(25): 1073-1080 (2017). |
| | Yoshioka et al. Frequency and role of NKp46 and NKG2A in hepatitis B virus |
| | infection. PLoS One. 12(3): e0174103 (2017). |
| | Vacca et al. Regulatory role of NKp44, NKp46, DNAM-1 and NKG2D |
| | receptors in the interaction between NK cells and trophoblast cells. Evidence for |
| | divergent functional profiles of decidual versus peripheral NK cells. |
| | International Immunology 20(11): 1395-1405 (2008). |
| DNAM | Okumura G, et al. Development and Characterization of Novel Monoclonal |
| (CD226) | Antibodies Against Human DNAM-1. |
| | Monoclon Antib Immunodiagn Immunother. |
| | 36(3): 135-139 (2017). |
| | Stein N et al. The paired receptors TIGIT and DNAM-1 as targets for |
| | therapeutic antibodies. Hum Antibodies. 25(3-4): 111-119 (2017). |
| | Elhai M et al. Targeting CD226/DNAX accessory molecule-1 (DNAM-1) in |
| | collagen-induced arthritis mouse models. J Inflamm (Lond). 12: 9 (2015). |
| | Laufer et al. CD4+ T cells and natural killer cells: Biomarkers for hepatic |
| | fibrosis in human immunodeficiency virus/hepatitis C virus-coinfected patients. |
| | World J Hepatol. 9(25): 1073-1080 (2017). |
| | Shibuya et al. Physical and Functional Association of LFA-1 with DNAM-1 |
| | Adhesion Molecule. Immunity. 11: 615-623 (1999). |
| | Li et al. CD155 loss enhances tumor suppression via combined host and tumor- |
| | intrinsic mechanisms. J Clin Invest. pii: 98769 (2018). |
| | Chen et al. Targeting chemotherapy-resistant leukemia by combining DNT |
| | cellular therapy with conventional chemotherapy. J Exp Clin Cancer Res. |
| | 37(1): 88 (2018). |
| | Rodrigues et al. Low-Density Lipoprotein Uptake Inhibits the Activation and |
| | Antitumor Functions of Human Vγ9Vδ2 T Cells. Cancer Immunol Res. 6(4): 448- |
| | 457 (2018). |
| | Rocca et al. Phenotypic and Functional Dysregulated Blood NK Cells in |
| | Colorectal Cancer Patients Can Be Activated by Cetuximab Plus IL-2 or IL-15. |
| | Front Immunol. 7: 413 (2016). |
| | Shibuya et al DNAM-1, a novel adhesion molecule involved in the cytolytic |
| | function of T lymphocytes. Immunity. 4(6): 573-81(1996). |

3. Macrophage Engaging Domains

In some embodiments, the immune cell engaging domain is a macrophage engaging domain. As used herein, a “macrophage” may refer to any cell of the mononuclear phagocytic system, such as grouped lineage-committed bone marrow precursors, circulating monocytes, resident macrophages, and dendritic cells (DC). Examples of resident macrophages can include Kupffer cells and microglia.

When the two macrophage engaging domains are associated together in the two-component system, they may bind to an antigen on the surface of the macrophage to engage these cells. In some embodiments, the antigen on the surface of the macrophage may be CD89 (Fc alpha receptor 1), CD64 (Fc gamma receptor 1), CD32 (Fc gamma receptor 2A) or CD16a (Fc gamma receptor 3A).

In some embodiments, having one half of the two-component system bind to a surface protein on the macrophage and having the other half of the system bind to cancer cells allows specific engagement of macrophages. Engagement of macrophages can lead the macrophage to phagocytose the cancer cell.

In some embodiments, inducing macrophage phagocytosis via binding to an antigen on the surface of the macrophages is independent of Fc receptor binding, which has been shown previously to be a method of tumor cell killing by macrophages. Normally, cancer cells are bound by whole antibodies and the Fc portion of the antibody binds to the Fc receptor and induces phagocytosis.

In some embodiments, engagement of toll-like receptors on the macrophage surface (see patent application US20150125397A1) leads to engagement of macrophages.

When the two macrophage engaging domains are associated together in the ATTAC, they may induce the macrophage to phagocytose the cancer cell bound by the cancer-specific ATTAC component.

In some embodiments, the first macrophage engaging domain comprises a VH domain and the second macrophage engaging domain comprises a VL domain. In other embodiments, the first macrophage engaging domain comprises a VL domain and the second macrophage engaging domain comprises a VH domain. In such embodiments, when paired together the first and second macrophage engaging domains may comprise an scFv (by this we mean equivalent to an scFv but for the fact that the VH and VL are not in a single-chain configuration).

If the first and second macrophage engaging domains are a pair of VH and VL domains, the VH and VL domains may be specific for an antigen expressed on the surface of a macrophage, such as CD89 (Fc alpha receptor 1), CD64 (Fc gamma receptor 1), CD32 (Fc gamma receptor 2A) and CD16a (Fc gamma receptor 3A), or toll-like receptors.

Table 8 presents selected publications on some exemplary antibodies specific for an antigen expressed on the surface of a macrophage.

TABLE 8
Selected References Showing Specificity of Exemplary
Antibodies for Surface Antigens on Macrophages

| | |
|---|---|
| CD89 (Fc | Xu et al. Critical Role of Kupffer Cell CD89 Expression in |
| alpha | Experimental IgA Nephropathy. PLoS One. 11(7): e0159426 (2016). |
| receptor | Deo et al. Bispecific molecules directed to the Fc receptor for IgA (Fc |
| 1) | alpha RI, CD89) and tumor antigens efficiently promote cell-mediated |
| | cytotoxicity of tumor targets in whole blood. J Immunol. 160(4): 1677-86 |
| | (1998). |
| | Hamre et al. Expression and modulation of the human |
| | immunoglobulin A Fc receptor (CD89) and the FcR gamma chain on |
| | myeloid cells in blood and tissue. Scand J Immunol. 57(6): 506-16 |
| | (2003). |
| | Mladenov et al. The Fc-alpha receptor is a new target antigen for |
| | immunotherapy of myeloid leukemia. Int J Cancer. 137(11): 2729-38 |
| | (2015). |
| | United States Patent Application US20110104145A1 Method for the |
| | treatment or prophylaxis of chronic inflammatory diseases. |
| | Smith et al. Intestinal macrophages lack CD14 and CD89 and |
| | consequently are down-regulated for LPS- and IgA-mediated activities. |
| | J Immunol. 167(5): 2651-6 (2001). |
| | Van Zandbergen et al. Crosslinking of the human Fc receptor for IgA |
| | (FcalphaRI/CD89) triggers FcR gamma-chain-dependent shedding of |
| | soluble CD89. J Immunol. 163(11): 5806-12 (1999). |
| | Cheeseman et al. Expression Profile of Human Fc Receptors in |
| | Mucosal Tissue: Implications for Antibody-Dependent Cellular Effector |
| | Functions Targeting HIV-1 Transmission. PLoS One. 11(5): e0154656 |
| | (2016). |
| | Geissman et al. A subset of human dendritic cells expresses IgA Fc |
| | receptor (CD89), which mediates internalization and activation upon |
| | cross-linking by IgA complexes. J Immunol. 166(1): 346-52 (2001). |
| | Reterink et al. Transforming growth factor-beta 1 (TGF-beta 1) down- |
| | regulates IgA Fc-receptor (CD89) expression on human monocytes. |
| | Clin Exp Immunol. 103(1): 161-6 (1996). |
| CD64 (Fc | Histodorov et al. Recombinant H22(scFv) blocks CD64 and prevents |
| gamma | the capture of anti-TNF monoclonal antibody. A potential strategy to |
| receptor | enhance anti-TNF therapy. MAbs. 6(5): 1283-9 (2014). |
| 1) | Cheeseman et al. Expression Profile of Human Fc Receptors in |
| | Mucosal Tissue: Implications for Antibody-Dependent Cellular Effector |
| | Functions Targeting HIV-1 Transmission. PLoS One. 11(5): e0154656 |
| | (2016). |
| | Moura et al. Co-association of methotrexate and SPIONs into anti- |
| | CD64 antibody-conjugated PLGA nanoparticles for theranostic |
| | application. Int J Nanomedicine. 9: 4911-22 (2014). |
| | Petricevic et al. Trastuzumab mediates antibody-dependent cell- |
| | mediated cytotoxicity and phagocytosis to the same extent in both |
| | adjuvant and metastatic HER2/neu breast cancer patients. J Transl Med. |
| | 11: 307 (2013). |
| | Miura et al. Paclitaxel enhances antibody-dependent cell-mediated |
| | cytotoxicity of trastuzumab by rapid recruitment of natural killer cells in |
| | HER2-positive breast cancer. J Nippon Med Sch. 81(4): 211-20 (2014). |
| | Schiffer et al. Targeted ex vivo reduction of CD64-positive monocytes |
| | in chronic myelomonocytic leukemia and acute myelomonocytic |
| | leukemia using human granzyme B-based cytolytic fusion proteins. |
| | Int J Cancer. 135(6): 1497-508 (2014). |
| | Matt et al. Elevated Membrane and Soluble CD64: A Novel Marker |
| | Reflecting Altered FcγR Function and Disease in Early Rheumatoid |
| | Arthritis That Can Be Regulated by Anti-Rheumatic Treatment. |
| | PLoS One. 10(9): e0137474 (2015). |
| | Haegel et al. A unique anti-CD115 monoclonal antibody which |
| | inhibits osteolysis and skews human monocyte differentiation from M2- |
| | polarized macrophages toward dendritic cells. MAbs. 5(5): 736-47 (2013). |
| | Mladenov et al. CD64-directed microtubule associated protein tau kills |
| | leukemic blasts ex vivo. Oncotarget. 7(41): 67166-67174 (2016). |
| | Wong et al. Monochromatic gating method by flow cytometry for high |
| | purity monocyte analysis. Cytometry B Clin Cytom. 84(2): 119-24 (2013). |
| CD32 (Fc | Cheeseman et al. Expression Profile of Human Fc Receptors in |
| gamma | Mucosal Tissue: Implications for Antibody-Dependent Cellular Effector |
| receptor | Functions Targeting HIV-1 Transmission. PLoS One. 11(5): e0154656 |
| 2A) | (2016). |
| | Bhatnagar et al. FcγRIII (CD16)-mediated ADCC by NK cells is |
| | regulated by monocytes and FcγRII (CD32). Eur J Immunol. |
| | 44(11): 3368-79 (2014). |
| | Veri et al. Monoclonal antibodies capable of discriminating the human |
| | inhibitory Fcgamma-receptor IIB (CD32B) from the activating |
| | Fcgamma-receptor IIA (CD32A): biochemical, biological and functional |
| | characterization. Immunology. 121(3): 392-404 (2007). |
| | Vivers et al. Divalent cation-dependent and -independent |
| | augmentation of macrophage phagocytosis of apoptotic neutrophils by |
| | CD44 antibody. Clin Exp Immunol. 138(3): 447-52 (2004). |
| | Athanasou et al. Immunophenotypic differences between osteoclasts |
| | and macrophage polykaryons: immunohistological distinction and |
| | implications for osteoclast ontogeny and function. J Clin Pathol. |
| | 43(12): 997-1003 (1990). |
| | Leidi et al. M2 macrophages phagocytose rituximab-opsonized |
| | leukemic targets more efficiently than m1 cells in vitro. J Immunol. |
| | 182(7): 4415-22 (2009). |
| | Shanaka et al. Differential Enhancement of Dengue Virus Immune |
| | Complex Infectivity Mediated by Signaling-Competent and Signaling- |
| | Incompetent Human FcγRIA (CD64) or FcγRIIA (CD32). J Virol. |
| | 80(20): 10128-10138 (2006). |
| | Lee et al. Isolation and immunocytochemical characterization of |
| | human bone marrow stromal macrophages in hemopoietic clusters. |
| | J Exp Med. 168(3): 1193-8 (1988). |
| | Dialynas et al. Phenotypic and functional characterization of a new |
| | human macrophage cell line K1m demonstrating immunophagocytic |
| | activity and signalling through HLA class II. Immunology. 90(4): 470-6 |
| | (1997). |
| | Athanasou et al. Immunocytochemical analysis of human synovial |
| | lining cells: phenotypic relation to other marrow derived cells. |
| | Ann Rheum Dis. 50(5): 311-315 (1991). |
| CD 16a | Zhou et al. CD14(hi)CD16+ monocytes phagocytose antibody- |
| (Fc | opsonised Plasmodium falciparum infected erythrocytes more efficiently |
| gamma | than other monocyte subsets, and require CD16 and complement to do |
| receptor | so. BMC Med. 13: 154(2015). |
| 3A) | Cheeseman et al. Expression Profile of Human Fc Receptors in |
| | Mucosal Tissue: Implications for Antibody-Dependent Cellular Effector |
| | Functions Targeting HIV-1 Transmission. PLoS One. 11(5): e0154656 |
| | (2016). |
| | Dialynas et al. Phenotypic and functional characterization of a new |
| | human macrophage cell line K1m demonstrating immunophagocytic |
| | activity and signalling through HLA class II. Immunology. 90(4): 470-6 |
| | (1997). |
| | Nazareth et al. Infliximab therapy increases the frequency of |
| | circulating CD16(+) monocytes and modifies macrophage cytokine |
| | response to bacterial infection. Clin Exp Immunol. 2014 September; 177(3): 703- |
| | 11. |
| | Pander et al. Activation of tumor-promoting type 2 macrophages by |
| | EGFR-targeting antibody cetuximab. Clin Cancer Res. 17(17): 5668-73 |
| | (2011). |
| | Boyle. Human macrophages kill human mesangial cells by Fas-L- |
| | induced apoptosis when triggered by antibody via CD16. |
| | Clin Exp Immunol. 137(3): 529-37 (2004). |
| | Korkosz et al. Monoclonal antibodies against macrophage colony- |
| | stimulating factor diminish the number of circulating intermediate and |
| | nonclassical (CD14(++)CD16(+)/CD14(+)CD16(++)) monocytes in |
| | rheumatoid arthritis patient. Blood. 119(22): 5329-30 (2012). |
| | Wang et al. Interleukin-10 induces macrophage apoptosis and |
| | expression of CD16 (FcgammaRIII) whose engagement blocks the cell |
| | death programme and facilitates differentiation. Immunology. |
| | 102(3): 331-7 (2001). |
| | Kramer et al. 17 beta-estradiol regulates cytokine release through |
| | modulation of CD16 expression in monocytes and monocyte-derived |
| | macrophages. Arthritis Rheum. 50(6): 1967-75 (2004). |
| | Tricas et al. Flow cytometry counting of bronchoalveolar lavage |
| | leukocytes with a new profile of monoclonal antibodies combination. |
| | Cytometry B Clin Cytom. 82(2): 61-6 (2012). |

4. Neutrophil Engaging Domains

In some embodiments, the immune cell engaging domain is a neutrophil engaging domain. When the two neutrophil engaging domains are associated together in the two-component system, they may bind to an antigen on the surface of the neutrophil to engage these cells. In some embodiments, the antigen on the surface of the neutrophil may be CD89 (FcαR1), FcγRI (CD64), FcγRIIA (CD32), FcγRIIIA (CD16a), CD11b (CR3, αMβ2), TLR2, TLR4, CLEC7A (Dectin1), formyl peptide receptor 1 (FPR1), formyl peptide receptor 2 (FPR2), or formyl peptide receptor 3 (FPR3).

In some embodiments, having one half of the two-component system bind to a surface protein on the neutrophil and having the other half of the system bind to cancer cells allows specific engagement of neutrophils. Engagement of neutrophils can lead to phagocytosis and cell uptake.

When the two neutrophil engaging domains are associated together in the ATTAC, the neutrophil may engulf the target cells.

In some embodiments, the first neutrophil engaging domain comprises a VH domain and the second neutrophil engaging domain comprises a VL domain. In other embodiments, the first neutrophil engaging domain comprises a VL domain and the second neutrophil engaging domain comprises a VH domain. In such embodiments, when paired together the first and second neutrophil engaging domains may comprise an scFv (by this we mean equivalent to an scFv but for the fact that the VH and VL are not in a single-chain configuration).

If the first and second neutrophil engaging domains are a pair of VH and VL domains, the VH and VL domains may be specific for an antigen expressed on the surface of a neutrophil, such as CD89 (FcαR1), FcγRI (CD64), FcγRIIA (CD32), FcγRIIIA (CD16a), CD11b (CR3, αMβ2), TLR2, TLR4, CLEC7A (Dectin1), FPR1, FPR2, or FPR3.

Table 9 presents selected publications on some exemplary antibodies specific for an antigen expressed on the surface of a neutrophil.

TABLE 9
Selected References Showing Specificity of Exemplary
Antibodies for Surface Antigens on Neutrophils

| CD89 (FcαR1) | Li B et al. CD89-mediated recruitment of macrophages via a |
|---|---|
| | bispecific antibody enhances anti-tumor efficacy. Oncoimmunology. |
| | 7(1) (2017) |
| | Valerius T et al. FcalphaRI (CD89) as a novel trigger molecule for |
| | bispecific antibody therapy. Blood 90(11): 4485-92 (1997) |
| FcγRI (CD64) | Honeychurch et al. Therapeutic efficacy of FcgammaRI/CD64- |
| | directed bispecific antibodies in B-cell lymphoma. Blood |
| | 96(10): 3544-52 (2000) |
| | James et al. A phase II study of the bispecific antibody MDX-H210 |
| | (anti-HER2 × CD64) with GM-CSF in HER2+ advanced prostate |
| | cancer. British Journal of Cancer 85(2): 152-156 (2001) |
| | Futosi K et al Neutrophil cell surface receptors and their |
| | intracellular signal transduction pathways. Int Immunopharmacol. |
| | 17(3): 638-50 (2013) |
| | Kasturirangan et al. Targeted Fcγ Receptor (FcγR)-mediated |
| | Clearance by a Biparatopic Bispecific Antibody. |
| | Journal of Biological Chemistry 292(10): |
| | 4361-4370 (2017) |
| FcγRIIA | Futosi K et al Neutrophil cell surface receptors and their |
| (CD32) | intracellular signal transduction pathways. Int Immunopharmacol. |
| | 17(3): 638-50 (2013) |
| | Nimmerjahn F and Ravetch J V. Antibodies, Fc receptors and |
| | cancer. Curr Opin Immunol. 19(2): 239-45 (2007) |
| | Ravetch J V: Fc receptors. In Fundamental Immunology, edn5. |
| | Edited by Paul W E. Lippincott-Raven; 685-700 (2003) |
| | Nimmerjahn F, Ravetch J V: Fcγ receptors: old friends and new |
| | family members. Immunity 24: 19-28 (2006) |
| FcγRIIIA | Futosi K et al Neutrophil cell surface receptors and their |
| (CD16a) | intracellular signal transduction pathways. Int Immunopharmacol. |
| | 17(3): 638-50 (2013) |
| | Nimmerjahn F and Ravetch J V. Antibodies, Fc receptors and |
| | cancer. Curr Opin Immunol. 19(2): 239-45 (2007) |
| | Ravetch J V: Fc receptors. In Fundamental Immunology, edn5. |
| | Edited by Paul W E. Lippincott-Raven; 685-700 (2003) |
| | Nimmerjahn F, Ravetch J V Fcγ receptors: old friends and new |
| | family members. Immunity 24: 19-28 (2006) |
| | Renner et al. Targeting properties of an anti-CD16/anti-CD30 |
| | bispecific antibody in an in vivo system. |
| | Cancer Immunol Immunother. 50(2): |
| | 102-8 (2001) |
| CD11b(CR3, | Gazendam R P et al. How neutrophils kill fungi. Immunol Rev. |
| (α_{M}β₂) | 273(1): 299-311 (2016) |
| | Urbaczek A C et al. Influence of FcγRIIIb polymorphism on its |
| | ability to cooperate with FcγRIIa and CR3 in mediating the oxidative |
| | burst of human neutrophils. Hum Immunol. 75(8): 785-90 (2014) |
| | Futosi K et al Neutrophil cell surface receptors and their |
| | intracellular signal transduction pathways. Int Immunopharmacol. |
| | 17(3): 638-50 (2013) |
| | van Spriel A B et al. Mac-1 (CD11b/CD18) is essential for Fc |
| | receptor-mediated neutrophil cytotoxicity and immunologic synapse |
| | formation. Blood. 97(8): 2478-86 (2001) |
| TLR2 | Kawasaki T and Kawai T. Toll-Like Receptor Signaling Pathways. |
| | Front Immunol. 5: 461 (2014) |
| | Beutler B A. TLRs and innate immunity. Blood. 113(7): 1399-407 |
| | (2009) |
| | Beutler B et al. Genetic analysis of host resistance: Toll-like |
| | receptor signaling and immunity at large. Annu Rev Immunol. 24: 353- |
| | 89 (2006) |
| TLR4 | Kawasaki T and Kawai T. Toll-Like Receptor Signaling Pathways. |
| | Front Immunol. 5: 461 (2014) |
| | Beutler B A. TLRs and innate immunity. Blood. 113(7): 1399-407 |
| | (2009) |
| | Beutler B et al. Genetic analysis of host resistance: Toll-like |
| | receptor signaling and immunity at large. Annu Rev Immunol. 24: 353- |
| | 89 (2006) |
| CLEC7A | Brown G D. Dectin-1: a signalling non-TLR pattern-recognition |
| (Dectin1) | receptor. Nat Rev Immunol. 6(1): 33-43 (2006) |
| | Pyz E et al. C-type lectin-like receptors on myeloid cells. Ann Med. |
| | 38(4): 242-51 (2006) |
| FPR1, FPR2, | Dahlgren C et al. Basic characteristics of the neutrophil receptors |
| FPR3 | that recognize formylated peptides, a danger-associated molecular |
| | pattern generated by bacteria and mitochondria. Biochem Pharmacol. |
| | 114: 22-39. doi: 10.1016/j.bcp.2016.04.014 (2016) |
| | Lee HY et al. Formyl Peptide Receptors in Cellular Differentiation |
| | and Inflammatory Diseases. J Cell Biochem. 118(6): 1300-1307 |
| | (2017) |

5. Eosinophil Engaging Domains

In some embodiments, the immune cell engaging domain is an eosinophil engaging domain. When the two eosinophil engaging domains are associated together in the two-component system, they may bind to an antigen on the surface of the eosinophil to engage these cells. In some embodiments, the antigen on the surface of the eosinophil may be CD89 (Fc alpha receptor 1), FcεRI, FcγRI (CD64), FcγRIIA (CD32), FcγRIIIB (CD16b), or TLR4.

In some embodiments, having one half of the two-component system bind to a surface protein on the eosinophil and having the other half of the system bind to cancer cells allows specific engagement of eosinophils. Engagement of eosinophils can lead to degranulation and release of preformed cationic proteins, such as EPO, major basic protein 1 (MBP1), and eosinophil-associated ribonucleases (EARs), known as ECP and eosinophil-derived neurotoxin.

When the two neutrophil engaging domains are associated together in the ATTAC, the neutrophil may phagocytose the target cell or secrete neutrophil extracellular traps (NETs); finally, they may activate their respiratory burst cascade to kill phagocytosed cells.

In some embodiments, the first eosinophil engaging domain comprises a VH domain and the second eosinophil engaging domain comprises a VL domain. In other embodiments, the first eosinophil engaging domain comprises a VL domain and the second eosinophil engaging domain comprises a VH domain. In such embodiments, when paired together the first and second eosinophil engaging domains may comprise an scFv (by this we mean equivalent to an scFv but for the fact that the VH and VL are not in a single-chain configuration).

If the first and second eosinophil engaging domains are a pair of VH and VL domains, the VH and VL domains may be specific for an antigen expressed on the surface of an eosinophil, such as CD89 (Fc alpha receptor 1), FcεRI, FcγRI (CD64), FcγRIIA (CD32), FcγRIIIB (CD16b), or TLR4.

Table 10 presents selected publications on some exemplary antibodies specific for an antigen expressed on the surface of an eosinophil.

TABLE 10
Selected References Showing Specificity of Exemplary
Antibodies for Surface Antigens on Eosinophils

| | |
|---|---|
| CD89 (Fc | Xu et al. Critical Role of Kupffer Cell CD89 Expression in |
| alpha | Experimental IgA Nephropathy. PLoS One. 11(7): e0159426 (2016) |
| receptor 1) | Monteiro R C et al. IgA Fc receptors. Annu Rev Immunol. 21: 177-204. |
| | (2003) |
| | Morton H C et al. CD89: the human myeloid IgA Fc receptor. |
| | Arch Immunol Ther Exp (Warsz). 49(3): 217-29 (2001) |
| FcεRI | Stone K D et al. IgE, mast cells, basophils, and eosinophils. |
| | J Allergy Clin Immunol. 125(2 Suppl 2): S73-80 (2010) |
| | Conner E R and Saini S S. The immunoglobulin E receptor: expression |
| | and regulation. Curr Allergy Asthma Rep. 5(3): 191-6 (2005) |
| FcγRI | Nimmerjahn F and Ravetch J V. Antibodies, Fc receptors and cancer. |
| (CD64) | Curr Opin Immunol. 19(2): 239-45 (2007) |
| | Ravetch J V: Fc receptors. In Fundamental Immunology, edn5. Edited |
| | by Paul W E. Lippincott-Raven; 685-700 (2003) |
| | Nimmerjahn F, Ravetch J V: Fcγ receptors: old friends and new family |
| | members. Immunity 24: 19-28 (2006) |
| FcγRIIA | Nimmerjahn F and Ravetch J V. Antibodies, Fc receptors and cancer. |
| (CD32) | Curr Opin Immunol. 19(2): 239-45 (2007) |
| | Ravetch J V: Fc receptors. In Fundamental Immunology, edn5. Edited |
| | by Paul W E. Lippincott-Raven; 685-700 (2003) |
| | Nimmerjahn F, Ravetch J V: Fcγ receptors: old friends and new family |
| | members. Immunity 24: 19-28 (2006) |
| FcγRIIIB | Nimmerjahn F and Ravetch J V. Antibodies, Fc receptors and cancer. |
| (CD 16b) | Curr Opin Immunol. 19(2): 239-45 (2007) |
| | Ravetch J V: Fc receptors. In Fundamental Immunology, edn5. Edited |
| | by Paul W E. Lippincott-Raven; 685-700 (2003) |
| | Nimmerjahn F, Ravetch J V: Fcγ receptors: old friends and new family |
| | members. Immunity 24: 19-28 (2006) |
| TLR4 | Beutler B A. TLRs and innate immunity. Blood 113(7): 1399-407 |
| | (2009) |
| | Beutler B et al. Genetic analysis of host resistance: Toll-like receptor |
| | signaling and immunity at large. Annu Rev Immunol. 24: 353-89 (2006) |

6. Basophil Engaging Domains

In some embodiments, the immune cell engaging domain is a basophil engaging domain. When the two basophil engaging domains are associated together in the two-component system, they may bind to an antigen on the surface of the basophil to engage these cells. In some embodiments, the antigen on the surface of the basophil may be CD89 (Fc alpha receptor 1) or FcεRI.

In some embodiments, having one half of the two-component system bind to a surface protein on the basophil and having the other half of the system bind to cancer cells allows specific engagement of basophils. Engagement of basophils can lead to the release of basophil granule components such as histamine, proteoglycans, and proteolytic enzymes. They also secrete leukotrienes (LTD-4) and cytokines.

When the two basophil engaging domains are associated together in the ATTAC, the basophil may degranulate.

In some embodiments, the first basophil engaging domain comprises a VH domain and the second basophil engaging domain comprises a VL domain. In other embodiments, the first basophil engaging domain comprises a VL domain and the second basophil engaging domain comprises a VH domain. In such embodiments, when paired together the first and second basophil engaging domains may comprise an scFv (by this we mean equivalent to an scFv but for the fact that the VH and VL are not in a single-chain configuration).

If the first and second basophil engaging domains are a pair of VH and VL domains, the VH and VL domains may be specific for an antigen expressed on the surface of a basophil, such as CD89 (Fc alpha receptor 1) or FcεRI.

Table 11 presents selected publications on some exemplary antibodies specific for an antigen expressed on the surface of a basophil.

TABLE 11
Selected References Showing Specificity of Exemplary
Antibodies for Surface Antigens on Basophils

| CD89 (Fc alpha | Xu et al. Critical Role of Kupffer Cell CD89 Expression in |
|---|---|
| receptor 1) | Experimental IgA Nephropathy. PLoS One. 11(7): e0159426 (2016). |
| | Monteiro R C et al. IgA Fc receptors. Annu Rev Immunol. 21: 177- |
| | 204 (2003) |
| | Morton H C et al. CD89: the human myeloid IgA Fc receptor. |
| | Arch Immunol Ther Exp (Warsz). 49(3): 217-29 (2001) |
| FcεRI | Stone K D et al. IgE, mast cells, basophils, and eosinophils. |
| | J Allergy Clin Immunol. 125(2 Suppl 2): S73-80 (2010) |
| | Conner E R and Saini S S. The immunoglobulin E receptor: |
| | expression and regulation. Curr Allergy Asthma Rep. 5(3): 191-6 |
| | (2005) |

7. γδ T Cells

In some embodiments, the immune cell engaging domain is a γδ T-cell engaging domain. As used herein, a γδ T cell refers to a T cell having a TCR made up of one gamma chain (γ) and one delta chain (δ).

When the two γδ T-cell engaging domains are associated together in the two-component system, they may bind to an antigen on the surface of the γδ T cell to engage these cells. In some embodiments, the antigen on the surface of the γδ T cell may be γδ TCR, NKG2D, CD3 Complex (CD3ε, CD3γ, CD3δ, CD3ζ, CD3η), 4-1BB, DNAM-1, or TLRs (e.g., TLR2, TLR6).

In some embodiments, having one half of the two-component system bind to a surface protein on the γδ T cell and having the other half of the system bind to cancer cells allows specific engagement of γδ T cells. Engagement of γδ T cell can lead to cytolysis of the target cell and release of proinflammatory cytokines such as TNFα and IFNγ.

When the two γδ T-cell engaging domains are associated together in the ATTAC, the γδ T cell may kill the target cell.

In some embodiments, the first γδ T-cell engaging domain comprises a VH domain and the second γδ T-cell engaging domain comprises a VL domain. In other embodiments, the first γδ T-cell engaging domain comprises a VL domain and the second γδ T-cell engaging domain comprises a VH domain. In such embodiments, when paired together the first and second γδ T-cell engaging domains may comprise an scFv (by this we mean equivalent to an scFv but for the fact that the VH and VL are not in a single-chain configuration).

If the first and second γδ T-cell engaging domains are a pair of VH and VL domains, the VH and VL domains may be specific for an antigen expressed on the surface of a γδ T cell, such as γδ TCR, NKG2D, CD3 Complex (CD3ε, CD3γ, CD3δ, CD3ζ, CD3η), 4-1BB, DNAM-1, or TLRs (TLR2, TLR6).

Table 12 presents selected publications on some exemplary antibodies specific for an antigen expressed on the surface of a γδ T cell.

TABLE 12
Selected References Showing Specificity of Exemplary Antibodies
for Surface Antigens on Gamma-delta (γδ) T cells

| | |
|---|---|
| γδ TCR | Vantourout P and Hayday A. Six-of-the-best: unique contributions |
| | of γδ T cells to immunology. Nat Rev Immunol. 13(2): 88-100 (2013) |
| | Hayday A and Tigelaar R. Immunoregulation in the tissues by |
| | gammadelta T cells. Nat Rev Immunol. 3(3): 233-42 (2003) |
| | Hayday A C. γδ cells: a right time and a right place for a conserved |
| | third way of protection. Annu Rev Immunol. 18: 975-1026 (2000) |
| NKG2D | Vantourout P and Hayday A. Six-of-the-best: unique contributions |
| | of γδ T cells to immunology. Nat Rev Immunol. 13(2): 88-100 (2013) |
| | Hayday A and Tigelaar R. Immunoregulation in the tissues by |
| | gammadelta T cells. Nat Rev Immunol. 3(3): 233-42 (2003) |
| | Hayday A C. γδ cells: a right time and a right place for a conserved |
| | third way of protection. Annu Rev Immunol. 18: 975-1026 (2000) |
| | Raulet D H et al. Regulation of ligands for the NKG2D activating |
| | receptor. Annu Rev Immunol. 31: 413-41 (2013) |
| CD3 | Vantourout P and Hayday A. Six-of-the-best: unique contributions |
| Complex | of γδ T cells to immunology. Nat Rev Immunol. 13(2): 88-100 (2013) |
| (CD3α, | Hayday A and Tigelaar R. Immunoregulation in the tissues by |
| CD3β, CD3γ, | gammadelta T cells. Nat Rev Immunol. 3(3): 233-42 (2003) |
| CD3γ, CD3ε) | Hayday A C. γδ cells: a right time and a right place for a conserved |
| | third way of protection. Annu Rev Immunol. 18: 975-1026 (2000) |
| 4-1BB | Ochoa M C et al. Antibody-dependent cell cytotoxicity: |
| | immunotherapy strategies enhancing effector NK cells. Immunol Cell |
| | Biol. 95(4): 347-355 (2017) |
| DNAM-1 | Niu C et al. Low-dose bortezomib increases the expression of |
| | NKG2D and DNAM-1 ligands and enhances induced NK and γδ T cell- |
| | mediated lysis in multiple myeloma. Oncotarget. 8(4): 5954-5964 |
| | (2017) |
| | Toutirais O et al. DNAX accessory molecule-1 (CD226) promotes |
| | human hepatocellular carcinoma cell lysis by Vgamma9Vdelta2 T cells. |
| | Eur J Immunol. 39(5): 1361-8 (2009) |
| TLRs (TLR2, | Beutler B A. TLRs and innate immunity. Blood. 113(7): 1399-407 |
| TLR6) | (2009) |
| | Beutler B et al. Genetic analysis of host resistance: Toll-like receptor |
| | signaling and immunity at large. Annu Rev Immunol. 24: 353-89 (2006) |

8. Natural Killer T Cells (NKT Cells)

In some embodiments, the immune cell engaging domain is a NKT engaging domain. NKT cells refers to T cells that express the Vα24 and Vβ11 TCR receptors.

When the two NKT engaging domains are associated together in the two-component system, they may bind to an antigen on the surface of the NKT to engage these cells. In some embodiments, the antigen on the surface of the NKT may be αβTCR, NKG2D, CD3 Complex (CD3ε, CD3γ, CD3δ, CD3ζ, CD3η), 4-1BB, or IL-12R.

In some embodiments, having one half of the two-component system bind to a surface protein on the NKT and having the other half of the system bind to cancer cells allows specific engagement of NKT. Engagement of NKTs can lead to cytolysis of the target cell.

When the two NKT engaging domains are associated together in the ATTAC, the NKT may cytolysis of the target cell and the release of proinflammatory cytokines.

In some embodiments, the first NKT engaging domain comprises a VH domain and the second NKT engaging domain comprises a VL domain. In other embodiments, the first NKT engaging domain comprises a VL domain and the second NKT engaging domain comprises a VH domain. In such embodiments, when paired together the first and second NKT engaging domains may comprise an scFv (by this we mean equivalent to an scFv but for the fact that the VH and VL are not in a single-chain configuration).

If the first and second NKT engaging domains are a pair of VH and VL domains, the VH and VL domains may be specific for an antigen expressed on the surface of a NKT, such as aβTCR, NKG2D, CD3 Complex (CD3ε, CD3γ, CD3δ, CD3ζ, CD3η), 4-1BB, or IL-12R.

Table 13 presents selected publications on some exemplary antibodies specific for an antigen expressed on the surface of a NKT.

TABLE 13
Selected References Showing Specificity of Exemplary
Antibodies for Surface Antigens on NKT cells

| | |
|---|---|
| αβTCR | Courtney A H et al. TCR Signaling: Mechanisms of Initiation and |
| | Propagation. Trends Biochem Sci. 43(2): 108-123 (2018) |
| | Davis M M et al. Ligand recognition by alpha beta T cell receptors. |
| | Annu. Rev. Immunol. 16: 523-544 (1998) |
| NKG2D | Sentman C L and Meehan K R. NKG2D CARs as cell therapy for |
| | cancer. Cancer J. 20(2): 156-9 (2014) |
| | Ullrich E et al. New prospects on the NKG2D/NKG2DL system for |
| | oncology. Oncoimmunology. 2(10): e26097 (2013) |
| CD3 Complex (CD3α, | Courtney A H et al. TCR Signaling: Mechanisms of Initiation and |
| CD3β, CD3γ, | Propagation. Trends Biochem Sci. 43(2): 108-123 (2018) |
| CD3γ, CD3ε) | |
| 4-1BB | Makkouk A et al. Rationale for anti-CD137 cancer immunotherapy. |
| | Eur J Cancer. 54: 112-119 (2016) |
| | Zhou S J. Strategies for Bispecific Single Chain Antibody in Cancer |
| | Immunotherapy. J Cancer. 8(18): 3689-3696 (2017) |
| IL-12R | Lasek W et al. Interleukin 12: still a promising candidate for tumor |
| | immunotherapy? Cancer Immunol Immunother. 63(5): 419-35 (2014) |

9. Engineered Immune Cells

In some embodiments, the immune cell engaging domain is an engineered immune cell engaging domain.

In some embodiments, the engineered immune cell is a chimeric antigen receptor (CAR) cell. In some embodiments, the CAR comprises an extracellular domain capable of tightly binding to a tumor antigen (for example, an scFv), fused to a signaling domain partly derived from a receptor naturally expressed by an immune cell. Exemplary CARs are described in Facts about Chimeric Antigen Receptor (CAR) T-Cell Therapy, Leukemia and Lymphoma Society, December 2017. CARs may comprise an scFV region specific for a tumor antigen, an intracellular co-stimulatory domain, and linker and transmembrane region. For example, a CAR in a CAR T cell may comprise an extracellular domain of a tumor antigen fused to a signaling domain partly derived from the T cell receptor. A CAR may also comprise a co-stimulatory domain, such as CD28, 4-1 BB, or OX40. In some embodiments, binding of the CAR expressed by an immune cell to a tumor target antigen results in immune cell activation, proliferation, and target cell elimination. Thus, a range of CARs may be used that differ in their scFV region, intracellular co-stimulatory domains, and linker and transmembrane regions to generate engineered immune cells.

Exemplary engineered immune cells include CAR T cells, NK cells, NKT cells, and γδ cells. In some embodiments, engineered immune cells are derived from the patient's own immune cells. In some embodiments, the patient's tumor expresses a tumor antigen that binds to the scFV of the CAR.

Potential CAR targets studied so far include CD19, CD20, CD22, CD30, CD33, CD123, ROR1, Igk light chain, BCMA, LNGFR, and NKG2D. However, the CAR technology would be available for developing engineered immune cells to a range of tumor antigens.

In some embodiments, the engineered immune cell is a genetically engineered immune cell.

When the two engineered immune cell engaging domains are associated together in the two-component system, they may bind to an antigen on the surface of the engineered immune cell to engage these cells. In some embodiments, the antigen on the surface of the engineered immune cell may be an engagement domain recited in this application with specificity for T cells, NK cells, NKT cells, or γδ cells.

In some embodiments, having one half of the two-component system bind to a surface protein on the engineered immune cell and having the other half of the system bind to cancer cells allows specific engagement of engineered immune cells. Engagement of engineered immune cells can lead to activation of the effector response of these cells such as cytolysis of their target and release of cytokines.

When the two engineered immune cell engaging domains are associated together in the ATTAC, the engineered immune cell may kill the target cell.

In some embodiments, the first engineered immune cell engaging domain comprises a VH domain and the second engineered immune cell engaging domain comprises a VL domain. In other embodiments, the first engineered immune cell engaging domain comprises a VL domain and the second engineered immune cell engaging domain comprises a VH domain. In such embodiments, when paired together the first and second engineered immune cell engaging domains may comprise an scFv (by this we mean equivalent to an scFv but for the fact that the VH and VL are not in a single-chain configuration).

If the first and second engineered immune cell engaging domains are a pair of VH and VL domains, the VH and VL domains may be specific for an antigen expressed on the surface of an engineered immune cell, based on the type of cell used for the engineering.

E. Inert Binding Partners

Inert binding partners are described in U.S. Pat. No. 10,035,856. A TEAC or ATTAC can comprise at least one inert binding partner capable of binding the first immune cell or T-cell engaging domain and preventing it from binding to a second immune cell or T-cell engaging domain unless certain conditions occur. In some embodiments, a first and second immune cell engaging domain, such as a T-cell engaging domain, can pair together when neither is bound to an inert binding partner.

In some embodiments, one or more first and second inert binding partners are capable of dissociation once at least one protease cleavage site for each inert binding partner has been cleaved and after dissociation the two immune cell or T-cell engaging domains that had been bound by the inert binding partners are capable of binding to each other and exhibiting immune cell or T-cell binding activity.

In some embodiments, cleavage of one or more protease cleavage site causes dissociation of one or more inert binding partner and complementation of the first and second immune cell or T-cell engaging domain. By complementation, it is meant that the first and second immune cell or T-cell engaging domain can bind to or pair with each together. In some embodiments, the paired (i.e., complemented) first and second immune cell or T-cell engaging domains can bind an antigen on an immune cell, such as a T cell, when neither immune cell or T-cell engaging domain is bound to an inert binding partner.

1. Inactivated VII or VL Domains as Inert Binding Partners

In some embodiments when an immune cell engaging domain comprises a VH or VL domain, the inert binding partner has homology to a corresponding VL or VH domain that can pair with the immune cell engaging domain to form a functional antibody and bind to an immune cell antigen. This immune cell antigen may be an antigen present on any immune cell, including a T cell, a macrophage, a natural killer cell, a neutrophil, eosinophil, basophil, γδ T cell, natural killer T cell (NKT cells), or engineered immune cell. In some embodiments, this immune cell antigen is CD3.

In some embodiments, the inert binding partner comprises a VH or VL that cannot specifically bind an antigen when paired with its corresponding VL or VH of the immune cell engaging domain because of one or more mutations made in the inert binding partner to inhibit binding to the target antigen. In some embodiments, the VH or VL of the inert binding partner may differ by one or more amino acids from a VH or VL specific for an immune cell antigen. In other words, one or more mutations may be made to a VH or VL specific for a target immune cell antigen to generate an inert binding partner.

These mutations may be, for example, a substitution, insertion, or deletion in the polypeptide sequence of a VH or VL specific for an immune cell antigen to generate an inert binding partner. In some embodiments, the mutation in a VH or VL specific for an immune cell antigen may be made within CDR1, CDR2, or CDR3 to generate an inert binding partner. In some embodiments, an VH or VL used as an inert binding partner may retain the ability to pair with an immune cell engaging domain, but the resulting paired VH/VL domains have reduced binding to the immune cell antigen. In some embodiments, an inert binding partner has normal affinity to bind its corresponding immune cell engaging domain, but the paired VH/VL has lower binding affinity for the immune cell antigen compared to a paired VH/VL that does not comprise the mutation of the inert binding partner. For example, this lower affinity may be a 20-fold, 100-fold, or 1000-fold lower binding to an immune cell antigen.

In some embodiments, the first immune cell engaging domain comprises a VH specific for an immune cell antigen and the inert binding partner comprises a VL domain for the same antigen that has one or more mutations such that the paired VH/VL has decreased or no binding to the antigen. In some embodiments, the first immune cell engaging domain comprises a VL specific for an immune cell antigen and the inert binding partner comprises a VH domain for the same antigen that has one or more mutations such that the paired VH/VL has decreased or no binding to the antigen.

In some embodiments, the second immune cell engaging domain comprises a VH specific for an immune cell antigen and the inert binding partner comprises a VL domain for the same antigen that has one or more mutations such that the paired VH/VL has decreased or no binding to the antigen. In some embodiments, the second immune cell engaging domain comprises a VL specific for an immune cell antigen and the inert binding partner comprises a VH domain for the same antigen that has one or more mutations such that the paired VH/VL has decreased or no binding to the antigen.

F. Inert Binding Partners Obtained from Unrelated Antibodies

In some embodiments, a VH or VL used as an inert binding partner is unrelated to the VL or VH of the immune cell engaging domain. In other words, the inert binding partner may have little or no sequence homology to the corresponding VH or VL that normally associates with the VL or VH of the immune cell engaging domain. In some embodiments, the VH or VL used as an inert binding partner may be from a different antibody or scFv than the VL or VH used as the immune cell engaging domain.

If both components have inert binding partner, in some embodiments, the VH inert binding partner of one component and the VL inert binding partner of the other component may be from different antibodies

G. Cleavage Site

A range of different cleavage sites can be used in TEACs and ATTACs. In some embodiments, wherein the cleavage site is cleaved by an enzyme expressed by the cancer cells; cleaved through a pH-sensitive cleavage reaction inside the cancer cell; cleaved by a complement-dependent cleavage reaction; or cleaved by a protease that is colocalized to the cancer cell by a targeting moiety that is the same or different from the targeting moiety in the agent. Cleavage sites are described in U.S. Pat. No. 10,035,856.

In some embodiments, at least one cleavage site may be cleaved by an enzyme expressed by the cancer or in the cancer microenvironment. As used herein, an enzyme (such as a protease) expressed “in the cancer microenvironment” refers to an enzyme that is localized to the vicinity of a cancer cell, but outside the cancer cell. Tumors depend on complex interactions between cancer cells and the surrounding stromal compartment. For example, interactions between cancer cells and its surrounding stroma can promote tumor progression by mechanisms such as remodeling of the extracellular matrix to enhance invasion, which may rely on proteases expressed by stromal cells. In some embodiments, cells in the cancer microenvironment have an altered phenotype that helps to promote tumor survival, growth, or metastasis, such as by increased or altered expression of proteases. In some embodiments, a protease in the cancer microenvironment is a protease expressed by a stromal cell in the vicinity of the cancer. In some embodiments, a protease is secreted by a stromal cell in the vicinity of the cancer. In some embodiments, a protease expressed in the cancer microenvironment is not expressed by the cancer cells themselves.

In addition, cancer cells are known to express certain enzymes, such as proteases, and these may be employed in this strategy to cleave the targeted T-cell engaging agent's cleavage site. In some embodiments, one or more protease cleavage sites are cleaved by a protease expressed by the cancer. By way of nonlimiting example, cathepsin B cleaves FR, FK, VA and VR amongst others; cathepsin D cleaves PRSFFRLGK (SEQ ID NO: 45), ADAM28 cleaves KPAKFFRL (SEQ ID NO: 1), DPAKFFRL (SEQ ID NO: 2), KPMKFFRL (SEQ ID NO: 3) and LPAKFFRL (SEQ ID NO: 4); and MMP2 cleaves AIPVSLR (SEQ ID NO: 46), SLPLGLWAPNFN (SEQ ID NO: 47), HPVGLLAR (SEQ ID NO: 48), GPLGVRGK (SEQ ID NO: 49) (also cleaved by MMP9), and GPLGLWAQ (SEQ ID NO: 50), for example. Other cleavage sites listed in Table 1A may also be employed. Protease cleavage sites and proteases associated with cancer are well known in the art. Oncomine (www.oncomine.org) is an online cancer gene expression database, so when the agent of the invention is for treating cancer, the skilled person may search the Oncomine database to identify a particular protease cleavage site (or two protease cleavage sites) that will be appropriate for treating a given cancer type. Alternative databases include the European Bioinformatic Institute (www.ebi.ac.uk), in particular (www.ebi.ac.uk/gxa). Protease databases include PMAP (www.proteolysis.org), ExPASy Peptide Cutter (ca.expasy.org/tools/peptidecutter) and PMAP.Cut DB (cutdb.burnham.org).

In some embodiments, at least one cleavage site may be cleaved through a pH-sensitive cleavage reaction inside the cancer cell. If the ATTAC or TEAC is internalized into the cell, the cleavage reaction may occur inside the cell and may be triggered by a change in pH between the microenvironment outside the unwanted cell and the interior of the cell. Specifically, some cancer types are known to have acidic environments in the interior of the cancer cells. Such an approach may be employed when the interior unwanted cell type has a characteristically different pH from the extracellular microenvironment, such as particularly the glycocalyx. Because pH cleavage can occur in all cells in the lysozymes, selection of a targeting agent when using a pH-sensitive cleavage site may require, when desired, more specificity. For example, when a pH-sensitive cleavage site is used, a targeting agent that binds only or highly preferably to cancer cells may be desired (such as, for example, an antibody binding to mesothelin for treatment of lung cancer).

In certain embodiments, at least one cleavage site may be cleaved by a complement-dependent cleavage reaction. Once a targeted ATTAC or TEAC binds to the unwanted cell, the patient's complement cascade may be triggered. In such a case, the complement cascade may also be used to cleave the inert binding partner from the first immune cell or T-cell engaging domain by using a cleavage site sensitive to a complement protease. For example, C1r and C1s and the C3 convertases (C4B,2a and C3b,Bb) are serine proteases. C3/C5 and C5 are also complement proteases. Mannose-associated binding proteins (MASP), serine proteases also involved in the complement cascade and responsible for cleaving C4 and C2 into C4b2b (a C3 convertase) may also be used. For example, and without limitation, C1s cleaves YLGRSYKV (SEQ ID NO: 213) and MQLGRX (SEQ ID NO: 214). MASP2 is believed to cleave SLGRKIQI (SEQ ID NO: 215). Complement component C2a and complement factor Bb are believed to cleave GLARSNLDE (SEQ ID NO: 216).

In some embodiments, at least one cleavage site may be cleaved by a protease that is colocalized to the unwanted cell by a targeting moiety that is the same or different from the targeting moiety in the ATTAC or TEAC. For example, any protease may be simultaneously directed to the microenvironment of the unwanted cells by conjugating the protease to a targeting agent that delivers the protease to that location. The targeting agent may be any targeting agent described herein. The protease may be affixed to the targeting agent through a peptide or chemical linker and may maintain sufficient enzymatic activity when bound to the targeting agent.

In some embodiments comprising two cleavage sites, the protease cleavage sites are different. In some embodiments comprising two cleavage sites, the protease cleavage sites are the same.

H. Linkers

In addition to the cleavage site, linkers may optionally be used to attach the separate parts of the ATTAC or TEAC together. By linker, we include any chemical moiety that attaches these parts together. In some embodiments, the linkers may be flexible linkers. Linkers include peptides, polymers, nucleotides, nucleic acids, polysaccharides, and lipid organic species (such as polyethylene glycol). In some embodiments, the linker is a peptide linker. Peptide linkers may be from about 2-100, 10-50, or 15-30 amino acids long. In some embodiments, peptide linkers may be at least 10, at least 15, or at least 20 amino acids long and no more than 80, no more than 90, or no more than 100 amino acids long. In some embodiments, the linker is a peptide linker that has a single or repeating GGGGS (SEQ ID NO: 85), GGGS (SEQ ID NO: 86), GS (SEQ ID NO: 87), GSGGS (SEQ ID NO: 88), GGSG (SEQ ID NO: 89), GGSGG (SEQ ID NO: 90), GSGSG (SEQ ID NO: 91), GSGGG (SEQ ID NO: 92), GGGSG (SEQ ID NO: 93), and/or GSSSG (SEQ ID NO: 94) sequence(s).

In some embodiments, the linker is a maleimide (MPA) or SMCC linker.

Linkers are also described in U.S. Pat. No. 10,035,856.

I. Two-Component TEAC or ATTAC Comprising Copies of Domains or Moieties

In some embodiments, one or both components comprise two copies of one or more domains or moieties. In some embodiments, the first component comprises two copies of one or more domains or moieties. In some embodiments, the second component comprises two copies of one or more domains or moieties. In some embodiments, both components comprise two copies of one or more domains or moieties.

In some embodiments, the first component comprises two copies of a first targeting moiety; two copies of a first T-cell engaging domain; and two copies of a first inert binding partner. In some embodiments, the second component comprises two copies of a second targeting moiety; two copies of a second T-cell engaging domain; and two copies of a second inert binding partner. In some embodiments, a protease cleavage site separates both inert binding partners from their respective T-cell engaging domains.

In some embodiments, the two copies of the targeting moiety are the same. In some embodiments, the two copies of the T-cell engaging domain are the same. In some embodiments, the two copies of the inert binding partner are the same. In some embodiments, the two copies of the protease cleavage site separating the inert binding partners from their respective T-cell engaging domains are the same. In some embodiments, the two copies of a protease cleavage site separating the inert binding partners from their respective T-cell engaging domains are different.

In some embodiments, a component comprising two copies of a first targeting moiety; two copies of a first T-cell engaging domain; and two copies of a first inert binding partner is generated in a cell via Fc pairing, as described in Examples 1 and 2.

J. Single-Component of a Two-Component TEAC or ATTAC

In some embodiments, cancer cells are targeted using a kit or composition comprising a component of a TEAC or ATTAC.

In some embodiments, a component of a TEAC or ATTAC comprising a half-life extending moiety can be administered with another component also comprising a half-life extending moiety.

In some embodiments, a component of a TEAC or ATTAC comprising a half-life extending moiety can be administered with another component that does not comprise a half-life extending moiety. In this way, only one component of a two-component TEAC or ATTAC has a half-life extending moiety.

In some embodiments, a component is a TEAC component. In some embodiments, a component for use in a kit or composition for treating cancer in a patient comprises a first targeted immune cell engaging agent comprising a targeting moiety that binds a tumor antigen expressed by the cancer; an immune cell engaging domain capable of immune cell binding activity when binding another immune cell engaging domain, wherein the other immune cell engaging domain is not part of the first component, and wherein the immune cell engaging domain comprises either a VH domain or VL domain, optionally wherein the immune cell engaging domain comprises a T-cell engaging domain; an inert binding partner for the immune cell engaging domain binding to the immune cell engaging domain such that the immune cell engaging domain does not bind to the other immune cell engaging domain unless the inert binding partner is removed, wherein if the immune cell engaging domain comprises a VH domain, the inert binding partner comprises a VL domain and if the immune cell engaging domain comprises a VL domain, the inert binding partner comprises a VH domain; a half-life extending moiety, wherein the half-life extending moiety is attached (directly or indirectly) to the inert binding partner; and a protease cleavage site separating the immune cell engaging domain and the inert binding partner, wherein the protease cleavage site is capable of releasing the inert binding partner and half-life extending moiety from the immune cell engaging domain in the presence of a protease (1) expressed by the cancer; or (2) colocalized to the cancer by a targeting moiety that binds a tumor antigen expressed by the cancer and that is the same or different from the targeting moiety in the agent, wherein cleavage of the protease cleavage site causes loss of the inert binding partner and allows for complementation with the other immune cell engaging domain that is not part of the agent, further wherein if the immune cell engaging domain comprises a VH domain, the other immune cell engaging domain comprises a VL domain and if the immune cell engaging domain comprises a VL domain, the other immune cell engaging domain comprises a VH domain.

In some embodiments, a component is an ATTAC component. In some embodiments, a component for use in a kit or composition for treating cancer in a patient comprises a first targeted immune cell engaging agent comprising an immune cell selection moiety capable of selectively targeting an immune cell; an immune cell engaging domain capable of immune cell binding activity when binding another immune cell engaging domain, wherein the other immune cell engaging domain is not part of the first component, and wherein the immune cell engaging domain comprises either a VH domain or VL domain, optionally wherein the immune cell engaging domain comprises a T-cell engaging domain; an inert binding partner for the immune cell engaging domain binding to the immune cell engaging domain such that the immune cell engaging domain does not bind to the other immune cell engaging domain unless the inert binding partner is removed, wherein if the immune cell engaging domain comprises a VH domain, the inert binding partner comprises a VL domain and if the immune cell engaging domain comprises a VL domain, the inert binding partner comprises a VH domain; a half-life extending moiety, wherein the half-life extending moiety is attached (directly or indirectly) to the inert binding partner; and a protease cleavage site separating the immune cell engaging domain and the inert binding partner, wherein the protease cleavage site is capable of releasing the inert binding partner and half-life extending moiety from the immune cell engaging domain in the presence of a protease (1) expressed by the cancer; or (2) colocalized to the cancer by a targeting moiety that binds a tumor antigen expressed by the cancer and that is the same or different from the targeting moiety in the agent, wherein cleavage of the protease cleavage site causes loss of the inert binding partner and allows for complementation with the other immune cell engaging domain that is not part of the agent, further wherein if the immune cell engaging domain comprises a VH domain, the other immune cell engaging domain comprises a VL domain and if the immune cell engaging domain comprises a VL domain, the other immune cell engaging domain comprises a VH domain.

### II. Single-Agent TEAC or ATTAC

This application also describes a TEAC or ATTAC that is comprised in a single agent. In other words, in a single-agent TEAC or ATTAC, all domains necessary for activity are included in a single molecule. A single-agent TEAC or ATTAC could allow dosing of a single agent as a treatment.

In some embodiments, one or more linker attaches different domains in a single-agent TEAC or ATTAC.

In some embodiments, a single-agent TEAC or ATTAC may also be designed to separate into two components after administration to the patient, where the two components are to be released before function. This separation may occur, for example, by protease cleavage in the blood or in the tumor microenvironment. After cleavage, this single-agent now becomes in the patient a two-component TEAC or ATTAC. At this initial point in separation, the inert binding partner is still attached to the T-cell or immune cell engaging domain.

In some embodiments, a single-agent TEAC or ATTAC may be comprised in a single polypeptide chain (i.e., a linker) designed to allow cleavage to generate two separate components, wherein the inert binding partners are on separate components after cleavage. In some embodiments, a single-agent TEAC or ATTAC comprises one or more cleavage site to allow release of one or more inert binding partners, but the other domains of the TEAC or ATTAC are not designed to promote cleavage and separation of other domains besides release of one or more inert binding partners from the T-cell or immune cell engaging domains.

In some embodiments, a cleavage site comprised within a linker covalently binding a first component and the second component is a protease cleavage site. SEQ ID NOs: 1-84 list some exemplary protease cleavage sites that may be used, but the invention is not limited to this set of proteases cleavage sites and other protease cleavage sites may be employed.

In some embodiments, a cleavage site comprised within a linker covalently binding a first component and the second component is a tumor-associated protease cleavage site. A tumor associated protease is one that is associated with a tumor. In some embodiments, a tumor-associated protease has higher expression in the tumor versus other regions of the body. Any protease with expression in a tumor may be used to select a tumor-associated protease cleavage site for the invention.

In some embodiments, a cleavage site comprised within a linker covalently binding a first component and the second component is a cleavage site for a protease found in the blood. Exemplary proteases found in the blood include thrombin, neutrophil elastase, and furin.

A. Single-Agent TEAC

In some embodiments, a TEAC is comprised in a single agent, wherein the agent is not meant to be cleaved outside of the site of action. In this embodiment, there is no protease cleavage site available to separate the agent into separate components of a two-component system, except for the protease cleavage site between the inert binding partner and the T-cell engaging domain. In some embodiments, an agent for treating cancer in a patient comprises a first targeting moiety that binds a tumor antigen expressed by the cancer; a first T-cell engaging domain capable of T-cell binding activity when binding a second T-cell engaging domain, wherein the first T-cell engaging domain comprises either a VH domain or VL domain; a second T-cell engaging domain capable of T-cell binding activity when binding a first T-cell engaging domain, wherein the second T-cell engaging domain comprises either a VH domain or VL domain; a first inert binding partner for the first T-cell engaging domain binding to the first T-cell engaging domain such that the first T-cell engaging domain does not bind to the second T-cell engaging domain unless the inert binding partner is removed, wherein if the first T-cell engaging domain comprises a VH domain, the inert binding partner comprises a VL domain and if the first T-cell engaging domain comprises a VL domain, the inert binding partner comprises a VH domain; and a protease cleavage site separating the first T-cell engaging domain and the first inert binding partner, wherein the protease cleavage site is capable of releasing the inert binding partner from the T-cell engaging domain in the presence of a protease (1) expressed by the cancer or in the cancer microenvironment; or (2) colocalized to the cancer by a targeting moiety that binds a tumor antigen expressed by the cancer and that is the same or different from the targeting moiety in the agent; wherein the first and second T-cell engaging domains are capable of binding a T cell when neither is bound to an inert binding partner, and further wherein if the first T-cell engaging domain comprises a VH domain, the second T-cell engaging domain comprises a VL domain and if the first T-cell engaging domain comprises a VL domain, the second T-cell engaging domain comprises a VH domain.

A single-agent TEAC that is not meant to be cleaved outside of the site of action and that comprises a linker comprising an Fc domain may be referred to as a “IgG-Duo TEAC” or “Duo IgG-TEAC.” Using the term Duo explains that the embodiment employs a single-agent that has all of the targeting and T-cell engaging domains necessary for a functional construct after protease cleavage, such as the construct in FIG. 2D.

In some embodiments, a single-agent TEAC comprises only one targeting moiety that is capable of targeting the cancer. In some embodiments, a single-agent TEAC further comprises a second targeting moiety that is capable of targeting the cancer.

In some embodiments, a single-agent TEAC (Duo TEAC) is generated via pairing of two TEACs comprising complementary T-cell engaging domains, that have different purification tags, as described in Examples 1 and 2. In some embodiments, TEACs comprising complementary T cell engaging domains and with different purification tags (such as EPEA and histidine) are used to generate a single-agent TEAC. In some embodiments, the two TEACs comprising complementary T cell engaging domains are synthesized on one plasmid separated by an entity such as T2A self cleaving peptide or by using different promoters for each TEAC so that the TEACs are made in the same cells, such as HEK 293T cells. The TEACs would then form the Duo TEAC in the cell by pairing of CH domains to form an Fc domain. In some embodiments, both protein chains contain different purification tags (such as EPEA and histidine) to allow specific purification of the Duo TEAC comprising two different TEACs.

B. Single-Agent ATTAC

In some embodiments, an ATTAC is comprised in a single agent, wherein the agent is not meant to be cleaved outside of the site of action. In this embodiment, there is no protease cleavage site available to separate the agent into separate components of a two-component system, except for the protease cleavage site between the inert binding partner and the immune cell engaging domain. An agent for treating cancer in a patient comprises an immune cell selection moiety capable of selectively targeting an immune cell; a first immune cell engaging domain capable of immune cell binding activity when binding a second immune cell engaging domain, wherein the first immune cell engaging domain comprises either a VH domain or VL domain, optionally wherein the first immune cell engaging domain comprises a T-cell engaging domain; a second immune cell engaging domain capable of immune cell binding activity when binding a first immune cell engaging domain, wherein the second immune cell engaging domain comprises either a VH domain or VL domain, optionally wherein the second immune cell engaging domain comprises a T-cell engaging domain; a first inert binding partner for the first immune cell engaging domain binding to the first immune cell engaging domain such that the first immune cell engaging domain does not bind to the second immune cell engaging domain unless the inert binding partner is removed, wherein if the first immune cell engaging domain comprises a VH domain, the inert binding partner comprises a VL domain and if the first immune cell engaging domain comprises a VL domain, the inert binding partner comprises a VH domain; and a protease cleavage site separating the first immune cell engaging domain and the first inert binding partner, wherein the protease cleavage site is capable of releasing the inert binding partner from the immune cell engaging domain in the presence of a protease (1) expressed by the cancer or in the cancer microenvironment; or (2) colocalized to the cancer by a targeting moiety that binds a tumor antigen expressed by the cancer and that is the same or different from the targeting moiety in the agent; wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

A single-agent ATTAC that is not meant to be cleaved outside of the site of action and that comprises a linker comprising an Fc domain may be referred to as a “IgG-Duo ATTAC” or “Duo IgG-ATTAC.” Using the term Duo explains that the embodiment employs a single-agent that has all of the targeting and immune cell engaging and binding domains necessary for a functional construct after protease cleavage.

In some embodiments, a single-agent ATTAC comprises only one targeting moiety that is capable of targeting the cancer. In some embodiments, a single-agent ATTAC further comprises a second targeting moiety that is capable of targeting the cancer.

In some embodiments, a single-agent ATTAC is generated and purified in a similar manner as that described for the single-agent TEAC.

C. Single-Agent TEAC or ATTAC Comprising a Second Inert Binding Partner

In some embodiments, a single-agent TEAC or ATTAC further comprises a second inert binding partner for the second immune cell engaging domain binding to the second immune cell engaging domain such that the second immune cell engaging domain does not bind to the first immune cell engaging domain unless the inert binding partner is removed, wherein if the second immune cell or engaging domain comprises a VH domain, the inert binding partner comprises a VL domain and if the second immune cell engaging domain comprises a VL domain, the inert binding partner comprises a VH domain, optionally wherein the second immune cell engaging domain comprises a T-cell engaging domain; and a protease cleavage site separating the second immune cell engaging domain and the second inert binding partner, wherein the protease cleavage site is capable of releasing the inert binding partner from the immune cell engaging domain in the presence of a protease (1) expressed by the cancer or in the cancer microenvironment; or (2) colocalized to the cancer by a targeting moiety that binds a tumor antigen expressed by the cancer and that is the same or different from the targeting moiety in the agent, wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

D. Single-Agent TEAC or ATTAC Comprising a Half-Life Extending Moiety

In some embodiments, different moieties of a single-agent TEAC or ATTAC are joined via a linker. In some embodiments, a linker attaches the first and second inert binding partners of a single-agent TEAC or ATTAC. In some embodiments, a linker is capable of dissociation with the first and/or second inert binding partner upon cleavage of a protease cleavage sites.

In some embodiments, a linker comprises a half-life extending moiety as described above.

### III. Pharmaceutical Compositions

The TEAC or ATTAC may be employed as pharmaceutical compositions. As such, they may be prepared along with a pharmaceutically acceptable carrier. If parenteral administration is desired, for instance, the TEAC or ATTAC may be provided in sterile, pyrogen-free water for injection or sterile, pyrogen-free saline. Alternatively, the TEAC or ATTAC may be provided in lyophilized form for resuspension with the addition of a sterile liquid carrier.

### IV. Methods of Making

The targeted TEAC and ATTAC as described herein can be made using genetic engineering techniques. Specifically, a nucleic acid may be expressed in a suitable host to produce an ATTAC or TEAC. For example, a vector may be prepared comprising a nucleic acid sequence that encodes the targeted ATTAC or TEAC including all of its component parts and linkers and that vector may be used to transform an appropriate host cell. Other aspects of methods of making and pharmaceutical compositions are described in U.S. Pat. No. 10,035,856. Similar methods can be used to make any of the described embodiments, whether they are two-component or single-component agents.

In some embodiments, one or more nucleic acid molecules encodes the agent or component.

Various regulatory elements may be used in the vector as well, depending on the nature of the host and the manner of introduction of the nucleic acid into the host, and whether episomal maintenance or integration is desired.

Chemical linkage techniques, such as using maleimide or SMCC linkers, may also be employed.

In instances where the binding partner comprises an aptamer, a person of ordinary skill in the art would appreciate how to conjugate an aptamer to a protein, namely the immune cell engaging domain. Aptamers may be conjugated using a thiol linkage or other standard conjugation chemistries. A maleimide, succinimide, or SH group may be affixed to the aptamer to attach it to the immune cell engaging domain.

### V. Methods of Treatment

These agents or components may be used to treat cancer. In some embodiments, this cancer expresses an antigen that one or more targeting moieties can bind.

In some embodiments, a method of treating cancer expressing a tumor antigen that binds the first targeting moiety in a patient comprises administering an agent or component to the patient.

In some embodiments, a method of targeting an immune response of a patient to cancer comprising administering the agent or component to the patient.

In some embodiments, the T cells of the patient express CD3 or TCR and the T cell engaging domain binds CD3 or TCR.

In some embodiments, if the patient has regulatory T cells in the tumor, the selective immune cell engaging agent does not target markers present on regulatory immune cells (including, but not limited to CD4 and CD25).

In some embodiments, a method of treating cancer expressing a tumor antigen in a patient comprises administering a composition comprising a component, wherein the first targeting moiety comprised in the component binds the tumor antigen, and a second component comprising a half-life extending moiety.

In some embodiments, a method of treating cancer expressing a tumor antigen in a patient comprises administering a composition comprising a component, wherein the first targeting moiety comprised in the component binds the tumor antigen, and a second component not comprising a half-life extending moiety.

Thus, components described herein may be provided as kits or compositions that comprise two separate components. In some embodiments, both components of a kit or composition comprise a half-life extending moiety. In some embodiments, one component of a kit or composition comprises a half-life extending moiety, while the other component does not.

The TEAC or ATTAC described herein may be used in a method of treating a disease in a patient characterized by the presence of cancer cells comprising administering a TEAC or ATTAC. Additionally, the agents described herein may also be used in a method of targeting a patient's own immune response to cancer cells comprising administering a TEAC or ATTAC to the patient.

In some embodiments, the patient has cancer or a recognized pre-malignant state. In some embodiments, the patient has undetectable cancer, but is at high risk of developing cancer, including having a mutation associated with an increased risk of cancer. In some embodiments, the patient at high risk of developing cancer has a premalignant tumor with a high risk of transformation. In some embodiments, the patient at high risk of developing cancer has a genetic profile associated with high risk. In some embodiments, the presence of cancer or a pre-malignant state in a patient is determined based on the presence of circulating tumor DNA (ctDNA) or circulating tumor cells. In some embodiments, treatment is pre-emptive or prophylactic. In some embodiments, treatment slow or blocks the occurrence or reoccurrence of cancer.

The amount of the agent administered to the patient may be chosen by the patient's physician so as to provide an effective amount to treat the condition in question. In a two-component TEAC or ATTAC, the first component and the second component of the TEAC or ATTAC may be administered in the same formulation or two different formulations within a sufficiently close period of time to be active in the patient.

The patient receiving treatment may be a human. The patient may be a primate or any mammal. Alternatively, the patient may be an animal, such as a domesticated animal (for example, a dog or cat), a laboratory animal (for example, a laboratory rodent, such as a mouse, rat, or rabbit), or an animal important in agriculture (such as horses, cattle, sheep, or goats).

The cancer may be a solid or non-solid malignancy. In some embodiments, the cancer is a cancer other than a leukemia or lymphoma. In some embodiments, the cancer may be any cancer such as breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, leukemia, myeloma, nonHodgkin lymphoma, Hodgkin lymphoma, acute myeloid leukemia, acute lymphoblastic leukemia, chronic lymphoblastic leukemia, lymphoproliferative disorder, myelodysplastic disorder, myeloproliferative disease, and premalignant disease.

In some embodiments, a patient treated with an ATTAC has a tumor characterized by the presence of high levels of regulatory T cells (see Fridman W H et al., Nature Reviews Cancer 12:298-306 (2012) at Table 1). In patients with tumors characterized by a high presence of regulatory T cells, ATTAC therapy may be advantageous over other therapies that non-selectively target T cells, such as unselective BiTEs. In some embodiments, ATTAC therapy avoids engagement of regulatory T cells. In some embodiments, at least 80%, 85%, 90%, 91%, 92%, 93%, 94%, 95%, 96%, 97%, 98%, 99% of activated T cells are not regulatory T cells. In some embodiments, no regulatory T cells are activated by ATTAC therapy.

In some embodiments, the presence of a biomarker is used to select patients for receiving the TEAC or ATTAC. A wide variety of tumor markers are known in the art, such as those described at www.cancer.gov/about-cancer/diagnosis-staging/diagnosis/tumor-markers-fact-sheet. In some embodiments, the tumor marker is ALK gene rearrangement or overexpression; alpha-fetoprotein; beta-2-microglobulin; beta-human chorionic gonadotropin; BRCA1 or BRCA2 gene mutations; BCR-ABL fusion genes (Philadelphia chromosome); BRAF V600 mutations; C-kit/CD117; CA15-3/CA27.29; CA19-9; CA-125; calcitonin; carcinoembryonic antigen (CEA); CD20; chromogranin A (CgA); chromosomes 3, 7, 17, or 9p21; circulating tumor cells of epithelial origin (CELLSEARCH®); cytokeratin fragment 21-1; EGFR gene mutation analysis; estrogen receptor (ER)/progesterone receptor (PR); fibrin/fibrinogen; HE4; HER2/neu gene amplification or protein overexpression; immunoglobulins; KRAS gene mutation analysis; lactate dehydrogenase; neuron-specific enolase (NSE); nuclear matrix protein 22; programmed death ligand 1 (PD-L1); prostate-specific antigen (PSA); thyroglobulin; urokinase plasminogen activator (uPA); plasminogen activator inhibitor (PAI-1); 5-protein signature (OVA1®); 21-gene signature (Oncotype DX®); or 70-gene signature (Mammaprint®).

The TEAC or ATTAC may be administered alone or in conjunction with other forms of therapy, including surgery, radiation, traditional chemotherapy, or immunotherapy.

In some embodiments, the immunotherapy is checkpoint blockade. Checkpoint blockade refers to agents that inhibit or block inhibitory checkpoint molecules that suppress immune functions. In some embodiments, the checkpoint blockade targets CTLA4, PD1, PD-L1, LAG3, CD40, TIGIT, TIM3, VISTA or HLA-G.

In some embodiments, the immunotherapy is immune cytokines or cytokine fusions. Cytokines refer to cell-signaling proteins naturally made by the body to activate and regulate the immune system. Cytokine fusions refer to engineered molecules comprising all or part of a cytokine. For example, a cytokine fusion may comprise all or part of a cytokine attached to an antibody that allows targeting to a tumor such as Darleukin (see Zegers et al. (2015) Clin. Cancer Res., 21, 1151-60), Teleukin (see WO2018087172).

In some embodiments, the immunotherapy is cancer treatment vaccination. In some embodiments, cancer treatment vaccination boosts the body's natural defenses to fight cancer. These can either be against shared tumor antigens (such as E6, E7, NY-ESO, MUC1, or HER2) or against personalized mutational neoantigens.

### VI. Embodiments

The following numbered items provide embodiments as described herein, though the embodiments recited here are not limiting.

Item 1. An agent for treating cancer in a patient comprising: a first component comprising a targeted T-cell engaging agent comprising: a first targeting moiety that binds a tumor antigen expressed by the cancer; a first T-cell engaging domain capable of T-cell engaging activity when binding a second T-cell engaging domain, wherein the second T-cell engaging domain is not part of the first component, and wherein the first T-cell engaging domain comprises either a VH domain or VL domain; a first inert binding partner for the first T-cell engaging domain binding to the first T-cell engaging domain such that the first T-cell engaging domain does not bind to the second T-cell engaging domain unless the inert binding partner is removed, wherein if the first T-cell engaging domain comprises a VH domain, the inert binding partner comprises a VL domain and if the first T-cell engaging domain comprises a VL domain, the inert binding partner comprises a VH domain; a first half-life extending moiety, wherein the first half-life extending moiety is attached (directly or indirectly) to the first inert binding partner; and a protease cleavage site separating the first T-cell engaging domain and the first inert binding partner, wherein the protease cleavage site is capable of releasing the inert binding partner and half-life extending moiety from the T-cell engaging domain in the presence of a protease: (1) expressed by the cancer or in the cancer microenvironment; (2) colocalized to the cancer by a targeting moiety that binds a tumor antigen expressed by the cancer and that is the same or different from the targeting moiety in the agent, and a second component comprising a targeted T-cell engaging agent comprising: a second targeting moiety that binds a tumor antigen expressed by the cancer; a second T-cell engaging domain capable of T-cell binding activity when binding a first T-cell engaging domain, wherein the first T-cell engaging domain is not part of the second component, and wherein the second T-cell engaging domain comprises either a VH domain or VL domain; a second inert binding partner for the second T-cell engaging domain binding to the second T-cell engaging domain such that the second T-cell engaging domain does not bind to the first T-cell engaging domain unless the inert binding partner is removed, wherein if the second T-cell engaging domain comprises a VH domain, the inert binding partner comprises a VL domain and if the second T-cell engaging domain comprises a VL domain, the inert binding partner comprises a VH domain; and a second half-life extending moiety, wherein the second half-life extending moiety is attached (directly or indirectly) to the second inert binding partner; and a protease cleavage site separating the second T-cell engaging domain and the second inert binding partner, wherein the protease cleavage site is capable of releasing the inert binding partner and half-life extending moiety from the T-cell engaging domain in the presence of a protease: (1) expressed by the cancer or in the cancer microenvironment; (2) colocalized to the cancer by a targeting moiety that binds a tumor antigen expressed by the cancer and that is the same or different from the targeting moiety in the agent, wherein the first and second T-cell engaging domains are capable of binding a T cell when neither is bound to an inert binding partner, and further wherein if the first T-cell engaging domain comprises a VH domain, the second T-cell engaging domain comprises a VL domain and if the first T-cell engaging domain comprises a VL domain, the second T-cell engaging domain comprises a VH domain.

Item 2. An agent for treating cancer in a patient comprising: a first component comprising a targeted immune cell engaging agent comprising: a targeting moiety capable of targeting the cancer; a first immune cell engaging domain capable of immune engaging activity when binding a second immune cell engaging domain, wherein the second immune cell engaging domain is not part of the first component, optionally wherein the first immune cell engaging domain comprises a T-cell engaging domain; a first inert binding partner for the first immune cell engaging domain binding to the first immune cell engaging domain such that the first immune cell engaging domain does not bind to the second immune cell engaging domain unless the inert binding partner is removed, wherein if the first immune cell engaging domain comprises a VH domain, the inert binding partner comprises a VL domain and if the first immune cell engaging domain comprises a VL domain, the inert binding partner comprises a VH domain; a first half-life extending moiety, wherein the first half-life extending moiety is attached (directly or indirectly) to the first inert binding partner; and a protease cleavage site separating the first immune cell engaging domain and the first inert binding partner, wherein the protease cleavage site is capable of releasing the inert binding partner and half-life extending moiety from the immune cell engaging domain in the presence of a protease: (1) expressed by the cancer or in the cancer microenvironment; or (2) colocalized to the cancer by a targeting moiety that binds a tumor antigen expressed by the cancer and that is the same or different from the targeting moiety in the agent, and a second component comprising a selective immune cell engaging agent comprising: an immune cell selection moiety capable of selectively targeting an immune cell; a second immune cell engaging domain capable of immune cell engaging activity when binding the first immune cell engaging domain, wherein the first and second immune cell engaging domains are capable of binding when neither is bound to an inert binding partner, optionally wherein the second immune cell engaging domain comprises a immune cell engaging domain; a second inert binding partner for the second immune cell engaging domain binding to the second immune cell engaging domain such that the second immune cell engaging domain does not bind to the first immune cell engaging domain unless the inert binding partner is removed, wherein if the second immune cell engaging domain comprises a VH domain, the inert binding partner comprises a VL domain and if the second immune cell engaging domain comprises a VL domain, the inert binding partner comprises a VH domain; and a second half-life extending moiety, wherein the second half-life extending moiety is attached (directly or indirectly) to the second inert binding partner; a protease cleavage site separating the second immune cell engaging domain and the second inert binding partner, wherein the protease cleavage site is capable of releasing the inert binding partner and half-life extending moiety from the immune cell engaging domain in the presence of a protease: (1) expressed by the cancer or in the cancer microenvironment; or (2) colocalized to the cancer by a targeting moiety that binds a tumor antigen expressed by the cancer and that is the same or different from the targeting moiety in the agent, wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

Item 3. A component for use in a kit or composition for treating cancer in a patient comprising a first targeted immune cell engaging agent comprising: a targeting moiety that binds a tumor antigen expressed by the cancer; an immune cell engaging domain capable of immune cell binding activity when binding another immune cell engaging domain, wherein the other immune cell engaging domain is not part of the first component, and wherein the immune cell engaging domain comprises either a VH domain or VL domain, optionally wherein the immune cell engaging domain comprises a T-cell engaging domain; an inert binding partner for the immune cell engaging domain binding to the immune cell engaging domain such that the immune cell engaging domain does not bind to the other immune cell engaging domain unless the inert binding partner is removed, wherein if the immune cell engaging domain comprises a VH domain, the inert binding partner comprises a VL domain and if the immune cell engaging domain comprises a VL domain, the inert binding partner comprises a VH domain; a half-life extending moiety, wherein the half-life extending moiety is attached (directly or indirectly) to the inert binding partner; and a protease cleavage site separating the immune cell engaging domain and the inert binding partner, wherein the protease cleavage site is capable of releasing the inert binding partner and half-life extending moiety from the immune cell engaging domain in the presence of a protease: (1) expressed by the cancer; or (2) colocalized to the cancer by a targeting moiety that binds a tumor antigen expressed by the cancer and that is the same or different from the targeting moiety in the agent, wherein cleavage of the protease cleavage site causes loss of the inert binding partner and allows for complementation with the other immune cell engaging domain that is not part of the agent, further wherein if the immune cell engaging domain comprises a VH domain, the other immune cell engaging domain comprises a VL domain and if the immune cell engaging domain comprises a VL domain, the other immune cell engaging domain comprises a VH domain.

Item 4. A component for use in a kit or composition for treating cancer in a patient comprising a first targeted immune cell engaging agent comprising: an immune cell selection moiety capable of selectively targeting an immune cell; an immune cell engaging domain capable of immune cell binding activity when binding another immune cell engaging domain, wherein the other immune cell engaging domain is not part of the first component, and wherein the immune cell engaging domain comprises either a VH domain or VL domain, optionally wherein the immune cell engaging domain comprises a T-cell engaging domain; an inert binding partner for the immune cell engaging domain binding to the immune cell engaging domain such that the immune cell engaging domain does not bind to the other immune cell engaging domain unless the inert binding partner is removed, wherein if the immune cell engaging domain comprises a VH domain, the inert binding partner comprises a VL domain and if the immune cell engaging domain comprises a VL domain, the inert binding partner comprises a VH domain; a half-life extending moiety, wherein the half-life extending moiety is attached (directly or indirectly) to the inert binding partner; and a protease cleavage site separating the immune cell engaging domain and the inert binding partner, wherein the protease cleavage site is capable of releasing the inert binding partner and half-life extending moiety from the immune cell engaging domain in the presence of a protease: (1) expressed by the cancer; or (2) colocalized to the cancer by a targeting moiety that binds a tumor antigen expressed by the cancer and that is the same or different from the targeting moiety in the agent, wherein cleavage of the protease cleavage site causes loss of the inert binding partner and allows for complementation with the other immune cell engaging domain that is not part of the agent, further wherein if the immune cell engaging domain comprises a VH domain, the other immune cell engaging domain comprises a VL domain and if the immune cell engaging domain comprises a VL domain, the other immune cell engaging domain comprises a VH domain.

Item 5. An agent for treating cancer in a patient comprising: a first targeting moiety that binds a tumor antigen expressed by the cancer; a first T-cell engaging domain capable of T-cell binding activity when binding a second T-cell engaging domain, wherein the first T-cell engaging domain comprises either a VH domain or VL domain; a second T-cell engaging domain capable of T-cell binding activity when binding a first T-cell engaging domain, wherein the second T-cell engaging domain comprises either a VH domain or VL domain; a first inert binding partner for the first T-cell engaging domain binding to the first T-cell engaging domain such that the first T-cell engaging domain does not bind to the second T-cell engaging domain unless the inert binding partner is removed, wherein if the first T-cell engaging domain comprises a VH domain, the inert binding partner comprises a VL domain and if the first T-cell engaging domain comprises a VL domain, the inert binding partner comprises a VH domain; and a protease cleavage site separating the first T-cell engaging domain and the first inert binding partner, wherein the protease cleavage site is capable of releasing the inert binding partner from the T-cell engaging domain in the presence of a protease: (1) expressed by the cancer or in the cancer microenvironment; or (2) colocalized to the cancer by a targeting moiety that binds a tumor antigen expressed by the cancer and that is the same or different from the targeting moiety in the agent; wherein the first and second T-cell engaging domains are capable of binding a T cell when neither is bound to an inert binding partner, and further wherein if the first T-cell engaging domain comprises a VH domain, the second T-cell engaging domain comprises a VL domain and if the first T-cell engaging domain comprises a VL domain, the second T-cell engaging domain comprises a VH domain.

Item 6. An agent for treating cancer in a patient comprising: an immune cell selection moiety capable of selectively targeting an immune cell; a first immune cell engaging domain capable of immune cell binding activity when binding a second immune cell engaging domain, wherein the first immune cell engaging domain comprises either a VH domain or VL domain, optionally wherein the first immune cell engaging domain comprises a T-cell engaging domain; a second immune cell engaging domain capable of immune cell binding activity when binding a first immune cell engaging domain, wherein the second immune cell engaging domain comprises either a VH domain or VL domain, optionally wherein the second immune cell engaging domain comprises a T-cell engaging domain; a first inert binding partner for the first immune cell engaging domain binding to the first immune cell engaging domain such that the first immune cell engaging domain does not bind to the second immune cell engaging domain unless the inert binding partner is removed, wherein if the first immune cell engaging domain comprises a VH domain, the inert binding partner comprises a VL domain and if the first immune cell engaging domain comprises a VL domain, the inert binding partner comprises a VH domain; and a protease cleavage site separating the first immune cell engaging domain and the first inert binding partner, wherein the protease cleavage site is capable of releasing the inert binding partner from the immune cell engaging domain in the presence of a protease: (1) expressed by the cancer or in the cancer microenvironment; or (2) colocalized to the cancer by a targeting moiety that binds a tumor antigen expressed by the cancer and that is the same or different from the targeting moiety in the agent; wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

Item 7. The agent of any one of items 5 or 6, wherein the agent further comprises a second targeting moiety that is capable of targeting the cancer.

Item 8. The agent of any one of items 5-7 further comprising: a second inert binding partner for the second immune cell engaging domain binding to the second immune cell engaging domain such that the second immune cell engaging domain does not bind to the first immune cell engaging domain unless the inert binding partner is removed, wherein if the second immune cell or engaging domain comprises a VH domain, the inert binding partner comprises a VL domain and if the second immune cell engaging domain comprises a VL domain, the inert binding partner comprises a VH domain, optionally wherein the second immune cell engaging domain comprises a T-cell engaging domain; and a protease cleavage site separating the second immune cell engaging domain and the second inert binding partner, wherein the protease cleavage site is capable of releasing the inert binding partner from the immune cell engaging domain in the presence of a protease: (1) expressed by the cancer or in the cancer microenvironment; or (2) colocalized to the cancer by a targeting moiety that binds a tumor antigen expressed by the cancer and that is the same or different from the targeting moiety in the agent, wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

Item 9. The agent of item 8, wherein a linker attaches the first and second inert binding partners.

Item 10. The agent of item 9, wherein the linker comprises a half-life extending moiety.

Item 11. The agent of any one of items 9-10, wherein the linker is capable of dissociation with the first and/or second inert binding partner upon cleavage of the protease cleavage sites.

Item 12. The agent of any one of items 1-11, wherein the first and/or second half-life extending moiety is directly attached to the first and/or second inert binding partner.

Item 13. The agent of any one of items 1-11, wherein the first and/or second half-life extending moiety is indirectly attached to the first and/or second inert binding partner via a linker.

Item 14. The agent of any one of items 1-2, wherein the first component comprises: two copies of a first targeting moiety; two copies of a first immune or T-cell engaging domain; and two copies of a first inert binding partner, wherein a protease cleavage site separates both inert binding partners from their respective immune or T-cell engaging domains.

Item 15. The agent of item 14, wherein one end of the half-life extending moiety is attached (directly or indirectly) to one copy of the first inert binding partner and the other end of the half-life extending moiety is attached (directly or indirectly) to the other copy of the first inert binding partner.

Item 16. The agent of any one of items 1-2, 14, or 15, wherein the second component comprises: two copies of a second targeting moiety; two copies of a second immune or T-cell engaging domain; and two copies of a second inert binding partner, wherein a protease cleavage sites separates both inert binding partners from their respective immune or T-cell engaging domains.

Item 17. The agent of item 16, wherein one end of the half-life extending moiety is attached (directly or indirectly) to one copy of the second inert binding partner and the other end of the half-life extending moiety is attached (directly or indirectly) to the other copy of the second inert binding partner.

Item 18. The agent of any one of items 14-17, wherein the two copies of the targeting moiety are the same.

Item 19. The agent of any one of items 14-18, wherein the two copies of the immune or T-cell engaging domain are the same.

Item 20. The agent of any one of items 14-19, wherein the two copies of the inert binding partner are the same.

Item 21. The agent of any one of items 14-20, wherein the two copies of the protease cleavage site separating the inert binding partners from their respective immune or T-cell engaging domains are the same.

Item 22. The agent of any one of items 14-20, wherein the two copies of a protease cleavage site separating the inert binding partners from their respective immune or T-cell engaging domains are different.

Item 23. The agent or component of any one of items 1-22, wherein the half-life is decreased after dissociation of one or more half-life extending moieties.

Item 24. The agent of any one of items 1-2 or 12-23, wherein the half-life of the first and/or second component is longer than the half-life of a complex formed by the association of the first and second immune cell or T-cell engaging domains in the form capable of binding to an immune or T cell.

Item 25. The agent of any one of items 1-2 or 12-24, wherein the first component and/or second component has a half-life greater or equal to 2 days, 4 days, or 7 days.

Item 26. The agent or component of any one of items 3-11, wherein the agent or component has a half-life greater or equal to 2 days, 4 days, or 7 days.

Item 27. The agent of any one of items 1, 2, 8-11 or 14-26, wherein the protease cleavage sites are different.

Item 28. The agent of any one of items 8-11 or 14-26, wherein the protease cleavage sites are the same.

Item 29. The agent or component of any one of items 1-28, wherein one or more protease cleavage sites are cleaved by a protease expressed by the cancer.

Item 30. The agent or component of any one of items 1-29, wherein one or more protease cleavage sites are cleaved by a protease that is colocalized to the cancer by a targeting moiety that binds a tumor antigen expressed by the cancer and that is the same or different from the targeting moiety in the agent.

Item 31. The agent or component of any one of items 1-30, wherein one or more first and second inert binding partners are capable of dissociation once at least one protease cleavage site for each inert binding partner has been cleaved and after dissociation the two immune cell or T-cell engaging domains that had been bound by the inert binding partners are capable of binding to each other and exhibiting immune cell or T-cell binding activity.

Item 32. The agent or component of any one of items 1-31, wherein one or more half-life extending moieties are capable of dissociation together with one or more inert binding partner to which it is attached.

Item 33. The agent or component of any one of items 1-4 or 10-32, wherein the one or more half-life extending moieties comprise all or part of an immunoglobulin constant (Fc) domain, serum albumin, serum albumin binding protein, an unstructured protein, and/or PEG.

Item 34. The agent or component of item 33, wherein the one or more half-life extending moieties comprise all or part of an immunoglobulin Fc domain.

Item 35. The agent or component of item 34, wherein the Fc domain comprises the sequence of a human immunoglobulin.

Item 36. The agent or component of item 34, wherein the immunoglobulin is IgG.

Item 37. The agent or component of item 36, wherein the IgG is IgG1, IgG2, or IgG4.

Item 38. The agent or component of item 34, wherein the Fc domain comprises a naturally occurring sequence.

Item 39. The agent or component of item 34, wherein the Fc domain comprises one or more mutations as compared to a naturally occurring sequence.

Item 40. The agent or component of item 39, wherein the Fc domain is an Fc domain with a longer half-life compared to a naturally occurring sequence.

Item 41. The agent or component of item 40, wherein the Fc domain with a longer half-life has increased FcRn binding.

Item 42. The agent or component of item 41, wherein the increased FcRn binding is measured at pH 6.0.

Item 43. The agent or component of item 40, wherein the Fc domain with a longer half-life comprises M252Y/S254T/T256E substitutions.

Item 44. The agent or component of item 40, wherein the Fc domain with a longer half-life comprises M428L/N434S substitutions.

Item 45. The agent or component of item 33, wherein one or more half-life extending moiety comprise all or part of serum albumin.

Item 46. The agent or component of item 45, wherein the serum albumin is human.

Item 47. The agent or component of item 33, wherein one or more half-life extending moiety comprise all or part of a serum albumin binding protein.

Item 48. The agent or component of item 47, wherein the serum albumin binding protein is a DARPin, a nanobody, a single-chain variable fragment (scFv), or an antigen-binding fragment (Fab).

Item 49. The agent or component of item 33, wherein the serum albumin binding protein comprises all or part of an albumin binding domain.

Item 50. The agent or component of item 33, wherein one or more half-life extending moiety comprise all or part of an unstructured protein.

Item 51. The agent or component of item 50, wherein the unstructured protein is an unstructured hydrophilic, biodegradable protein polymer.

Item 52. The agent or component of item 51, wherein the unstructured protein is XTEN.

Item 53. The agent or component of item 33, wherein one or more half-life extending moiety comprise all or part of PEG.

Item 54. The agent of any one of items 1-2 and 12-53, wherein the first and second half-life extending moieties are different.

Item 55. The agent of any one of items 1-2 and 12-53, wherein the first and second half-life extending moieties are the same.

Item 56. The agent of any one of items 1-2 and 12-55, wherein the first component is not covalently bound to the second component.

Item 57. The agent of any one of items 1-2 and 12-55, wherein the first component is covalently bound to the second component.

Item 58. The agent of item 57, wherein the first component is covalently bound to the second component by a linker comprising a protease cleavage site.

Item 59. The agent or component of item 2, 4, or 6-58, wherein the immune cell selection moiety capable of selectively targeting an immune cell selectively targets a T cell, a macrophage, a natural killer cell, a neutrophil, an eosinophil, a basophil, a γδ T cell, a natural killer T cell (NKT cells), or an engineered immune cell.

Item 60. The agent or component of item 59, wherein the immune cell selection moiety capable of selectively targeting an immune cell selectively targets a T cell, optionally where the T cell is a CD8+ or CD4+ T cell.

Item 61. The agent or component of item 59, wherein the immune cell selection moiety targets CD8, CD4, or CXCR3, or does not specifically bind regulatory T cells.

Item 62. The agent or component of any one of items 59-61, wherein the immune cell selection moiety comprises an aptamer or an antibody or antigen-specific binding fragment thereof.

Item 63. The agent or component of item 62, wherein the aptamer or antibody or antigen-specific binding fragment thereof specifically binds an antigen on a T cell.

Item 64. The agent or component of any one of items 1-2 or 5-63, wherein the first and second T-cell or immune cell engaging domains are capable of binding CD3 or the T cell receptor (TCR) when neither is bound to an inert binding partner.

Item 65. The agent or component of any one of items 1-2 or 5-64, wherein the first and second T-cell or immune cell engaging domains are capable of forming a Fv when not bound to an inert binding partner.

Item 66. The agent or component of any one of items 1-5 or 7-65, wherein one or more targeting moieties are an antibody or antigen-binding fragment thereof.

Item 67. The agent or component of item 66, wherein the antibody or antigen-binding fragment thereof is (i) specific for any of 4-1BB, 5T4, ACVRL1, ALK1, AXL, B7-H3, BCMA, c-MET, CD133, C4.4a, CA6, CA9, Cadherin-6, CD123, CD133, CD138, CD19, CD20, CD22, CD25, CD27L, CD30, CD33, CD37, CD38, CD44v6, CD56, CD70, CD74(TROP2), CD79b, CEA, CEACAM5, cKit, CLL-1, Cripto, CS1, DLL3, EDNRB, EFNA4, EGFR, EGFRvIII, ENPP3, EpCAM, EPHA2, FGFR2, FGFR3, FLT3, FOLR, FOLR1, GD2, gpA33, GPC3, GPNMB, GUCY2C, HER2, HER3, HLAA2, IGF1-r, IL13RA2, Integrin alpha, LAMP-1, LewisY, LIV-1, LRRC15, MMP9, MSLN, MUC1, MUC16, NaPi2b, Nectin-4, NOTCH3, p-CAD, PD-L1, PSMA, PTK7, ROR1, SLC44A4, SLITRK6, SSTR2, STEAP1, TAG72, TF, TIM-1, or TROP-2, or (ii) an anti-epidermal growth factor receptor antibody; an anti-Her2 antibody; an anti-CD20 antibody; an anti-CD22 antibody; an anti-CD70 antibody; an anti-CD33 antibody; an anti-MUC1 antibody; an anti-CD40 antibody; an anti-CD74 antibody; an anti-P-cadherin antibody; an anti-EpCAM antibody; an anti-CD138 antibody; an anti-E-cadherin antibody; an anti-CEA antibody; an anti-FGFR3 antibody; an anti-mucin core protein antibody; an anti-transferrin antibody; an anti-gp95/97 antibody; an anti-p-glycoprotein antibody; an anti-TRAIL-R1 antibody; an anti-DR5 antibody; an anti-IL-4 antibody; an anti-IL-6 antibody; an anti-CD19 antibody; an anti-PSMA antibody; an anti-PSCA antibody; an anti-Cripto antibody; an anti-PD-L1 antibody; an anti-IGF-1R antibody; an anti-CD38 antibody; an anti-CD133 antibody; an anti-CD123 antibody; an anti-CDE49d antibody; an anti-glypican 3 antibody; an anti-cMET antibody; or an anti-IL-13R antibody.

Item 68. The agent or component of item 66, wherein the antibody or antigen-binding fragment comprises all or part of the amino acid sequence of 1C1, (GS) 5745, ABBV-085, ABBV-399, ABBV-838, AbGn-107, ABT-414, ADCT-301, ADCT-402, AGS-16C3F, AGS62P1, AGS67E, AMG 172d, AMG 595d, Andecaliximab, Anetumab ravtansine, ARX788, ASG-15MEd, ASG-5MEk, Atezolizumab, AVE1642, AVE9633e, Avelumab, BAY1129980, BAY1187982e, BAY79-4620b, BIIB015d, Bivatuzumab mertansineb, BMS-986148, Brentuximab vedotin, Cantuzumab mertansine, CC49, CDX-014, Cirmtuzumab, Coltuximab ravtansine, DEDN6526Ae, Denintuzumab mafodotin, Depatuxizumab, DFRF4539Ad, DMOT4039Ae, DS-8201A, Durvalumab, Enfortumab vedotin, Farletuzumab, FLYSYN, Gatipotuzumab, Gemtuzumab ozogamicin, Glembatumumab vedotin, GSK2857916, HKT288, Hu3F8, HuMax-AXL-ADC, IDEC-159, IMGN289b, IMGN388a, IMGN529, Indatuximab ravtansine, Inotuzumab ozogamicin, Istiratumab, Labetuzumab govitecan, Lifastuzumab vedotin, LOP628h, Lorvotuzumab mertansine, LY3076226, MCLA-117 (CLEC-12AxCD3), MDX-1203d, MEDI-4276, MEDI-547b, Milatuzumab-doxorubicin, Mirvetuximab soravtansine, MLN0264, MLN2704e, MM-302i, Mosunetuzumab, MOv18 IgE, Ocrelizumab, Oportuzumab, Patritumab, PCA-062, PF-03446962, PF-06263507a, PF-06647020, PF-06647263, PF-06650808d, Pinatuzumab vedotin, Polatuzumab vedotin, PSMA ADC 301c, RC48-ADC, Rituximab, Rovalpituzumab tesirine, Sacituzumab, Sacituzumab govitecan, SAR408701, SAR428926, SAR566658, SC-002, SC-003, SGN-15a, SGN-CD123A, SGN-CD19B, SGN-CD70A, SGN-LIV1A, Sofituzumab vedotin, Solitomab, SSTR2xCD3 XmAb18087, STRO-002, SYD-985, Talacotuzumab, Tisotumab vedotin, Trastuzumab emtansine, U3-1402, Ublituximab, Vadastuximab talirine, Vandortuzumab vedotin, Vorsetuzumab mafodotin, XMT-1522, or Zenocutuzumab.

Item 69. The agent or component of any one of items 1-68, wherein one or more targeting moieties are an aptamer.

Item 70. The agent or component of item 69, wherein the aptamer comprises DNA.

Item 71. The agent or component of item 69, wherein the aptamer comprises RNA.

Item 72. The agent or component of any one of items 69-71, wherein the aptamer is single-stranded.

Item 73. The agent or component of any one of items 69-72, wherein the aptamer is a target cell-specific aptamer chosen from a random candidate library.

Item 74. The agent or component of any one of items 69-73, wherein the aptamer is an anti-EGFR aptamer.

Item 75. The agent or component of any one of items 69-74, wherein the aptamer binds to the antigen on the cancer cell with a Kd from 1 picomolar to 500 nanomolar.

Item 76. The agent or component of item 75, wherein the aptamer binds to the cancer with a Kd from 1 picomolar to 100 nanomolar.

Item 77. The agent or component of any one of items 1-5 or 7-76, wherein one or more targeting moiety comprise IL-2, IL-4, IL-6, α-MSH, transferrin, folic acid, EGF, TGF, PD1, IL-13, stem cell factor, insulin-like growth factor (IGF), or CD40.

Item 78. The agent or component of any one of items 1-5 or 7-77, wherein one or more targeting moiety comprise a full-length sequence of IL-2, IL-4, IL-6, α-MSH, transferrin, folic acid, EGF, TGF, PD1, IL-13, stem cell factor, insulin-like growth factor (IGF), or CD40.

Item 79. The agent or component of any one of items 1-5 or 7-77, wherein one or more targeting moiety comprise a truncated form, analog, variant, or derivative of IL-2, IL-4, IL-6, α-MSH, transferrin, folic acid, EGF, TGF, PD1, IL-13, stem cell factor, insulin-like growth factor (IGF), or CD40.

Item 80. The agent or component of any one of items 1-5 or 7-79, wherein one or more targeting moiety bind a target on the cancer comprising IL-2 receptor, IL-4, IL-6, melanocyte stimulating hormone receptor (MSH receptor), transferrin receptor (TR), folate receptor 1 (FOLR), folate hydroxylase (FOLH1), EGF receptor, PD-L1, PD-L2, IL-13R, CXCR4, IGFR, or CD40L.

Item 81. The agent of any one of items 1, 7-58, or 64-80, wherein the first and second targeting moieties bind the same antigen.

Item 82. The agent of item 81, wherein the first and second targeting moieties bind the same epitope.

Item 83. The agent of item 82, wherein the first and second targeting moieties are the same.

Item 84. The agent of any one of items 1, 7-58, or 64-80, wherein the first and second targeting moieties are different.

Item 85. The agent of item 84, wherein the first and second targeting moieties bind different antigens.

Item 86. The agent of item 84, wherein the first and second targeting moieties bind different epitopes of the same antigen.

Item 87. A method of treating cancer expressing a tumor antigen that binds the first targeting moiety in a patient comprising administering the agent or component of any one of items 1-86 to the patient.

Item 88. The method of item 87, wherein the cancer expressing a tumor antigen that binds the first targeting moiety is any one of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, leukemia, myeloma, nonHodgkin lymphoma, Hodgkin lymphoma, acute myeloid leukemia, acute lymphoblastic leukemia, chronic lymphoblastic leukemia, lymphoproliferative disorder, myelodysplastic disorder, myeloproliferative disease or premalignant disease.

Item 89. A method of targeting an immune response of a patient to cancer comprising administering the agent or component of any one of items 87-88 to the patient.

Item 90. The method of any one of items 87-89, wherein the T cells express CD3 or TCR and the T cell engaging domain binds CD3 or TCR.

Item 91. The method of any one of items 2, 4, or 6-80, wherein if the patient has regulatory T cells in the tumor, the selective immune cell engaging agent does not target markers present on regulatory immune cells (including, but not limited to CD4 and CD25).

Item 92. A method of treating cancer expressing a tumor antigen in a patient comprising administering a composition comprising the component of any one of items 3, 4, 23, 26, 29-53, or 59-80, wherein the first targeting moiety binds the tumor antigen and a second component comprising a half-life extending moiety.

Item 93. A method of treating cancer expressing a tumor antigen in a patient comprising administering a composition comprising the component of any one of items 3, 4, 23, 26, 29-53, or 59-80, wherein the first targeting moiety binds the tumor antigen and a second component not comprising a half-life extending moiety.

Item 94. One or more nucleic acid molecules encoding the agent or component of any of items 1-80.

Item 95. An agent for treating cancer in a patient comprising: a first component comprising: a targeting antibody or antigen-specific binding fragment thereof that binds a tumor antigen expressed by the cancer; a first VH or VL domain capable of T-cell engaging activity when binding a second VH or VL domain, wherein the second VH or VL domain is not part of the first component; a first inert binding partner for the first VH or VL domain such that the first VH or VL domain does not bind to the second VH or VL domain unless the inert binding partner is removed, wherein a first VH domain can bind an inert binding partner comprising a VL domain and a first VL domain can bind an inert binding partner comprising a VH domain; a first half-life extending moiety, wherein the first half-life extending moiety is attached (directly or indirectly) to the first inert binding partner; and a protease cleavage site separating the first VH or VL domain and the first inert binding partner, wherein the protease cleavage site is capable of releasing the inert binding partner and half-life extending moiety from the rest of the agent in the presence of a protease: (1) expressed by the cancer or in the cancer microenvironment; (2) colocalized to the cancer by a targeting antibody or antigen-specific binding fragment thereof that binds a tumor antigen expressed by the cancer and that is the same or different from the targeting antibody or antigen-specific binding fragment thereof in the agent, and a second component comprising: a second targeting antibody or antigen-specific binding fragment thereof that binds a tumor antigen expressed by the cancer; a second VH or VL domain capable of T-cell binding activity when binding a first VH or VL domain, wherein the first VH or VL domain is not part of the second component; a second inert binding partner for the second VH or VL domain binding such that the second VH or VL domain does not bind to the first VH or VL domain unless the inert binding partner is removed, wherein if the second VH or VL domain comprises a VH domain, the inert binding partner comprises a VL domain and if the second VH or VL domain comprises a VL domain, the inert binding partner comprises a VH domain; and a second half-life extending moiety, wherein the second half-life extending moiety is attached (directly or indirectly) to the second inert binding partner; a protease cleavage site separating the second VH or VL domain and the second inert binding partner, wherein the protease cleavage site is capable of releasing the inert binding partner and half-life extending moiety from the rest of the agent in the presence of a protease: (1) expressed by the cancer or in the cancer microenvironment; (2) colocalized to the cancer by a targeting antibody or antigen-specific binding fragment thereof that binds a tumor antigen expressed by the cancer and that is the same or different from the targeting antibody or antigen-specific binding fragment thereof in the agent, wherein the first and second VH or VL domains are capable of binding a T cell when neither is bound to an inert binding partner, and further wherein if the first VH or VL domain comprises a VH domain, the second VH or VL domain comprises a VL domain and if the first VH or VL domain comprises a VL domain, the second VH or VL comprises a VH domain.

### EXAMPLES

### Example 1. Anti-CD33/Anti-CD123 TEACs Comprising Half-Life Extending Moieties

Two-component dual IgG TEACs were developed wherein each component of the two-component system comprises an IgG TEAC. The dual IgG TEAC was designed comprising a first component that was an IgG TEAC comprising two targeting moieties that are each an anti-CD33 antibody and a second component that was an IgG TEAC comprising two targeting moieties that are each an anti-CD123 antibody. Each IgG TEAC also comprises two copies of T-cell engaging domain, two copies of an inert binding partners, and two copies of a cleavage site between the T-cell engaging domains and the inert binding partners.

Further, both the first and second components together also comprise a linker comprising a half-life extending moiety. The half-life extending moiety comprises an Fc domain from IgG4, wherein the Fc domain is directly linked to the inert binding partner. When expressed in HEK293T cells, one copy of the TEAC (CD33 binding region (scFv)-anti-CD3 domain-cleavage sequence-inert binding partner-Fc domain) will recombine with a second copy of the TEAC via covalent interactions of the Fc domains to form the IgG TEAC.

As an example, a TEAC was composed of VH-VL specific for a tumor antigen (either CD33 or CD123) linked to a second scFv composed of VL-VH with the VL specific for CD3 and the VH being the inert binding partner or with the VH specific for CD3 and VL being the inert binding partner (SEQ ID NO: 199 for anti-CD33 TEAC component and SEQ ID NO: 200 for anti-CD123 TEAC component). For the IgG TEACs, conventional TEAC components were used as the base and the CH domains from IgG4 were added to allow pairing. This made the resulting IgG TEAC into a more conventional antibody shape with two TEAC acting as the “arms,” and the CH regions acting as the conventional CH regions of an antibody.

When the TEACs were produced in the IgG format in 293T cells, the DNA plasmid that was transfected into the cells contained one single sequence that was produced multiple times, and the proteins paired to form a single antibody-like protein, the IgG TEAC. Each IgG TEAC was generated in this manner separately in a different population of cells, and then the CD33 and CD123 IgG TEACs could be used together as a pair of two different IgG TEACs, i.e., a Dual IgG TEAC.

A single-agent TEAC (Duo TEAC) comprising a half-life extending moiety was also designed that comprises SEQ ID NOs: 199 and 200, such that it comprises one targeting moiety which is an anti-CD33 antibody and one targeting moiety that is an anti-CD123 antibody.

The first and second inert binding partners of the single-agent TEAC were connected via a linker comprising a half-life extending moiety. The half-life extending moiety comprises an Fc domain from IgG4 wherein the inert binding partner is directly linked to the Fc domain.

For the Duo-TEAC, two different sequences were needed that coded for each “arm” or half of the antibody-like protein. Therefore, the construct comprised a CH domain, followed by the inert binding domain, the anti-CD3 domain, and then the tumor binding domain (e.g., CD33). In the same DNA construct, a second sequence was added for a construct that comprised a CH domain followed by the inert binding partner, the complementary anti-CD3 domain, and then the second tumor binding domain (e.g., CD123).

When expressed in HEK293T cells, one chain of the protein (CD33 binding region (scFv)-anti-CD3 domain-cleavage sequence-inert binding partner-Fc domain) will recombine with a second chain of the protein (containing the CD123 binding domain) via covalent interactions of the CH domains to form the Duo IgG TEAC. Once this DNA construct was transfected into the 293T cells, both proteins were produced by the cells and there would be three possible protein pairs (i) CD33 and CD33; (ii) CD123 and CD123; (iii) CD33 and CD123.

In order to ensure that only the Duo TEAC was purified, one of the chains had a His-tag and the other chain had an EPEA tag. The CD33 dimer and CD33 protein itself would only express the His tag, the CD123 dimer and the CD123 protein itself would only express the EPEA tag when using SEQ ID NOs: 199 and 200. In contrast, the properly assembled Duo-TEAC would express one His tag and one EPEA tag. Therefore, the protein mixture was purified first through a His column and the eluate was then purified through an EPEA column. Thus, the purified protein would only contain the CD33/CD123 Duo-TEAC and not the CD33/CD33 or CD123/CD123 TEACs.

Next, the CD33/CD123 TEACs were evaluated in an in vitro model. An AML tumor cell line, which had previously been confirmed to be CD33+ and CD123+ by flow cytometry, was washed in serum-free media and re-suspended to 10⁵/ml in serum free media. The Dual IgG TEAC or Duo-TEAC was added at varying concentrations between 1-1000 nM and incubated at room temperature for 30 minutes. Excess, unbound TEAC was removed by washing in serum free media, and labelled tumor cells were re-suspended to 10⁶/ml. 100 μl tumor cells were added to triplicate wells of 96 well U-bottom plate. CD3+ T cells were washed twice in serum-free media and re-suspended to 2.5×10⁵/ml. 100 μl T cells were added to each well and the co-culture incubated overnight at 37° C. The following day, supernatant was assayed for the presence of IFN gamma by ELISA (ThermoFisher, USA), and the assay stopped by addition of 10% hydrochloric acid. The plate was read at absorbance of 450 nm in 96 well plate reader (Neo 2, Synergy, USA). Results shown in FIG. 2B show efficacy of both the dual Ig-TEAC and the Duo-TEAC.

A treatment plan could be designed for treating patients by infusing with an anti-CD33/anti-CD123 TEAC (either as a two-component composition or a single-agent). Patients with acute myeloid leukemia (AML) express both CD33 and CD123. Data suggest that an estimated 69.5% of AMLs had simultaneous presence of both antigens (See Ehninger et al., Blood Cancer Journal 4:e218 (2014)). The presence of the half-life extending moiety would be expected to increase the half-life of the molecule in patients to days instead of hours and therefore may allow less-frequent dosing compared to a TEAC without a half-life extension moiety.

### Example 2. Anti-EpCAM TEACs Comprising Half-Life Extending Moieties

A variety of TEACs with half-life extending moieties can be designed with EpCAM targeting moieties.

A two-component Dual IgG TEAC was be designed comprising a first IgG TEAC comprising two targeting moieties that are each an anti-EpCAM antibody and a second IgG TEAC comprising two targeting moieties that are each an anti-EpCAM antibody. Each IgG TEAC also comprises two copies of T-cell engaging domain, two copies of an inert binding partners, and two copies of a protease cleavage site between each T-cell engaging domain and its inert binding partner.

Further, both the first and second components also comprise a linker comprising a half-life extending moiety (SEQ ID NOs: 201 and 202). The half-life extending moiety comprises an Fc domain, wherein one end of the linker is directly attached to the inert binding partner. When expressed in HEK293T cells, one TEAC (EpCAM binding region (scFv)-anti-CD3 domain-cleavage sequence-inert binding partner-Fc domain) will recombine with a second copy of the TEAC via covalent interactions and form the IgG TEAC.

A single-agent TEAC comprising a half-life extending moiety was also designed that comprises two anti-EpCAM targeting moieties, (SEQ ID NOs: 201 and 202).

The first and second inert binding partners of the single-agent TEAC are connected via a linker comprising a half-life extending moiety. The half-life extending moiety comprises an Fc domain, from IgG4 wherein the inert binding partner is directly linked to the Fc domain. When expressed in HEK293T cells, one TEAC (EpCAM binding region (scFv)-anti-CD3 VH domain-cleavage sequence-inert binding partner-Fc domain) will recombine with the second TEAC (containing the same EpCAM binding domain with the corresponding anti-CD3 VL) via covalent interactions and form the Duo IgG TEAC. Both protein chains contain different purification tags (EPEA and histidine) which allows purification of the Duo-TEAC which contains one arm with anti-EpCAMxanti-CD3-VH and the second arm with anti-EpCAMxanti-CD3-VL. The CD3-VH/CD3-VH or CD3-VL/CD3-VL will not be purified.

Constructs were generated and purified in a similar way to the CD33 and CD123 IgG TEAC and CD33/CD123 Duo-TEAC in Example 1. For the IgG TEACs, the EpCAM scFv TEACs was used as a starting construct and the CH domains from an IgG4 antibody sequence were added to the C-terminal end of the inert binding partner. These TEACs were then produced in HEK 293T cells and purified from the supernatant.

For the EpCAM Duo-TEAC, two genes were added into a single plasmid with the scFv sequence of EpCAM TEAC with the IgG4 CH domains. Importantly, each TEAC gene sequence of the Duo-TEAC had a different tag (one had His tag and the other had EPEA tag) to allow purification. Once the proteins had been produced, constructs were run over a His column and then the eluate was run over an EPEA column so that the only protein purified would have both His and EPEA tags and therefore only Duo-TEAC would be used in the assay

A lung cancer tumor cell line (NCI-H2009), which had previously been confirmed to be EpCAM+ by flow cytometry, was washed in serum-free media and re-suspended to 10⁵/ml in serum free media. Anti-EpCAM IgG TEAC or Duo-TEAC was added at varying concentrations between 1-1000 nM and incubated at room temperature for 30 minutes. Excess, unbound TEAC was removed by washing in serum free media, and labelled tumor cells were re-suspended to 10⁶/ml. 100 μl tumor cells were added to triplicate wells of 96 well U-bottom plate. CD3+ T cells were washed twice in serum free media and re-suspended to 2.5×10⁵/ml. 100 μl T cells were added to each well, and the co-culture incubated overnight at 37° C. The following day, supernatant was assayed for the presence of IFN gamma by ELISA (ThermoFisher, USA) and the assay stopped by addition of 10% hydrochloric acid. The plate was read at absorbance of 450 nm in 96 well plate reader (Neo 2, Synergy, USA). Results shown in FIG. 3 show efficacy of both the Ig-TEAC and the Duo-TEAC.

A treatment plan could be designed for treating patients by infusing with an anti-EpCAM TEAC (either a two-component composition or a single-agent). Multiple types of solid tumor cancers express EpCAM, including colorectal, prostate, breast and ovarian cancers. In this way the TEACs with half-life extending moieties and EpCAM targeting moieties may have efficacy against a wide range of solid tumors. The presence of the half-life extending moiety would be expected to increase the half-life of the molecule in patients to days instead of hours and therefore may allow less-frequent dosing compared to a TEAC without a half-life extension moiety.

### Example 3. ATTACs Comprising Half-Life Extending Moieties

An anti-EpCAM ATTAC (containing the anti-CD3 VH domain) can be designed as an IgG TEAC/ATTAC comprising 2 copies of an anti-EpCAM scFv as targeting moieties, 2 copies of an anti-CD3e VH as immune cell engaging domains, 2 copies of an Ig VL domain as inert binding partners, and MMP2 cleavage sequences between each inert binding partner and immune cell engaging domains.

An anti-CD8 ATTAC (containing the anti-CD3e VL domain) can be designed as an IgG TEAC/ATTAC comprising 2 copies of an anti-CD8 VHH as targeting moieties, 2 copies of an anti-CD3e VL as immune cell engaging domains, 2 copies of an Ig VH domain as inert binding partners, and MMP2 cleavage sequences between each inert binding partner and immune cell engaging domains.

Both the first and second components may comprise a linker comprising a half-life extending moiety. The half-life extending moiety comprises an Fc domain, wherein one end of the linker is directly attached to one copy of the inert binding partner.

Further, a single-agent EpCAM/CD8 ATTAC can be designed with these same moieties. Constructs can be generated and purified in a similar way to the CD33 and CD123 IgG TEAC and CD33/CD123 Duo-TEAC in Example 1. An exemplary single-agent ATTAC could be generated via co-expression of an anti-EpCAM TEAC with a HIS tag (SEQ ID NO: 202) and an anti-CD8 VL ATTAC with an EPEA tag (SEQ ID NO: 212).

Conventional TEACs have been designed to target two different antigens on tumor cells where two antigens would better target tumor cells compared with healthy cells. In tumor cells where a single antigen could be used to target tumor cells, the second targeting moiety could be used to target specific subsets of T cells. In patients with prostate cancer the surface antigen PSMA is a good single target for tumor cells. A treatment plan could be designed for treating patients by infusing patients with EpCAM/CD8 ATTAC (either a two-component composition or a single-agent). In this way the ATTACs with half-life extending moieties may have efficacy against a wide range of solid tumors. The presence of the half-life extending moiety would be expected to increase the half-life of the molecule in patients to days instead of hours and therefore may allow less-frequent dosing compared to a TEAC without a half-life extension moiety.

### Example 4. Model of Changes in Half-Life with Half-Life Extension Moiety

Half-life extension (HLE) of TEAC or ATTAC molecules could be achieved by various strategies. One strategy is fusion of the TEAC or ATTAC to a protein with long serum half-life, such as the Fc of an antibody or serum albumin. Another strategy is fusion of the TEAC or ATTAC to a binding moiety that binds to a protein with long half-life such as serum albumin.

In principal, this half-life extension by ways of a fusion protein can be done in two opposing ways, which differ in how the TEAC or ATTAC and the half-life extending moiety are fused. First, the fusion protein can be engineered such that the TEAC or ATTAC has extended half-life irrespective of proteolytic activation. Second, the fusion protein can be engineered such that only the prodrug has long half-life, but proteolytic activation of the molecule leads to removal of the half-life extending moiety. The latter can be accomplished by fusing the half-life extending moiety to an inert binding partner of the TEAC or ATTAC.

Quantitative systems pharmacology modelling of TEAC or ATTAC activation has revealed that extending the half-life of the cleaved (active) TEAC or ATTAC could lead to a decreased therapeutic index. This is due to the fact that cleaved molecules could accumulate over time if the rate of cleavage is greater than the rate of clearance of these molecules, and molecules activated by cleavage in the tumor microenvironment could diffuse into circulation and cause off-tumor toxicities.

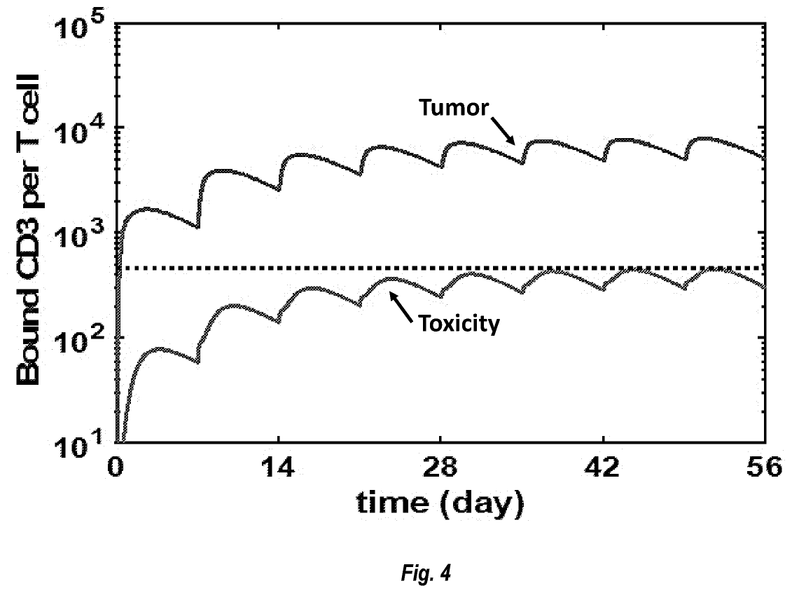

However, when the half-life extending moiety is fused to an inert binding partner and therefore removed during proteolytic activation of the TEAC or ATTAC, the active compounds have short serum half-live and are quickly eliminated. This prevents accumulation of activated molecules over time and limits unwanted activity of TEACs or ATTACs outside of the tumor microenvironment, where the activation occurs. This is shown in FIG. 4 comparing “tumor” levels versus “toxicity” (unwanted activity of TEACs or ATTACs outside the tumor microenvironment). Thus, in order to restrict activity to the tumor microenvironment and maintain a high therapeutic index, the TEACs or ATTACs with half-life extending moieties were designed such that the half-life extending moiety is fused to the inert binding partner and removed by proteolytic activation.

### Example 5. Expression and Proteolytic Cleavage of a TEAC Comprising an Effectorless Fc as a Half-Life Extension Moiety

A TEAC comprising a half-life extension moiety was engineered as an Fc fusion protein using the Fc domain of human IgG1. In this example, the sequence of human IgG1 was modified by mutation of Asparagine 297 to Glutamine (N297Q, EU numbering), which mutates the consensus sequence for an N-linked glycosylation at this site, thereby abrogating glycosylation and producing an aglycosylated Fc domain. Aglycosylated immunoglobulin Fc domains have reduced effector function (Wang X et al. Protein Cell 9(1):63-73 (2018)). The Fc-TEAC molecules were constructed such that the Fc domain is linked to the inert binding partner via a flexible linker, and the FC domain and the inert binding partner are released together following proteolytic activation (FIG. 5A). TEACs with Fc domains incorporated in this way confer stabilizing, half-life extending properties of the Fc-fusion to the intact TEAC but not to the proteolytically activated TEAC.

Fc-TEAC fusion proteins were constructed with various linkers between the Fc and the inert binding partner (Table 14). The proteins were expressed in transient HEK293 cultures (30-50 ml) in shake-flasks by co-transfection of the MP251, MP252, MP253, or MP254 constructs (SEQ ID NOs: 175-178, respectively) comprising heavy chains of the targeting moiety with the corresponding light chain construct (MP058, SEQ ID NO: 171) of the targeting moiety to generate TEACs. The expressed proteins were purified from supernatant by FPLC using protein A columns (Mab Select PrismA, GE). Analysis of the purified proteins by SDS-PAGE showed that homogenous products were produced of the expected molecular weight (200 kDa) with all the linker lengths tested in this experiment (FIG. 5B).

TABLE 14
Fc-TEAC linker designs

| | Heavy chain | | Protease |
|---|---|---|---|
| Protein | peptide | Fc linker | linker length |
| RO258 | MP251 | SG₃SG₄S | 19-mer |
| (SEQ ID | (SEQ ID | (SEQ ID | (SEQ ID |
| NO: 203) | NO: 175) | NO 185) | NO 181) |
| RO259 | MP252 | SG₃SG₄S | 19-mer |
| (SEQ ID | (SEQ ID | (SEQ ID | (SEQ ID |
| NO: 204) | NO: 176) | NO 185) | NO 181) |
| RO260 | MP253 | SG₃SG₄AG₄S | 11-mer |
| (SEQ ID | (SEQ ID | (SEQ ID | (SEQ ID |
| NO: 205) | NO: 177) | NO 184) | NO 182) |
| RO261 | MP254 | SG₃SG₄AG₄S | 9-mer |
| (SEQ ID | (SEQ ID | (SEQ ID | (SEQ ID |
| NO: 206) | NO: 178) | NO 184) | NO 183) |

For testing proteolytic cleavage of the Fc-TEAC proteins, the purified proteins were incubated with recombinant Factor Xa (New England Biolabs) at room temperature for 2 hours. The Fc-TEACs were engineered with an MMP2/9 cleavage sequence (SEQ ID NO: 49) in the protease linker, which also contains a cryptic FXa-cleavage site, and thus can be cleaved with recombinant FXa. The digested samples were analyzed by SDS-PAGE, which showed that cleavage produced the expected fragments from the TEACs corresponding to the molecular weight of partially and fully cleaved fragments (FIG. 5C). The efficiency of cleavage by FXa appeared higher in samples with longer protease linkers (RO258-260 comprising SEQ ID NOs: 181-182), and cleavage was not detected in samples with a 9-mer protease linker (RO261 comprising SEQ ID NO: 183).

### Example 6. Activity of a Half-Life Extended TEAC

In another example, a two-component TEAC system using the anti-EGFR antibody GA201 (Gerdes and Umana, Clin Cancer Res. 15; 20(4):1055 (2014)) as the targeting moiety was designed, wherein each TEAC component comprises a Fc domain as a half-life extension moiety (i.e. Fc-TEAC components). In this experiment, both components of the two-component TEAC system were constructed using identical Fc-linker and protease linker sequences, as well as the same anti-EGFR antibody GA201 for the targeting moiety.

Two TEAC components were designed, wherein each component comprised the heavy chain of the GA201 antibody. The difference between the two components is that one of components comprises the VH of the CD3 antibody and a corresponding VL domain as an inert binding partner. This component comprising the VH of the CD3 antibody is referred to here as “H-TEAC” or “VH TEAC.” The other component comprises the VL of the CD3 antibody and a corresponding VH domain as an inert binding partner. This component comprising the VL of the CD3 antibody is referred to as “L-TEAC” or “VL TEAC.” The sequence of the H-TEAC heavy chain is SEQ ID NO: 179 (MP268) and the sequence of the L-TEAC heavy chain is SEQ ID NO: 180 (MP269).

To produce the TEAC proteins, these TEACs components comprising the heavy chains of the targeting moiety anti-EGFR antibody GA201 antibody were co-expressed with the light chain MP113 (SEQ ID NO: 174) of GA201. The co-expression produced Fc-TEAC molecules recombinantly in HEK293 cells. The parental unstabilized TEAC molecules lacking the Fc were also produced by transient transfection in HEK293 cells by co-transfection of the heavy chains MP111 (SEQ ID NO: 172) or MP112 (SEQ ID NO: 173) with the light chain MP113 (SEQ ID NO: 174). These TEAC molecules contain a hexa-histidine tag at the C-terminus of the heavy chain to facilitate purification of the proteins by Ni-affinity chromatography. The combination of MP111 (SEQ ID NO: 172) and MP113 (SEQ ID NO: 174) generates construct RO130, while the combination of MP112 (SEQ ID NO: 173) and MP113 (SEQ ID NO: 174) generates construct RO131.

The ability of Fc-TEAC and parental TEAC to bind to EGFR was tested in an ELISA assay. Assay plates were coated with recombinant EGFR ectodomain (2 μg/ml, at 4° C. overnight) and incubated with TEACs at various dilutions. Bound TEAC protein was detected by an HRP-coupled anti-His antibody (Cell Signaling Catalog No. D3I1O).

Results of the EGFR binding ELISA are shown in FIG. 6. Analysis of the binding affinity demonstrates that the Fc-TEAC RO268 (formed by coexpression of MP268 (SEQ ID NO: 207) and MP113 (SEQ ID NO: 174)) and RO269 (formed by coexpression of MP269 (SEQ ID NO: 208) and MP113 (SEQ ID NO: 174)) bind 3-fold more tightly to EGFR than the parental TEAC constructs RO130 and RO131 or a corresponding CD3-EGFR bispecific molecule RO132 (SEQ ID NO: 209, K_{D} 0.24 nM versus 0.78-0.93 nM), which is a consequence of the avidity inherent to the bivalent Fc fusion proteins that is not observed with monovalent TEAC constructs. Thus, the bivalency (i.e., two targeting moieties in a single TEAC component) allows an Fc-TEAC to bind more tightly to tumor antigens.

The T-cell redirection activity of Fc-TEAC and TEAC constructs was tested in an in vitro T-cell activation assay. Breast cancer cells (MDA-MB-231) were seeded in 96-well plates at a density of 10000 cells/well and allowed to adhere overnight. Peripheral blood mononuclear cells (PBMCs) were added at an effector-to-target ratio of 10:1, and the cells were treated with serial dilutions of TEAC molecules. Secreted interferon gamma was detected in the media 24 hours after addition of TEAC using an IFNgamma ELISA kit (Invitrogen Catalog No. 88-7316-88), and target cell killing was determined 48 hours after the start of treatment using a cytotoxicity assay that quantitatively measures lactate dehydrogenase (LDH) (CytoTox96, Promega). Results of a T cell activation assay (FIG. 7A) and killing assay (FIG. 7B) demonstrated that Fc TEACs RO268+RO269 and the parental TEAC constructs RO130+RO131 induced T cell redirection against cancer cells with similar potency. The corresponding conventional CD3-EGFR bispecific molecule RO132 serves as positive control for T cell activation and cancer cell killing in this assay. These data confirm the activity of two-component kits wherein each component comprises an Fc domain.

### Example 7. In Vivo Half-Life Extension

The serum stability of TEAC and Fc-TEAC molecules was tested in BALB/c mice. Mice were dosed intravenously with 50 μg of Fc-TEAC (RO269) or the corresponding parental TEAC (RO131). Concentration of TEAC in mouse plasma was determined by ELISA using an anti-human Fab capture antibody (Jackson Laboratories #109-005-006) followed by detection with an anti-His secondary antibody coupled to HRP (Cell Signaling Catalog No. D3I1O). The resulting pharmacokinetic profile (FIG. 8A) showed significant extension of the serum half-life of the Fc-TEAC (T_{1/2}˜44 h) over the parental TEAC construct (T_{1/2}˜5). A similar experiment performed in BALB/c mice with TEAC or Fc-TEAC injected intraperitoneally at 100 μg per mouse showed a similar extended half-life of the Fc-TEAC molecule (FIG. 8B).

These data confirm that TEAC constructs comprising half-life extension moieties, such as Fc domains, can have improved pharmacokinetic profiles compared to TEAC constructs without half-life-extension moieties.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. An agent for treating cancer in a patient comprising:
i. a first targeting moiety that binds a tumor antigen expressed by the cancer;
ii. a first T-cell engaging domain capable of T-cell binding activity when binding a second T-cell engaging domain, wherein the first T-cell engaging domain comprises either a VH domain or VL domain;
iii. a second T-cell engaging domain capable of T-cell binding activity when binding a first T-cell engaging domain, wherein the second T-cell engaging domain comprises either a VH domain or VL domain;
iv. a first inert binding partner for the first T-cell engaging domain binding to the first T-cell engaging domain such that the first T-cell engaging domain does not bind to the second T-cell engaging domain unless the inert binding partner is removed, wherein if the first T-cell engaging domain comprises a VH domain, the inert binding partner comprises a VL domain and if the first T-cell engaging domain comprises a VL domain, the inert binding partner comprises a VH domain; and
v. a protease cleavage site separating the first T-cell engaging domain and the first inert binding partner, wherein the protease cleavage site is capable of releasing the inert binding partner from the T-cell engaging domain in the presence of a protease:
(1) expressed by the cancer or in the cancer microenvironment; or
(2) colocalized to the cancer by a targeting moiety that binds a tumor antigen expressed by the cancer and that is the same or different from the targeting moiety in the agent;
wherein the first and second T-cell engaging domains are capable of binding a T cell when neither is bound to an inert binding partner, and further wherein if the first T-cell engaging domain comprises a VH domain, the second T-cell engaging domain comprises a VL domain and if the first T-cell engaging domain comprises a VL domain, the second T-cell engaging domain comprises a VH domain.

2. An agent for treating cancer in a patient comprising:
i. an immune cell selection moiety capable of selectively targeting an immune cell;
ii. a first immune cell engaging domain capable of immune cell binding activity when binding a second immune cell engaging domain, wherein the first immune cell engaging domain comprises either a VH domain or VL domain, optionally wherein the first immune cell engaging domain comprises a T-cell engaging domain;
iii. a second immune cell engaging domain capable of immune cell binding activity when binding a first immune cell engaging domain, wherein the second immune cell engaging domain comprises either a VH domain or VL domain, optionally wherein the second immune cell engaging domain comprises a T-cell engaging domain;
iv. a first inert binding partner for the first immune cell engaging domain binding to the first immune cell engaging domain such that the first immune cell engaging domain does not bind to the second immune cell engaging domain unless the inert binding partner is removed, wherein if the first immune cell engaging domain comprises a VH domain, the inert binding partner comprises a VL domain and if the first immune cell engaging domain comprises a VL domain, the inert binding partner comprises a VH domain; and
v. a protease cleavage site separating the first immune cell engaging domain and the first inert binding partner, wherein the protease cleavage site is capable of releasing the inert binding partner from the immune cell engaging domain in the presence of a protease:
(1) expressed by the cancer or in the cancer microenvironment; or
(2) colocalized to the cancer by a targeting moiety that binds a tumor antigen expressed by the cancer and that is the same or different from the targeting moiety in the agent;
wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

3. The agent of any one of claim 1 or 2, wherein the agent further comprises a second targeting moiety that is capable of targeting the cancer, optionally wherein the first and second targeting moieties bind different antigens or wherein the first and second targeting moieties bind different epitopes of the same antigen.

4. The agent of any one of claims 1-3, further comprising:
i. a second inert binding partner for the second immune cell engaging domain binding to the second immune cell engaging domain such that the second immune cell engaging domain does not bind to the first immune cell engaging domain unless the inert binding partner is removed, wherein if the second immune cell or engaging domain comprises a VH domain, the inert binding partner comprises a VL domain and if the second immune cell engaging domain comprises a VL domain, the inert binding partner comprises a VH domain, optionally wherein the second immune cell engaging domain comprises a T-cell engaging domain; and
ii. a protease cleavage site separating the second immune cell engaging domain and the second inert binding partner, wherein the protease cleavage site is capable of releasing the inert binding partner from the immune cell engaging domain in the presence of a protease:
(1) expressed by the cancer or in the cancer microenvironment; or
(2) colocalized to the cancer by a targeting moiety that binds a tumor antigen expressed by the cancer and that is the same or different from the targeting moiety in the agent,
wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

5. The agent of claim 4, wherein a linker attaches the first and second inert binding partners, optionally wherein the linker is capable of dissociation with the first and/or second inert binding partner upon cleavage of the protease cleavage sites.

6. The agent of claim 5, wherein the linker comprises a half-life extending moiety, optionally wherein the agent has a half-life greater or equal to 2 days, 4 days, or 7 days.

7. The agent of claim 6, wherein the half-life is decreased after dissociation of the half-life extending moiety.

8. The agent of any one of claims 6-7, wherein the half-life extending moiety comprises all or part of an immunoglobulin constant (Fc) domain, serum albumin, serum albumin binding protein, an unstructured protein, and/or PEG; optionally wherein the one or more half-life extending moieties comprise all or part of an immunoglobulin Fc domain and wherein:
i. the Fc domain comprises the sequence of a human immunoglobulin;
ii. the immunoglobulin is IgG, optionally wherein the IgG is IgG1, IgG2, or IgG4; and/or
iii. the Fc domain comprises a naturally occurring sequence.

9. The agent of claim 8, wherein the Fc domain comprises one or more mutations as compared to a naturally occurring sequence, optionally wherein the Fc domain is an Fc domain with a longer half-life compared to a naturally occurring sequence, optionally wherein the Fc domain with a longer half-life:
i. has increased FcRn binding, optionally wherein the increased FcRn binding is measured at pH 6.0; and/or
ii. comprises M252Y/S254T/T256E substitutions or M428L/N434S substitutions.

10. The agent of any one of claims 2-9, wherein the immune cell selection moiety capable of selectively targeting an immune cell selectively targets a T cell, a macrophage, a natural killer cell, a neutrophil, an eosinophil, a basophil, a γδ T cell, a natural killer T cell (NKT cells), or an engineered immune cell, optionally wherein immune cell selection moiety:
i. selectively targets a T cell, optionally where the T cell is a CD8+ or CD4+ T cell;
ii. targets CD8, CD4, or CXCR3, or does not specifically bind regulatory T cells; and/or
iii. comprises an aptamer or an antibody or antigen-specific binding fragment thereof, optionally wherein the aptamer or antibody or antigen-specific binding fragment thereof specifically binds an antigen on a T cell.

11. The agent of any one of claims 1-10, wherein the first and second T-cell or immune cell engaging domains are capable of binding CD3 or the T cell receptor (TCR) when neither is bound to an inert binding partner and/or wherein the first and second T-cell or immune cell engaging domains are capable of forming a Fv when not bound to an inert binding partner.

12. The agent of any one of claim 1 or 3-11, wherein one or more targeting moieties are an antibody or antigen-binding fragment thereof, optionally wherein the antibody or antigen-binding fragment thereof:
i. is specific for any of 4-1BB, 5T4, ACVRL1, ALK1, AXL, B7-H3, BCMA, c-MET, CD133, C4.4a, CA6, CA9, Cadherin-6, CD123, CD133, CD138, CD19, CD20, CD22, CD25, CD27L, CD30, CD33, CD37, CD38, CD44v6, CD56, CD70, CD74(TROP2), CD79b, CEA, CEACAM5, cKit, CLL-1, Cripto, CS1, DLL3, EDNRB, EFNA4, EGFR, EGFRvIII, ENPP3, EpCAM, EPHA2, FGFR2, FGFR3, FLT3, FOLR, FOLR1, GD2, gpA33, GPC3, GPNMB, GUCY2C, HER2, HER3, HLAA2, IGF1-r, IL13RA2, Integrin alpha, LAMP-1, LewisY, LIV-1, LRRC15, MMP9, MSLN, MUC1, MUC16, NaPi2b, Nectin-4, NOTCH3, p-CAD, PD-L1, PSMA, PTK7, ROR1, SLC44A4, SLITRK6, SSTR2, STEAP1, TAG72, TF, TIM-1, or TROP-2,
ii. is an anti-epidermal growth factor receptor antibody; an anti-Her2 antibody; an anti-CD20 antibody; an anti-CD22 antibody; an anti-CD70 antibody; an anti-CD33 antibody; an anti-MUC1 antibody; an anti-CD40 antibody; an anti-CD74 antibody; an anti-P-cadherin antibody; an anti-EpCAM antibody; an anti-CD138 antibody; an anti-E-cadherin antibody; an anti-CEA antibody; an anti-FGFR3 antibody; an anti-mucin core protein antibody; an anti-transferrin antibody; an anti-gp95/97 antibody; an anti-p-glycoprotein antibody; an anti-TRAIL-R1 antibody; an anti-DR5 antibody; an anti-IL-4 antibody; an anti-IL-6 antibody; an anti-CD19 antibody; an anti-PSMA antibody; an anti-PSCA antibody; an anti-Cripto antibody; an anti-PD-L1 antibody; an anti-IGF-1R antibody; an anti-CD38 antibody; an anti-CD133 antibody; an anti-CD123 antibody; an anti-CDE49d antibody; an anti-glypican 3 antibody; an anti-cMET antibody; or an anti-IL-13R antibody, and/or
iii. comprises all or part of the amino acid sequence of 1C1, (GS) 5745, ABBV-085, ABBV-399, ABBV-838, AbGn-107, ABT-414, ADCT-301, ADCT-402, AGS-16C3F, AGS62P1, AGS67E, AMG 172d, AMG 595d, Andecaliximab, Anetumab ravtansine, ARX788, ASG-15MEd, ASG-5MEk, Atezolizumab, AVE1642, AVE9633e, Avelumab, BAY1129980, BAY1187982e, BAY79-4620b, BIIB015d, Bivatuzumab mertansineb, BMS-986148, Brentuximab vedotin, Cantuzumab mertansine, CC49, CDX-014, Cirmtuzumab, Coltuximab ravtansine, DEDN6526Ae, Denintuzumab mafodotin, Depatuxizumab, DFRF4539Ad, DMOT4039Ae, DS-8201A, Durvalumab, Enfortumab vedotin, Farletuzumab, FLYSYN, Gatipotuzumab, Gemtuzumab ozogamicin, Glembatumumab vedotin, GSK2857916, HKT288, Hu3F8, HuMax-AXL-ADC, IDEC-159, IMGN289b, IMGN388a, IMGN529, Indatuximab ravtansine, Inotuzumab ozogamicin, Istiratumab, Labetuzumab govitecan, Lifastuzumab vedotin, LOP628h, Lorvotuzumab mertansine, LY3076226, MCLA-117 (CLEC-12AxCD3), MDX-1203d, MEDI-4276, MEDI-547b, Milatuzumab-doxorubicin, Mirvetuximab soravtansine, MLN0264, MLN2704e, MM-302i, Mosunetuzumab, MOv18 IgE, Ocrelizumab, Oportuzumab, Patritumab, PCA-062, PF-03446962, PF-06263507a, PF-06647020, PF-06647263, PF-06650808d, Pinatuzumab vedotin, Polatuzumab vedotin, PSMA ADC 301c, RC48-ADC, Rituximab, Rovalpituzumab tesirine, Sacituzumab, Sacituzumab govitecan, SAR408701, SAR428926, SAR566658, SC-002, SC-003, SGN-15a, SGN-CD123A, SGN-CD19B, SGN-CD70A, SGN-LIV1A, Sofituzumab vedotin, Solitomab, SSTR2xCD3 XmAb18087, STRO-002, SYD-985, Talacotuzumab, Tisotumab vedotin, Trastuzumab emtansine, U3-1402, Ublituximab, Vadastuximab talirine, Vandortuzumab vedotin, Vorsetuzumab mafodotin, XMT-1522, or Zenocutuzumab.

13. The agent of any one of claims 1-12, wherein one or more targeting moieties or an immune cell selection moiety is an aptamer, optionally wherein the aptamer:
i. comprises DNA or RNA;
ii. is single-stranded;
iii. is a target cell-specific aptamer chosen from a random candidate library;
iv. is an anti-EGFR aptamer; and/or
v. binds to the antigen on the cancer cell with a Kd from 1 picomolar to 500 nanomolar, optionally wherein the aptamer binds to the cancer with a Kd from 1 picomolar to 100 nanomolar.

14. The agent of any one of claim 1 or 3-13, wherein one or more targeting moieties comprise IL-2, IL-4, IL-6, α-MSH, transferrin, folic acid, EGF, TGF, PD1, IL-13, stem cell factor, insulin-like growth factor (IGF), or CD40, optionally wherein one or more targeting moiety:
i. comprise a full-length sequence of IL-2, IL-4, IL-6, α-MSH, transferrin, folic acid, EGF, TGF, PD1, IL-13, stem cell factor, insulin-like growth factor (IGF), or CD40;
ii. comprise a truncated form, analog, variant, or derivative of IL-2, IL-4, IL-6, α-MSH, transferrin, folic acid, EGF, TGF, PD1, IL-13, stem cell factor, insulin-like growth factor (IGF), or CD40; and/or
iii. bind a target on the cancer comprising IL-2 receptor, IL-4, IL-6, melanocyte stimulating hormone receptor (MSH receptor), transferrin receptor (TR), folate receptor 1 (FOLR), folate hydroxylase (FOLH1), EGF receptor, PD-L1, PD-L2, IL-13R, CXCR4, IGFR, or CD40L.

15. The agent of any one of claim 3-9 or 11-14, wherein the first and second targeting moieties:
i. bind the same antigen;
ii. bind the same epitope; and/or
iii. are the same or are different.

16. A method of treating cancer expressing a tumor antigen that binds the first targeting moiety in a patient comprising administering the agent of any one of claims 1-15 to the patient, optionally wherein the cancer expressing a tumor antigen that binds the first targeting moiety is any one of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, leukemia, myeloma, nonHodgkin lymphoma, Hodgkin lymphoma, acute myeloid leukemia, acute lymphoblastic leukemia, chronic lymphoblastic leukemia, lymphoproliferative disorder, myelodysplastic disorder, myeloproliferative disease or premalignant disease.

17. A method of targeting an immune response of a patient to cancer comprising administering the agent of any one of claims 1-15 to the patient.

18. The method of claim 17, wherein the T cells express CD3 or TCR and the T cell engaging domain binds CD3 or TCR.

19. The method of claim 18, wherein if the patient has regulatory T cells in the tumor, the selective immune cell engaging agent does not target markers present on regulatory immune cells (including, but not limited to CD4 and CD25).

20. One or more nucleic acid molecules encoding the agent of any of claims 1-19.

21. An agent for treating cancer in a patient comprising:
a. a first component comprising a targeted T-cell engaging agent comprising:
i. a first targeting moiety that binds a tumor antigen expressed by the cancer;
ii. a first T-cell engaging domain capable of T-cell engaging activity when binding a second T-cell engaging domain, wherein the second T-cell engaging domain is not part of the first component, and wherein the first T-cell engaging domain comprises either a VH domain or VL domain;
iii. a first inert binding partner for the first T-cell engaging domain binding to the first T-cell engaging domain such that the first T-cell engaging domain does not bind to the second T-cell engaging domain unless the inert binding partner is removed, wherein if the first T-cell engaging domain comprises a VH domain, the inert binding partner comprises a VL domain and if the first T-cell engaging domain comprises a VL domain, the inert binding partner comprises a VH domain;
iv. a first half-life extending moiety, wherein the first half-life extending moiety is attached (directly or indirectly) to the first inert binding partner; and
v. a protease cleavage site separating the first T-cell engaging domain and the first inert binding partner, wherein the protease cleavage site is capable of releasing the inert binding partner and half-life extending moiety from the T-cell engaging domain in the presence of a protease: (1) expressed by the cancer or in the cancer microenvironment; (2) colocalized to the cancer by a targeting moiety that binds a tumor antigen expressed by the cancer and that is the same or different from the targeting moiety in the agent, and
b. a second component comprising a targeted T-cell engaging agent comprising:
i. a second targeting moiety that binds a tumor antigen expressed by the cancer;
ii. a second T-cell engaging domain capable of T-cell binding activity when binding a first T-cell engaging domain, wherein the first T-cell engaging domain is not part of the second component, and wherein the second T-cell engaging domain comprises either a VH domain or VL domain;
iii. a second inert binding partner for the second T-cell engaging domain binding to the second T-cell engaging domain such that the second T-cell engaging domain does not bind to the first T-cell engaging domain unless the inert binding partner is removed, wherein if the second T-cell engaging domain comprises a VH domain, the inert binding partner comprises a VL domain and if the second T-cell engaging domain comprises a VL domain, the inert binding partner comprises a VH domain;
iv. a second half-life extending moiety, wherein the second half-life extending moiety is attached (directly or indirectly) to the second inert binding partner; and
v. a protease cleavage site separating the second T-cell engaging domain and the second inert binding partner, wherein the protease cleavage site is capable of releasing the inert binding partner and half-life extending moiety from the T-cell engaging domain in the presence of a protease: (1) expressed by the cancer or in the cancer microenvironment; (2) colocalized to the cancer by a targeting moiety that binds a tumor antigen expressed by the cancer and that is the same or different from the targeting moiety in the agent, wherein the first and second T-cell engaging domains are capable of binding a T cell when neither is bound to an inert binding partner, and further wherein if the first T-cell engaging domain comprises a VH domain, the second T-cell engaging domain comprises a VL domain and if the first T-cell engaging domain comprises a VL domain, the second T-cell engaging domain comprises a VH domain.

22. An agent for treating cancer in a patient comprising:
a. a first component comprising a targeted immune cell engaging agent comprising:
i. a targeting moiety capable of targeting the cancer;
ii. a first immune cell engaging domain capable of immune engaging activity when binding a second immune cell engaging domain, wherein the second immune cell engaging domain is not part of the first component, optionally wherein the first immune cell engaging domain comprises a T-cell engaging domain;
iii. a first inert binding partner for the first immune cell engaging domain binding to the first immune cell engaging domain such that the first immune cell engaging domain does not bind to the second immune cell engaging domain unless the inert binding partner is removed, wherein if the first immune cell engaging domain comprises a VH domain, the inert binding partner comprises a VL domain and if the first immune cell engaging domain comprises a VL domain, the inert binding partner comprises a VH domain;
iv. a first half-life extending moiety, wherein the first half-life extending moiety is attached (directly or indirectly) to the first inert binding partner; and
v. a protease cleavage site separating the first immune cell engaging domain and the first inert binding partner, wherein the protease cleavage site is capable of releasing the inert binding partner and half-life extending moiety from the immune cell engaging domain in the presence of a protease: (1) expressed by the cancer or in the cancer microenvironment; or (2) colocalized to the cancer by a targeting moiety that binds a tumor antigen expressed by the cancer and that is the same or different from the targeting moiety in the agent, and
b. a second component comprising a selective immune cell engaging agent comprising:
i. an immune cell selection moiety capable of selectively targeting an immune cell;
ii. a second immune cell engaging domain capable of immune cell engaging activity when binding the first immune cell engaging domain, wherein the first and second immune cell engaging domains are capable of binding when neither is bound to an inert binding partner, optionally wherein the second immune cell engaging domain comprises a immune cell engaging domain;
iii. a second inert binding partner for the second immune cell engaging domain binding to the second immune cell engaging domain such that the second immune cell engaging domain does not bind to the first immune cell engaging domain unless the inert binding partner is removed, wherein if the second immune cell engaging domain comprises a VH domain, the inert binding partner comprises a VL domain and if the second immune cell engaging domain comprises a VL domain, the inert binding partner comprises a VH domain;
iv. a second half-life extending moiety, wherein the second half-life extending moiety is attached (directly or indirectly) to the second inert binding partner; and
v. a protease cleavage site separating the second immune cell engaging domain and the second inert binding partner, wherein the protease cleavage site is capable of releasing the inert binding partner and half-life extending moiety from the immune cell engaging domain in the presence of a protease: (1) expressed by the cancer or in the cancer microenvironment; or (2) colocalized to the cancer by a targeting moiety that binds a tumor antigen expressed by the cancer and that is the same or different from the targeting moiety in the agent, wherein the first and second immune cell engaging domains are capable of binding an immune cell when neither is bound to an inert binding partner, and further wherein if the first immune cell engaging domain comprises a VH domain, the second immune cell engaging domain comprises a VL domain and if the first immune cell engaging domain comprises a VL domain, the second immune cell engaging domain comprises a VH domain.

23. A component for use in a kit or composition for treating cancer in a patient comprising a first targeted immune cell engaging agent comprising:
a. a targeting moiety that binds a tumor antigen expressed by the cancer;
b. an immune cell engaging domain capable of immune cell binding activity when binding another immune cell engaging domain, wherein the other immune cell engaging domain is not part of the first component, and wherein the immune cell engaging domain comprises either a VH domain or VL domain, optionally wherein the immune cell engaging domain comprises a T-cell engaging domain;
c. an inert binding partner for the immune cell engaging domain binding to the immune cell engaging domain such that the immune cell engaging domain does not bind to the other immune cell engaging domain unless the inert binding partner is removed, wherein if the immune cell engaging domain comprises a VH domain, the inert binding partner comprises a VL domain and if the immune cell engaging domain comprises a VL domain, the inert binding partner comprises a VH domain;
d. a half-life extending moiety, wherein the half-life extending moiety is attached (directly or indirectly) to the inert binding partner; and
e. a protease cleavage site separating the immune cell engaging domain and the inert binding partner, wherein the protease cleavage site is capable of releasing the inert binding partner and half-life extending moiety from the immune cell engaging domain in the presence of a protease: (1) expressed by the cancer; or (2) colocalized to the cancer by a targeting moiety that binds a tumor antigen expressed by the cancer and that is the same or different from the targeting moiety in the agent, wherein cleavage of the protease cleavage site causes loss of the inert binding partner and allows for complementation with the other immune cell engaging domain that is not part of the agent, further wherein if the immune cell engaging domain comprises a VH domain, the other immune cell engaging domain comprises a VL domain and if the immune cell engaging domain comprises a VL domain, the other immune cell engaging domain comprises a VH domain.

24. A component for use in a kit or composition for treating cancer in a patient comprising a first targeted immune cell engaging agent comprising:
a. an immune cell selection moiety capable of selectively targeting an immune cell;
b. an immune cell engaging domain capable of immune cell binding activity when binding another immune cell engaging domain, wherein the other immune cell engaging domain is not part of the first component, and wherein the immune cell engaging domain comprises either a VH domain or VL domain, optionally wherein the immune cell engaging domain comprises a T-cell engaging domain;
c. an inert binding partner for the immune cell engaging domain binding to the immune cell engaging domain such that the immune cell engaging domain does not bind to the other immune cell engaging domain unless the inert binding partner is removed, wherein if the immune cell engaging domain comprises a VH domain, the inert binding partner comprises a VL domain and if the immune cell engaging domain comprises a VL domain, the inert binding partner comprises a VH domain;
d. a half-life extending moiety, wherein the half-life extending moiety is attached (directly or indirectly) to the inert binding partner; and
e. a protease cleavage site separating the immune cell engaging domain and the inert binding partner, wherein the protease cleavage site is capable of releasing the inert binding partner and half-life extending moiety from the immune cell engaging domain in the presence of a protease: (1) expressed by the cancer; or (2) colocalized to the cancer by a targeting moiety that binds a tumor antigen expressed by the cancer and that is the same or different from the targeting moiety in the agent, wherein cleavage of the protease cleavage site causes loss of the inert binding partner and allows for complementation with the other immune cell engaging domain that is not part of the agent, further wherein if the immune cell engaging domain comprises a VH domain, the other immune cell engaging domain comprises a VL domain and if the immune cell engaging domain comprises a VL domain, the other immune cell engaging domain comprises a VH domain.
